SEQUENCE LISTING (698 sequences)

SEQ ID NO: 1 - protein, 40 aa, Enterococcus faecalis
WLPPAGLLGRCGRWFRPWLLWLQSGAQYKWLGNLFGLGPK

SEQ ID NO: 2 - protein, 5 aa, synthetic construct
  REGION 1..5: note = N-terminal motif of class terminal IIa
  VARIANT 3: note = Xaa = any amino acid
YGXGV

SEQ ID NO: 3 - protein, 135 aa, synthetic construct
  REGION 1..135: note = Hybrid bacteriocin Ent35-MccV
GKYYGNGVSCNKKGCSVDWGRAIGIIGNNSAANLATGGAAGWKSGGGASGRDIAMAIGTLSGQFVAGGIGAAAGGVAGGAIYDYASTHKPNPAMSPSGLGGTIKQKPEGIPSEAWNYAAGRLCNWSPNNLSDVCL

SEQ ID NO: 4 - protein, 46 aa, Lactobacillus acidophilus
MISSHQKTLTDKELALISGGKTHYPTNAWKSLWKGFWESLRYTDGF

SEQ ID NO: 5 - DNA, 141 bp, Lactobacillus acidophilus
  source 1..141: mol_type = genomic DNA
atgatttcatctcatcaaaaaacgttaactgataaagaattagcattaatttctggggggaaaacgcactacccgactaatgcatggaaaagtctttggaaaggtttctgggaaagccttcgttatactgacggtttttag

SEQ ID NO: 6 - protein, 81 aa, Lactobacillus acidophilus
MISMISSHQKTLTDKELALISGGKTYYGTNGVHCTKKSLWGKVRLKNVIPGTLCRKQSLPIKQDLKILLGWATGAFGKTFH

SEQ ID NO: 7 - DNA, 246 bp, Lactobacillus acidophilus
  source 1..246: mol_type = genomic DNA
atgatttcaatgatttcatctcatcaaaaaacgttaactgataaagaattagcattaatttctggggggaaaacgtactatggtactaatggtgtgcattgtactaaaaagagtctttggggtaaagtacgcttaaaaaacgtgattcctggaactctttgtcgtaagcaatcgttgccgatcaaacaggatttaaaaattttactgggctgggctacaggtgcttttggcaagacatttcattaa

SEQ ID NO: 8 - protein, 60 aa, Lactobacillus acidophilus
MDKKTKILFEVLYIICIIGPQFILFVTAKNNMYQLVGSFVGIVWFSYIFWYIFFKQHKKM

SEQ ID NO: 9 - DNA, 183 bp, Lactobacillus acidophilus
  source 1..183: mol_type = genomic DNA
atggataagaaaacaaaaatattatttgaagtattatacatcatctgtataataggccctcaatttatattatttgtgactgcaaaaaacaatatgtatcagttggtgggttcgtttgttggaatagtatggttttcgtatattttttggtatatttttttcaaacaacataaaaaaatgtag

SEQ ID NO: 10 - protein, 65 aa, Lactobacillus gasseri
MALKTLEKHELRNVMGGNKWGNAVIGAATGATRGVSWCRGFGPWGMTACALGGAAIGGYLGYKSN

SEQ ID NO: 11 - DNA, 198 bp, Lactobacillus gasseri
  source 1..198: mol_type = genomic DNA
atggctttaaaaacattagaaaaacatgaattaagaaatgtaatgggtggaaacaagtgggggaatgctgtaataggagctgctacgggagctactcgcggagtaagttggtgcagaggattcggaccatggggaatgactgcctgtgcgttaggaggtgctgcaattggaggatatctgggatataagagtaattaa

SEQ ID NO: 12 - protein, 51 aa, Staphylococcus aureus [sequence omitted]

SEQ ID NO: 13 - DNA, 156 bp, Staphylococcus aureus
  source 1..156: mol_type = genomic DNA
atgagttggttaaattttttaaaatacatcgctaaatatggcaaaaaagcggtatctgctgcttggaagtacaaaggtaaagtattagaatggcttaatgttggtcctactcttgaatgggtatggcaaaaattaaagaaaattgctggattataa

SEQ ID NO: 14 - protein, 61 aa, Enterococcus avium
MTRSKKLNLREMKNVVGGTYYGNGVSCNKKGCSVDWGKAISIIGNNSAANLATGGAAGWKS

SEQ ID NO: 15 - DNA, 186 bp, Enterococcus avium
  source 1..186: mol_type = genomic DNA
atgacaagatcaaaaaaattaaatttacgcgaaatgaagaatgttgttggtggtacctactatggaaatggtgtatcttgtaacaagaaaggctgttcagttgactggggcaaagccatcagtattataggaaataattccgcagcaaacttagcaactggtggtgctgctggttggaagtcataa

SEQ ID NO: 16 - protein, 67 aa, Enterococcus faecalis
MKKKLVICGIIGIGFTALGTNVEAATYYGNGLYCNKQKCWVDWNKASREIGKIIVNGWVQHGPWAPR

SEQ ID NO: 17 - DNA, 204 bp, Enterococcus faecalis
  source 1..204: mol_type = genomic DNA
atgaaaaagaaattagttatttgtggcattattgggattggttttacagcattaggaacaaatgtagaagctgctacgtattacggaaatggtttatattgtaataagcaaaaatgttgggtagactggaataaagcttcaagggaaattggaaaaattattgttaatggttgggtacaacatggcccttgggctcctagatag

SEQ ID NO: 18 - protein, 51 aa, Lactococcus lactis
MKEQNSFNLLQEVTESELDLILGAKGGSGVIHTISHEVIYNSWNFVFTCCS

SEQ ID NO: 19 - DNA, 156 bp, Lactococcus lactis
  source 1..156: mol_type = genomic DNA
atgaaagaacaaaactcttttaatcttcttcaagaagtgacagaaagtgaattggaccttattttaggtgcaaaaggcggcagtggagttattcatacaatttctcatgaagtaatatataatagctggaactttgtatttacttgctgctcttaa

SEQ ID NO: 20 - protein, 74 aa, Enterococcus faecium
MKKKVLKHCVILGILGTCLAGIGTGIKVDAATYYGNGLYCNKEKCWVDWNQAKGEIGKIIVNGWVNHGPWAPRR

SEQ ID NO: 21 - DNA, 225 bp, Enterococcus faecium
  source 1..225: mol_type = genomic DNA
atgaaaaagaaagtattaaaacattgtgttattctaggaatattaggaacttgtctagctggcatcggtacaggaataaaagttgatgcagctacttactatggaaatggtctttattgtaacaaagaaaaatgttgggtagattggaatcaagctaaaggagaaattggaaaaattattgttaatggttgggttaatcatggtccatgggcacctagaaggtag

SEQ ID NO: 22 - protein, 50 aa, Clostridium botulinum
MQKPEIISADLGLCAVNEFVALAAIPGGAATFAVCQMPNLDEIVSNAAYV

SEQ ID NO: 23 - DNA, 153 bp, Clostridium botulinum
  source 1..153: mol_type = genomic DNA
atgcaaaaaccagaaattattagtgctgatttagggctttgtgcagttaatgaatttgtagctcttgctgccattcctggtggtgctgctacatttgcagtatgccaaatgccaaacttggatgagattgttagtaatgcagcatatgtttaa

SEQ ID NO: 24 - protein, 58 aa, Streptococcus equinus
MMNATENQIFVETVSDQELEMLIGGADRGWIKTLTKDCPNVISSICAGTIITACKNCA

SEQ ID NO: 25 - DNA, 177 bp, Streptococcus equinus
  source 1..177: mol_type = genomic DNA
atgatgaatgctactgaaaaccaaatttttgttgagactgtgagtgaccaagaattagaaatgttaattggtggtgcagatcgtggatggattaagactttaacaaaagattgtccaaatgtaatttcttcaatttgtgcaggtacaattattacagcttgtaaaaattgtgcttaa

SEQ ID NO: 26 - protein, 77 aa, Brochothrix campestris
MHKVKKLNNQELQQIVGGYSSKDCLKDIGKGIGAGTVAGAAGGGLAAGLGAIPGAFVGAHFGVIGGSAACIGGLLGN

SEQ ID NO: 27 - DNA, 234 bp, Brochothrix campestris
  source 1..234: mol_type = genomic DNA
atgcacaaggtaaaaaaattaaacaatcaagagttacaacagatcgtgggaggttacagttcaaaagattgtctaaaagatattggtaaaggaattggtgctggtacagtagctggggcagccggcggtggcctagctgcaggattaggtgctatcccaggagcattcgttggagcacattttggagtaatcggcggatctgccgcatgcattggtggattattaggtaactag

SEQ ID NO: 28 - protein, 80 aa, Butyrivibrio fibrisolvens
MSKKQIMSNCISIALLIALIPNIYFIADKMGIQLAPAWYQDIVNWVSAGGTLTTGFAIIVGVTVPAWIAEAAAAFGIASA

SEQ ID NO: 29 - DNA, 243 bp, Butyrivibrio fibrisolvens
  source 1..243: mol_type = genomic DNA
atgagtaaaaaacaaattatgagtaactgtatatcaattgcattattaatagcactaattcctaatatctattttattgcagataaaatgggaattcagttagcacctgcttggtatcaagatattgtgaattgggtatctgctggtggaacacttactactggttttgcgattattgtaggagttacagtaccggcatggatagcagaagcagctgcagcttttggtatagcttcagcatga

SEQ ID NO: 30 - protein, 48 aa, Butyrivibrio fibrisolvens
MNKELNALTNPIDEKELEQILGGGNGVIKTISHECHMNTWQFIFTCCS

SEQ ID NO: 31 - DNA, 147 bp, Butyrivibrio fibrisolvens
  source 1..147: mol_type = genomic DNA
atgaacaaagaacttaatgcacttacaaatcctattgacgagaaggagcttgagcagatcctcggtggtggcaatggtgtcatcaagacaatcagccacgagtgccacatgaacacatggcagttcattttcacatgttgctcttaa

SEQ ID NO: 32 - protein, 66 aa, Carnobacterium maltaromaticum
MNSVKELNVKEMKQLHGGVNYGNGVSCSKTKCSVNWGQAFQERYTAGINSFVSGVASGAGSIGRRP

SEQ ID NO: 33 - DNA, 201 bp, Carnobacterium maltaromaticum
  source 1..201: mol_type = genomic DNA
atgaatagcgtaaaagaattaaacgtgaaagaaatgaaacaattacacggtggagtaaattatggtaatggtgtttcttgcagtaaaacaaaatgttcagttaactggggacaagcctttcaagaaagatacacagctggaattaactcatttgtaagtggagtcgcttctggggcaggatccattggtaggagaccgtaa

SEQ ID NO: 34 - protein, 61 aa, Carnobacterium maltaromaticum
MKSVKELNKKEMQQINGGAISYGNGVYCNKEKCWVNKAENKQAITGIVIGGWASSLAGMGH

SEQ ID NO: 35 - DNA, 186 bp, Carnobacterium maltaromaticum
  source 1..186: mol_type = genomic DNA
atgaaaagcgttaaagaactaaataaaaaagaaatgcaacaaattaatggtggagctatctcttatggcaatggtgtttattgtaacaaagagaaatgttgggtaaacaaggcagaaaacaaacaagctattactggaatagttatcggtggatgggcttctagtttagcaggaatgggacattaa

SEQ ID NO: 36 - protein, 71 aa, Carnobacterium maltaromaticum
MNNVKELSIKEMQQVTGGDQMSDGVNYGKGSSLSKGGAKCGLGIVGGLATIPSGPLGWLAGAAGVINSCMK

SEQ ID NO: 37 - DNA, 216 bp, Carnobacterium maltaromaticum
  source 1..216: mol_type = genomic DNA
atgaataatgtaaaagagttaagtattaaagaaatgcaacaagttactggtggagaccaaatgtcagatggtgtaaattatggaaaaggctctagcttatcaaaaggtggtgccaaatgtggtttagggatcgtcggcggattagctactatcccttcaggtcctttaggctggttagccggagcagcaggtgtaattaatagctgtatgaaataa

SEQ ID NO: 38 - protein, 64 aa, Carnobacterium maltaromaticum
MLYELVAYGIAQGTAEKVVSLINAGLTVGSIISILGGVTVGLSGVFTAVKAAIAKQGIKKAIQL

SEQ ID NO: 39 - DNA, 195 bp, Carnobacterium maltaromaticum
  source 1..195: mol_type = genomic DNA
atgttatatgaattagttgcatatggtatcgcacaaggtacagctgaaaaggttgtaagtctaattaacgcaggtttaacagtagggtctattatttcaattttgggtggggtcacagtcggtttatcaggtgtcttcacagcagttaaagcagcaattgctaaacaaggaataaaaaaagcaattcaattataa

SEQ ID NO: 40 - protein, 836 aa, Pectobacterium carotovorum
  source 1..836: note = subsp. carotovorum
MIKYRLYAPNDGDTMTVSGGGGWVSNDDRKGGNDRDNGKGGSAVDFSKNPEKQAIVNPYLAIAIPMPVYPLYGKLGFTINTTAIETELANVRAAINTKLATLSAVIGRSLPVVGRVFGVTAAGMWPSSTAPSSLDSIYNQAHQQALAQLAAQQGVLNKGYNVTAMPAGFVSSLPVSEIKSLPTAPASLLAQSVINTELSQRQLALTQPTTNAPVANIPVVKAEKTAMPGVYSAKIIAGEPAFQIKVDNTKPALAQNPPKVKDDIQVSSFLSSPVADTHHAFIDFGSDHEPVYVSLSKIVTAEEEKKQVEEAKRREQEWLLRHPITAAERKLTEIRQVISFAQQLKESSVATISEKTKTVAVYQEQVNTAAKNRDNFYNQNRGLLSAGITGGPGYPIYLALWQTMNNFHQAYFRANNALEQESHVLNLARSDLAKAEQLLAENNRLQVETERTLAEEKEIKRNRVNVSTFGTVQTQLSKLLSDFYAVTSLSQSVPSGALASFSYNPQGMIGSGKIVGKDVDVLFSIPVKDIPGYKSPINLDDLAKKNGSLDLPIRLAFSDENGERVLRAFKADSLRIPSSVRGVAGSYDKNTGIFSAEIDGVSSRLVLENPAFPPTGNVGNTGNTAPDYKALLNTGVDVKPVDKITVTVTPVADPVDIDDYIIWLPTASGSGVEPIYVVFNSNPYGGTEKGKYSKRYYNPDKAGGPILELDWKNVKIDHAGVDNVKLHTGRFKASVENKVMIERLENILNGQITATDTDKRFYTHELRELNRYRNLGIKDGEVPSSIQEESAVWNDTHTATLEDYKINEKEQPLYTDAALQAAYEQELKDALGGKHG

SEQ ID NO: 41 - DNA, 2511 bp, Pectobacterium carotovorum
  source 1..2511: mol_type = genomic DNA; note = subsp. carotovorum
atgattaaataccgtttatatgctccaaatgatggagacaccatgacagtgagtggtggtggtggttgggtttcaaacgatgatcgcaaaggtggtaatgacagggacaatggcaaaggtggttctgccgttgattttagtaaaaatccagaaaagcaggctatcgttaatccctatttggcaatcgcgataccgatgccggtctaccctctttatggaaagctagggttcacaataaatacgacggcaattgagactgaactcgcaaatgtcagagcagcaattaacactaaacttgcaacactcagtgcagtgattggcagatcacttccggtcgttgggcgggtatttggtgttactgccgccggaatgtggccttctagtaccgctcccagtagtctcgattctatatacaatcaagcacatcagcaggctttagcccagttagctgctcaacagggagtattaaataaagggtataacgttacagcaatgcctgcaggtttcgtcagcagtttgcctgttagtgaaatcaaatcattgccaacagctcccgccagtttactggcacaaagtgtgattaataccgaactttcccagcgtcaactggctcttactcagcccacgacgaatgcaccagtcgcgaatattcccgtagttaaagcagagaaaacagcaatgccaggtgtgtattcagcgaaaattattgctggtgagcctgcattccaaatcaaggtcgataataccaaacctgctttggcacagaatccgccgaaagtaaaagatgatattcaggtatcttctttcctttcctcgccagtagctgatacgcaccatgcatttattgattttggcagcgatcatgaaccggtatacgtgtctctttcaaagatcgtgacagccgaggaggagaaaaaacaggttgaagaggccaagcgccgtgagcaggagtggttgttgcgtcatccaattacagctgcggagcgaaaattaactgaaatccgccaagtgatctcttttgctcaacagctaaaagaaagctctgtcgcaaccatttcagaaaaaactaaaactgttgcggtttaccaagaacaggtgaataccgctgcaaaaaatcgcgacaatttttataatcaaaatagaggtctgttaagtgcgggtataactgggggaccgggatatcctatttatcttgctttatggcaaacgatgaataactttcatcaggcttatttcagagcaaataatgcattggaacaagagagtcatgttctgaacctggctcgttctgatctggctaaggctgagcaattgcttgctgagaataatcgacttcaggttgaaacggagcgaacgcttgccgaagaaaaagagataaaacgcaacagggttaatgtatcaacatttggcacagtgcaaactcaacttagtaaattgctgtcagatttttatgctgttacatcactttcccaaagtgttccttcgggggcattagcctctttttcatataatccacaagggatgattggcagcggtaagattgttgggaaggatgtcgatgttttattttccatcccagtaaaagatattccgggatataaatctcctattaacttggacgatttagccaagaaaaatggaagtctggatcttcccattcgtctggcattttctgatgagaatggagaaagggttcttcgggcattcaaagcggatagtctgcgaatcccttcgagtgtcagaggtgtagcgggcagttatgacaaaaatacgggtatttttagtgcagaaattgatggtgtttcatctcgccttgtactggaaaacccagcgtttcctccgaccggaaatgtcggtaatacgggtaatactgcacctgactataaagcattactgaatactggtgttgatgttaaacctgttgataaaatcacagttacggtaacaccagttgctgatccagtggatattgatgactatataatctggttgccaactgcgtctggttctggcgtggaacccatttatgtcgtgtttaacagtaatccgtatggtgggacggaaaaaggaaaatatagcaaacgttattataatccagataaggcaggcggtccgatcttggagctggattggaaaaacgttaagattgaccatgcaggtgtggacaatgttaaattacacacagggcgtttcaaagcgtcggttgaaaacaaagtgatgattgaacgtttggaaaacatactgaatggtcaaatcacggccacggatactgacaagcgattctatacgcatgaattaagagagttaaaccgctacagaaatttaggcatcaaagacggtgaagtgcctagtagcattcaagaagaaagcgctgtttggaacgacacacacacagcgacgcttgaagactacaaaattaatgagaaagagcaaccgttgtacactgatgctgctttgcaggcagcctacgaacaggaactcaaagacgcattaggagggaaacatggctaa

SEQ ID NO: 42 - protein, 74 aa, Bacillus cereus
MENLQMLTEEELMEIEGGGWWNSWGKCVAGTIGGAGTGGLGGAAAGSAVPVIGTGIGGAIGGVSGGLTGAATFC

SEQ ID NO: 43 - DNA, 225 bp, Bacillus cereus
  source 1..225: mol_type = genomic DNA
atggaaaacttacaaatgttaactgaagaagaattaatggaaattgaaggtggaggctggtggaatagctggggtaaatgtgttgctggaactatcggtggagctggaactggtggtttaggtggagctgctgcaggttcagctgttccggttattggtactggtattggtggcgctattggtggagttagcggtggccttacaggtgcagctactttttgctaa

SEQ ID NO: 44 - protein, 78 aa, unidentified
  source 1..78: note = Streptoverticillium griseoverticillatum
MTASILQQSVVDADFRAALLENPAAFGASAAALPTPVEAQDQASLDFWTKDIAATEAFACRQSCSFGPFTFVCDGNTK

SEQ ID NO: 45 - DNA, 237 bp, unidentified
  source 1..237: mol_type = genomic DNA; note = Streptoverticillium griseoverticillatum
atgaccgcttccattcttcagcagtccgtcgtggacgccgacttccgcgcggcgctgcttgagaaccccgccgccttcggcgcttccgccgcggccctgcccacgcccgtcgaggcccaggaccaggcgtcccttgacttctggaccaaggacatcgccgccacggaagccttcgcctgccgccagagctgcagcttcggcccgttcaccttcgtgtgcgacggcaacaccaagtaa

SEQ ID NO: 46 - protein, 76 aa, Geobacillus kaustophilus
MSLLALVAGTLGVSQSIATTVVSIVLTGSTLISIILGITAILSGGVDAILEIGWSAFVATVKKIVAERGKAAAIAW

SEQ ID NO: 47 - DNA, 231 bp, Geobacillus kaustophilus
  source 1..231: mol_type = genomic DNA
atgagtttgctggcgcttgttgccgggacgctcggcgtgtcacagtcaatcgcgacgacggttgtttcgattgtgttgaccggctccactctcatttctattattcttgggatcaccgctattttgtcaggtggagtcgacgccattttggaaattgggtggtcagcttttgtcgcgacggtgaaaaaaatagtggcggaacgaggaaaagcggcagcgattgcatggtaa

SEQ ID NO: 48 - protein, 309 aa, Clostridium tyrobutyricum
MRKVFLRSIISTLVMCAFVSSSFSVNADESKPNDEKIINNIENVTTTKDIVKSNKNNIVYLDEGVMSIPLSGRKPIAIKDDNNKEDLTVTLPIKNTGDISKISSNGTILYKNNSSNSSNIALQPKNDGFKALININDKLANKEYEFTFNLPKNSKLISAATYLGKEYDTKEVFVVDKNNIITSIISPAWAKDANGHNVSTYYKIVSNNKLVQVVEFTENTAFPVVADPNWTKIGKCAGSIAWAIGSGLFGGAKLIKIKKYIAELGGLQKAAKLLVGATTWEEKLHAGGYALINLAAELTGVAGIQANCF

SEQ ID NO: 49 - DNA, 930 bp, Clostridium tyrobutyricum
  source 1..930: mol_type = genomic DNA
ttgagaaaagtatttttaagatcaataatttcaacattagttatgtgtgcatttgtttcaagcagcttttcagtaaatgcggatgaaagcaaaccaaatgatgaaaaaataattaataacatagaaaacgttactactactaaagatattgtaaaaagtaataaaaataatattgtatatttagatgaaggtgtaatgagtattccattgtctgggagaaaacccattgctattaaagatgataataataaagaagatttaactgttacattacctattaagaatactggagatatatctaaaattagtagtaatggtactattctgtataaaaataatagtagtaattcatctaatatagctttacaacctaaaaatgatggatttaaggctttaataaatattaatgataagttagctaataaagaatatgaatttacatttaatttacccaaaaacagtaaattaattagtgctgccacatatttgggtaaagaatatgatacaaaagaagtatttgtagtagacaaaaataatataattacgagtattattagtccagcttgggctaaagatgcaaatggacataatgtttctacttattataagatagtatcgaataataaattagtacaagttgttgaattcacagaaaatactgcattcccggtggtagctgatcctaattggactaaaattgggaaatgcgctgggtcaatagcatgggctataggttctggcctttttggtggagcaaagctaattaaaataaaaaaatatatagcagagcttggaggacttcaaaaagcagctaaattattagttggtgcaaccacttgggaagaaaaattacacgcaggcggttatgcattaattaacttagctgctgagctaacaggtgtagcaggtatacaagcaaattgtttttaa

SEQ ID NO: 50 - protein, 62 aa, Bacillus coagulans
MKKIEKLTEKEMANIIGGKYYGNGVTCGKHSCSVDWGKATTCIINNGAMAWATGGHQGTHKC

SEQ ID NO: 51 - DNA, 189 bp, Bacillus coagulans
  source 1..189: mol_type = genomic DNA
atgaaaaaaattgaaaaattaactgaaaaagaaatggccaatatcattggtggtaaatactacggtaatggggttacttgtggcaaacattcctgctctgttgactggggtaaggctaccacctgcataatcaataatggagctatggcatgggctactggtggacatcaaggtactcataaatgctag

SEQ ID NO: 52 - protein, 490 aa, Escherichia coli
MDKVTDNSPDVESTESTEGSFPTVGVDTGDTITATLATGTENVGGGGGAFGGASESSAAIHATAKWSTAQLKKHQAEQAARAAAAEAALAKAKSQRDALTQRLKDIVNDALRANAARSPSVTDLAHANNMAMQAEAERLRLAKAEQKAREEAEAAEKALREAERQRDEIARQQAETAHLLAMAEAAEAEKNRQDSLDEEHRAVEVAEKKLAEAKAELAKAESDVQSKQAIVSRVAGELENAQKSVDVKVTGFPGWRDVQKKLERQLQDKKNEYSSVTNALNSAVSIRDAKKTEVQNAEIKLKEAKDALEKSQVKDSVDTMVGFYQYITEQYGEKYSRIAQDLAEKAKGSKFNSVDEALAAFEKYKNVLDKKFSKVDRDDIFNALESITYDEWAKHLEKISRALKVTGYLSFGYDVWDGTLKGLKTGDWKPLFVTLEKSAVDFGVAKIVALMFSFIVGAPLGFWGIAIITGIVSSYIGDDELNKLNELLGI

SEQ ID NO: 53 - DNA, 1473 bp, Escherichia coli
  source 1..1473: mol_type = genomic DNA
atggataaagtcactgataattctccagatgtggagagcacagaatctactgaggggtcattcccaactgttggggttgatactggcgatacgattacagcgacgcttgcaactggaactgaaaatgttggtggaggcggtggagcatttggtggggccagtgaaagttctgctgcgatacatgcaaccgctaaatggtctaccgcgcagttgaaaaaacatcaggctgaacaggctgcccgtgctgctgcggctgaggcagcattggcaaaagcgaaatctcagcgtgatgccctgactcaacgtctcaaggatattgttaatgacgctttacgtgctaatgccgctcgtagtccatcagtaactgaccttgctcatgccaataatatggcaatgcaggcagaggctgagcgtttgcgccttgcgaaggcagagcaaaaagcccgtgaagaagctgaagcagcagaaaaagcgctccgggaagcagaacgccaacgtgatgagattgcccgccaacaggctgaaaccgcgcatttgttagcaatggcggaggcagcagaggctgagaaaaatcgacaggattctcttgatgaagagcatcgggctgtggaagtggcagagaagaagctggctgaggctaaagctgaactggcgaaggccgaaagcgatgtacagagtaagcaagcgattgtttccagagttgcaggggagcttgaaaacgctcaaaaaagtgttgatgtgaaggttaccggatttcctggatggcgtgatgttcagaaaaaactggagagacaattgcaggataagaagaatgaatattcgtcagtgacgaatgctcttaattctgctgttagcattagagatgctaaaaaaacagaagttcagaatgctgagataaaattaaaagaagctaaggatgctcttgagaagagtcaggtaaaagactctgttgatactatggttgggttttatcaatatataaccgaacaatatggggaaaaatattccagaatagctcaggatttagctgaaaaggcgaagggtagtaaatttaatagtgttgatgaagcacttgctgcatttgaaaagtataaaaatgtactggataagaaattcagtaaggttgatagggatgatatttttaatgctttagagtctattacttatgatgagtgggccaagcatctagaaaagatctctagggctcttaaggttactggatatttgtctttcgggtatgatgtatgggatggtaccctaaagggattaaaaacaggagactggaagcctttatttgtcactctggagaagagcgcggtagatttcggcgtggcaaaaattgtggcattaatgtttagttttattgttggtgcgcctcttggcttctggggaattgcaattatcacaggtattgtttcttcttacataggggatgatgagttgaacaagcttaatgaattactaggtatttaa

SEQ ID NO: 54 - protein, 522 aa, Escherichia coli
METAVAYYKDGVPYDDKGQVIITLLNGTPDGSGSGGGGGKGGSKSESSAAIHATAKWSTAQLKKTQAEQAARAKAAAEAQAKAKANRDALTQRLKDIVNEALRHNASRTPSATELAHANNAAMQAEDERLRLAKAEEKARKEAEAAEKAFQEAEQRRKEIEREKAETERQLKLAEAEEKRLAALSEEAKAVEIAQKKLSAAQSEVVKMDGEIKTLNSRLSSSIHARDAEMKTLAGKRNELAQASAKYKELDELVKKLSPRANDPLQNRPFFEATRRRVGAGKIREEKQKQVTASETRINRINADITQIQKAISQVSNNRNAGIARVHEAEENLKKAQNNLLNSQIKDAVDATVSFYQTLTEKYGEKYSKMAQELADKSKGKKIGNVNEALAAFEKYKDVLNKKFSKADRDAIFNALASVKYDDWAKHLDQFAKYLKITGHVSFGYDVVSDILKIKDTGDWKPLFLTLEKKAADAGVSYVVALLFSLLAGTTLGIWGIAIVTGILCSYIDKNKLNTINEVLGI

SEQ ID NO: 55 - DNA, 1569 bp, Escherichia coli
  source 1..1569: mol_type = genomic DNA
atggaaaccgcggtagcgtactataaagatggtgttccttatgatgataagggacaggtaattattactcttttgaatggtactcctgacgggagtggctctggcggcggaggtggaaaaggaggcagtaaaagtgaaagttctgcagctattcatgcaactgctaaatggtctactgctcaattaaagaaaacacaggcagagcaggctgcccgggcaaaagctgcagcggaagcacaggcgaaagcaaaggcaaacagggatgcgctgactcagcgcctgaaggatatcgtgaatgaggctcttcgtcacaatgcctcacgtacgccttcagcaacagagcttgctcatgctaataatgcagctatgcaggcggaagacgagcgtttgcgccttgcgaaagcagaagaaaaagcccgtaaagaagcggaagcagcagaaaaggcttttcaggaagcagaacaacgacgtaaagagattgaacgggagaaggctgaaacagaacgccagttgaaactggctgaagctgaagagaaacgactggctgcattgagtgaagaagctaaagctgttgagatcgcccaaaaaaaactttctgctgcacaatctgaagtggtgaaaatggatggagagattaagactctcaattctcgtttaagctccagtatccatgcccgtgatgcagaaatgaaaacgctcgctggaaaacgaaatgaactggctcaggcatccgctaaatataaagaactggatgagctggtcaaaaaactatcaccaagagccaatgatccgcttcagaaccgtcctttttttgaagcaaccagacgacgggttggggccggtaagattagagaagaaaaacaaaaacaggtaacagcatcagaaacacgtattaaccggataaatgctgatataactcagatccagaaggctatttctcaggtcagtaataatcgtaatgccggtatcgctcgtgttcatgaagctgaagaaaatttgaaaaaagcacagaataatctccttaattcacagattaaggatgctgttgatgcaacagttagcttttatcaaacgctgactgaaaaatatggtgaaaaatattcgaaaatggcacaggaacttgctgataagtctaaaggtaagaaaatcggcaatgtgaatgaagctctcgctgcttttgaaaaatacaaggatgttttaaataagaaattcagcaaagccgatcgtgatgctatttttaatgcgttggcatcggtgaagtatgatgactgggctaaacatttagatcagtttgccaagtacttgaagattacggggcatgtttcttttggatatgatgtggtatctgatatcctaaaaattaaggatacaggtgactggaagccactatttcttacattagagaagaaagctgcagatgcaggggtgagttatgttgttgctttactttttagcttgcttgctggaactacattaggtatttggggtattgctattgttacaggaattctatgctcctatattgataagaataaacttaatactataaatgaggtgttagggatttaa

SEQ ID NO: 56 - protein, 626 aa, Escherichia coli
MSDPVRITNPGAESLGYDSDGHEIMAVDIYVNPPRVDVFHGTPPAWSSFGNKTIWGGNEWVDDSPTRSDIEKRDKEITAYKNTLSAQQKENENKRTEAGKRLSAAIAAREKDENTLKTLRAGNADAADITRQEFRLLQAELREYGFRTEIAGYDALRLHTESRMLFADADSLRISPREARSLIEQAEKRQKDAQNADKKAADMLAEYERRKGILDTRLSELEKNGGAALAVLDAQQARLLGQQTRNDRAISEARNKLSSVTESLNTARNALTRAEQQLTQQKNTPDGKTIVSPEKFPGRSSTNHSIVVSGDPRFAGTIKITTSAVIDNRANLNYLLSHSGLDYKRNILNDRNPVVTEDVEGDKKIYNAEVAEWDKLRQRLLDARNKITSAESAVNSARNNLSARTNEQKHANDALNALLKEKENIRNQLSGINQKIAEEKRKQDELKATKDAINFTTEFLKSVSEKYGAKAEQLAREMAGQAKGKKIRNVEEALKTYEKYRADINKKINAKDRAAIAAALESVKLSDISSNLNRFSRGLGYAGKFTSLADWITEFGKAVRTENWRPLFVKTETIIAGNAATALVALVFSILTGSALGIIGYGLLMAVTGALIDESLVEKANKFWGI

SEQ ID NO: 57 - DNA, 1881 bp, Escherichia coli
  source 1..1881: mol_type = genomic DNA
atgtctgaccctgtacgtattacaaatcccggtgcagaatcgctggggtatgattcagatggccatgaaattatggccgttgatatttatgtaaaccctccacgtgtcgatgtctttcatggtaccccgcctgcatggagttccttcgggaacaaaaccatctggggcggaaacgagtgggttgatgattccccaacccgaagtgatatcgaaaaaagggacaaggaaatcacagcgtacaaaaacacgctcagcgcgcagcagaaagagaatgagaataagcgtactgaagccggaaaacgcctctctgcggcgattgctgcaagggaaaaagatgaaaacacactgaaaacactccgtgccggaaacgcagatgccgctgatattacacgacaggagttcagactcctgcaggcagagctgagagaatacggattccgtactgaaatcgccggatatgacgccctccggctgcatacagagagccggatgctgtttgctgatgctgattctcttcgtatatctccccgggaggccaggtcgttaatcgaacaggctgaaaaacggcagaaggatgcgcagaacgcagacaagaaggccgctgatatgcttgctgaatacgagcgcagaaaaggtattctggacacccggttgtcagagctggaaaaaaatggcggggcagcccttgccgttcttgatgcacaacaggcccgtctgctcgggcagcagacacggaatgacagggccatttcagaggcccggaataaactcagttcagtgacggaatcgcttaacacggcccgtaatgcattaaccagagctgaacaacagctgacgcaacagaaaaacacgcctgacggcaaaacgatagtttcccctgaaaaattcccggggcgttcatcaacaaatgattctattgttgtgagcggtgatccgagatttgccggtacgataaaaatcacaaccagcgcagtcatcgataaccgtgcaaacctgaattatcttctgagccattccggtctggactataaacgcaatattctgaatgaccggaatccggtggtgacagaggatgtggaaggtgacaagaaaatttataatgctgaagttgctgaatgggataagttacggcaaagattgcttgatgccagaaataaaatcacctctgctgaatctgcggtaaattcggcgagaaataacctcagtgccagaacaaatgagcaaaagcatgcaaatgacgctcttaatgccctgttgaaggaaaaagagaatatccgtaaccagctttccggcatcaatcagaagatagcggaagagaaaagaaaacaggatgaactgaaggcaacgaaagacgcaattaatttcacaacagagttcctgaaatcagtttcagaaaaatatggtgcaaaagctgagcagttagccagagagatggccgggcaggctaaagggaagaaaatacgtaatgttgaagaggcattaaaaacgtatgaaaagtaccgggctgacattaacaaaaaaattaatgcaaaagatcgtgcagcgattgccgcagcccttgagtctgtgaagctgtctgatatatcgtctaatctgaacagattcagtcggggactgggatatgcaggaaaatttacaagtcttgctgactggatcactgagtttggtaaggctgtccggacagagaactggcgtcctctttttgttaaaacagaaaccatcatagcaggcaatgccgcaacggctcttgtggcactggtcttcagtattcttaccggaagcgctttaggcattatcgggtatggtttactgatggctgtcaccggtgcgctgattgatgaatcgcttgtggaaaaagcgaataagttctggggtatttaa

SEQ ID NO: 58 - protein, 626 aa, Escherichia coli
MSDPVRITNPGAESLGYDSDGHEIMAVDIYVNPPRVDVFHGTPPAWSSFGNKTIWGGNEWVDDSPTRSDIEKRDKEITAYKNTLSAQQKENENKRTEAGKRLSAAIAAREKDENTLKTLRAGNADAADITRQEFRLLQAELREYGFRTEIAGYDALRLHTESRMLFADADSLRISPREARSLIEQAEKRQKDAQNADKKAADMLAEYERRKGILDTRLSELEKNGGAALAVLDAQQARLLGQQTRNDRAISEARNKLSSVTESLKTARNALTRAEQQLTQQKNTPDGKTIVSPEKFPGRSSTNHSIVVSGDPRFAGTIKITTSAVIDNRANLNYLLTHSGLDYKRNILNDRNPVVTEDVEGDKKIYNAEVAEWDKLRQRLLDARNKITSAESAINSARNNVSARTNEQKHANDALNALLKEKENIRSQLADINQKIAEEKRKRDEINMVKDAIKLTSDFYRTIYDEFGKQASELAKELASVSQGKQIKSVDDALNAFDKFRNNLNKKYNIQDRMAISKALEAINQVHMAENFKLFSKAFGFTGKVIERYDVAVELQKAVKTDNWRPFFVKLESLAAGRAASAVTAWAFSVMLGTPVGILGFAIIMAAVSALVNDKFIEQVNKLIGI

SEQ ID NO: 59 - DNA, 1881 bp, Escherichia coli
  source 1..1881: mol_type = genomic DNA
atgtctgaccctgtacgtattacaaatcccggtgcagaatcgctgggatatgattcagatggccatgaaattatggccgttgatatttatgtaaaccctccacgtgtcgatgtctttcatggtaccccgcctgcatggagttccttcgggaacaaaaccatctggggtggaaacgagtgggtcgatgattccccaacccgaagtgatatcgaaaaaagggacaaggaaatcacagcgtacaaaaacacgctcagcgcgcagcagaaagagaatgagaataagcgtactgaagctggaaaacgcctttctgcggcaattgctgcaagggaaaaagatgaaaacacactgaaaacactccgtgccggaaacgcagatgccgctgatattacacgacaggagttcagactcctgcaggcagagctgagagaatacggattccgtactgaaatcgccggatatgatgccctccggctgcatacagagagccggatgctgtttgctgatgctgattctcttcgtatatctccccgcgaggccaggtcgttaatcgaacaggctgaaaaacggcagaaggatgcgcagaacgcagacaagaaggccgctgatatgcttgctgaatacgagcgcagaaaaggtattctggacacgcggttgtcagagctggaaaaaaatggcggggcagcccttgccgttcttgatgcacaacaggcccgtctgctcgggcagcagacacggaatgacagggccatttcagaggcccggaataaactcagttcggtgacggaatcgcttaagacggcccgtaatgcattaaccagagctgaacaacagctgacgcaacagaaaaacacgcctgacggcaaaacgatagtttcccctgaaaaattcccggggcgttcatcaacaaatcattctattgttgtgagtggtgatccgaggtttgccggtacgataaaaatcacaaccagcgcggtcatcgataaccgtgcaaacctgaattatcttctgacccattccggtctggactataaacgcaatattctgaatgaccggaatccggtggtgacagaggatgtggaaggtgacaagaaaatttataatgctgaagttgctgaatgggataagttacggcaacgattgcttgatgccagaaataaaatcacctctgctgaatctgcgataaattcggcgagaaataacgtcagtgccagaacaaatgaacaaaagcatgcaaatgacgctcttaatgccctgttgaaggaaaaagagaatatccgtagccagcttgctgacatcaatcagaaaatagctgaagagaaaagaaaaagggatgaaataaatatggtaaaggatgccataaaactcacctctgatttctacagaacgatatatgatgagttcggtaaacaagcatccgaacttgctaaggagctggcttctgtatctcaagggaaacagattaagagtgtggatgatgcactgaacgcttttgataaattccgtaataatctgaacaagaaatataacatacaagatcgcatggccatttctaaagccctggaagctattaatcaggtccatatggcggagaattttaagctgttcagtaaggcatttggttttaccggaaaagttattgaacgttatgatgttgctgtggagttacaaaaggctgtaaaaacggacaactggcgtccattttttgtaaaacttgaatcactggcagcaggaagagctgcttcagcagttacagcatgggcgttttccgtcatgctgggaacccctgtaggtattctgggttttgcaattattatggcggctgtgagtgcgcttgttaatgataagtttattgagcaggtcaataaacttattggtatctga

SEQ ID NO: 60 - protein, 271 aa, Escherichia coli
METLTVHAPSPSTNLPSYGNGAFSLSAPHVPGAGPLLVQVVYSFFQSPNMCLQALTQLEDYIKKHGASNPLTLQIISTNIGYFCNADRNLVLHPGISVYDAYHFAKPAPSQYDYRSMNMKQMSGNVTTPIVALAHYLWGNGAERSVNIANIGLKISPMKINQIKDIIKSGVVGTFPVSTKFTHATGDYNVITGAYLGNITLKTEGTLTISANGSWTYNGVVRSYDDKYDFNASTHRGIIGESLTRLGAMFSGKEYQILLPGEIHIKESGKR

SEQ ID NO: 61 - DNA, 816 bp, Escherichia coli
  source 1..816: mol_type = genomic DNA
atggaaaccttaactgttcatgcaccatcaccatcaactaacttaccaagttatggcaatggtgcattttctctttcagcaccacatgtgcctggtgctggccctcttttagtccaggttgtttatagttttttccagagtccaaacatgtgtcttcaggctttaactcaacttgaggattacatcaaaaaacatggggccagcaaccctctcacattgcagatcatatcgacaaatattggttacttctgtaacgccgaccgaaatctggttcttcaccctggaataagcgtttatgacgcttaccacttcgcaaaaccagcgccaagtcaatatgactatcgctcaatgaatatgaaacaaatgagcggtaatgtcactacaccaattgtggcgcttgctcactatttatggggtaatggcgctgaaaggagcgttaatatcgccaacattggtcttaaaatttcccctatgaaaattaatcagataaaagacattataaaatctggtgtagtaggcacattccctgtttctacaaagttcacacatgccactggtgattataatgttattaccggtgcatatcttggtaatatcacactgaaaacagaaggtactttaactatctctgccaatggctcctggacttacaatggcgttgttcgttcatatgatgataaatacgattttaacgccagcactcaccgtggcattatcggagagtcgctcacaaggctcggggcgatgttttctggtaaagagtaccagatactgcttcctggtgaaattcacattaaagaaagtggtaagcgataa

SEQ ID NO: 62 - protein, 387 aa, Escherichia coli
MGSNGADNAHNNAFGGGKNPGIGNTSGAGSNGSASSNRGNSNGWSWSNKPHKNDGFHSDGSYHITFHGDNNSKPKPGGNSGNRGNNGDGASAKVGEITITPDNSKPGRYISSNPEYSLLAKLIDAESIKGTEVYTFHTRKGQYVKVTVPDSNIDKMRVDYVNWKGPKYNNKLVKRFVSQFLLFRKEEKEKNEKEALLKASELVSGMGDKLGEYLGVKYKNVAKEVANDIKNFHGRNIRSYNEAMASLNKVLANPKMKVNKSDKDAIVNAWKQVNAKDMANKIGNLGKAFKVADLAIKVEKIREKSIEGYNTGNWGPLLLEVESWIIGGVVAGVAISLFGAVLSFLPISGLAVTALGVIGIMTISYLSSFIDANRVSNINNIISSVIR

SEQ ID NO: 63 - DNA, 51 bp, Escherichia coli
  source 1..51: mol_type = genomic DNA
gcaaatcgagtttcgaatataaataacattatatctagtgttattcgatga

SEQ ID NO: 64 - protein, 103 aa, Escherichia coli
MRTLTLNELDSVSGGASGRDIAMAIGTLSGQFVAGGIGAAAGGVAGGAIYDYASTHKPNPAMSPSGLGGTIKQKPEGIPSEAWNYAAGRLCNWSPNNLSDVCL

SEQ ID NO: 65 - DNA, 312 bp, Escherichia coli
  source 1..312: mol_type = genomic DNA
atgagaactctgactctaaatgaattagattctgtttctggtggtgcttcagggcgtgatattgcgatggctataggaacactatccggacaatttgttgcaggaggaattggagcagctgctgggggtgtggctggaggtgcaatatatgactatgcatccactcacaaacctaatcctgcaatgtctccatccggtttaggaggaacaattaagcaaaaacccgaagggataccttcagaagcatggaactatgctgcgggaagattgtgtaattggagtccaaataatcttagtgatgtttgtttataa

SEQ ID NO: 66 - protein, 58 aa, Enterococcus columbae
MMNATENQIFVETVSDQELEMLIGGAGRGWIKTLTKDCPNVISSICAGTIITACKNCA

SEQ ID NO: 67 - DNA, 177 bp, Enterococcus columbae
  source 1..177: mol_type = genomic DNA
atgatgaatgctactgaaaaccaaatttttgttgagactgtgagtgaccaagaattagaaatgttaattggtggtgcaggtcgtggatggattaagactttaacaaaagattgtccaaatgtgatttcttcaatttgtgcaggtacaattattacagcttgtaaaaattgtgcttaa

SEQ ID NO: 68 - protein, 59 aa, Lactobacillus curvatus
MNNVKELSMTELQTITGGARSYGNGVYCNNKKCWVNRGEATQSIIGGMISGWASGLAGM

SEQ ID NO: 69 - DNA, 180 bp, Lactobacillus curvatus
  source 1..180: mol_type = genomic DNA
atgaataatgtaaaagaattaagtatgacagaattacaaacaattaccggcggtgctagatcatatggcaacggtgtttactgtaataataaaaaatgttgggtaaatcggggtgaagcaacgcaaagtattattggtggtatgattagcggctgggctagtggtttagctggaatgtaa

SEQ ID NO: 70 - protein, 64 aa, Streptomyces sp.
MRSEMTLTSTNSAEALAAQDFANTVLSAAAPGFHADCETPAMATPATPTVAQFVIQGSTICLVC

SEQ ID NO: 71 - DNA, 195 bp, Streptomyces sp.
  source 1..195: mol_type = genomic DNA
gtgcgatctgagatgactcttacgagcacgaattccgctgaggctctggcggcgcaggactttgcgaacaccgttctcagcgcggcggccccgggcttccacgcggactgcgagacgccggccatggccaccccggccacgccgaccgtcgcccagttcgtgatccagggcagcacgatctgcctggtctgctga

SEQ ID NO: 72 - protein, 65 aa, Bacillus halodurans
MVNSKDLRNPEFRKAQGLQFVDEVNEKELSSLAGSGDVHAQTTWPCATVGVSVALCPTTKCTSQC

SEQ ID NO: 73 - DNA, 198 bp, Bacillus halodurans
  source 1..198: mol_type = genomic DNA
atggtaaattcaaaagatttgcgtaatcctgaattccgcaaagcccaaggtctacaattcgttgacgaggtgaacgagaaggaactttcgtctctagctggttcaggagatgtgcatgcacaaacaacttggccttgcgctacagttggtgtctccgtagccttgtgcccaactacaaagtgtacaagccagtgctaa

SEQ ID NO: 74 - protein, 66 aa, Carnobacterium divergens
MKNLKEGSYTAVNTDELKSINGGTKYYGNGVYCNSKKCWVDWGQASGCIGQTVVGGWLGGAIPGKC

SEQ ID NO: 75 - DNA, 201 bp, Carnobacterium divergens
  source 1..201: mol_type = genomic DNA
atgaaaaacttaaaagaaggttcatacactgctgttaatactgatgaattaaaaagtatcaatggtggaacaaaatattatgggaatggcgtttattgcaattctaaaaaatgttgggtagattggggacaagcttcaggttgtatcggtcaaactgttgttggcggatggctaggcggagctataccaggtaaatgctaa

SEQ ID NO: 76 - protein, 63 aa, Carnobacterium divergens
MIKREKNRTISSLGYEEISNHKLQEIQGGKGILGKLGVVQAGVDFVSGVWAGIKQSAKDHPNA

SEQ ID NO: 77 - DNA, 192 bp, Carnobacterium divergens
  source 1..192: mol_type = genomic DNA
atgattaaaagagaaaagaacagaacaatttcttcccttggttatgaagaaatttctaatcataaattgcaagaaatacaaggtggaaaaggaattcttggtaaactaggagtagtacaggcaggagtggattttgtatcaggagtgtgggctggaataaaacagtctgccaaagatcatcctaatgcgtaa

SEQ ID NO: 78 - protein, 75 aa, Carnobacterium divergens
MKKQILKGLVIVVCLSGATFFSTPQQASAAAPKITQKQKNCVNGQLGGMLAGALGGPGGVVLGGIGGAIAGGCFN

SEQ ID NO: 79 - DNA, 228 bp, Carnobacterium divergens
  source 1..228: mol_type = genomic DNA
atgaaaaaacaaattttaaaagggttggttatagttgtttgtttatctggggcaacatttttctcaacaccacaacaagcttctgctgctgcaccgaaaattactcaaaaacaaaaaaattgtgttaatggacaattaggtggaatgcttgctggagctttgggtggacctggcggagttgtgttaggtggtataggtggtgcaatagcaggaggttgttttaattaa

SEQ ID NO: 80 - protein, 72 aa, Enterococcus durans
MQTIKELNTMELQEIIGGENDHRMPYELNRPNNLSKGGAKCAAGILGAGLGAVGGGPGGFISAGISAVLGCM

SEQ ID NO: 81 - DNA, 219 bp, Enterococcus durans
  source 1..219: mol_type = genomic DNA
atgcaaacgatcaaagaattgaacacgatggaattacaagaaataattggaggtgaaaatgaccatcggatgccttacgaattgaaccgtccaaataatttatccaaaggtggggctaagtgtgctgctggaatacttggcgctggactaggcgcagtaggcggtggacctggcggatttattagtgccggaatcagtgctgttcttggttgtatgtaa

SEQ ID NO: 82 - protein, 72 aa, Enterococcus durans
MQTIKELNTMELQKIIGGENDHRMPYELNRPNNLSKGGAKCAAGILGAGLGAVGGGPGGFISAGISAVLGCM

SEQ ID NO: 83 - DNA, 219 bp, Enterococcus durans
  source 1..219: mol_type = genomic DNA
atgcaaacgatcaaagaattgaacacgatggaattacaaaaaataattggaggtgaaaatgaccatcggatgccttacgaattgaaccgtccaaataatttatccaaaggtggagctaagtgcgctgccggaatacttggtgctggattaggcgcagtaggcggtggacctggcggatttattagtgccggaatcagtgctgttcttggttgtatgtaa

SEQ ID NO: 84 - protein, 220 aa, Streptococcus dysgalactiae
  source 1..220: note = subsp. equisimilis
MKKLKRLVISLVTSLLVISSTVPALVYANETNNFAETQKEITTNSEATLTNEDYTKLTSEVKTIYTNLIQYDQTKNKFYVDEDKTEQYYNYDDESIKGVYLMKDSLNDELNNNNSSNYSEIINQKISEIDYVLQGNDINNLIPSNTRVKRSADFSWIQRCLEEAWGYAISLVTLKGIINLFKAGKFEAAAAKLASATAGRIAGMAALFAFVATCGATTVS

SEQ ID NO: 85 - DNA, 663 bp, Streptococcus dysgalactiae
  source 1..663: mol_type = genomic DNA; note = subsp. equisimilis
atgaaaaaattaaaacgtcttgttatctctcttgttacttcattactagtaatttcaagtacagttccagcacttgtttacgctaatgaaacaaataactttgcagaaactcaaaaagaaattacaacaaattcagaagcaacattaaccaatgaagactacactaaattaacttccgaagtaaaaacaatttatacaaatctgattcaatacgaccaaacaaaaaacaaattttacgtcgatgaagacaaaactgaacaatattataactacgatgatgaaagtataaaaggggtttatctcatgaaagatagtttgaacgatgagttaaacaataataactcttcaaactattctgaaataattaatcaaaaaatctctgaaattgactatgtccttcaaggaaacgatataaataatttaattcctagcaataccagagtaaaaagatcagcagatttttcttggattcaaagatgtctagaagaagcatggggatatgctattagtctagttactctaaaaggaataatcaatctatttaaagcaggaaaatttgaagctgctgctgctaaattagcttctgctacagcaggtagaatcgctggaatggctgccttatttgctttcgtagcaacttgcggtgcgacaactgtatcataa

SEQ ID NO: 86 - protein, 57 aa, Enterococcus faecalis
MKQYKVLNEKEMKKPIGGESVFSKIGNAVGPAAYWILKGLGNMSDVNQADRINRKKH

SEQ ID NO: 87 - DNA, 174 bp, Enterococcus faecalis
  source 1..174: mol_type = genomic DNA
atgaagcaatataaagtattgaatgaaaaagaaatgaaaaaacctattgggggagagtcggtttttagtaaaataggtaatgctgtaggtccagctgcttattggattttaaaaggattaggtaatatgagtgatgtaaaccaagctgatagaattaatagaaagaaacattaa

SEQ ID NO: 88 - protein, 44 aa, Enterococcus faecalis
MGAIAKLVAKFGWPIVKKYYKQIMQFIGEGWAINKIIDWIKKHI

SEQ ID NO: 89 - DNA, 135 bp, Enterococcus faecalis
  source 1..135: mol_type = genomic DNA
atgggagcaatcgcaaaattagtagcaaagtttggatggccaattgttaaaaagtattacaaacaaattatgcaatttattggagaaggatgggcaattaacaaaattattgattggatcaaaaaacatatttaa

SEQ ID NO: 90 - protein, 43 aa, Enterococcus faecalis
MGAIAKLVAKFGWPFIKKFYKQIMQFIGQGWTIDQIEKWLKRH

SEQ ID NO: 91 - DNA, 132 bp, Enterococcus faecalis
  source 1..132: mol_type = genomic DNA
atgggagcaatcgcaaaattagtagcaaagtttggatggccatttattaaaaaattctacaaacaaattatgcagtttatcggacaaggatggacaatagatcaaattgaaaaatggttaaaaagacattga

SEQ ID NO: 92 - protein, 74 aa, Enterococcus faecalis
MLNKKLLENGVVNAVTIDELDAQFGGMSKRDCNLMKACCAGQAVTYAIHSLLNRLGGDSSDPAGCNDIVRKYCK

SEQ ID NO: 93 - DNA, 225 bp, Enterococcus faecalis
  source 1..225: mol_type = genomic DNA
atgttaaataaaaaattattagaaaatggtgtagtaaatgctgtaacaattgatgaacttgatgctcaatttggtggaatgagcaaacgtgattgtaacttgatgaaggcgtgttgtgctggacaagcagtaacatatgctattcatagtcttttaaatcgattaggtggagactctagtgatccagctggttgtaatgatattgtaagaaaatattgtaaataa

SEQ ID NO: 94 - protein, 65 aa, Enterococcus faecium
MKHLKILSIKETQLIYGGTTHSGKYYGNGVYCTKNKCTVDWAKATTCIAGMSIGGFLGGAIPGKC

SEQ ID NO: 95 - DNA, 195 bp, Enterococcus faecium
  source 1..195: mol_type = genomic DNA
atgaaacatttaaaaattttgtctattaaagagacacaacttatctatgggggtaccactcatagtggaaaatattatggaaatggagtgtattgcactaaaaataaatgtacggtcgattgggccaaggcaactacttgtattgcaggaatgtctataggtggttttttaggtggagcaattccagggaagtgc

SEQ ID NO: 96 - protein, 105 aa, Enterococcus faecalis
MVKENKFSKIFILMALSFLGLALFSASLQFLPIAHMAKEFGIPAAVAGTVLNVVEAGGWVTTIVSILTAVGSGGLSLLAAAGRESIKAYLKKEIKKKGKRAVIAW

SEQ ID NO: 97 - DNA, 318 bp, Enterococcus faecalis
  source 1..318: mol_type = genomic DNA
atggttaaagaaaataaattttctaagatttttattttaatggctttgagttttttggggttagccttgtttagtgcaagtcttcagtttttgcccattgcacatatggctaaagagttcggtataccagcagcagttgcaggaactgtgcttaatgtagttgaagctggtggatgggtcactactattgtatcaattcttactgctgtaggtagcggaggtctttctttactcgctgcagcaggaagagagtcaattaaagcataccttaagaaagaaattaagaaaaaaggaaaaagagcagttattgcttggtaa

SEQ ID NO: 98 - protein, 71 aa, Enterococcus faecium
MQNVKELSTKEMKQIIGGENDHRMPNELNRPNNLSKGGAKCGAAIAGGLFGIPKGPLAWAAGLANVYSKCN

SEQ ID NO: 99 - DNA, 216 bp, Enterococcus faecium
  source 1..216: mol_type = genomic DNA
atgcaaaatgtaaaagaattaagtacgaaagagatgaaacaaattatcggtggagaaaatgatcacagaatgcctaatgagttaaatagacctaacaacttatctaaaggtggagcaaaatgtggtgctgcaattgctgggggattatttggaatcccaaaaggaccactagcatgggctgctgggttagcaaatgtatactctaaatgcaactaa

SEQ ID NO: 100 - protein, 58 aa, Enterococcus mundtii
MKKLTSKEMAQVVGGKYYGNGVSCNKKGCSVDWGKAIGIIGNNSAANLATGGAAGWKS

SEQ ID NO: 101 - DNA, 177 bp, Enterococcus mundtii
  source 1..177: mol_type = genomic DNA
ttgaagaaattaacatcaaaagaaatggcacaagtagtaggtggaaaatactacggtaatggagtctcatgtaataaaaaagggtgcagtgttgattggggaaaagctattggcattattggaaataattctgctgcgaatttagctactggtggagcagctggttggaaaagttaa

SEQ ID NO: 102 - protein, 44 aa, Enterococcus faecalis
MLAKIKAMIKKFPNPYTLAAKLTTYEINWYKQQYGRYPWERPVA

SEQ ID NO: 103 - DNA, 135 bp, Enterococcus faecalis
  source 1..135: mol_type = genomic DNA
atgttagcaaaaattaaagcgatgattaagaagtttccgaacccttatactttagcagctaagctaacgacttacgaaattaattggtataaacaacaatacggtcgttatccttgggagcgccctgtagcataa

SEQ ID NO: 104 - protein, 71 aa, Enterococcus faecium
MRKKLFSLALIGIFGLVVTNFGTKVDAATRSYGNGVYCNNSKCWVNWGEAKENIAGIVISGWASGLAGMGH

SEQ ID NO: 105 - DNA, 216 bp, Enterococcus faecium
  source 1..216: mol_type = genomic DNA
atgagaaaaaaattatttagtttagctcttattggaatatttgggttagttgtgacaaattttggtacaaaagttgatgcagctacgcgttcatatggtaatggtgtttattgtaataatagtaaatgctgggttaactggggagaagctaaagagaatattgcaggaatcgttattagtggctgggcttctggtttggcaggtatgggacattaa

SEQ ID NO: 106 - protein, 34 aa, Enterococcus faecium
MNFLKNGIAKWMTGAELQAYKKKYGCLPWEKISC

SEQ ID NO: 107 - DNA, 105 bp, Enterococcus faecium
  source 1..105: mol_type = genomic DNA
atgaattttcttaaaaatggtatcgcaaaatggatgaccggtgctgaattgcaagcgtataaaaagaaatatggatgcttgccatgggaaaaaatttcttgttaa

SEQ ID NO: 108 - protein, 76 aa, Enterococcus faecalis
MKKKLVKGLVICGMIGIGFTALGTNVEAATYYGNGVYCNKQKCWVDWSRARSEIIDRGVKAYVNGFTKVLGGIGGR

SEQ ID NO: 109 - DNA, 231 bp, Enterococcus faecalis
  source 1..231: mol_type = genomic DNA
atgaaaaagaaattagttaaaggcttagttatttgtggcatgattgggattggttttacagcattaggaacaaatgtagaagccgccacgtattacggaaatggtgtctattgcaataagcaaaaatgttgggtagattggagtagagcacgttctgaaattatagacagaggcgtaaaagcatacgtcaatggatttacgaaagtgttaggtggtataggtggaagataa

SEQ ID NO: 110 - protein, 60 aa, Enterococcus faecalis
MKKEELVGMAKEDFLNVICENDNKLENSGAKCPWWNLSCHLGNDGKICTYSHECTAGCNA

SEQ ID NO: 111 - DNA, 183 bp, Enterococcus faecalis
  source 1..183: mol_type = genomic DNA
atgaaaaaagaagaattagtaggaatggctaaggaagactttttaaatgttatttgtgaaaatgacaacaaactagaaaatagtggagcaaaatgtccttggtggaatctttcttgtcatttaggcaatgatggtaaaatttgcacttattcacatgaatgtaccgcaggttgtaatgcataa

SEQ ID NO: 112 - protein, 61 aa, Enterococcus faecalis
MTELNKRLQLKRDVSTENSLKKISNTDETHGGVTTSIPCTVMVSAAVCPTLVCSNKCGGRG

SEQ ID NO: 113 - DNA, 186 bp, Enterococcus faecalis
  source 1..186: mol_type = genomic DNA
atgactgaacttaacaaaagattacaattaaaaagagatgtttcaacagaaaatagtttgaaaaaaatttctaatactgatgaaacacatgggggagttactacatcaattccatgtacagtaatggttagtgcggcagtatgtcctacccttgtttgctcgaataaatgtggcggtagaggctag

SEQ ID NO: 114 - protein, 58 aa, Enterococcus faecium
MQNVKEVSVKEMKQIIGGSNDSLWYGVGQFMGKQANCITNHPVKHMIIPGYCLSKILG

SEQ ID NO: 115 - DNA, 177 bp, Enterococcus faecium
  source 1..177: mol_type = genomic DNA
atgcaaaatgtaaaagaagtttctgtaaaagagatgaaacaaattatcggtggttctaatgatagtctttggtatggtgtaggacaatttatgggtaaacaagcaaactgtataacaaaccatcctgttaaacacatgataattcctggatattgtttatcgaaaattttagggtaa

SEQ ID NO: 116 - protein, 55 aa, Enterococcus faecium
MKKYNELSKKELLQIQGGIAPIIVAGLGYLVKDAWDHSDQIISGFKKGWNGGRRK

SEQ ID NO: 117 - DNA, 168 bp, Enterococcus faecium
  source 1..168: mol_type = genomic DNA
atgaaaaaatataatgagttatctaaaaaagaacttctacagattcaaggaggaatagcacctattatagttgctggccttggctatttagtaaaagatgcatgggatcactcagatcaaataatctcaggatttaaaaaaggttggaatggtggacgtagaaaataa

SEQ ID NO: 118 - protein, 343 aa, Enterococcus faecalis
MKNILLSILGVLSIVVSLAFSSYSVNAASNEWSWPLGKPYAGRYEEGQQFGNTAFNRGGTYFHDGFDFGSAIYGNGSVYAVHDGKILYAGWDPVGGGSLGAFIVLQAGNTNVIYQEFSRNVGDIKVSTGQTVKKGQLIGKFTSSHLHLGMTKKEWRSAHSSWNKDDGTWFNPIPILQGGSTPTPPNPGPKNFTTNVRYGLRVLGGSWLPEVTNFNNTNDGFAGYPNRQHDMLYIKVDKGQMKYRVHTAQSGWLPWVSKGDKSDTVNGAAGMPGQAIDGVQLNYITPKGEKLSQAYYRSQTTKRSGWLKVSADNGSIPGLDSYAGIFGEPLDRLQIGISQSNPF

SEQ ID NO: 119 - DNA, 1032 bp, Enterococcus faecalis
  source 1..1032: mol_type = genomic DNA
atgaaaaatattttactttctattctaggggtattatctatcgttgtttctttggcgttttcttcttattctgtcaacgcagcttctaatgagtggtcgtggccactgggcaaaccatatgcgggaagatatgaagaaggacaacaattcgggaacactgcatttaaccgaggaggtacttatttccatgatgggtttgactttggttctgctatttatggaaatggcagtgtgtatgctgtgcatgatggtaaaattttatatgctggttgggatcctgtaggtggaggctcattaggtgcatttattgtactacaagcgggaaacacaaatgtgatttatcaagaatttagccgaaatgttggagatattaaagttagcactggacaaactgttaaaaaaggacagctgataggaaagtttacttctagtcatttacatttaggaatgacaaaaaaagaatggcgttctgctcattcttcttggaataaagatgatggcacttggtttaacccaattcctatacttcaaggaggatctacgcctacgcctccaaatccaggaccaaaaaatttcacaacaaatgttcgttacggattgcgggtcctcggaggttcatggttaccagaagtaaccaactttaacaataccaatgatggtttcgcaggttaccctaatcgtcaacatgatatgctttatataaaggtagataaagggcaaatgaaatatcgtgttcacacggctcaaagtggatggttgccttgggtaagtaaaggggataagagcgatacagtaaatggagcggcaggtatgcctggacaagcaattgatggtgttcagctaaactatataactcctaagggagaaaaattatcacaggcttactatcgttcacaaactacgaaacgatcaggctggttaaaagtaagtgcagataatggttctattcctggactagacagttatgcaggaatctttggagaaccgttggatcgcttgcaaataggtatttcacagtcaaatccattttaa

SEQ ID NO: 120 - protein, 56 aa, Staphylococcus epidermidis
MENKKDLFDLEIKKDNMENNNELEAQSLGPAIKATRQVCPKATRFVTVSCKKSDCQ

SEQ ID NO: 121 - DNA, 171 bp, Staphylococcus epidermidis
  source 1..171: mol_type = genomic DNA
atggaaaacaaaaaagatttatttgatttagaaatcaaaaaagataatatggaaaataataatgaattagaagctcaatctcttggtcctgcaattaaggcaactagacaggtatgtcctaaagcaacacgttttgttacagtttcttgtaaaaaaagtgattgtcaatag

SEQ ID NO: 122 - protein, 51 aa, Staphylococcus epidermidis
MAAFMKLIQFLATKGQKYVSLAWKHKGTILKWINAGQSFEWIYKQIKKLWA

SEQ ID NO: 123 - DNA, 156 bp, Staphylococcus epidermidis
  source 1..156: mol_type = genomic DNA
atggcagcatttatgaagttaattcagttcttagcaactaaaggtcaaaagtatgtttcacttgcatggaaacataaaggtactattttaaaatggattaacgccggtcaaagttttgaatggatttataaacaaatcaaaaaattatgggcataa

SEQ ID NO: 124 - protein, 52 aa, Staphylococcus epidermidis
MEAVKEKNDLFNLDVKVNAKESNDSGAEPRIASKFICTPGCAKTGSFNSYCC

SEQ ID NO: 125 - DNA, 159 bp, Staphylococcus epidermidis
  source 1..159: mol_type = genomic DNA
atggaagcagtaaaagaaaaaaatgatctttttaatcttgatgttaaagttaatgcaaaagaatctaacgattcaggagctgaaccaagaattgctagtaaatttatatgtactcctggatgtgcaaaaacaggtagttttaacagttattgttgttaa

SEQ ID NO: 126 - protein, 55 aa, Staphylococcus epidermidis
MNNSLFDLNLNKGVETQKSDLSPQSASVLKTSIKVSKKYCKGVTLTCGCNITGGK

SEQ ID NO: 127 - DNA, 168 bp, Staphylococcus epidermidis
  source 1..168: mol_type = genomic DNA
atgaataactcattattcgatttaaacctaaacaaaggtgtagaaactcaaaagagtgatttaagtccgcaatctgctagtgtcttgaagacttctattaaagtatctaaaaaatattgtaaaggtgttactttaacatgcggttgcaatattactggtggtaaataa

SEQ ID NO: 128 - protein, 52 aa, Staphylococcus gallinarum
MEAVKEKNELFDLDVKVNAKESNDSGAEPRIASKFLCTPGCAKTGSFNSYCC

SEQ ID NO: 129 - DNA, 159 bp, Staphylococcus gallinarum
  source 1..159: mol_type = genomic DNA
atggaagcagtaaaagagaaaaatgaactttttgatcttgacgttaaagtaaatgcaaaagagtctaatgattcaggcgcagaaccacgaattgctagtaaatttttatgtactcctggatgtgccaaaacaggtagcttcaatagctactgttgttaa

SEQ ID NO: 130 - protein, 63 aa, Lactococcus garvieae
MENNNYTVLSDEELQKIDGGIGGALGNALNGLGTWANMMNGGGFVNQWQVYANKGKINQYRPY

SEQ ID NO: 131 - DNA, 192 bp, Lactococcus garvieae
  source 1..192: mol_type = genomic DNA
atggaaaacaacaattacacagtactttcagatgaagaactacaaaaaattgatggtggaatcggcggggctcttggtaatgctctcaacggattaggtacctgggcaaacatgatgaacggtggaggatttgttaatcagtggcaagtttatgctaataaaggaaaaataaatcaataccgtccgtattaa

SEQ ID NO: 132 - protein, 63 aa, Lactococcus garvieae
MFDLVATGMAAGVAKTIVNAVSAGMDIATALSLFSGAFTAAGGIMALIKKYAQKKLWKQLIAA

SEQ ID NO: 133 - DNA, 192 bp, Lactococcus garvieae
  source 1..192: mol_type = genomic DNA
atgtttgatttagtcgcgactggaatggctgcaggtgtagcaaaaactattgttaatgccgttagtgctggtatggatattgccactgctttatcattgttctcaggagcttttactgcagctgggggaattatggcactcattaaaaaatatgctcaaaagaaattatggaaacagcttattgctgcataa

SEQ ID NO: 134 - protein, 91 aa, Lactobacillus gasseri
MVTKYGRNLGLNKVELFAIWAVLVVALLLTTANIYWIADQFGIHLATGTARKLLDAMASGASLGTAFAAILGVTLPAWALAAAGALGATAA

SEQ ID NO: 135 - DNA, 276 bp, Lactobacillus gasseri
  source 1..276: mol_type = genomic DNA
atggttactaagtacggacgtaatttaggtttgaacaaggtagagttgtttgcaatttgggcggttttagtagttgctcttttattgaccacagcgaacatttattggattgctgatcaattcgggattcatttagcgactggaacagcccgtaagttattagatgcaatggcttctggtgcctcattgggaactgcctttgctgctattttgggcgtgacattacctgcatgggctttggcagctgcaggagcattgggagcgactgcagcctag

SEQ ID NO: 136 - protein, 75 aa, Lactobacillus gasseri
MKNFNTLSFETLANIVGGRNNWAANIGGVGGATVAGWALGNAVCGPACGFVGAHYVPIAWAGVTAATGGFGKIRK

SEQ ID NO: 137 - DNA, 228 bp, Lactobacillus gasseri
  source 1..228: mol_type = genomic DNA
atgaaaaattttaatacattatcatttgaaacattggctaacatagttggtgggagaaataattgggctgctaatataggtggagtaggtggagcgacagtcgctggatgggctcttggaaatgcagtttgcggtcctgcttgtggctttgttggagcacactatgttccaatagcatgggctggcgtaacggcagctactggtggattcggaaagataagaaagtag

SEQ ID NO: 138 - protein, 64 aa, Lactobacillus plantarum
MSKLVKTLTISEISKAQNNGGKPAWCWYTLAMCGAGYDSGTCDYMYSHCFGIKHHSSGSSSYHC

SEQ ID NO: 139 - DNA, 195 bp, Lactobacillus plantarum
  source 1..195: mol_type = genomic DNA
atgagtaaattggttaagacacttactataagtgaaatttctaaggctcaaaacaacggtggaaaacctgcatggtgttggtatactttagcaatgtgtggtgctggttatgattcgggaacctgtgattatatgtattcgcattgttttggtataaagcatcatagtagtggtagtagcagttatcattgttag

SEQ ID NO: 140 - protein, 359 aa, Haloferax mediterranei
MSKDRDGRRTSRRGTLKKIGGFSLGALSFGAVGRTQAATGSSVTTADIAPPGPNGDPKSVQIDDKYTGAEMYGEGDFRVGLGTDLTMYPPVYRESLGNGSGGWEFDFTVCGSTACRFVDSNGDVKEDDKAKEMWWQEINFNDINQDLYSRNDSDWVGSTPADTQPEFDYTEFALARDGVTLALTALNPAMGSLALGATYFLSDMVNWIASQHEDDSSLKRKWDYDGLSGPLYADSSTYLLARDEMTSNSYESFTIDNIAVAFPEFPVRTKYYVTFTAPDDPSTQSISTLEEEGIYRVPATEVAAARPPGSRRSKSAADEMVYVADPKKFIEVEPVKNPSIPDRIYEEIEQKKKQRSRKQ

SEQ ID NO: 141 - DNA, 1080 bp, Haloferax mediterranei
  source 1..1080: mol_type = genomic DNA
atgtcgaaagacagagatgggagaaggacaagtcggcgaggcacgttaaagaaaatcggcggtttcagtctcggagcgcttagtttcggggcagtcggacgaactcaagcggcgaccggctcatcggttacgaccgctgatatcgcacctcccggaccgaacggagacccgaagagtgttcagatagatgataaatacaccggagccgagatgtacggcgagggtgacttcagagtcggtctcggaactgacctgacgatgtatccgcccgtgtaccgtgagagtcttggaaatggaagcgggggttgggaattcgacttcaccgtttgtgggtccactgcctgtcgatttgtggacagtaacggtgacgtcaaagaggacgacaaggcgaaagaaatgtggtggcaggaaattaacttcaacgacataaatcaggatttatacagtcggaacgattccgactgggtcgggtcgacccctgccgatacccaaccggagttcgattacaccgactttgcgctcgctcgggacggagtgacgctcgctctcacggcactcaaccccgcaatggggagtcttgcactcggtgccacgtacttcctcagcgacatggtgaactggattgcgagccagcacgaagacgacagttcgctcaagagaaaatgggattacgacgggctaagtgggccgttgtacgccgattcgtcgacgtacctactggcacgcgacgagatgacttcgaactcgtacgaatcattcacgatcgataacatcgccgttgccttcccagagttccccgtccggaccaagtactacgtcacattcactgcgccggatgacccgtcaacgcagtcgatatctacgctcgaagaggagggaatctaccgagtgcccgctacggaagtggctgcggccagaccaccggggtcccgacgttccaaatcggcagccgacgagatggtgtacgttgccgatccgaagaagttcatagaggtcgagccggtgaagaacccaagtatcccggaccgaatctacgaggagatagagcaaaaaaagaaacaacggagtaggaaacagtag

SEQ ID NO: 142 - protein, 311 aa, unidentified
  source 1..311: note = Haloarchaeon S8a
MSDKDSINRRNVLRKIGGIGVASAVGFSGLASGESLSDDEKQDVIDTIYKSQRVEQIKKKFGGVNIEPKKVQSVTTNQSGDLVTAKLSVSDGDLVYSSVKDTTVIVQFDRSASEIGESWPKNTEAFIKSTSSGVDLLRTATDEEIKDVTEGVNTSEIESADAVNIFIDPESQTYYMEKYDFNNKVLEMFELATGGTSSGKISPTREDQNHEYNVREHKVFNSEKQNIQLQSDCNINSNTAADVILCFNQVGSCALCSPTLVGGPVPTVACLLVVCFGTPNAVSAILEEVDNSCFNLIKDVISCWDEWTSFW

SEQ ID NO: 143 - DNA, 936 bp, unidentified
  source 1..936: mol_type = genomic DNA; note = Haloarchaeon S8a
atgtcggataaagacagcattaacagaagaaatgtattaagaaaaattggcggtatcggtgtggcttcagctgtcggattttctggtttggcaagcggggaaagtcttagcgatgatgagaaacaagatgttattgacacaatttacaaatcacaaagagttgaacagataaagaaaaagttcggaggagtgaatattgagccgaaaaaggttcaatctgtaacgaccaatcagagcggagatcttgttacggcgaagctgtcggttagtgatggggatttggtatattcgagtgtcaaagatacaactgtaatagttcagttcgatagatcggcttctgaaattggtgaaagttggcccaagaatactgaggcattcatcaaatcgacgtcctctggggtcgatcttctacgtacagcaactgatgaagaaataaaggacgttactgagggagtcaacacatctgaaattgaatctgcggatgctgttaacatatttattgatcctgaatcacagacatactatatggagaaatatgactttaataataaggtacttgagatgtttgaattagcgacaggtgggacaagtagtggtaaaatctcccccacacgtgaagaccagaatcacgaatataatgttagggaacataaagtatttaactcagaaaaacagaatatacaacttcagagtgactgtaatataaacagtaacaccgctgctgatgttattctatgcttcaaccaggttggttcttgtgcactctgctccccgactttagtcggaggtccagtccctacagttgcatgtctcttagtcgtctgtttcggcactccaaatgctgtgtccgcgatacttgaagaagtcgataattcttgctttaacttgatcaaggatgtaatttcgtgttgggatgaatggactagcttctggtga

SEQ ID NO: 144 - protein, 333 aa, Lactobacillus helveticus
MKHLNETTNVRILSQFDMDTGYQAVVQKGNVGSKYVYGLQLRKGATTILRGYRGSKINNPILELSGQAGGHTQTWEFAGDRKDINGEERAGQWFIGVKPSKIEGSKIIWAKQIARVDLRNQMGPHYSNTDFPRLSYLNRAGSNPFAGNKMTHAEAAVSPDYTKFLIATVENNCIGHFTIYNLDTINEKLDEKGNSEDVNLETVKYEDSFIIDNLYGDDNNSIVNSIQGYDLDNDGNIYISSQKAPDFDGSYYAHHKQIVKIPYYARSKESEDQWRAVNLSEFGGLDIPGKHSEVESIQIIGENHCYLTVAYHSKNKAGENKTTLNEIYELSWN

SEQ ID NO: 145 - DNA, 1002 bp, Lactobacillus helveticus
  source 1..1002: mol_type = genomic DNA
atgaagcatttaaatgaaacaactaatgttagaattttaagtcaatttgatatggatactggctatcaagcagtagttcaaaaaggcaatgtaggttcaaaatatgtatatggattacaacttcgcaaaggtgctactactatcttgcgtggttaccgtggaagtaaaattaataaccctattcttgaattatctggtcaagcaggtggtcacacacagacatgggaatttgctggtgatcgtaaagacattaatggtgaagaaagagcaggtcaatggtttataggtgttaaaccatcgaaaattgaaggaagcaaaattatttgggcaaagcaaattgcaagagttgatcttagaaatcaaatgggacctcattattcaaatactgactttcctcgattatcctacttgaatcgcgccggttctaatccatttgctggtaataagatgacgcatgccgaagccgcagtatcacctgattatactaagtttttaattgctactgttgaaaataactgtattggtcattttactatatacaatttagatacaattaatgaaaaacttgatgaaaagggaaatagtgaagatgttaatctcgaaactgttaaatacgaagatagttttatcattgataatttatatggtgatgataataattctattgtaaattcaattcaagggtatgatttggataatgatggaaatatttatatttccagtcaaaaagcgccagattttgatggctcttattatgcacatcataagcagattgttaagattccatattatgctcggtctaaagaaagcgaagaccaatggagagctgtaaatttaagcgaattcggtggcttggatattccaggtaaacatagtgaagttgaaagcatccaaattattggtgagaatcattgttacttaactgttgcatatcattctaaaaataaagcgggtgaaaataaaactactttgaatgagatttatgaattatcttggaattag

SEQ ID NO: 146 - protein, 74 aa, Enterococcus hirae; identical to SEQ ID NO: 20

SEQ ID NO: 147 - DNA, 225 bp, Enterococcus hirae; identical to SEQ ID NO: 21
  source 1..225: mol_type = genomic DNA

SEQ ID NO: 148 - protein, 75 aa, Lactobacillus johnsonii
MKQFNYLSHKDLAVVVGGRNNWQTNVGGAVGSAMIGATVGGTICGPACAVAGAHYLPILWTAVTAATGGFGKIRK

SEQ ID NO: 149 - DNA, 228 bp, Lactobacillus johnsonii
  source 1..228: mol_type = genomic DNA
atgaaacaatttaattatttatcacataaagatttagcagtcgttgttggtggaagaaataattggcaaacaaatgtgggaggagcagtgggatcagctatgattggggctacagttggtggtacaatttgtggacctgcatgtgctgtagctggtgcccattatcttcctattttatggacagcggttacagctgcaacaggtggttttggcaagataagaaagtag

SEQ ID NO: 150 - protein, 62 aa, Lactobacillus johnsonii
MKLNDKELSKIVGGNRWGDTVLSAASGAGTGIKACKSFGPWGMAICGVGGAAIGGYFGYTHN

SEQ ID NO: 151 - DNA, 189 bp, Lactobacillus johnsonii
  source 1..189: mol_type = genomic DNA
atgaaattaaatgacaaagaattatcaaagattgttggtggaaatcgatggggagatactgttttatcagctgctagtggcgcaggaactggtattaaagcatgtaaaagttttggcccatggggaatggcaatttgtggtgtaggaggtgcagcaataggaggttattttggctatactcataattaa

SEQ ID NO: 152 - protein, 59 aa, Lactococcus lactis
  source 1..59: note = subsp. lactis
MNKNEIETQPVTWLEEVSDQNFDEDVFGACSTNTFSLSDYWGNNGAWCTLTHECMAWCK

SEQ ID NO: 153 - DNA, 180 bp, Lactococcus lactis
  source 1..180: mol_type = genomic DNA; note = subsp. lactis
atgaacaaaaatgaaattgaaacacaaccagttacatggttggaagaagtatctgatcaaaattttgatgaagatgtatttggtgcgtgtagtactaacacattctcgctcagtgattactggggaaataacggggcttggtgtacactcactcatgaatgtatggcttggtgtaaataa

SEQ ID NO: 154 - protein, 65 aa, Lactococcus lactis
  source 1..65: note = subsp. lactis
MKEKNMKKNDTIELQLGKYLEDDMIELAEGDESHGGTTPATPAISILSAYISTNTCPTTKCTRAC

SEQ ID NO: 155 - DNA, 198 bp, Lactococcus lactis
  source 1..198: mol_type = genomic DNA; note = subsp. lactis
atgaaagaaaaaaatatgaaaaagaatgacactattgaattacaattgggaaaataccttgaagatgatatgattgaattagctgaaggggatgagtctcatggaggaacaacaccagcaactcctgcaatctctattctcagtgcatatattagtaccaatacttgtccaacaacaaaatgtacacgtgcttgttaa

SEQ ID NO: 156 - protein, 51 aa, Lactococcus lactis
  source 1..51: note = subsp. lactis
MKEQNSFNLLQEVTESELDLILGAKGGSGVIHTISHECNMNSWQFVFTCCS

SEQ ID NO: 157 - DNA, 156 bp, Lactococcus lactis
  source 1..156: mol_type = genomic DNA; note = subsp. lactis
atgaaagaacaaaactcttttaatcttcttcaagaagtgacagaaagtgaattggaccttattttaggtgcaaaaggcggcagtggagttattcatacaatttctcatgaatgtaatatgaatagctggcaatttgtatttacttgctgctcttaa

SEQ ID NO: 158 - protein, 53 aa, Lactococcus lactis
MAGFLKVVQLLAKYGSKAVQWAWANKGKILDWLNAGQAIDWVVSKIKQILGIK

SEQ ID NO: 159 - DNA, 162 bp, Lactococcus lactis
  source 1..162: mol_type = genomic DNA
atggcagggtttttaaaagtagttcaattactagctaaatatggttctaaagctgtacaatgggcttgggcaaacaagggtaagattttagattggcttaatgcaggtcaggctattgattgggtagtttcgaaaattaagcaaattttaggtattaagtaa

SEQ ID NO: 160 - protein, 53 aa, Lactococcus lactis
MAGFLKVVQILAKYGSKAVQWAWANKGKILDWINAGQAIDWVVEKIKQILGIK

SEQ ID NO: 161 - DNA, 162 bp, Lactococcus lactis
  source 1..162: mol_type = genomic DNA
atggcagggtttttaaaagtagtccaaattttggctaagtatggttctaaagccgtacaatgggcatgggcaaataaaggaaaaatcttagattggattaatgcaggtcaagctattgactgggtagttgaaaagattaagcaaattttgggtattaaataa

SEQ ID NO: 162 - protein, 65 aa, Lactobacillus amylovorus
MKQLNSEQLQNIIGGNRWTNAYSAALGCAVPGVKYGKKLGGVWGAVIGGVGGAAVCGLAGYVRKG

SEQ ID NO: 163 - DNA, 198 bp, Lactobacillus amylovorus
  source 1..198: mol_type = genomic DNA
atgaaacaattgaattcagaacaattacaaaatattatcggtggaaatagatggactaatgcatacagcgcagctttgggatgcgctgtccctggagttaaatatggaaaaaaacttggtggcgtatggggtgctgtaattggtggcgtaggcggtgcagcagtctgtggcttggcgggttatgttcgtaaaggctaa

SEQ ID NO: 164 - protein, 68 aa, Lactobacillus sakei
  source 1..68: note = L45
MKTEKKVLDELSLHASAKMGARDVESSMNADSTPVLASVAVSMELLPTASVLYSDVAGCFKYSAKHHC

SEQ ID NO: 165 - DNA, 207 bp, Lactobacillus sakei
  source 1..207: mol_type = genomic DNA; note = L45
atgaaaacagaaaaaaaggttttagatgaactgagcttacacgcttctgcaaaaatgggagcacgtgatgttgaatccagcatgaatgcagactcaacaccagttttagcatcagtcgctgtatccatggaattattgccaactgcgtctgttctttattcggatgttgcaggttgcttcaaatattctgcaaaacatcattgttag

SEQ ID NO: 166 - protein, 91 aa, Lactococcus lactis
  source 1..91: note = subsp. lactis
MKTKSLVLALSAVTLFSAGGIVAQAEGTWQHGYGVSSAYSNYHHGSKTHSATVVNNNTGRQGKDTQRAGVWAKATVGRNLTEKASFYYNFW

SEQ ID NO: 167 - DNA, 276 bp, Lactococcus lactis
  source 1..276: mol_type = genomic DNA; note = subsp. lactis
atgaaaaccaagtctctcgtattggcattatctgcggttacgttattctctgccggaggaattgtagctcaagctgaaggaacatggcaacatggatatggtgttagttcggcatattcaaattatcatcatggtagcaaaactcattcagccacagttgtaaataataatactggccgacaaggtaaggatacacaacgtgccggtgtttgggcaaaagctactgttggacgtaacttaactgaaaaagcttcattttattataacttttggtaa

SEQ ID NO: 168 - protein, 75 aa, Lactococcus lactis
  source 1..75: note = subsp. cremoris
MKNQLNFNIVSDEELSEANGGKLTFIQSTAAGDLYYNTNTHKYVYQQTQNAFGAAANTIVNGWMGGAAGGFGLHH

SEQ ID NO: 169 - DNA, 228 bp, Lactococcus lactis
  source 1..228: mol_type = genomic DNA; note = subsp. cremoris
atgaaaaatcaattaaattttaatattgtttcagatgaagaactttcagaagctaacggaggaaaattaacatttattcaatcgacagcggctggagatttatattacaatactaatacacacaaatatgtttaccaacaaactcaaaacgcttttggggctgctgctaataccattgttaatggatggatgggtggcgctgctggaggtttcgggttgcaccattga

SEQ ID NO: 170 - protein, 68 aa, Lactococcus lactis
  source 1..68: note = subsp. cremoris
MKNQLNFNIVSDEELAEVNGGSLQYVMSAGPYTWYKDTRTGKTICKQTIDTASYTFGVMAEGWGKTFH

SEQ ID NO: 171 - DNA, 207 bp, Lactococcus lactis
  source 1..207: mol_type = genomic DNA; note = subsp. cremoris
atgaaaaatcaattaaattttaatattgtttctgatgaagaacttgcagaagttaatggaggaagcttgcagtatgttatgagtgctggaccatatacttggtataaagatactagaacaggaaaaacaatatgtaaacagacaattgacacagcaagttatacatttggtgtaatggcagaaggatggggaaaaacattccactaa

SEQ ID NO: 172 - protein, 63 aa, Lactococcus sp.
  source 1..63: note = QU 12
MKLIDHLGAPRWAVDTILGAIAVGNLASWVLALVPGPGWAVKAGLATAAAIVKHQGKAAAAAW

SEQ ID NO: 173 - DNA, 192 bp, Lactococcus sp.
  source 1..192: mol_type = genomic DNA; note = QU 12
atgaaattaattgatcatttaggtgctccaagatgggccgttgatactattttaggtgcaatcgcagttgggaacttagcaagttgggttctagcgcttgtccctggtccagggtgggcagtaaaagctggtttagcaactgctgctgccatcgttaaacatcaaggtaaagctgccgctgctgcttggtaa

SEQ ID NO: 174 - protein, 50 aa, Brevibacillus sp.
  source 1..50: note = GI-9
MACQCPDAISGWTHTDYQCHGLENKMYRHVYAICMNGTQVYCRTEWGSSC

SEQ ID NO: 175 - DNA, 153 bp, Brevibacillus sp.
  source 1..153: mol_type = genomic DNA; note = GI-9
atggcttgccaatgtccagatgcgatctcaggttggacgcatacagattaccagtgtcacggtttggagaataaaatgtatagacatgtttatgcaatttgcatgaacggtactcaagtatattgcagaacagagtggggtagcagctgctag

SEQ ID NO: 176 - protein, 57 aa, Leuconostoc pseudomesenteroides
MNKEYNSISNFKKITNKDLQNINGGFIGRAIGDFVYFGAKGLRESGKLLNYYYKHKH

SEQ ID NO: 177 - DNA, 174 bp, Leuconostoc pseudomesenteroides
  source 1..174: mol_type = genomic DNA
atgaataaagaatataatagcattagcaattttaaaaaaattactaataaagacttgcaaaacataaatggtggatttattggtagggcaataggtgactttgtgtactttggagcgaagggactaagagaatctggtaaactacttaattattactataagcataagcattga

SEQ ID NO: 178 - protein, 53 aa, Leuconostoc pseudomesenteroides
MKNQLMSFEVISEKELSTVQGGKGLGKLIGIDWLLGQAKDAVKQYKKDYKRWH

SEQ ID NO: 179 - DNA, 162 bp, Leuconostoc pseudomesenteroides
  source 1..162: mol_type = genomic DNA
atgaaaaatcagttaatgtctttcgaagtgatatcagaaaaagaattgtccacggtacaaggtggcaaaggcttaggtaaactcataggaattgattggcttttgggtcaagctaaggacgctgttaaacagtacaagaaggattacaaacgttggcactaa

SEQ ID NO: 180 - protein, 61 aa, Leuconostoc gelidum
MMNMKPTESYEQLDNSALEQVVGGKYYGNGVHCTKSGCSVNWGEAFSAGVHRLANGGNGFW

SEQ ID NO: 181 - DNA, 186 bp, Leuconostoc gelidum
  source 1..186: mol_type = genomic DNA
atgatgaacatgaaacctacggaaagctatgagcaattggataatagtgctctcgaacaagtcgtaggaggtaagtattatggtaacggagttcattgcacaaaaagtggttgttctgtaaactggggagaagccttttcagctggagtacatcgtttagcaaatggtggaaatggtttctggtaa

SEQ ID NO: 182 - protein, 61 aa, Leuconostoc carnosum
MNNMKSADNYQQLDNNALEQVVGGKYYGNGVHCTKSGCSVNWGEAFSAGVHRLANGGNGFW

SEQ ID NO: 183 - DNA, 186 bp, Leuconostoc carnosum
  source 1..186: mol_type = genomic DNA
atgaataacatgaaatctgcggataattatcagcaattggataataatgctctcgaacaagtcgtaggaggtaagtattatggtaacggagttcattgcacaaaaagtggttgttctgtaaactggggagaagccttttcagctggagtacatcgtttagcaaatggtggaaatggtttctggtaa

SEQ ID NO: 184 - protein, 63 aa, Leuconostoc mesenteroides
MFLVNQLGISKSLANTILGAIAVGNLASWLLALVPGPGWATKAALATAETIVKHEGKAAAIAW

SEQ ID NO: 185 - DNA, 192 bp, Leuconostoc mesenteroides
  source 1..192: mol_type = genomic DNA
atgttcttggtaaatcagttagggatttcaaaatcgttagctaatactattcttggtgcaattgctgttggtaatttggccagttggttattagctttggttcctggtccgggttgggcaacaaaagcagcacttgcgacagctgaaacaattgtgaagcatgaaggaaaagcagctgctattgcgtggtaa

SEQ ID NO: 186 - protein, 74 aa, Bacillus licheniformis
MSKKEMILSWKNPMYRTESSYHPAGNILKELQEEEQHSIAGGTITLSTCAILSKPLGNNGYLCTVTKECMPSCN

SEQ ID NO: 187 - DNA, 225 bp, Bacillus licheniformis
  source 1..225: mol_type = genomic DNA
atgtcaaaaaaggaaatgattctttcatggaaaaatcctatgtatcgcactgaatcttcttatcatccagcagggaacatccttaaagaactccaggaagaggaacagcacagcatcgccggaggcacaatcacgctcagcacttgtgccatcttgagcaagccgttaggaaataacggatacctgtgtacagtgacaaaagaatgcatgccaagctgtaactaa

SEQ ID NO: 188 - protein, 266 aa, Brevibacterium linens
MNNLYRELAPIPGPAWAEIEEEARRTFKRNIAGRRIVDVAGPTGFETSAVTTGHIRDVQSETSGLQVKQRIVQEYIELRTPFTVTRQAIDDVARGSGDSDWQPVKDAATTIAMAEDRAILHGLDAAGIGGIVPGSSNAAVAIPDAVEDFADAVAQALSVLRTVGVDGPYSLLLSSAEYTKVSESTDHGYPIREHLSRQLGAGEIIWAPALEGALLVSTRGGDYELHLGQDLSIGYYSHDSETVELYLQETFGFLALTDESSVPLSL

SEQ ID NO: 189 - DNA, 801 bp, Brevibacterium linens
  source 1..801: mol_type = genomic DNA
gtgaataacctctatcgcgagcttgcccccatccccggcccggcctgggcggagatcgaggaggaggctcgacggacattcaaacgcaatatcgccggccgccggatcgtcgatgtcgcagggcccacgggcttcgagacctccgcggtgaccactggccacatccgagacgtccagtcggagacgagcggactgcaggttaagcagcgcatcgtgcaggaatacatcgagctgcggaccccattcaccgtgactcggcaggccatcgatgacgtggcccgcgggtccggtgactcggactggcagcccgtcaaggatgcggccacgacgatcgcgatggctgaagatcgggccattctccacgggctcgatgcggccgggatcggcggaatcgttcccggcagctcgaatgccgcagtggccatccccgacgccgtcgaggacttcgcggacgccgtcgcccaggcgctgagtgtgctgcgcacggtgggagtcgacgggccctacagcctgttgctctcctccgcggagtacaccaaggtctccgagtccaccgaccacggctacccgatccgcgagcacctctcccggcagctcggcgccggagagatcatctgggcgcccgcgctcgaaggggcgctgctcgtctccacgcgcgggggtgactacgagctccacctcggccaggacctgtcgatcggttactacagccacgacagcgagaccgtcgaactctatctgcaggagaccttcggattcctcgcgctgaccgacgaatccagtgtgcctttgagcctctga

SEQ ID NO: 190 - protein, 71 aa, Listeria innocua
MKKAALKFIIVIAILGFSFSFFSIQSEAKSYGNGVQCNKKKCWVDWGSAISTIGNNSAANWATGGAAGWKS

SEQ ID NO: 191 - DNA, 216 bp, Listeria innocua
  source 1..216: mol_type = genomic DNA
ttgaagaaggcagcgttaaaatttattattgttattgctattctaggtttcagtttttctttctttagcatacaatctgaagctaaatcttatggaaatggagttcagtgtaataagaaaaaatgttgggtagattggggtagtgctataagtactattggaaataattctgcagcgaattgggctacaggtggagcagctggttggaaaagctga

SEQ ID NO: 192 - protein, 68 aa, Bacillus sp.
MSQEAIIRSWKDPFSRENSTQNPAGNPFSELKEAQMDKLVGAGDMEAACTFTLPGGGGVCTLTSECIC

SEQ ID NO: 193 - DNA, 207 bp, Bacillus sp.
  source 1..207: mol_type = genomic DNA
atgagtcaagaagctatcattcgttcatggaaagatcctttttcccgtgaaaattctacacaaaatccagctggtaacccattcagtgagctgaaagaagcacaaatggataagttagtaggtgcgggagacatggaagcagcatgtacttttacattgcctggtggcggcggtgtttgtactctaacttctgaatgtatttgttaa

SEQ ID NO: 194 - protein, 61 aa, Leuconostoc mesenteroides
MTNMKSVEAYQQLDNQNLKKVVGGKYYGNGVHCTKSGCSVNWGEAASAGIHRLANGGNGFW

SEQ ID NO: 195 - DNA, 186 bp, Leuconostoc mesenteroides
  source 1..186: mol_type = genomic DNA
atgacgaatatgaagtctgtggaagcatatcagcaattagataaccagaatctcaagaaagttgttggtggaaagtattatgggaatggtgttcactgtacaaaaagtggatgctctgttaactggggagaagctgcctcagctggcatacatcgtttggccaatggtggaaatggattttggtaa

SEQ ID NO: 196 - protein, 68 aa, Clavibacter michiganensis
  source 1..68: note = subsp. michiganensis
MNDILETETPVMVSPRWDMLLDAGEDTSPSVQTQIDAEFRRVVSPYMSSSGWLCTLTIECGTIICACR

SEQ ID NO: 197 - DNA, 207 bp, Clavibacter michiganensis
  source 1..207: mol_type = genomic DNA; note = subsp. michiganensis
atgaacgacatcctcgagacggagacccccgtcatggtcagcccccggtgggacatgctgctcgacgcgggcgaggacaccagcccgtccgtccagacccagatcgacgcggagttccgtcgcgtcgtgagcccgtacatgtccagcagcggctggctctgcacgctcaccatcgaatgtggcaccatcatctgcgcgtgtcgctga

SEQ ID NO: 198 - protein, 69 aa, Escherichia coli
MELKASEFGVVLSVDALKLSRQSPLGVGIGGGGGGGGGGSCGGQGGGCGGCSNGCSGGNGGSGGSGSHI

SEQ ID NO: 199 - DNA, 207 bp, Escherichia coli
  source 1..207: mol_type = genomic DNA
atggaattaaaagcgagtgaatttggtgtagttttgtccgttgatgctcttaaattatcacgccagtctccattaggtgttggcattggtggtggtggcggcggcggcggcggcggtagctgcggtggtcaaggtggcggttgtggtggttgcagcaacggttgtagtggtggaaacggtggcagcggcggaagtggttcacatatc

SEQ ID NO: 200 - protein, 7 aa, Escherichia coli
MRTGNAN

SEQ ID NO: 201 - DNA, 24 bp, Escherichia coli
  source 1..24: mol_type = genomic DNA
atgcgtactggtaatgcaaactaa

SEQ ID NO: 202 - protein, 99 aa, Klebsiella pneumoniae
MREISQKDLNLAFGAGETDPNTQLLNDLGNNMAWGAALGAPGGLGSAALGAAGGALQTVGQGLIDHGPVNVPIPVLIGPSWNGSGSGYNSATSSSGSGS

SEQ ID NO: 203 - DNA, 300 bp, Klebsiella pneumoniae
  source 1..300: mol_type = genomic DNA
atgagagaaattagtcaaaaggacttaaatcttgcttttggtgcaggagagaccgatccaaatactcaacttctaaacgaccttggaaataatatggcatggggtgctgctcttggcgctcctggcggattaggatcagcagctttgggggccgcgggaggtgcattacaaactgtagggcaaggattaattgaccatggtcctgtaaatgtccccatccctgtactcatcgggccaagctggaatggtagcggtagtggttataacagcgcaacatccagttccggtagtggtagttaa

SEQ ID NO: 204 - protein, 75 aa, Escherichia coli
MREITESQLRYISGAGGAPATSANAAGAAAIVGALAGIPGGPLGVVVGAVSAGLTTAIGSTVGSGSASSSAGGGS

SEQ ID NO: 205 - DNA, 228 bp, Escherichia coli
  source 1..228: mol_type = genomic DNA
atgcgagaaataacagaatcacagttaagatatatttccggggcgggaggtgcgccagcgacttcagctaatgccgcaggtgctgcagctattgttggagctctcgccggaatacctggtggtccacttggggttgtagttggagccgtatctgccggtttgacaacagcaattggctcgaccgtgggaagtggtagtgccagttcttctgctggtggcggtagctaa

SEQ ID NO: 206 - protein, 58 aa, Escherichia coli
MIKHFHFNKLSSGKKNNVPSPAKGVIQIKKSASQLTKGGAGHVPEYFVGIGTPISFYG

SEQ ID NO: 207 - DNA, 177 bp, Escherichia coli
  source 1..177: mol_type = genomic DNA
atgattaagcattttcattttaataaactgtcttctggtaaaaaaaataatgttccatctcctgcaaagggggttatacaaataaaaaaatcagcatcgcaactcacaaaaggtggtgcaggacatgtgcctgagtattttgtggggattggtacacctatatctttctatggctga

SEQ ID NO: 208 - protein, 90 aa, Escherichia coli
MYMRELDREELNCVGGAGDPLADPNSQIVRQIMSNAAWGPPLVPERFRGMAVGAAGGVTQTVLQGAAAHMPVNVPIPKVPMGPSWNGSKG

SEQ ID NO: 209 - DNA, 273 bp, Escherichia coli
  source 1..273: mol_type = genomic DNA
atgtatatgagagagttagatagagaggaattaaattgcgttggtggggctggagatccgcttgcagatcctaattcccaaattgtaagacagataatgtctaatgcggcatggggcccgcctttggtgccagagcggtttaggggaatggctgttggagccgcaggtggggttacgcagacagttcttcaaggagcagcagctcatatgccggttaatgtccctatacctaaagttccgatgggaccctcatggaacggaagtaaaggataa

SEQ ID NO: 210 - protein, 50 aa, Enterococcus mundtii
MSQVVGGKYYGNGVSCNKKGCSVDWGKAIGIIGNNSAANLATGGAAGWKS

SEQ ID NO: 211 - DNA, 153 bp, Enterococcus mundtii
  source 1..153: mol_type = genomic DNA
atgtcacaggtagtaggtggaaaatactacggtaatggagtctcatgtaataaaaaagggtgcagtgttgattggggaaaagcgattggcattattggaaataattctgctgcgaatttagctactggtggagcagctggttggaaaagttaa

SEQ ID NO: 212 - protein, 58 aa, Enterococcus mundtii
MKKLTSKEMAQVVGGKYYGNGLSCNKKGCSVDWGKAIGIIGNNSAANLATGGAAGWKS

SEQ ID NO: 213 - DNA, 177 bp, Enterococcus mundtii
  source 1..177: mol_type = genomic DNA
ttgaagaaattaacatcaaaagaaatggcacaagtagtaggtgggaaatactacggtaatggattatcatgtaataaaaaagggtgcagtgttgattggggaaaagctattggcattattggaaataattctgctgcgaatttagctactggtggagcagctggttggaaaagttaa

SEQ ID NO: 214 - protein, 63 aa, Streptococcus mutans
MSNTQLLEVLGTETFDVQEDLFAFDTTDTTIVASNDDPDTRFKSWSLCTPGCARTGSFNSYCC

SEQ ID NO: 215 - DNA, 192 bp, Streptococcus mutans
  source 1..192: mol_type = genomic DNA
atgtcaaacacacaattattagaagtccttggtactgaaacttttgatgttcaagaagatctctttgcttttgatacaacagatactactattgtggcaagcaacgacgatccagatactcgtttcaaaagttggagcctttgtacgcctggttgtgcaaggacaggtagtttcaatagttactgttgctga

SEQ ID NO: 216 - protein, 53 aa, Streptococcus mutans
MNKLNSNAVVSLNEVSDSELDTILGGNRWWQGVVPTVSYECRMNSWQHVFTCC

SEQ ID NO: 217 - DNA, 162 bp, Streptococcus mutans
  source 1..162: mol_type = genomic DNA
atgaacaagttaaacagtaacgcagtagtttctttgaatgaagtttcagattctgaattggatactattttgggtggtaatcgttggtggcaaggtgttgtgccaacggtctcatatgagtgtcgcatgaattcatggcaacatgttttcacttgctgttaa

SEQ ID NO: 218 - protein, 57 aa, Lactococcus lactis
  source 1..57: note = subsp. lactis
MSTKDFNLDLVSVSKKDSGASPRITSISLCTPGCKTGALMGCNMKTATCHCSIHVSK

SEQ ID NO: 219 - DNA, 174 bp, Lactococcus lactis
  source 1..174: mol_type = genomic DNA; note = subsp. lactis
atgagtacaaaagattttaacttggatttggtatctgtttcgaagaaagattcaggtgcatcaccacgcattacaagtatttcgctatgtacacccggttgtaaaacaggagctctgatgggttgtaacatgaaaacagcaacttgtcattgtagtattcacgtaagcaaataa

SEQ ID NO: 220 - protein, 57 aa, Lactococcus lactis
MSTKDFNLDLVSVSKKDSGASPRITSISLCTPGCKTGALMGCNMKTATCNCSVHVSK

SEQ ID NO: 221 - DNA, 171 bp, Lactococcus lactis
  source 1..171: mol_type = genomic DNA
atgagtacaaaagatttcaacttggatttggtatctgtttcgaagaaagattcaggtgcatcaccacgcattacaagtatttcgctatgtacacccggttgtaaaacaggagctctgatgggttgtaacatgaaaacagcaacttgtaattgtagcgttcacgtaagcaaa

SEQ ID NO: 222 - protein, 57 aa, Lactococcus lactis
MSTKDFNLDLVSVSKTDSGASTRITSISLCTPGCKTGVLMGCNLKTATCNCSVHVSK

SEQ ID NO: 223 - DNA, 174 bp, Lactococcus lactis
  source 1..174: mol_type = genomic DNA
atgagtacaaaagatttcaacttagatttggtatctgtttcaaaaacagattctggcgcttcaacacgtattaccagcatttcgctttgtacaccaggttgtaaaacaggtgttctgatgggatgtaacctgaaaacagcaacttgtaattgtagcgttcacgtaagcaaataa

SEQ ID NO: 224 - protein, 55 aa, Streptococcus uberis
MNNEDFNLDLIKISKENNSGASPRITSKSLCTPGCKTGILMTCPLKTATCGCHFG

SEQ ID NO: 225 - DNA, 168 bp, Streptococcus uberis
  source 1..168: mol_type = genomic DNA
atgaacaatgaagattttaatttggatctcatcaaaatctcaaaggaaaacaactcaggagcttcacctcgaataactagtaaatcattatgtactcctggatgtaagacgggtattttgatgacttgtccactaaaaactgcaacctgtggttgtcattttggataa

SEQ ID NO: 226 - protein, 57 aa, Lactococcus lactis
  source 1..57: note = subsp. lactis
MSTKDFNLDLVSVSKKDSGASPRITSISLCTPGCKTGALMGCNMKTATCNCSIHVSK

SEQ ID NO: 227 - DNA, 174 bp, Lactococcus lactis
  source 1..174: mol_type = genomic DNA; note = subsp. lactis
atgagtacaaaagattttaacttggatttggtatctgtttcgaagaaagattcaggtgcatcaccacgcattacaagtatttcgctatgtacacccggttgtaaaacaggagctctgatgggttgtaacatgaaaacagcaacttgtaattgtagtattcacgtaagcaaataa

SEQ ID NO: 228 - protein, 57 aa, Staphylococcus warneri
MENSKVMKDIEVANLLEEVQEDELNEVLGAKKKSGVIPTVSHDCHMNSFQFVFTCCS

SEQ ID NO: 229 - DNA, 174 bp, Staphylococcus warneri
  source 1..174: mol_type = genomic DNA
atggaaaattctaaagttatgaaggacattgaagtagcaaatttattagaagaggttcaagaagatgaattgaatgaagtcttaggagctaagaaaaagtcaggagtaatcccaactgtgtcacacgattgccatatgaattctttccaatttgtatttacttgttgttcataa

SEQ ID NO: 230 - protein, 58 aa, Paenibacillus polymyxa
MAENLFDLDIQVNKSQGSVEPQVLSIVACSSGCGSGKTAASCVETCGNRCFTNVGSLC

SEQ ID NO: 231 - DNA, 177 bp, Paenibacillus polymyxa
  source 1..177: mol_type = genomic DNA
atggctgaaaacttatttgatctggacattcaagtaaacaaatctcaaggttctgtagagcctcaggttctgagcattgttgcatgttctagcggatgtggtagcggtaaaacagctgccagttgtgttgaaacttgtggcaaccggtgctttactaacgttggttcactctgctaa

SEQ ID NO: 232 - protein, 62 aa, Pediococcus acidilactici
MKKIEKLTEKEMANIIGGKYYGNGVTCGKHSCSVDWGKATTCIINNGAMAWATGGHQGNHKC

SEQ ID NO: 233 - DNA, 189 bp, Pediococcus acidilactici
  source 1..189: mol_type = genomic DNA
atgaaaaaaattgaaaaattaactgaaaaagaaatggccaatatcattggtggtaaatactacggtaatggggttacttgtggcaaacattcctgctctgttgactggggtaaggctaccacttgcataatcaataatggagctatggcatgggctactggtggacatcaaggtaatcataaatgctag

SEQ ID NO: 234 - protein, 60 aa, Pediococcus pentosaceus
MTEIKVLNDKELKNVVGGKYYGNGVHCGKKTCYVDWGQATASIGKIIVNGWTQHGPWAHR

SEQ ID NO: 235 - DNA, 183 bp, Pediococcus pentosaceus
  source 1..183: mol_type = genomic DNA
atgactgaaattaaagtactaaacgataaggaactaaaaaatgtcgtaggaggaaagtattacggtaacggagtgcattgtggtaaaaagacttgctatgtggactggggacaagctacagctagcattggaaaaattatagtgaacggatggacacaacacgggccttgggcacatagataa

SEQ ID NO: 236 - protein, 60 aa, Staphylococcus epidermidis
MKNNKNLFDLEIKKETSQNTDELEPQTAGPAIRASVKQCQKTLKATRLFTVSCKGKNGCK

SEQ ID NO: 237 - DNA, 183 bp, Staphylococcus epidermidis
  source 1..183: mol_type = genomic DNA
atgaaaaataacaaaaatttatttgatttagaaattaaaaaagaaacaagtcaaaacactgatgaacttgaacctcaaactgctggaccagcgattagagcttctgtgaaacaatgtcagaaaactttgaaagctacgcgtttatttacagtgtcttgcaaaggaaaaaacggatgtaaatag

SEQ ID NO: 238 - protein, 62 aa, Carnobacterium maltaromaticum
MKTVKELSVKEMQLTTGGKYYGNGVSCNKNGCTVDWSKAIGIIGNNAAANLTTGGAAGWNKG

SEQ ID NO: 239 - DNA, 189 bp, Carnobacterium maltaromaticum
  source 1..189: mol_type = genomic DNA
atgaaaactgttaaagaacttagcgttaaagaaatgcaactaactacaggaggtaagtattacggaaatggcgtttcctgtaataaaaatggttgtactgtagattggagcaaagctattgggattataggaaacaatgcagcagcaaatttgactacaggtggagccgctggttggaacaaaggataa

SEQ ID NO: 240 - protein, 69 aa, Lactobacillus plantarum
  VARIANT 40: note = Xaa = any amino acid
MYKELTVDELALIDGGKKKKKKVACTWGNAATAAASGAVXGILGGPTGALAGAIWGVSQCASNNLHGMH

SEQ ID NO: 241 - DNA, 210 bp, Lactobacillus plantarum
  misc_feature 119: note = n = a, c, t or g
  source 1..210: mol_type = genomic DNA
atgtataaagaattaacagttgatgaattagcattgattgatggaggaaaaaagaagaagaaaaaagtagcttgtacttggggaaatgcagcaacagccgctgcttctggtgcagttangggtattcttggtgggcctactggtgcactggctggagctatctggggcgtttcacaatgcgcgtctaacaacttacacggcatgcactaa

SEQ ID NO: 242 - protein, 56 aa, Lactobacillus plantarum
MMKKIEKLTEKEMANIIGGKYYGNGVTCGKHSCSVNWGQAFSCSVSHLANFGHGKC

SEQ ID NO: 243 - DNA, 171 bp, Lactobacillus plantarum
  source 1..171: mol_type = genomic DNA
atgatgaaaaaaattgaaaaattaactgaaaaagaaatggccaatatcattggtggtaaatactatggtaatggggttacttgtggtaaacattcctgctctgttaactggggccaagcattttcttgtagtgtgtcacatttagctaacttcggtcatggaaagtgctaa

SEQ ID NO: 244 - protein, 64 aa, Lactobacillus plantarum
MSKLVKTLTVDEISKIQTNGGKPAWCWYTLAMCGAGYDSGTCDYMYSHCFGVKHSSGGGGSYHC

SEQ ID NO: 245 - DNA, 195 bp, Lactobacillus plantarum
  source 1..195: mol_type = genomic DNA
atgagtaaactagttaaaacattaactgtcgatgaaatctctaagattcaaaccaatggtggaaaacctgcatggtgttggtacacattggcaatgtgcggtgctggttatgattcaggcacttgtgattatatgtattcacactgctttggtgtaaaacactctagcggtggtggcggtagctaccattgttag

SEQ ID NO: 246 - protein, 56 aa, Lactobacillus plantarum
MLQFEKLQYSRLPQKKLAKISGGFNRGGYNFGKSVRHVVDAIGSVAGIRGILKSIR

SEQ ID NO: 247 - DNA, 171 bp, Lactobacillus plantarum
  source 1..171: mol_type = genomic DNA
atgctacagtttgagaaattacaatattccaggttgccgcaaaaaaagcttgccaaaatatctggtggttttaatcggggcggttataactttggtaaaagtgttcgacatgttgttgatgcaattggttcagttgcaggcattcgtggtattttgaaaagtattcgttaa

SEQ ID NO: 248 - protein, 52 aa, Lactobacillus plantarum
MKKFLVLRDRELNAISGGVFHAYSARGVRNNYKSAVGPADWVISAVRGFIHG

SEQ ID NO: 249 - DNA, 159 bp, Lactobacillus plantarum
  source 1..159: mol_type = genomic DNA
atgaaaaaatttctagttttgcgtgaccgtgaattaaatgctatttcaggtggcgttttccatgcctatagcgcgcgtggcgttcggaataattataaaagtgctgttgggcctgccgattgggtcattagcgctgtccgaggattcatccacggatag

SEQ ID NO: 250 - protein, 55 aa, Lactobacillus plantarum
MTVNKMIKDLDVVDAFAPISNNKLNGVVGGGAWKNFWSSLRKGFYDGEAGRAIRR

SEQ ID NO: 251 - DNA, 168 bp, Lactobacillus plantarum
  source 1..168: mol_type = genomic DNA
atgactgtgaacaaaatgattaaggatttggatgtagtagatgcatttgcacctatttctaataataagttgaacggggttgttgggggaggcgcttggaaaaatttctggtctagtttaagaaaaggattttatgatggcgaagctggcagagcaatccgtcgttaa

SEQ ID NO: 252 - protein, 57 aa, Lactobacillus plantarum
MKIKLTVLNEFEELTADAEKNISGGRRSRKNGIGYAIGYAFGAVERAVLGGSRDYNK

SEQ ID NO: 253 - DNA, 174 bp, Lactobacillus plantarum
  source 1..174: mol_type = genomic DNA
atgaaaattaaattaactgttttaaatgaatttgaagaattaactgctgacgctgaaaagaatatttctggtggccgtcggagtcgtaaaaatggaattggatacgctattggttatgcgtttggcgcggttgaacgggccgtgcttggtggttcaagggattataataagtga

SEQ ID NO: 254 - protein, 47 aa, Lactobacillus plantarum
MDKFEKISTSNLEKISGGDLTTKLWSSWGYYLGKKARWNLKHPYVQF

SEQ ID NO: 255 - DNA, 141 bp, Lactobacillus plantarum
  source 1..141: mol_type = genomic DNA
atggataaatttgaaaaaattagtacatctaacctagaaaagatctctggcggtgatttaacaaccaagttatggagctcttggggatattatcttggcaagaaagcacgttggaatttaaagcacccatatgttcaattt

SEQ ID NO: 256 - protein, 55 aa, Lactobacillus plantarum
MNNLNKFSTLGKSSLSQIEGGSVPTSVYTLGIKILWSAYKHRKTIEKSFNKGFYH

SEQ ID NO: 257 - DNA, 168 bp, Lactobacillus plantarum
  source 1..168: mol_type = genomic DNA
atgaataacttgaataaattttctactctaggcaagagtagcttgtctcaaattgagggcggatcagtcccaacttcagtatatacgcttggaattaaaattctatggtctgcgtataagcatcgcaaaacgattgaaaaaagttttaataaaggcttttatcattaa

SEQ ID NO: 258 - protein, 55 aa, Lactobacillus plantarum
MNNALSFEQQFTDFSTLSDSELESVEGGRNKLAYNMGHYAGKATIFGLAAWALLA

SEQ ID NO: 259 - DNA, 168 bp, Lactobacillus plantarum
  source 1..168: mol_type = genomic DNA
atgaataacgcattaagttttgaacaacaatttacagacttcagcaccttatcggactctgaattagaatccgttgagggtggccgaaataagcttgcatataatatggggcattacgctggtaaggcaaccatttttggacttgcagcatgggcactccttgcatga

SEQ ID NO: 260 - protein, 47 aa, Lactobacillus plantarum
MDKIIKFQGISDDQLNAVIGGKKKKQSWYAAAGDAIVSFGEGFLNAW

SEQ ID NO: 261 - DNA, 144 bp, Lactobacillus plantarum
  source 1..144: mol_type = genomic DNA
atggataagattattaagtttcaagggatttctgatgatcaattaaatgctgttatcggtgggaaaaagaaaaaacaatcttggtacgcagcagctggtgatgcaatcgttagttttggtgaaggatttttaaatgcttggtaa

SEQ ID NO: 262 - protein, 59 aa, Lactobacillus plantarum
MKISKIEAQARKDFFKKIDTNSNLLNVNGAKCKWWNISCDLGNNGHVCTLSHECQVSCN

SEQ ID NO: 263 - DNA, 180 bp, Lactobacillus plantarum
  source 1..180: mol_type = genomic DNA
atgaaaatttctaagattgaagctcaggctcgtaaagatttttttaaaaaaatcgatactaactcgaacttattaaatgtaaatggtgccaaatgcaagtggtggaatatttcgtgtgatttaggaaataatggccatgtttgtaccttgtcacatgaatgccaagtatcttgtaactaa

SEQ ID NO: 264 - protein, 67 aa, Lactobacillus plantarum
MTKTSRRKNAIANYLEPVDEKSINESFGAGDPEARSGIPCTIGAAVAASIAVCPTTKCSKRCGKRKK

SEQ ID NO: 265 - DNA, 204 bp, Lactobacillus plantarum
  source 1..204: mol_type = genomic DNA
atgactaaaactagtcgtcgtaagaatgctattgctaattatttagaaccagtcgacgaaaaaagtattaatgaatcttttggggctggggatccggaagcaagatccggaattccatgtacaatcggcgcagctgtcgcagcatcaattgcagtttgtccaactactaagtgtagtaaacgttgtggcaagcgtaagaaataa

SEQ ID NO: 266 - protein, 48 aa, Lactobacillus plantarum
MKIQIKGMKQLSNKEMQKIVGGKSSAYSLQMGATAIKQVKKLFKKWGW

SEQ ID NO: 267 - DNA, 147 bp, Lactobacillus plantarum
  source 1..147: mol_type = genomic DNA
atgaaaattcaaattaaaggtatgaagcaacttagtaataaggaaatgcaaaaaatagtaggtggaaagagtagtgcgtattctttgcagatgggggcaactgcaattaaacaggtaaagaaactgtttaaaaaatggggatggtaa

SEQ ID NO: 268 - protein, 207 aa, Propionibacterium jensenii
MNKTHKMATLVIAAILAAGMTAPTAYADSPGNTRITASEQSVLTQILGHKPTQTEYNRYVETYGSVPTEADINAYIEASESEGSSSQTAAHDDSTSPGTSTEIYTQAAPARFSMFFLSGTWITRSGVVSLSLKPRKGGIGNEGDERTWKTVYDKFHNAGQWTRYKNNGVDASMKKQYMCHFKYGMVKTPWNLEPHKKAADVSPVKCN

SEQ ID NO: 269 - DNA, 624 bp, Propionibacterium jensenii
  source 1..624: mol_type = genomic DNA
atgaacaaaacacacaaaatggcgacgctggtaattgccgcgatcttggccgccggaatgaccgcaccaactgcctatgcagattctcctggaaacaccagaattacagccagcgagcaaagcgtccttacccagatactcggccacaaacctacacaaactgaatataaccgatacgttgagacttacggaagcgtaccgaccgaagcagacatcaacgcatatatagaagcgtctgaatctgagggatcatcaagtcaaacggctgctcacgatgactcgacatcacccggcacgagtaccgaaatctacacgcaggcagcccctgccaggttctcaatgtttttcctgtccggaacttggatcactaggagtggtgtagtatcgctctccttgaagccaaggaagggtggtattggcaacgagggggacgagcgtacctggaagactgtatacgacaaattccataacgctgggcaatggacacgatacaagaacaacggcgtagacgccagcatgaaaaagcagtacatgtgccacttcaagtacgggatggtgaagacgccatggaatctggagccccacaagaaggctgcagacgtcagtccagtcaagtgcaactag

SEQ ID NO: 270 - protein, 96 aa, Propionibacterium thoenii
MKKTLLRSGTIALATAAAFGASLAAAPSAMAVPGGCTYTRSNRDVIGTCKTGSGQFRIRLDCNNAPDKTSVWAKPKVMVSVHCLVGQPRSISFETK

SEQ ID NO: 271 - DNA, 291 bp, Propionibacterium thoenii
  source 1..291: mol_type = genomic DNA
atgaagaagaccctcctgcgaagtggaacgatcgcactggcgaccgcggctgcatttggcgcatcattggcagccgccccatctgccatggccgttcctggtggttgcacgtacacaagaagcaatcgcgacgtcatcggtacctgcaagactggaagcggccagttccgaatccgacttgactgcaacaacgctccagacaaaacttcagtctgggccaagcccaaggtaatggtgtcggttcactgtcttgttggtcaaccgaggtccatctcgttcgagaccaagtga

SEQ ID NO: 272 - protein, 255 aa, Propionibacterium freudenreichii
  source 1..255: note = subsp. freudenreii
MNTKAVNLKSENTTKLVSYLTENQLDEFIRRIRIDGALVEEVSQNAKQALDNTGLNGWINTDCDEGLLSDFISKIASARWIPLAESIRPAVTDRDKYRVSCWFYQGMNIAIYANIGGVANIIGYTEAAVATLLGAVVAVAPVVPGTPTPPKDKSSQYKEVPLAVRLSETYHEEGVRGLFDELNYSESRMISTLRRASTDGVLINSWNDGQDTILLKKYNFQDLQLTVRSRIVGNQTIIEECKITDGRKTLSDETV

SEQ ID NO: 273 - DNA, 768 bp, Propionibacterium freudenreichii
  source 1..768: mol_type = genomic DNA; note = subsp freudenreii
atgaataccaaagctgtaaatctgaagtcagaaaacacgactaagttggtgagctaccttacggaaaatcaattggatgagtttattagaaggattcgcattgatggcgctcttgtggaagaggtcagtcaaaatgctaagcaggccttagataatactgggctcaatggctggataaatactgattgcgatgaaggccttctctctgatttcatttcaaagatagcaagtgctagatggattccattagctgagtcaattcgacctgcggtgactgacagggataagtatcgagtaagttgctggttctaccaggggatgaatatagcaatttacgcaaatatcggtggcgtggccaatattatcggctatacggaggccgcagtcgcaacactccttggtgcagttgtggcggtagctcctgtggtccctggaactccaacccctccaaaggacaagagttcgcaatataaggaggttccccttgccgttcgtctttccgaaacataccacgaagagggagtacgaggtctattcgacgagctgaactactccgagagccgtatgatctctactctaaggcgagcatcaaccgatggagtcctaattaattcttggaacgatgggcaggatacaattctgcttaagaagtacaatttccaagacttgcaactgactgtcaggagccgcattgttgggaatcaaacaataattgaagaatgcaaaatcactgatggtagaaaaactctttcagacgagactgtgtag

SEQ ID NO: 274 - protein, 618 aa, Pseudomonas aeruginosa
MARPIADLIHFNSTTVTASGDVYYGPGGGTGIGPIARPIEHGLDSSTENGWQEFESYADVGVDPRRYVPLQVKEKRREIELQFRDAEKKLEASVQAELDKADAALGPAKNLAPLDVINRSLTIVGNALQQKNQKLLLNQKKITSLGAKNFLTRTAEEIGEQAVREGNINGPEAYMRFLDREMEGLTAAYNVKLFTEAISSLQIRMNTLTAAKASIEAAAANKAREQAAAEAKRKAEEQARQQAAIRAANTYAMPANGSVVATAAGRGLIQVAQGAASLAQAISDAIAVLGRVLASAPSVMAVGFASLTYSSRTAEQWQDQTPDSVRYALGMDAAKLGLPPSVNLNAVAKASGTVDLPMRLTNEARGNTTTLSVVSTDGVSVPKAVPVRMAAYNATTGLYEVTVPSTTAEAPPLILTWTPASPPGNQNPSSTTPVVPKPVPVYEGATLTPVKATPETYPGVITLPEDLIIGFPADSGIKPIYVMFRDPRDVPGAATGKGQPVSGNWLGAASQGEGAPIPSQIADKLRGKTFKNWRDFREQFWIAVANDPELSKQFNPGSLAVMRDGGAPYVRESEQAGGRIKIEIHHKVRVADGGGVYNMGNLVAVTPKRHIEIHKGGK

SEQ ID NO: 275 - DNA, 1857 bp, Pseudomonas aeruginosa
  source 1..1857: mol_type = genomic DNA
atggcacgacccattgctgaccttatccacttcaactctacaactgtcacggcaagcggagacgtttattacggccctgggggaggtaccggcattggccccattgccagacctatagagcacggcttggattcgtccactgaaaatggctggcaagagtttgaaagttatgctgatgtgggcgttgaccccagacgctatgttcctcttcaggttaaagaaaaacgcagggagatcgagcttcagttccgagatgccgagaaaaaacttgaggcgtcggtacaagccgagctggataaggctgatgccgctcttggtccggcaaagaatcttgcaccattggacgtcatcaaccgcagtctgaccatcgttggaaacgccctccagcaaaagaatcaaaaactactgctgaatcagaagaagattaccagcctgggtgcaaagaatttccttacccgtacggcggaagagatcggtgaacaagcggtgcgagaaggcaatattaacgggcctgaagcctatatgcgcttcctcgacagggaaatggaaggtctcacggcagcttataacgtaaaactcttcaccgaagcgatcagtagtctccagatccgcatgaatacgttgaccgccgccaaagcaagtattgaggcggccgcagcaaacaaggcgcgtgaacaagcagcggctgaggccaaacgcaaagccgaagagcaggcccgccagcaagcggcgataagagctgccaatacctatgccatgccggccaatggcagcgttgtcgccaccgccgcaggccggggtctgatccaggtcgcacaaggcgccgcatcccttgctcaagcgatctccgatgcgattgccgtcctgggccgggtcctggcttcagcaccctcggtgatggccgtgggctttgccagtctgacctactcctcccggactgccgagcaatggcaggaccaaacgcccgatagcgttcgttacgccctgggcatggatgccgctaaattggggcttcccccaagcgtaaacctgaacgcggttgcaaaagccagcggtaccgtcgatctgccgatgcgcctgaccaacgaggcacgaggcaacacgacgaccctttcggtggtcagcaccgatggtgtgagcgttccgaaagccgttccggtccggatggcggcctacaatgccacgacaggcctgtacgaggttacggttccctctacgaccgcagaagcgccgccactgatcctgacctggacgccggcgagtcctccaggaaaccagaacccttcgagtaccactccggtcgtaccgaagccggtgccggtatatgagggagcgacccttacaccggtgaaggctaccccggaaacctatcctggggtgattacactaccggaagacctgatcatcggcttcccggccgactcggggatcaagccgatctatgtgatgttcagggatccgcgggatgtacctggtgctgcgactggcaagggacagcccgtcagcggtaattggctcggcgccgcctctcaaggtgagggggctccaattccaagccagattgcggataaactacgtggtaagacattcaaaaactggcgggactttcgggaacaattctggatagctgtggctaatgatcctgagttaagtaaacagtttaatcctggtagtttagctgtaatgagagatggaggggctccttatgtcagagagtcagaacaggctggcgggagaataaagatcgaaatccaccacaaggttcgagtagcagatggaggcggcgtttacaatatggggaaccttgttgcagtaacgccaaaacgtcatatagaaatccacaagggagggaagtga

SEQ ID NO: 276 - protein, 689 aa, Pseudomonas aeruginosa
MAVNDYEPGSMVITHVQGGGRDIIQYIPARSSYGTPPFVPPGPSPYVGTGMQEYRKLRSTLDKSHSELKKNLKNETLKEVDELKSEAGLPGKAVSANDIRDEKSIVDALMDAKAKSLKAIEDRPANLYTASDFPQKSESMYQSQLLASRKFYGEFLDRHMSELAKAYSADIYKAQIAILKQTSQELENKARSLEAEAQRAAAEVEADYKARKANVEKKVQSELDQAGNALPQLTNPTPEQWLERATQLVTQAIANKKKLQTANNALIAKAPNALEKQKATYNADLLVDEIASLQARLDKLNAETARRKEIARQAAIRAANTYAMPANGSVVATAAGRGLIQVAQGAASLAQAISDAIAVLGRVLASAPSVMAVGFASLTYSSRTAEQWQDQTPDSVRYALGMDAAKLGLPPSVNLNAVAKASGTVDLPMRLTNEARGNTTTLSVVSTDGVSVPKAVPVRMAAYNATTGLYEVTVPSTTAEAPPLILTWTPASPPGNQNPSSTTPVVPKPVPVYEGATLTPVKATPETYPGVITLPEDLIIGFPADSGIKPIYVMFRDPRDVPGAATGKGQPVSGNWLGAASQGEGAPIPSQIADKLRGKTFKNWRDFREQFWIAVANDPELSKQFNPGSLAVMRDGGAPYVRESEQAGGRIKIEIHHKVRIADGGGVYNMGNLVAVTPKRHIEIHKGGK

SEQ ID NO: 277 - DNA, 2070 bp, Pseudomonas aeruginosa
  source 1..2070: mol_type = genomic DNA
atggctgtcaatgattacgaacctggttcgatggttattacacatgtgcagggtggtgggcgtgacataatccagtatattcctgctcgatcaagctacggtactccaccatttgtcccaccaggaccaagtccgtatgtcggtactggaatgcaggagtacaggaagctaagaagtacgcttgataagtcccattcagaactcaagaaaaacctgaaaaatgaaaccctgaaggaggttgatgaactcaagagtgaagcggggttgccaggtaaagcggtcagtgccaatgacatccgcgatgaaaagagtatcgttgatgcactcatggatgccaaagcaaaatcgctaaaggccattgaggatcgcccggccaatctttatacggcttcagactttcctcagaagtcagagtcgatgtaccagagtcagttgctggccagccgaaaattctatggagagttcctggatcgccatatgagtgagctggccaaagcgtacagcgccgatatctataaggcgcaaatcgctatcttgaaacaaacgtctcaagagctggagaataaagcccggtcattggaagcagaagcccagcgagccgctgctgaggtggaggcggactacaaggccaggaaggcaaatgtcgagaaaaaagtgcagtccgagcttgaccaggctgggaatgctttgcctcaactgaccaatccaacgccagagcagtggcttgaacgcgctactcaactggttacgcaggcgatcgccaataagaagaaattgcagactgcaaacaatgccttgattgccaaggcacccaatgcactggagaaacaaaaggcaacctacaacgccgatctcctagtggatgaaatcgccagcctgcaagcacggctggacaagctgaacgccgaaacggcaaggcgcaaggaaatcgctcgtcaagcggcgatcagggctgccaatacttatgccatgccagccaatggcagcgttgtcgccaccgccgcaggccggggtctgatccaggtcgcacaaggcgccgcatcccttgctcaagcgatctccgatgcgattgccgtcctgggccgggtcctggcttcagcaccctcggtgatggccgtgggctttgccagtctgacctactcctcccggactgccgagcaatggcaggaccaaacgcccgatagcgttcgttacgccctgggcatggatgccgctaaattggggcttcccccaagcgtaaacctgaacgcggttgcaaaagccagcggtaccgtcgatctgccgatgcgcctgaccaacgaggcacgaggcaacacgacgaccctttcggtggtcagcaccgatggtgtgagcgttccgaaagccgttccggtccggatggcggcctacaatgccacgacaggcctgtacgaggttacggttccctctacgaccgcagaagcgccgccactgatcctgacctggacgccggcgagtcctccaggaaaccagaacccttcgagtaccactccggtcgtaccgaagccggtgccggtatatgagggagcgacccttacaccggtgaaggctaccccggaaacctatcctggggtgattacactaccggaagacctgatcatcggcttcccggccgactcggggatcaagccgatctatgtgatgttcagggatccgcgggatgtacctggtgctgcgactggcaagggacagcccgtcagcggtaattggctcggcgccgcctctcaaggtgagggggctccaattccaagccagattgcggataaactacgtggtaagacattcaaaaactggcgggactttcgggaacaattctggatagctgtggctaatgatcctgagttaagtaaacagtttaatcctggtagtttagctgtaatgagagatggaggggctccttatgtcagagagtcagaacaggctggcgggagaataaagatcgaaatccaccacaaggttcgaatagcagatggaggcggcgtttacaatatggggaaccttgttgcagtaacgccaaaacgtcatatagaaatccacaagggagggaagtga

SEQ ID NO: 278 - protein, 47 aa, Ruminococcus gnavus
MRNDVLTLTNPMEEKELEQILGGGNGVLKTISHECNMNTWQFLFTCC

SEQ ID NO: 279 - DNA, 144 bp, Ruminococcus gnavus
  source 1..144: mol_type = genomic DNA
atgagaaatgacgtattaacattaacaaacccaatggaagagaacgaactggagcagatcttaggtggtggcaatggtgtgttaaaaacgattagccacgaatgcaatatgaacacatggcagttcctgtttacttgttgctaa

SEQ ID NO: 280 - protein, 55 aa, Lactobacillus sakei
MKNAKSLTIQEMKSITGGKYYGNGVSCNSHGCSVNWGQAWTCGVNHLANGGHGVC

SEQ ID NO: 281 - DNA, 168 bp, Lactobacillus sakei
  source 1..168: mol_type = genomic DNA
atgaaaaacgcaaaaagcctaacaattcaagaaatgaaatctattacaggtggtaaatactatggtaatggcgttagctgtaactctcacggctgttcagtaaattgggggcaagcatggacttgtggagtaaaccatctagctaatggcggtcatggagtttgttaa

SEQ ID NO: 282 - protein, 59 aa, Lactobacillus sakei; identical to SEQ ID NO: 68

SEQ ID NO: 283 - DNA, 180 bp, Lactobacillus sakei; identical to SEQ ID NO: 69
  source 1..180: mol_type = genomic DNA

SEQ ID NO: 284 - protein, 61 aa, Lactobacillus sakei
MEKFIELSLKEVTAITGGKYYGNGVHCGKHSCTVDWGTAIGNIGNNAAANWATGGNAGWNK

SEQ ID NO: 285 - DNA, 186 bp, Lactobacillus sakei
  source 1..186: mol_type = genomic DNA
atggaaaagtttattgaattatctttaaaagaagtaacagcaattacaggtggaaaatattatggtaacggtgtacactgtggaaaacattcatgtaccgtagactggggaacagctattggaaatatcggaaataatgcagctgcaaactgggccacaggcggaaacgctggctggaataaataa

SEQ ID NO: 286 - protein, 56 aa, Streptococcus salivarius
MKSTNNQSIAEIAAVNSLQEVSMEELDQIIGAGNGVVLTLTHECNLATWTKKLKCC

SEQ ID NO: 287 - DNA, 171 bp, Streptococcus salivarius
  source 1..171: mol_type = genomic DNA
atgaaatcaacaaataatcaaagtatcgcagaaattgcagcagtaaactcactacaagaagtaagtatggaggaactagaccaaattattggtgccggaaacggagtggttcttactcttactcatgaatgtaacctagcaacttggacaaaaaaactaaaatgttgctaa

SEQ ID NO: 288 - protein, 51 aa, Streptococcus pyogenes
  source 1..51: note = serotype M28
MSFMKNSKDILTNAIEEVSEKELMEVAGGKKGSGWFATITDDCPNSVFVCC

SEQ ID NO: 289 - DNA, 156 bp, Streptococcus pyogenes
  source 1..156: mol_type = genomic DNA; note = serotype M28
atgagttttatgaaaaattcaaaggatattttgactaatgctatcgaagaagtttctgaaaaagaacttatggaagtagctggtggtaaaaaaggttccggttggtttgcaactattactgatgactgtccgaactcagtattcgtttgttgttaa

SEQ ID NO: 290 - protein, 48 aa, Streptococcus salivarius
MKNSKDVLNNAIEEVSEKELMEVAGGKKGPGWIATITDDCPNSIFVCC

SEQ ID NO: 291 - DNA, 147 bp, Streptococcus salivarius
  source 1..147: mol_type = genomic DNA
atgaaaaactcaaaagatgttttgaacaatgctatcgaagaggtttctgaaaaagaacttatggaagtagctggtggtaaaaaaggtccaggttggattgcaactattactgatgactgtccaaactcaatattcgtttgttgttaa

SEQ ID NO: 292 - protein, 48 aa, Streptococcus salivarius
MKNSKDILNNAIEEVSEKELMEVAGGKRGSGWIATITDDCPNSVFVCC

SEQ ID NO: 293 - DNA, 147 bp, Streptococcus salivarius
  source 1..147: mol_type = genomic DNA
atgaaaaactcaaaagatattttgaacaatgctatcgaagaagtttctgaaaaagaacttatggaagtagctggtggtaaaagaggttcaggttggattgcaactattactgatgactgtccaaactcagtattcgtttgttgttaa

SEQ ID NO: 294 - protein, 62 aa, Staphylococcus aureus
MKSSFLEKDIEEQVTWFEEVSEQEFDDDIFGACSTNTFSLSDYWGNKGNWCTATHECMSWCK

SEQ ID NO: 295 - DNA, 189 bp, Staphylococcus aureus
  source 1..189: mol_type = genomic DNA
atgaaaagttcttttttagaaaaagatatagaagaacaagtgacatggttcgaggaagtttcagaacaagaatttgacgatgatatttttggagcttgtagtacaaacactttttctttgagtgactattggggtaataaaggaaattggtgtactgctactcacgaatgtatgtcttggtgtaaataa

SEQ ID NO: 296 - protein, 67 aa, Staphylococcus aureus
MKNELGKFLEENELELGKFSESDMLEITDDEVYAAGTPLALLGGAATGVIGYISNQTCPTTACTRAC

SEQ ID NO: 297 - DNA, 204 bp, Staphylococcus aureus
  source 1..204: mol_type = genomic DNA
atgaaaaatgaattaggtaagtttttagaagaaaacgaattagagttaggtaaattttcagaatcagacatgctagaaattactgatgatgaagtatatgcagctggaacacctttagccttattgggtggagctgccaccggggtgataggttatatttctaaccaaacatgtccaacaactgcttgtacacgcgcttgctag

SEQ ID NO: 298 - protein, 46 aa, Streptococcus pyogenes
MNNTIKDFDLDLKTNKKDTATPYVGSRYLCTPGSCWKLVCFTTTVK

SEQ ID NO: 299 - DNA, 141 bp, Streptococcus pyogenes
  source 1..141: mol_type = genomic DNA
atgaataacacaattaaagactttgatctcgatttgaaaacaaataaaaaagacactgctacaccttatgttggtagccgttacctatgtacccctggttcttgttggaaattagtttgctttacaacaactgttaaataa

SEQ ID NO: 300 - protein, 51 aa, Streptococcus pyogenes
MEKNNEVINSIQEVSLEELDQIIGAGKNGVFKTISHECHLNTWAFLATCCS

SEQ ID NO: 301 - DNA, 156 bp, Streptococcus pyogenes
  source 1..156: mol_type = genomic DNA
atggaaaaaaataatgaagtaatcaactctattcaagaagttagtcttgaagaactcgatcaaattatcggtgctggaaaaaatggtgtgtttaaaacaatttctcatgagtgtcatttgaatacatgggcattccttgctacttgttgttcataa

SEQ ID NO: 302 - protein, 51 aa, Streptococcus pyogenes
  source 1..51: note = serotype M49
MTKEHEIINSIQEVSLEELDQIIGAGKNGVFKTISHECHLNTWAFLATCCS

SEQ ID NO: 303 - DNA, 156 bp, Streptococcus pyogenes
  source 1..156: mol_type = genomic DNA; note = serotype M49
atggaaaaaaataatgaagtaatcaactctattcaagaagttagtcttgaagaactcgatcaaattatcggtgctggaaaaaatggtgtgtttaaaacaatttctcatgagtgtcatttgaatacatgggcattccttgctacttgttgctcataa

SEQ ID NO: 304 - protein, 56 aa, Bacillus subtilis
MEKLFKEVKLEELENQKGSGLGKAQCAALWLQCASGGTIGCGGGAVACQNYRQFCR

SEQ ID NO: 305 - DNA, 171 bp, Bacillus subtilis
  source 1..171: mol_type = genomic DNA
atggaaaagctatttaaagaagttaaactagaggaactcgaaaaccaaaaaggtagtggattaggaaaagctcagtgtgctgcgttgtggctacaatgtgctagtggcggtacaattggttgtggtggcggagctgttgcttgtcaaaactatcgtcaattctgcagataa

SEQ ID NO: 306 - protein, 56 aa, Bacillus subtilis
MSKFDDFDLDVVKVSKQDSKITPQWKSESLCTPGCVTGALQTCFLQTLTCNCKISK

SEQ ID NO: 307 - DNA, 171 bp, Bacillus subtilis
  source 1..171: mol_type = genomic DNA
atgtcaaagttcgatgatttcgatttggatgttgtgaaagtctctaaacaagactcaaaaatcactccgcaatggaaaagtgaatcactttgtacaccaggatgtgtaactggtgcattgcaaacttgcttccttcaaacactaacttgtaactgcaaaatctctaaataa

SEQ ID NO: 308 - protein, 50 aa, Bacillus subtilis
MKLPVQQVYSVYGGKDLPKGHSHSTMPFLSKLQFLTKIYLLDIHTQPFFI

SEQ ID NO: 309 - DNA, 153 bp, Bacillus subtilis
  source 1..153: mol_type = genomic DNA
ttgaaattgccggtgcaacaggtctattcggtctatgggggtaaggatctcccaaaagggcatagtcattctactatgccctttttaagtaaattacaatttttaactaaaatctacctcttggatatacatacacaaccgtttttcatttga

SEQ ID NO: 310 - protein, 43 aa, Bacillus subtilis
MKKAVIVENKGCATCSIGAACLVDGPIPDFEIAGATGLFGLWG

SEQ ID NO: 311 - DNA, 132 bp, Bacillus subtilis
  source 1..132: mol_type = genomic DNA
atgaaaaaagctgtcattgtagaaaacaaaggttgtgcaacatgctcgatcggagccgcttgtctagtggacggtcctatccctgattttgaaattgccggtgcaacaggtctattcggtctatgggggtaa

SEQ ID NO: 312 - protein, 58 aa, Streptococcus thermophilus; identical to SEQ ID NO: 24

SEQ ID NO: 313 - DNA, 177 bp, Streptococcus thermophilus
  source 1..177: mol_type = genomic DNA
atgatgaatgctactgaaaaccaaatttttgttgagactgtgagtgaccaagaattagaaatgttaattggtggtgcagatcgtggatggattaagactttaacaaaagattgtccaaatgtaatttcttcaatttgtgcaggtacaattattacagcctgtaaaaattgtgcttaa

SEQ ID NO: 314 - protein, 64 aa, Streptococcus thermophilus
MKQYNGFEVLHELDLANVTGGQINWGSVVGHCIGGAIIGGAFSGGAAAGVGCLVGSGKAIINGL

SEQ ID NO: 315 - DNA, 195 bp, Streptococcus thermophilus
  source 1..195: mol_type = genomic DNA
atgaagcagtataatggttttgaggttctacatgaacttgacttagcaaatgtaactggcggtcaaattaattggggatcagttgtaggacactgtataggtggagctattatcggaggtgcattttcaggaggtgcagcggctggagtaggatgccttgttgggagcggaaaggcaatcataaatggattataa

SEQ ID NO: 316 - protein, 85 aa, Streptococcus thermophilus
MNTITICKFDVLDAELLSTVEGGYSGKDCLKDMGGYALAGAGSGALWGAPAGGVGALPGAFVGAHVGAIAGGFACMGGMIGNKFN

SEQ ID NO: 317 - DNA, 258 bp, Streptococcus thermophilus
  source 1..258: mol_type = genomic DNA
atgaatacaataactatttgtaaatttgatgttttagatgctgaacttctttcgacagttgagggtggatactctggtaaggattgtttaaaagacatgggaggatatgcattggcaggagctggaagtggagctctgtggggagctccagcaggaggtgttggagcacttccaggtgcatttgtcggagctcatgttggggcaattgcaggaggctttgcatgtatgggtggaatgattggtaataagtttaactaa

SEQ ID NO: 318 - protein, 52 aa, Bacillus cereus
MSEIKKALNTLEIEDFDAIEMVDVDAMPENEALEIMGASCTTCVCTCSCCTT

SEQ ID NO: 319 - DNA, 159 bp, Bacillus cereus
  source 1..159: mol_type = genomic DNA
atgagtgaaattaaaaaagcattaaatacgcttgaaattgaagattttgatgcaattgaaatggttgatgttgatgctatgccagaaaacgaagcgcttgaaattatgggagcgtcatgtacgacatgcgtatgtacatgcagttgttgtacaacttga

SEQ ID NO: 320 - protein, 47 aa, Bacillus cereus
MEVMNNALITKVDEEIGGNAACVIGCIGSCVISEGIGSLVGTAFTLG

SEQ ID NO: 321 - DNA, 144 bp, Bacillus cereus
  source 1..144: mol_type = genomic DNA
atggaagttatgaacaatgctttaattacaaaagtagatgaggagattggaggaaacgctgcttgtgtaattggttgtattggcagttgcgtaattagtgaaggaattggttcacttgtaggaacagcatttactttaggttaa

SEQ ID NO: 322 - protein, 49 aa, Bacillus cereus
MEVLNKQNVNIIPESEEVGGWVACVGACGTVCLASGGVGTEFAAASYFL

SEQ ID NO: 323 - DNA, 150 bp, Bacillus cereus
  source 1..150: mol_type = genomic DNA
atggaagttttaaacaaacaaaatgtaaatattattccagaatctgaagaagtaggtggatgggtagcatgtgttggagcatgtggtacagtatgtcttgctagtggtggtgttggaacagagtttgcagctgcatcttatttcctataa

SEQ ID NO: 324 - protein, 40 aa, Bacillus thuringiensis
METPVVQPRDWTCWSCLVCAACSVELLNLVTAATGASTAS

SEQ ID NO: 325 - DNA, 123 bp, Bacillus thuringiensis
  source 1..123: mol_type = genomic DNA
atggaaacaccagtagtacaaccaagggattggacttgttggagttgcttagtatgtgcagcatgttctgtggaattattaaatttagttactgcggcaacaggggctagtactgcaagctaa

SEQ ID NO: 326 - protein, 42 aa, Rhizobium leguminosarum
  source 1..42: note = bv. trifolii
MDNKVAKNVEVKKGSIKATFKAAVLKSKTKVDIGGSRQGCVA

SEQ ID NO: 327 - DNA, 129 bp, Rhizobium leguminosarum
  source 1..129: mol_type = genomic DNA; note = bv. trifolii
atggataacaaggttgcgaagaatgtcgaagtgaagaagggctccatcaaggcgaccttcaaggctgctgttctgaagtcgaagacgaaggtcgacatcggaggtagccgtcagggctgcgtcgcttaa

SEQ ID NO: 328 - protein, 70 aa, Streptococcus uberis
MNTIEKFENIKLFSLKKIIGGKTVNYGNGLYCNQKKCWVNWSETATTIVNNSIMNGLTGGNAGWHSGGRA

SEQ ID NO: 329 - DNA, 213 bp, Streptococcus uberis
  source 1..213: mol_type = genomic DNA
atgaatacaattgaaaaatttgaaaatattaaacttttttcactaaagaaaattatcggtggcaaaactgtaaattatggtaatggcctttattgtaaccaaaaaaaatgctgggtaaactggtcagaaactgctacaacaatagtaaataattccatcatgaacgggctcacaggtggtaatgcgggttggcactcaggcgggagagcataa

SEQ ID NO: 330 - protein, 76 aa, Streptococcus uberis
MDILLELAGYTGIASGTAKKVVDAIDKGAAAFVIISIISTVISAGALGAVSASADFIILTVKNYISRNLKAQAVIW

SEQ ID NO: 331 - DNA, 231 bp, Streptococcus uberis
  source 1..231: mol_type = genomic DNA
atggacattttattagaactcgcaggatatactgggatagcctcaggtactgcaaaaaaagttgttgatgccattgataaaggagctgcagcctttgttattatttcaattatctcaacagtaattagtgcgggagcattgggagcagtttcagcctcagctgattttattattttaactgtaaaaaattacattagtagaaatttaaaagcacaagctgtcatttggtaa

SEQ ID NO: 332 - protein, 64 aa, Clostridium perfringens
MDSELFKLMATQGAFAILFSYLLFYVLKENSKREDKYQNIIEELTELLPKIKEDVEDIKEKLNK

SEQ ID NO: 333 - DNA, 195 bp, Clostridium perfringens
  source 1..195: mol_type = genomic DNA
atggatagtgaattatttaagttaatggcaacacaaggagcctttgcaatattattttcgtatttattgttttatgttttaaaagagaatagtaaaagagaagataagtatcaaaatataatagaggagcttacagaattattgccaaaaataaaagaagatgtagaagatataaaagaaaaacttaataaatag

SEQ ID NO: 334 - protein, 47 aa, Micrococcus sp.
  source 1..47: note = Micrococcus varians
MTNAFQALDEVTDAELDAILGGGSGVIPTISHECHMNSFQFVFTCCS

SEQ ID NO: 335 - DNA, 144 bp, Micrococcus sp.
  source 1..144: mol_type = genomic DNA; note = Micrococcus varians
atgacgaacgcatttcaggcactggacgaagtcacggacgccgagctcgacgccatccttggcgggggcagtggtgttattcccacgatcagccacgagtgccacatgaactccttccagttcgtgttcacctgctgctcctga

SEQ ID NO: 336 - protein, 285 aa, Streptococcus equi
  source 1..285: note = subsp. zooepidemicus
MKRIFFAFLSLCLFIFGTQTVSAATYTRPLDTGNITTGFNGYPGHVGVDYAVPVGTPVRAVANGTVKFAGNGANHPWMLWMAGNCVLIQHADGMHTGYAHLSKISVSTDSTVKQGQIIGYTGATGQVTGPHLHFEMLPANPNWQNGFSGRIDPTGYIANAPVFNGTTPTEPTTPTTNLKIYKVDDLQKINGIWQVRNNILVPTDFTWVDNGIAADDVIEVTSNGTRTSDQVLQKGGYFVINPNNVKSVGTPMKGSGGLSWAQVNFTTGGNVWLNTTSKDNLLYGK

SEQ ID NO: 337 - DNA, 858 bp, Streptococcus equi
  source 1..858: mol_type = genomic DNA; note = subsp. zooepidemicus
atgaaacgtatattttttgctttcttaagtttatgcttatttatattcggaacacaaacggtatctgcagctacttatactcggccattagatacgggaaatatcactacagggtttaacggataccctggtcatgttggagtcgattatgcagtacccgttggaactccggttagagcagttgcaaatggtacagtcaaatttgcaggtaatggggctaatcacccatggatgctttggatggctggaaactgtgttctaattcaacatgctgacgggatgcatactggatatgcacacttatcaaaaatttcagttagcacagatagtacagttaaacaaggacaaatcataggttatactggtgccaccggccaagttaccggtccacatttgcattttgaaatgttgccagcaaatcctaactggcaaaatggtttttctggaagaatagatccaaccggatacatcgctaatgcccctgtatttaatggaacaacacctacagaacctactactcctacaacaaatttaaaaatctataaagttgatgatttacaaaaaattaatggtatttggcaagtaagaaataacatacttgtaccaactgatttcacatgggttgataatggaattgcagcagatgatgtaattgaagtaactagcaatggaacaagaacctctgaccaagttcttcaaaaaggtggttattttgtcatcaatcctaataatgttaaaagtgttggaactccgatgaaaggtagtggtggtctatcttgggctcaagtaaactttacaacaggtggaaatgtctggttaaatactactagcaaagacaacttactttacggaaaataa

SEQ ID NO: 338 - protein, 45 aa, Myxococcus fulvus
ANCSCSTASDYCPILTFCTTGTACSYTPTGCGTGWVYCACNGNFY

SEQ ID NO: 339 - DNA, 135 bp, synthetic construct
  misc_feature 1..135: note = Polynucleotide encoding the polypeptide of SEQ IDNO: 338
  source 1..135: mol_type = other DNA
gcgaactgcagctgcagcaccgcgagcgattattgcccgattctgaccttttgcaccaccggcaccgcgtgcagctataccccgaccggctgcggcaccggctgggtgtattgcgcgtgcaacggcaacttttat

SEQ ID NO: 340 - protein, 19 aa, Streptomyces griseoluteus
CANSCSYGPLTWSCDGNTK

SEQ ID NO: 341 - DNA, 57 bp, synthetic construct
  misc_feature 1..57: note = Polynucleotide encoding the polypeptide of SEQ IDNO: 340
  source 1..57: mol_type = other DNA
tgcgcgaacagctgcagctatggcccgctgacctggagctgcgatggcaacaccaaa

SEQ ID NO: 342 - protein, 19 aa, unidentified
  source 1..19: note = Streptoverticillium griseoverticillatum
CKQSCSFGPFTFVCDGNTK

SEQ ID NO: 343 - DNA, 57 bp, synthetic construct
  misc_feature 1..57: note = Polynucleotide encoding the polypeptide of SEQ IDNO: 342
  source 1..57: mol_type = other DNA
tgcaaacagagctgcagctttggcccgtttacctttgtgtgcgatggcaacaccaaa

SEQ ID NO: 344 - protein, 7 aa, Carnobacterium sp.
GSEIQPR

SEQ ID NO: 345 - DNA, 21 bp, synthetic construct
  misc_feature 1..21: note = Polynucleotide encoding the polypeptide of SEQ IDNO: 344
  source 1..21: mol_type = other DNA
ggcagcgaaattcagccgcgc

SEQ ID NO: 346 - protein, 39 aa, Lactococcus lactis
  source 1..39: note = subsp. lactis
GTWDDIGQGIGRVAYWVGKAMGNMSDVNQASRINRKKKH

SEQ ID NO: 347 - DNA, 117 bp, synthetic construct
  misc_feature 1..117: note = Polynucleotide encoding the polypeptide of SEQ IDNO: 346
  source 1..117: mol_type = other DNA
ggcacctgggatgatattggccagggcattggccgcgtggcgtattgggtgggcaaagcgatgggcaacatgagcgatgtgaaccaggcgagccgcattaaccgcaaaaaaaaacat

SEQ ID NO: 348 - protein, 35 aa, Lactococcus lactis
  source 1..35: note = subsp. lactis
KKWGWLAWVDPAYEFIKGFGKGAIKEGNKDKWKNI

SEQ ID NO: 349 - DNA, 105 bp, synthetic construct
  misc_feature 1..105: note = Polynucleotide encoding the polypeptide of SEQ IDNO: 348
  source 1..105: mol_type = other DNA
aaaaaatggggctggctggcgtgggtggatccggcgtatgaatttattaaaggctttggcaaaggcgcgattaaagaaggcaacaaagataaatggaaaaacatt

SEQ ID NO: 350 - protein, 19 aa, Streptomyces sp.
CVQSCSFGPLTWSCDGNTK

SEQ ID NO: 351 - DNA, 57 bp, synthetic construct
  misc_feature 1..57: note = Polynucleotide encoding the polypeptide of SEQ IDNO: 350
  source 1..57: mol_type = other DNA
tgcgtgcagagctgcagctttggcccgctgacctggagctgcgatggcaacaccaaa

SEQ ID NO: 352 - protein, 19 aa, Actinoplanes sp.
  source 1..19: note = Actinoplanes liguriae
SSGWVCTLTIECGTVICAC

SEQ ID NO: 353 - DNA, 57 bp, synthetic construct
  misc_feature 1..57: note = Polynucleotide encoding the polypeptide of SEQ IDNO: 352
  source 1..57: mol_type = other DNA
agcagcggctgggtgtgcaccctgaccattgaatgcggcaccgtgatttgcgcgtgc

SEQ ID NO: 354 - protein, 38 aa, Lactobacillus curvatus
  VARIANT 37: note = Xaa = any amino acid
  VARIANT 34: note = Xaa = any amino acid
YTAKQCLQAIGSCGIAGTGAGAAGGPAGAFVGAXVVXI

SEQ ID NO: 355 - DNA, 114 bp, synthetic construct
  misc_feature 1..114: note = Polynucleotide encoding the polypeptide of SEQ IDNO: 354
  misc_feature 100..102: note = nnn = any amino acid-coding triplet
  misc_feature 109..111: note = nnn = any amino acid-coding triplet
  source 1..114: mol_type = other DNA
tataccgcgaaacagtgcctgcaggcgattggcagctgcggcattgcgggcaccggcgcgggcgcggcgggcggcccggcgggcgcgtttgtgggcgcgnnngtggtgnnnatt

SEQ ID NO: 356 - protein, 42 aa, Lactobacillus sakei
  VARIANT 32: note = Xaa = any amino acid
TKYYGNGVYCNSKKCWVDWGQAAGGIGQTVVXGWLGGAIPGK

SEQ ID NO: 357 - DNA, 126 bp, synthetic construct
  misc_feature 1..126: note = Polynucleotide encoding the polypeptide of SEQ IDNO: 356
  misc_feature 94..96: note = nnn = any amino acid-coding triplet
  source 1..126: mol_type = other DNA
accaaatattatggcaacggcgtgtattgcaacagcaaaaaatgctgggtggattggggccaggcggcgggcggcattggccagaccgtggtgnnnggctggctgggcggcgcgattccgggcaaa

SEQ ID NO: 358 - protein, 22 aa, Streptococcus mutans
FKSWSFCTPGCAKTGSFNSYCC

SEQ ID NO: 359 - DNA, 132 bp, synthetic construct
  misc_feature 1..132: note = Polynucleotide encoding the polypeptide of SEQ IDNO: 358
  source 1..132: mol_type = other DNA
tttaaaagctggagcttttgcaccccgggctgcgcgaaaaccggcagctttaacagctattgctgctttaaaagctggagcttttgcaccccgggctgcgcgaaaaccggcagctttaacagctattgctgc

SEQ ID NO: 360 - protein, 43 aa, Enterococcus mundtii
KYYGNGVSCNKKGCSVDWGKAIGIIGNNSAANLATGGAAGWSK

SEQ ID NO: 361 - DNA, 129 bp, synthetic construct
  misc_feature 1..129: note = Polynucleotide encoding the polypeptide of SEQ IDNO: 360
  source 1..129: mol_type = other DNA
aaatattatggcaacggcgtgagctgcaacaaaaaaggctgcagcgtggattggggcaaagcgattggcattattggcaacaacagcgcggcgaacctggcgaccggcggcgcggcgggctggagcaaa

SEQ ID NO: 362 - protein, 41 aa, Lactobacillus sakei
  VARIANT 9: note = Xaa = any amino acid
  VARIANT 14: note = Xaa = any amino acid
  VARIANT 33: note = Xaa = any amino acid
  VARIANT 37: note = Xaa = any amino acid
KYYGNGVHXGKHSXTVDWGTAIGNIGNNAAANXATGXNAGG

SEQ ID NO: 363 - DNA, 123 bp, synthetic construct
  misc_feature 1..123: note = Polynucleotide encoding the polypeptide of SEQ IDNO: 362
  misc_feature 25..27: note = nnn = any amino acid-coding triplet
  misc_feature 40..42: note = nnn = any amino acid-coding triplet
  misc_feature 97..99: note = nnn = any amino acid-coding triplet
  misc_feature 109..111: note = nnn = any amino acid-coding triplet
  source 1..123: mol_type = other DNA
aaatattatggcaacggcgtgcatnnnggcaaacatagcnnnaccgtggattggggcaccgcgattggcaacattggcaacaacgcggcggcgaacnnngcgaccggcnnnaacgcgggcggc

SEQ ID NO: 364 - protein, 31 aa, Lactobacillus paracasei
GMSGYIQGIPDFLKGYLHGISAANKHKKGRL

SEQ ID NO: 365 - DNA, 93 bp, synthetic construct
  misc_feature 1..93: note = Polynucleotide encoding the polypeptide of SEQ IDNO: 364
  source 1..93: mol_type = other DNA
ggcatgagcggctatattcagggcattccggattttctgaaaggctatctgcatggcattagcgcggcgaacaaacataaaaaaggccgcctg

SEQ ID NO: 366 - protein, 31 aa, Leuconostoc mesenteroides
KGKGFWSWASKATSWLTGPQQPGSPLLKKHR

SEQ ID NO: 367 - DNA, 93 bp, synthetic construct
  misc_feature 1..93: note = Polynucleotide encoding the polypeptide of SEQ IDNO: 366
  source 1..93: mol_type = other DNA
aaaggcaaaggcttttggagctgggcgagcaaagcgaccagctggctgaccggcccgcagcagccgggcagcccgctgctgaaaaaacatcgc

SEQ ID NO: 368 - protein, 43 aa, Leuconostoc mesenteroides
KNYGNGVHCTKKGCSVDWGYAWTNIANNSVMNGLTGGNAGWHN

SEQ ID NO: 369 - DNA, 129 bp, synthetic construct
  misc_feature 1..129: note = Polynucleotide encoding the polypeptide of SEQ IDNO: 368
  source 1..129: mol_type = other DNA
aaaaactatggcaacggcgtgcattgcaccaaaaaaggctgcagcgtggattggggctatgcgtggaccaacattgcgaacaacagcgtgatgaacggcctgaccggcggcaacgcgggctggcataac

SEQ ID NO: 370 - protein, 47 aa, Bacillus subtilis
AIKLVQSPNGNFAASFVLDGTKWIFKSKYYDSSKGYWVGIYEVWDRK

SEQ ID NO: 371 - DNA, 141 bp, synthetic construct
  misc_feature 1..141: note = Polynucleotide encoding the polypeptide of SEQ IDNO: 370
  source 1..141: mol_type = other DNA
gcgattaaactggtgcagagcccgaacggcaactttgcggcgagctttgtgctggatggcaccaaatggatttttaaaagcaaatattatgatagcagcaaaggctattgggtgggcatttatgaagtgtgggatcgcaaa

SEQ ID NO: 372 - protein, 12 aa, Bacillus licheniformis
  VARIANT 7: note = Xaa = any amino acid
ISLEICXIFHDN

SEQ ID NO: 373 - DNA, 36 bp, synthetic construct
  misc_feature 1..36: note = Polynucleotide encoding the polypeptide of SEQ IDNO: 372
  misc_feature 19..21: note = nnn = any amino acid-coding triplet
  source 1..36: mol_type = other DNA
attagcctggaaatttgcnnnatttttcatgataac

SEQ ID NO: 374 - protein, 37 aa, Lactococcus lactis
  source 1..37: note = subsp. lactis
TSYGNGVHCNKSKCWIDVSELETYKAGTVSNPKDILW

SEQ ID NO: 375 - DNA, 111 bp, synthetic construct
  misc_feature 1..111: note = Polynucleotide encoding the polypeptide of SEQ IDNO: 374
  source 1..111: mol_type = other DNA
accagctatggcaacggcgtgcattgcaacaaaagcaaatgctggattgatgtgagcgaactggaaacctataaagcgggcaccgtgagcaacccgaaagatattctgtgg

SEQ ID NO: 376 - protein, 9 aa, Serratia plymuthica
DYHHGVRVL

SEQ ID NO: 377 - DNA, 27 bp, synthetic construct
  misc_feature 1..27: note = Polynucleotide encoding the polypeptide of SEQ IDNO: 376
  source 1..27: mol_type = other DNA
gattatcatcatggcgtgcgcgtgctg

SEQ ID NO: 378 - protein, 15 aa, Halobacterium sp.
DIDITGCSACKYAAG

SEQ ID NO: 379 - DNA, 45 bp, synthetic construct
  misc_feature 1..45: note = Polynucleotide encoding the polypeptide of SEQ IDNO: 378
  source 1..45: mol_type = other DNA
gatattgatattaccggctgcagcgcgtgcaaatatgcggcgggc

SEQ ID NO: 380 - protein, 12 aa, Bacillus subtilis
  VARIANT 1: note = Xaa = any amino acid
  VARIANT 2: note = Xaa = any amino acid
  VARIANT 6: note = Xaa = any amino acid
XXKEIXHIFHDN

SEQ ID NO: 381 - DNA, 36 bp, synthetic construct
  misc_feature 1..36: note = Polynucleotide encoding the polypeptide of SEQ IDNO: 380
  misc_feature 1..3: note = nnn = any amino acid-coding triplet
  misc_feature 4..6: note = nnn = any amino acid-coding triplet
  misc_feature 16..18: note = nnn = any amino acid-coding triplet
  source 1..36: mol_type = other DNA
nnnnnnaaagaaattnnncatatttttcatgataac

SEQ ID NO: 382 - protein, 12 aa, Lactobacillus curvatus
TPVVNPPFLQQT

SEQ ID NO: 383 - DNA, 36 bp, synthetic construct
  misc_feature 1..36: note = Polynucleotide encoding the polypeptide of SEQ IDNO: 382
  source 1..36: mol_type = other DNA
accccggtggtgaacccgccgtttctgcagcagacc

SEQ ID NO: 384 - protein, 10 aa, Lactobacillus curvatus
  VARIANT 10: note = Xaa = any amino acid
VAPFPEQFLX

SEQ ID NO: 385 - DNA, 30 bp, synthetic construct
  misc_feature 1..30: note = Polynucleotide encoding the polypeptide of SEQ IDNO: 384
  misc_feature 28..30: note = nnn = any amino acid-coding triplet
  source 1..30: mol_type = other DNA
gtggcgccgtttccggaacagtttctgnnn

SEQ ID NO: 386 - protein, 9 aa, Lactobacillus curvatus
NIPQLTPTP

SEQ ID NO: 387 - DNA, 27 bp, synthetic construct
  misc_feature 1..27: note = Polynucleotide encoding the polypeptide of SEQ IDNO: 386
  source 1..27: mol_type = other DNA
aacattccgcagctgaccccgaccccg

SEQ ID NO: 388 - protein, 18 aa, Bacillus thuringiensis
  VARIANT 4: note = Xaa = any amino acid; note = Xaa = any amino acid
  source 1..18: note = subsp. entomocidus
  VARIANT 7: note = Xaa = any amino acid
  VARIANT 10: note = Xaa = any amino acid
DWTXWSXLVXAACSVELL

SEQ ID NO: 389 - DNA, 54 bp, synthetic construct
  misc_feature 1..54: note = Polynucleotide encoding the polypeptide of SEQ IDNO: 388
  misc_feature 10..12: note = nnn = any amino acid-coding triplet
  misc_feature 19..21: note = nnn = any amino acid-coding triplet
  misc_feature 28..30: note = nnn = any amino acid-coding triplet
  source 1..54: mol_type = other DNA
gattggaccnnntggagcnnnctggtgnnngcggcgtgcagcgtggaactgctg

SEQ ID NO: 390 - protein, 30 aa, Lactobacillus curvatus
AYPGNGVHCGKYSCTVDKQTAIGNIGNNAA

SEQ ID NO: 391 - DNA, 90 bp, synthetic construct
  misc_feature 1..90: note = Polynucleotide encoding the polypeptide of SEQ IDNO: 390
  source 1..90: mol_type = other DNA
gcgtatccgggcaacggcgtgcattgcggcaaatatagctgcaccgtggataaacagaccgcgattggcaacattggcaacaacgcggcg

SEQ ID NO: 392 - protein, 43 aa, Carnobacterium divergens
TKYYGNGVYCNSKKCWVDWGTAQGCIDVVIGQLGGGIPGKGKC

SEQ ID NO: 393 - DNA, 129 bp, synthetic construct
  misc_feature 1..129: note = Polynucleotide encoding the polypeptide of SEQ IDNO: 392
  source 1..129: mol_type = other DNA
accaaatattatggcaacggcgtgtattgcaacagcaaaaaatgctgggtggattggggcaccgcgcagggctgcattgatgtggtgattggccagctgggcggcggcattccgggcaaaggcaaatgc

SEQ ID NO: 394 - protein, 62 aa, Enterococcus sp.
NRWYCNSAAGGVGGAAVCGLAGYVGEAKENIAGEVRKGWGMAGGFTHNKACKSFPGSGWASG

SEQ ID NO: 395 - DNA, 186 bp, synthetic construct
  misc_feature 1..186: note = Polynucleotide encoding the polypeptide of SEQ IDNO: 394
  source 1..186: mol_type = other DNA
aaccgctggtattgcaacagcgcggcgggcggcgtgggcggcgcggcggtgtgcggcctggcgggctatgtgggcgaagcgaaagaaaacattgcgggcgaagtgcgcaaaggctggggcatggcgggcggctttacccataacaaagcgtgcaaaagctttccgggcagcggctgggcgagcggc

SEQ ID NO: 396 - protein, 39 aa, Enterococcus faecium
TTKNYGNGVCNSVNWCQCGNVWASCNLATGCAAWLCKLA

SEQ ID NO: 397 - DNA, 117 bp, synthetic construct
  misc_feature 1..117: note = Polynucleotide encoding the polypeptide of SEQ IDNO: 396
  source 1..117: mol_type = other DNA
accaccaaaaactatggcaacggcgtgtgcaacagcgtgaactggtgccagtgcggcaacgtgtgggcgagctgcaacctggcgaccggctgcgcggcgtggctgtgcaaactggcg

SEQ ID NO: 398 - protein, 30 aa, Paenibacillus polymyxa
ASIIKTTIKVSKAVCKTLTCICTGSCSNCK

SEQ ID NO: 399 - DNA, 90 bp, synthetic construct
  misc_feature 1..90: note = Polynucleotide encoding the polypeptide of SEQ IDNO: 398
  source 1..90: mol_type = other DNA
gcgagcattattaaaaccaccattaaagtgagcaaagcggtgtgcaaaaccctgacctgcatttgcaccggcagctgcagcaactgcaaa

SEQ ID NO: 400 - protein, 31 aa, Staphylococcus epidermidis
SASIVKTTIKASKKLCRGFTLTCGCHFTGKK

SEQ ID NO: 401 - DNA, 93 bp, synthetic construct
  misc_feature 1..93: note = Polynucleotide encoding the polypeptide of SEQ IDNO: 400
  source 1..93: mol_type = other DNA
agcgcgagcattgtgaaaaccaccattaaagcgagcaaaaaactgtgccgcggctttaccctgacctgcggctgccattttaccggcaaaaaa

SEQ ID NO: 402 - protein, 43 aa, Enterococcus faecium
KYYGNGVSCNKKGCSVDWGKAIGIIGNNAAANLTTGGKAAWAC

SEQ ID NO: 403 - DNA, 129 bp, synthetic construct
  misc_feature 1..129: note = Polynucleotide encoding the polypeptide of SEQ IDNO: 402
  source 1..129: mol_type = other DNA
aaatattatggcaacggcgtgagctgcaacaaaaaaggctgcagcgtggattggggcaaagcgattggcattattggcaacaacgcggcggcgaacctgaccaccggcggcaaagcggcgtgggcgtgc

SEQ ID NO: 404 - protein, 39 aa, Paenibacillus polymyxa
ATYYGNGLYCNKQKHYTWVDWNKASREIGKITVNGWVQH

SEQ ID NO: 405 - DNA, 117 bp, synthetic construct
  misc_feature 1..117: note = Polynucleotide encoding the polypeptide of SEQ IDNO: 404
  source 1..117: mol_type = other DNA
gcgacctattatggcaacggcctgtattgcaacaaacagaaacattatacctgggtggattggaacaaagcgagccgcgaaattggcaaaattaccgtgaacggctgggtgcagcat

SEQ ID NO: 406 - protein, 35 aa, Bacillus circulans
VNYGNGVSCSKTKCSVNWGIITHQAFRVTSGVASG

SEQ ID NO: 407 - DNA, 105 bp, synthetic construct
  misc_feature 1..105: note = Polynucleotide encoding the polypeptide of SEQ IDNO: 406
  source 1..105: mol_type = other DNA
gtgaactatggcaacggcgtgagctgcagcaaaaccaaatgcagcgtgaactggggcattattacccatcaggcgtttcgcgtgaccagcggcgtggcgagcggc

SEQ ID NO: 408 - protein, 30 aa, Paenibacillus polymyxa
FVYGNGVTSILVQAQFLVNGQRRFFYTPDK

SEQ ID NO: 409 - DNA, 90 bp, synthetic construct
  misc_feature 1..90: note = Polynucleotide encoding the polypeptide of SEQ IDNO: 408
  source 1..90: mol_type = other DNA
tttgtgtatggcaacggcgtgaccagcattctggtgcaggcgcagtttctggtgaacggccagcgccgctttttttataccccggataaa

SEQ ID NO: 410 - protein, 13 aa, Lactobacillus rhamnosus
AVPAVRKTNETLD

SEQ ID NO: 411 - DNA, 39 bp, synthetic construct
  misc_feature 1..39: note = Polynucleotide encoding the polypeptide of SEQ IDNO: 410
  source 1..39: mol_type = other DNA
gcggtgccggcggtgcgcaaaaccaacgaaaccctggat

SEQ ID NO: 412 - protein, 69 aa, Bacillus licheniformis
MKNSAAREAFKGANHPAGMVSEEELKALVGGNDVNPETTPATTSSWTCITAGVTVSASLCPTTKCTSRC

SEQ ID NO: 413 - DNA, 207 bp, synthetic construct
  misc_feature 1..207: note = Polynucleotide encoding the polypeptide of SEQ IDNO: 412
  source 1..207: mol_type = other DNA
atgaaaaacagcgcggcgcgcgaagcgtttaaaggcgcgaaccatccggcgggcatggtgagcgaagaagaactgaaagcgctggtgggcggcaacgatgtgaacccggaaaccaccccggcgaccaccagcagctggacctgcattaccgcgggcgtgaccgtgagcgcgagcctgtgcccgaccaccaaatgcaccagccgctgc

SEQ ID NO: 414 - protein, 36 aa, Lactobacillus plantarum
KYYGNGLSCSKKGCTVNWGQAFSCGVNRVATAGHGK

SEQ ID NO: 415 - DNA, 108 bp, synthetic construct
  misc_feature 1..108: note = Polynucleotide encoding the polypeptide of SEQ IDNO: 414
  source 1..108: mol_type = other DNA
aaatattatggcaacggcctgagctgcagcaaaaaaggctgcaccgtgaactggggccaggcgtttagctgcggcgtgaaccgcgtggcgaccgcgggccatggcaaa

SEQ ID NO: 416 - protein, 24 aa, Lactobacillus acidophilus
GNPKVAHCASQIGRSTAWGAVSGA

SEQ ID NO: 417 - DNA, 72 bp, synthetic construct
  misc_feature 1..72: note = Polynucleotide encoding the polypeptide of SEQ IDNO: 416
  source 1..72: mol_type = other DNA
ggcaacccgaaagtggcgcattgcgcgagccagattggccgcagcaccgcgtggggcgcggtgagcggcgcg

SEQ ID NO: 418 - protein, 40 aa, Enterococcus faecalis; identical to SEQ ID NO: 1

SEQ ID NO: 419 - DNA, 120 bp, synthetic construct
  misc_feature 1..120: note = Polynucleotide encoding the polypeptide of SEQ IDNO: 418
  source 1..120: mol_type = other DNA
tggctgccgccggcgggcctgctgggccgctgcggccgctggtttcgcccgtggctgctgtggctgcagagcggcgcgcagtataaatggctgggcaacctgtttggcctgggcccgaaa

SEQ ID NO: 420 - protein, 40 aa, Anabaena variabilis
NLDQWLTEQVHEFQDMYLEPQAISNQDITFKLSDLDFIHN

SEQ ID NO: 421 - DNA, 124 bp, Anabaena variabilis
  source 1..124: mol_type = genomic DNA
taatttagatcagtggttaacagaacaagttcatgagtttcaagatatgtacttggaaccacaagcaatatccaatcaagacattaccttcaaactatctgacctagattttattcataattga

SEQ ID NO: 422 - protein, 40 aa, Nostoc sp.; identical to SEQ ID NO: 420

SEQ ID NO: 423 - DNA, 123 bp, Nostoc sp.
  source 1..123: mol_type = genomic DNA
aatttagatcaatggttaacagaacaagttcatgagtttcaagatatgtacttggaaccacaagcaatatccaatcaagacattaccttcaaactgtcagacctagattttattcataattga

SEQ ID NO: 424 - protein, 7 aa, Nostoc sp.
  source 1..7: note = Nostoc azollae
HREKKSA

SEQ ID NO: 425 - DNA, 24 bp, Nostoc sp.
  source 1..24: mol_type = genomic DNA; note = Nostoc azollae
cacagagagaaaaaatcagcatag

SEQ ID NO: 426 - protein, 23 aa, unidentified
  source 1..23: note = Acaryochloris marina
TSNNWLAKNYLSMWNKKSSNPNL

SEQ ID NO: 427 - DNA, 72 bp, unidentified
  source 1..72: mol_type = genomic DNA; note = Acaryochloris marina
acaagcaataactggctagccaaaaactatctttctatgtggaataaaaagagcagtaatccaaacctttag

SEQ ID NO: 428 - protein, 6 aa, unidentified
  source 1..6: note = Cyanothece sp.
FRYFWW

SEQ ID NO: 429 - DNA, 21 bp, unidentified
  source 1..21: mol_type = genomic DNA; note = Cyanothece sp.
tttagatatttttggtggtaa

SEQ ID NO: 430 - protein, 6 aa, unidentified
  source 1..6: note = Cyanothece sp.
FRYFWW

SEQ ID NO: 431 - DNA, 21 bp, unidentified
  source 1..21: mol_type = genomic DNA; note = Cyanothece sp.
tttagatatttttggtggtaa

SEQ ID NO: 432 - protein, 9 aa, unidentified
  source 1..9: note = Cyanothece sp.
CGEKWRIFS

SEQ ID NO: 433 - DNA, 27 bp, unidentified
  source 1..27: mol_type = genomic DNA; note = Cyanothece sp.
tgtggagaaaaatggagaatttttagc

SEQ ID NO: 434 - protein, 8 aa, unidentified
  source 1..8: note = Cyanothece sp.
FRLQLWQF

SEQ ID NO: 435 - DNA, 24 bp, unidentified
  source 1..24: mol_type = genomic DNA; note = Cyanothece sp.
tttcgcttacaactgtggcaattt

SEQ ID NO: 436 - protein, 16 aa, unidentified
  source 1..16: note = Cyanothece sp.
LGCNQSSIWSIFFWNH

SEQ ID NO: 437 - DNA, 51 bp, unidentified
  source 1..51: mol_type = genomic DNA; note = Cyanothece sp.
ctaggatgtaaccagagcagtatctggtcaatttttttctggaatcattaa

SEQ ID NO: 438 - protein, 40 aa, Microcoleus chthonoplastes
YNLQGLPAIESEDCIPDSVAPSDDWFSGVSSLFNRLTGLG

SEQ ID NO: 439 - DNA, 123 bp, Microcoleus chthonoplastes
  source 1..123: mol_type = genomic DNA
tataacctacaggggttgccagcaattgagtcagaagactgtatcccagattctgtagcgccttcggatgattggttttcaggcgtatcgtctctgtttaaccgcttgactgggttgggttag

SEQ ID NO: 440 - protein, 37 aa, Nostoc sp.
WMAIRRILRCHPFHPGGYDPVPELGEHCCHHDSGNKG

SEQ ID NO: 441 - DNA, 114 bp, Nostoc sp.
  source 1..114: mol_type = genomic DNA
tggatggcgattcgccgcattttgcgttgtcatccattccacccagggggttatgatcctgtaccagagttgggtgagcattgttgtcatcatgatagcgggaataaggggtga

SEQ ID NO: 442 - protein, 35 aa, Anabaena variabilis
WMGIRRILRCHPFHPGGYDPVPEVGEHCCHHDSGK

SEQ ID NO: 443 - DNA, 108 bp, Anabaena variabilis
  source 1..108: mol_type = genomic DNA
tggatggggattcgccgcattttgcgttgtcatccattccacccaggcggttatgatcctgtaccagaggtgggtgagcattgttgtcatcatgatagcgggaagtag

SEQ ID NO: 444 - protein, 48 aa, Nodularia spumigena
WMATRRILRCHPFHPGGYDPVPEVKHNCCDQHLSDSGKQTTEDHHKGS

SEQ ID NO: 445 - DNA, 147 bp, Nodularia spumigena
  source 1..147: mol_type = genomic DNA
tggatggcgactcggcggattttgcgttgtcatcccttccatcctggtggatatgatccagttccagaggtaaaacacaattgctgcgatcagcatctgtccgattctgggaaacagaccacagaagaccatcacaaaggctcgtag

SEQ ID NO: 446 - protein, 34 aa, Nostoc sp.
  source 1..34: note = Nostoc azollae
WMATLRILRCHPFHPGGYDPVPGLAEKSCCDHHD

SEQ ID NO: 447 - DNA, 105 bp, Nostoc sp.
  source 1..105: mol_type = genomic DNA; note = Nostoc azollae
tggatggcaactttgcggattttacgctgtcatcctttccatcctggtggttatgatcctgtaccaggactagcggaaaaatcctgttgtgaccatcatgattga

SEQ ID NO: 448 - protein, 28 aa, Synechococcus sp.
WLTAKRFCRCHPLHPGGYDPVPEKKSVL

SEQ ID NO: 449 - DNA, 87 bp, Synechococcus sp.
  source 1..87: mol_type = genomic DNA
tggctaacagccaagcgcttttgtcgctgtcatccgcttcatcctggcgggtatgatccggtaccggagaagaaatcggtactctaa

SEQ ID NO: 450 - protein, 23 aa, Prochlorococcus marinus
WLTLRRLSRCHPFTPCGCDPVPD

SEQ ID NO: 451 - DNA, 72 bp, Prochlorococcus marinus
  source 1..72: mol_type = genomic DNA
tggctcaccctgcggcgcctgtctcgttgccatccttttaccccctgtggttgcgacccggtgcctgattaa

SEQ ID NO: 452 - protein, 69 aa, Escherichia coli
MSYKKLYQLTAIFSLPLTILLVSLSSLRIVGEGNSYVDVFLSFIIFLGFIELIHGIRKILVWSGWKNGS

SEQ ID NO: 453 - DNA, 210 bp, Escherichia coli
  source 1..210: mol_type = genomic DNA
atgagttataaaaaactgtaccaattgacggctatatttagtttacctcttactatcttattggtttcactttcatcccttcggattgttggcgaagggaattcttatgttgacgtttttctaagctttataatatttcttggttttattgagctgattcatgggattcgaaagattttggtctggtcaggctggaaaaacggaagttaa

SEQ ID NO: 454 - protein, 85 aa, Escherichia coli
MGLKLDLTWFDKSTEDFKGEEYSKDFGDDGSVMESLGVPFKDNVNNGCFDVIAEWVPLLQPYFNHQIDISDNEYFVSFDYRDGDW

SEQ ID NO: 455 - DNA, 258 bp, Escherichia coli
  source 1..258: mol_type = genomic DNA
atgggacttaaattggatttaacttggtttgataaaagtacagaagattttaagggtgaggagtattcaaaagattttggagatgacggttcagttatggaaagtctaggtgtgccttttaaggataatgttaataacggttgctttgatgttatagctgaatgggtacctttgctacaaccatactttaatcatcaaattgatatttccgataatgagtattttgtttcgtttgattatcgtgatggtgattggtga

SEQ ID NO: 456 - protein, 113 aa, Escherichia coli
MSLRYYIKNILFGLYCTLIYIYLITKNSEGYYFLVSDKMLYAIVISTILCPYSKYAIEYIAFNFIKKDFFERRKNLNNAPVAKLNLFMLYNLLCLVLAIPFGLLGLFISIKNN

SEQ ID NO: 457 - DNA, 342 bp, Escherichia coli
  source 1..342: mol_type = genomic DNA
atgagcttaagatactacataaaaaatattttatttggcctgtactgcacacttatatatatataccttataacaaaaaacagcgaagggtattatttccttgtgtcagataagatgctatatgcaatagtgataagcactattctatgtccatattcaaaatatgctattgaatacatagcttttaacttcataaagaaagattttttcgaaagaagaaaaaacctaaataacgcccccgtagcaaaattaaacctatttatgctatataatctactttgtttggtcctagcaatcccatttggattgctaggactttttatatcaataaagaataattaa

SEQ ID NO: 458 - protein, 85 aa, Escherichia coli
MGLKLHIHWFDKKTEEFKGGEYSKDFGDDGSVIESLGMPLKDNINNGWFDVEKPWVSILQPHFKNVIDISKFDYFVSFVYRDGNW

SEQ ID NO: 459 - DNA, 258 bp, Escherichia coli
  source 1..258: mol_type = genomic DNA
atggggcttaaattacatattcattggtttgataagaaaaccgaagagtttaaaggcggtgaatactcaaaagacttcggtgatgatggttctgtcattgaaagtctggggatgcctttaaaggataatattaataatggttggtttgatgttgaaaaaccatgggtttcgatattacagccacactttaaaaatgtaatcgatattagtaaatttgattactttgtatcctttgtttaccgggatggtaactggtaa

SEQ ID NO: 460 - protein, 86 aa, Escherichia coli
MELKHSISDYTEAEFLEFVKKICRAEGATEEDDNKLVREFERLTEHPDGSDLIYYPRDDREDSPEGIVKEIKEWRAANGKSGFKQG

SEQ ID NO: 461 - DNA, 261 bp, Escherichia coli
  source 1..261: mol_type = genomic DNA
atggaactgaaacatagtattagtgattataccgaggctgaatttctggagtttgtaaaaaaaatatgtagagctgaaggtgctactgaagaggatgacaataaattagtgagagagtttgagcgattaactgagcacccagatggttcagatctgatttattatcctcgcgatgacagggaagatagtcctgaagggattgtcaaggaaattaaagaatggcgagctgctaacggtaagtcaggatttaaacagggctga

SEQ ID NO: 462 - protein, 178 aa, Citrobacter freundii
MMNEHSIDTDNRKANNALYLFIIIGLIPLLCIFVVYYKTPDALLLRKIATSTENLPSITSSYNPLMTKVMDIYCKTAPFLALILYILTFKIRKLINNTDRNTVLRSCLLSPLVYAAIVYLFCFRNFELTTAGRPVRLMATNDATLLLFYIGLYSIIFFTTYITLFTPVTAFKLLKKRQ

SEQ ID NO: 463 - DNA, 537 bp, Citrobacter freundii
  source 1..537: mol_type = genomic DNA
atgatgaatgaacactcaatagatacggacaacagaaaggccaataacgcattgtatttatttataataatcggattaataccattattgtgcatttttgttgtttactacaaaacgccagacgctttacttttacgtaaaattgctacaagcactgagaatctcccgtcaataacatcctcctacaacccattaatgacaaaggttatggatatttattgtaaaacagcgcctttccttgccttaatactatacatcctaacctttaaaatcagaaaattaatcaacaacaccgacaggaacactgtacttagatcttgtttattaagtccattggtctatgcagcaattgtttatctattctgcttccgaaattttgagttaacaacagccggaaggcctgtcagattaatggccaccaatgacgcaacactattgttattttatattggtctgtactcaataattttctttacaacctatatcacgctattcacaccagtcactgcatttaaattattaaaaaaaaggcagtaa

SEQ ID NO: 464 - protein, 111 aa, Escherichia coli
MNRKYYFNNMWWGWVTGGYMLYMSWDYEFKYRLLFWCISLCGMVLYPVAKWYIEDTALKFTRPDFWNSGFFADTPGKMGLLAVYTGTVFILSLPLSMIYILSVIIKRLSVR

SEQ ID NO: 465 - DNA, 336 bp, Escherichia coli
  source 1..336: mol_type = genomic DNA
atgaacagaaaatattattttaataatatgtggtggggatgggtgacggggggatatatgctgtatatgtcatgggattatgagtttaaatacagattactgttctggtgtatttctctctgcggaatggttttgtatccggttgcaaaatggtatattgaagatacagctctaaaatttacccggcctgatttctggaacagcggtttttttgctgatacacctggaaaaatggggttgcttgcggtttatacgggtactgttttcatattatctcttccgttaagtatgatatatattctttctgttattataaaaaggctgtctgtaagatag

SEQ ID NO: 466 - protein, 115 aa, Escherichia coli
MKLDISVKYLLKSLIPILIILTVFYLGWKDNQENARMFYAFIGCIISAITFPFSMRIIQKMVIRFTGKEFWQKDFFTNPVGGSLTAIFELFCFVISVPVVAIYLIFILCKALSGK

SEQ ID NO: 467 - DNA, 348 bp, Escherichia coli
  source 1..348: mol_type = genomic DNA
atgaaactggatatatctgtaaagtatttactgaaaagcctgataccaatcctcattattcttacagttttttatctgggatggaaagataaccaggaaaatgcaagaatgttttatgcgttcatcggatgcattatcagtgccattacttttcctttttcaatgaggataatacagaaaatggtaataaggtttacagggaaagaattctggcaaaaagacttctttacaaatccagttggcggaagcttaactgcaatatttgaattattctgtttcgttatatcagttcctgtggttgccatttacttaatttttatactctgcaaagccctttcaggaaaatga

SEQ ID NO: 468 - protein, 131 aa, Escherichia coli
MHNTLLEKIIAYLSLPGFHSLNNPPLSEAFNLYVHTAPLAATSLFIFTHKELELKPKSSPLRALKILTPFTILYISMIYCFLLTDTELTLSSKTFVLIVKKRSVFVFFLYNTIYWDIYIHIFVLLVPYRNI

SEQ ID NO: 469 - DNA, 396 bp, Escherichia coli
  source 1..396: mol_type = genomic DNA
atgcacaatacactcctcgaaaaaatcatcgcatacctatccctaccaggatttcattcattaaacaacccgcccctaagcgaagcattcaatctctatgttcatacagcccctttagctgcaaccagcttattcatattcacacacaaagaattagagttaaaaccaaagtcgtcacctctgcgggcactaaagatattaactcctttcactattctttatatatccatgatatactgtttcttgctaactgacacagaactaaccttgtcatcaaaaacatttgtattaatagtcaaaaaacgatctgtttttgtcttttttctatataacactatatattgggatatatatattcacatatttgtacttttggttccttataggaacatataa

SEQ ID NO: 470 - protein, 85 aa, Escherichia coli
MELKNSISDYTETEFKKIIEDIINCEGDEKKQDDNLEHFISVTEHPSGSDLIYYPEGNNDGSPEAVIKEIKEWRAANGKSGFKQG

SEQ ID NO: 471 - DNA, 258 bp, Escherichia coli
  source 1..258: mol_type = genomic DNA
atggaactgaaaaacagcattagtgattacactgaaactgaattcaaaaaaattattgaagacatcatcaattgtgaaggtgatgaaaaaaaacaggatgataacctcgagcattttataagtgttactgagcatcctagtggttctgatctgatttattacccagaaggtaataatgatggtagccctgaagctgttattaaagagattaaagaatggcgagctgctaacggtaagtcaggatttaaacagggctga

SEQ ID NO: 472 - protein, 98 aa, Lactococcus lactis
  source 1..98: note = subsp. lactis
MKKKQIEFENELRSMLATALEKDISQEERNALNIAEKALDNSEYLPKIILNLRKALTPLAINRTLNHDLSELYKFITSSKASNKNLGGGLIMSWGRLF

SEQ ID NO: 473 - DNA, 297 bp, Lactococcus lactis
  source 1..297: mol_type = genomic DNA; note = subsp. lactis
atgaaaaaaaaacaaatagaatttgaaaacgagctaagaagtatgttggctaccgcccttgaaaaagacattagtcaagaggaaagaaatgctctgaatattgcagaaaaggcgcttgacaattctgaatatttaccaaaaattattttaaacctcagaaaagccctaactccattagctataaatcgaacacttaaccatgatttatctgaactgtataaattcattacaagttccaaagcatcaaacaaaaatttaggtggtggtttaattatgtcgtggggacgactattctaa

SEQ ID NO: 474 - protein, 98 aa, Lactococcus lactis; identical to SEQ ID NO: 472
  source 1..98: note = subsp. cremoris

SEQ ID NO: 475 - DNA, 297 bp, Lactococcus lactis; identical to SEQ ID NO: 473
  source 1..297: mol_type = genomic DNA; note = subsp. cremoris

SEQ ID NO: 476 - protein, 87 aa, Escherichia coli
MNKMAMIDLAKLFLASKITAIEFSERICVERRRLYGVKDLSPNILNCGEELFMAAERFEPDADRANYEIDDNGLKVEVRSILEKFKL

SEQ ID NO: 477 - DNA, 249 bp, Escherichia coli
  source 1..249: mol_type = genomic DNA
atgatcgatttggcgaaattatttttagcttcgaaaattacagtgattgagttttcagagcgaatttgtgttgaacggagaagattgtatggtgttaaggatttgtctccgaatatattaaattgtggggaagagttgtctatggctgctgagcgatttgagcctgatgcagatagggctaattatgaaattgatgataatggacttaaggtcgaggtccgatctatcttggaaaaacttaaatcataa

SEQ ID NO: 478 - protein, 83 aa, Escherichia coli
MKLSPKAAIEVCNEAAKKGLWILGIDGGHWLNPGFRIDSSASWTYDMPEEYKSKIPENNRLAIENIKDDIENGYTAFIITLKM

SEQ ID NO: 479 - DNA, 243 bp, Escherichia coli
  source 1..243: mol_type = genomic DNA
atgaagttatcaccaaaagctgcaatagaagtttgtaatgaagcagcgaaaaaaggcttatggattttgggcattgatggtgggcattggctgaatcctggattcaggatagatagttcagcatcatggacatatgatatgccggagaatacaaatcaaaaatccctgaaaataatagattggctattgaaaatattaaagatgatattgagaatggatacactgctttcattatcacgttaa

SEQ ID NO: 480 - protein, 85 aa, Escherichia coli
MGLKLHINWFDKRTEEFKGGEYSKDFGDDGSVIERLGMPFKDNINNGWFDVIAEWVPLLQPYFNHQIDISDNEYFVSFDYRDGDW

SEQ ID NO: 481 - DNA, 258 bp, Escherichia coli
  source 1..258: mol_type = genomic DNA
atggggcttaaattacatattaattggtttgataagacgaccgaggaatttaaaggtggtgagtattcaaaagattttggagatgatggctcggtcattgaacgtcttggaatgcctttaaaagataatatcaataatggttggtttgatgttatagctgaatgggtacctttgctacaaccatactttaatcatcaaattgatatttccgataatgagtattttgtttcgtttgattatcgtgatggtgattggtga

SEQ ID NO: 482 - protein, 85 aa, Escherichia coli
MELKKSIGDYTETEFKKIIENIINCEGDEKKQDDNLEHFISVTEHPSGSDLIYYPEGNNDGSPEAVIKEIKEWRAANGKSGFKQG

SEQ ID NO: 483 - DNA, 258 bp, Escherichia coli
  source 1..258: mol_type = genomic DNA
gtggagctaaagaaaagtattggtgattacactgaaaccgaattcaaaaaaattattgaaaacatcatcaattgtgaaggtgatgaaaaaaaacaggatgataacctcgagcattttataagtgttactgagcatcctagtggttctgatctgatttattacccagaaggtaataatgatggtagccctgaagctgttattaaagagattaaagaatggcgagctgctaacggtaagtcaggatttaaacagggctga

SEQ ID NO: 484 - protein, 86 aa, Escherichia coli [sequence omitted]

SEQ ID NO: 485 - DNA, 261 bp, Escherichia coli
  source 1..261: mol_type = genomic DNA
atggaactgaagcatagcattagtgattatacagaagctgaatttttacaacttgtaacaacaatttgtaatgcgaacacttccagtgaagaagaactggttaaattggttacacactttgaggaaatgactgagcaccctagtggtagtgatttaatatattacccaaaagaaggtgatgatgactcaccttcaggtattgtaaacacagtaaaacaatggcgagccgctaacggtaagtcaggatttaaacagggctaa

SEQ ID NO: 486 - protein, 141 aa, Escherichia coli
MLTLYGYIRNVFLYRMNDRSCGDFMKVISMKFIFILTIIALAAVFFWSEDKGPACYQVSDEQARTFVKNDYLQRMKRWDNDVQLLGTEIPKITWEKIERSLTDVEDEKTLLVPFKAEGPDGKRMYYGMYHCEEGYVEYAND

SEQ ID NO: 487 - DNA, 354 bp, Escherichia coli
  source 1..354: mol_type = genomic DNA
atgaaagtaattagcatgaaatttatttttattttaacgattattgctcttgctgctgtttttttctggtctgaagataaaggtccggcatgctatcaggtcagcgatgaacaggccagaacgtttgtaaaaaatgattacctgcaaagaatgaaacgctgggacaacgatgtacaacttcttggtacagaaatcccgaaaattacatgggaaaagattgagagaagtttaacagatgttgaagatgaaaaaacacttcttgtcccatttaaagctgaaggcccggacggtaagagaatgtattatggcatgtaccattgtgaggagggatatgttgaatatgcgaatgactaa

SEQ ID NO: 488 - protein, 175 aa, Escherichia coli
MTSNKDKNKKANEILYAFSIIGIIPLMAILILRINDPYSQVLYYLYNKVAFLPSITSLHDPVMTTLMSNYNKTAPVMGILVFLCTYKTREIIKPVTRKLVVQSCFWGPVFYAILIYITLFYNLELTTAGGFFKLLSHNVITLFILYCSIYFTVLTMTYAILLMPLLVIKYFKGRQ

SEQ ID NO: 489 - DNA, 528 bp, Escherichia coli
  source 1..528: mol_type = genomic DNA
atgaccagcaataaagataagaacaagaaagcaaacgaaatattatatgcattttccataatcgggattattccattaatggctatattaatacttcgaataaatgatccatattctcaagtgctgtactacttatataataaggtggcatttctcccttctattacatcattgcatgatcccgtcatgacaacacttatgtcaaactacaacaagacagcgccagttatgggtattctcgtttttctttgcacatataagacaagagaaatcataaagccagtaacaagaaaacttgttgtgcaatcctgtttctgggggcccgttttttatgccattctgatttatatcacactgttctataatctggaactaacaacagcaggtggtttttttaaattattatctcataatgtcatcactctgtttattttatattgctccatttactttactgttttaaccatgacatatgcgattttactgatgccattacttgtcattaaatattttaaagggaggcagtaa

SEQ ID NO: 490 - protein, 78 aa, Escherichia coli
MDRKRTKLELLFAFIINATAIYIALAIYDCVFRGKDFLSMHTFCFSALMSAICYFVGDNYYSISDKIKRRSYENSDSK

SEQ ID NO: 491 - DNA, 237 bp, Escherichia coli
  source 1..237: mol_type = genomic DNA
atggatagaaaaagaacaaaattagagttgttatttgcatttataataaatgccaccgcaatatatattgcattagctatatatgattgtgtttttagaggaaaggactttttatccatgcatacattttgcttctctgcattaatgtctgcaatatgttactttgttggtgataattattattcaatatccgataagataaaaaggagatcatatgagaactctgactctaaatga

SEQ ID NO: 492 - protein, 113 aa, Shigella sonnei
MSLRYYIKNILFGLYCALIYIYLITKNNEGYYFLASDKMLYAIVISTILCPYSKYAIEHIFFKFIKKDFFRKRKNLNKCPRGKIKPYLCVYNLLCLVLAIPFGLLGLVYINKE

SEQ ID NO: 493 - DNA, 342 bp, Shigella sonnei
  source 1..342: mol_type = genomic DNA
atgagtttaagatactacataaaaaatattttgtttggcctatactgcgcacttatatatatataccttataacaaaaaacaacgaagggtattatttcctagcgtcagataagatgctatacgcaatagtgataagcactattctatgcccatattcaaaatatgctattgaacacatattttttaagttcataaagaaagattttttcagaaaaagaaaaaacctaaataaatgcccccgtggcaaaattaaaccgtatttatgcgtatacaatctactttgtttggtcctagcaatcccatttggattgctaggacttgtttatatcaataaagaataa

SEQ ID NO: 494 - protein, 113 aa, Escherichia coli
MSLRYYIKNILFGLYCTLIYIYLITKNSEEYYFLVTDKMLYAIVISTILCPYSKYAIEHIAFNFIKKHFFERRKNLNNAPVAKLNLFMLYNLLCLVLAIPFGLLGLFISIKNN

SEQ ID NO: 495 - DNA, 342 bp, Escherichia coli
  source 1..342: mol_type = genomic DNA
atgagcttaagatactacataaaaaatattttatttggcctgtactgcacacttatatatatataccttataacaaaaaacagcgaagagtattatttccttgtgacagataagatgctatatgcaatagtgataagcactattctatgtccatattcaaaatatgctattgaacacatagcttttaacttcataaagaaacattttttcgaaagaagaaaaaacctaaataacgcccccgtagcaaaattaaacctatttatgctatataatctactttgtttggtcctagcaatcccatttggattgctaggactttttatatcaataaagaataattaa

SEQ ID NO: 496 - protein, 113 aa, Leuconostoc gelidum
MRKNNILLDDAKIYTNKLYLLLIDRKDDAGYGDICDVLFQVSKKLDSTKNVEALINRLVNYIRITASTNRIKFSKDEEAVIIELGVIGQKAGLNGQYMADFSDKSQFYSIFER

SEQ ID NO: 497 - DNA, 342 bp, Leuconostoc gelidum
  source 1..342: mol_type = genomic DNA
ttgagaaaaaataacattttattggacgatgctaaaatatacacgaacaaactctatttgctattaatcgatagaaaagatgacgctgggtatggagatatttgtgatgttttgtttcaggtatccaaaaaattagatagcacaaaaaatgtagaagcattgattaaccgattggtcaattatatacgaattaccgcttcaacaaacagaattaagttttcaaaagatgaagaggctgtaattatagaacttggtgtaattggtcagaaggctggattaaacggccaatacatggctgatttttctgacaaatctcagttttatagtatctttgaaagataa

SEQ ID NO: 498 - protein, 91 aa, Lactococcus lactis
  source 1..91: note = subsp. cremoris
MKKKVDTEKQITSWASDLASKNETKVQEKLILSSYIQDIENHVYFPKAMISLEKKLRDQNNICALSKEVNQFYFKVVEVNQRKSWMVGLIV

SEQ ID NO: 499 - DNA, 276 bp, Lactococcus lactis
  source 1..276: mol_type = genomic DNA; note = subsp. cremoris
atgaaaaaaaaagttgatacagaaaaacaaattacttcttgggcatctgacttagcttccaaaaatgaaacaaaggttcaagaaaaattaatactgtcttcttatattcaggacatcgaaaaccatgtttactttccaaaagcaatgatttctttagaaaaaaaattacgagaccaaaataatatttgcgctttatcaaaagaagtcaatcagttttattttaaagttgttgaagtaaatcaaagaaaatcctggatggtaggtttgatagtttaa

SEQ ID NO: 500 - protein, 112 aa, Pediococcus acidilactici
MNKTKSEHIKQQALDLFTRLQFLLQKHDTIEPYQYVLDILETGISKTKHNQQTPERQARVVYNKIASQALVDKLHFTAEENKVLAAINELAHSQKGWGEFNMLDTTNTWPSQ

SEQ ID NO: 501 - DNA, 339 bp, Pediococcus acidilactici
  source 1..339: mol_type = genomic DNA
atgaataagactaagtcggaacatattaaacaacaagctttggacttatttactaggctacagtttttactacagaagcacgatactatcgaaccttaccagtacgttttagatattctggagactggtatcagtaaaactaaacataaccagcaaacgcctgaacgacaagctcgtgtagtctacaacaagattgccagccaagcgttagtagataagttacattttactgccgaagaaaacaaagttctagcagccatcaatgaattggcgcattctcaaaaagggtggggcgagtttaacatgctagatactaccaatacgtggcctagccaatag

SEQ ID NO: 502 - protein, 88 aa, Carnobacterium maltaromaticum
MIKDEKINKIYALVKSALDNTDVKNDKKLSLLLMRIQETSINGELFYDYKKELQPAISMYSIQHNFRVPDDLVKLLALVQTPKAWSGF

SEQ ID NO: 503 - DNA, 267 bp, Carnobacterium maltaromaticum
  source 1..267: mol_type = genomic DNA
atgataaaagatgaaaaaataaataaaatctatgctttagttaagagcgcacttgataatacggatgttaagaatgataaaaaactttctttacttcttatgagaatacaagaaacatcaattaatggagaactattttacgattataaaaaagaattacagccagctattagtatgtactctattcaacataactttcgggttcctgacgatctagtaaaactgttagcattagttcaaacacctaaagcttggtcagggttttaa

SEQ ID NO: 504 - protein, 111 aa, Carnobacterium maltaromaticum
MDIKSQTLYLNLSEAYKDPEVKANEFLSKLVVQCAGKLTASNSENSYIEVISLLSRGISSYYLSHKRIIPSSMLTIYTQIQKDIKNGNIDTEKLRKYEIAKGLMSVPYIYF

SEQ ID NO: 505 - DNA, 336 bp, Carnobacterium maltaromaticum
  source 1..336: mol_type = genomic DNA
atggatataaagtctcaaacattatatttgaatctaagcgaggcatataaagaccctgaagtaaaagctaatgaattcttatcaaaattagttgtacaatgtgctgggaaattaacagcttcaaacagtgagaacagttatattgaagtaatatcattgctatctaggggtatttctagttattatttatcccataaacgtataattccttcaagtatgttaactatatatactcaaatacaaaaggatataaaaaacgggaatattgacaccgaaaaattaaggaaatatgagatagcaaaaggattaatgtccgttccttatatatatttctaa

SEQ ID NO: 506 - protein, 245 aa, Lactococcus lactis
  source 1..245: note = subsp. lactis
MRRYLILIVALIGITGLSGCYQTSHKKVRFDEGSYTNFIYDNKSYFVTDKEIPQENVNNSKVKFYKLLIVDMKSEKLLSSSNKNSVTLVLNNIYEASDKSLCMGINDRYYKILPESDKGAVKALRLQNFDVTSDISDDNFVIDKNDSRKIDYMGNIYSISDTTVSDEELGEYQDVLAEVRVFDSVSGKSIPRSEWGRIDKDGSNSKQSRTEWDYGEIHSIRGKSLTEAFAVEINDDFKLATKVGN

SEQ ID NO: 507 - DNA, 738 bp, Lactococcus lactis
  source 1..738: mol_type = genomic DNA; note = subsp. lactis
atgagaagatatttaatacttattgtggccttaatagggataacaggtttatcagggtgttatcaaacaagtcataaaaaggtgaggtttgacgaaggaagttatactaattttatttatgataataaatcgtatttcgtaactgataaggagattcctcaggagaacgttaacaattccaaagtaaaattttataagctgttgattgttgacatgaaaagtgagaaacttttatcaagtagcaacaaaaatagtgtgactttggtcttaaataatatttatgaggcttctgacaagtcgctatgtatgggtattaacgacagatactataagatacttccagaaagtgataagggggcggtcaaagctttgagattacaaaactttgatgtgacaagcgatatttctgatgataattttgttattgataaaaatgattcacgaaaaattgactatatgggaaatatttacagtatatcggacaccaccgtatctgatgaagaattgggagaatatcaggatgttttagctgaagtacgtgtgtttgattcagttagtggcaaaagtatcccgaggtctgaatgggggagaattgataaggatggttcaaattccaaacagagtaggacggaatgggattatggcgaaatccattctattagaggaaaatctcttactgaagcatttgccgttgagataaatgatgattttaagcttgcaacgaaggtaggaaactag

SEQ ID NO: 508 - protein, 261 aa, Rhizobium leguminosarum
  source 1..261: note = bv. trifolii
MNDEICLTGGGRTTVTRRGGVVYREGGPWSSTVISLLRHLEASGFAEAPSVVGTGFDERGRETLSFIEGEFVHPGPWSEEAFPQFGMMLRRLHDATASFKPPENSMWRDWFGRNLGEGQHVIGHCDTGPWNIVCRSGLPVGLIDWEVAGPVRADIELAQACWLNAQLYDDDIAERVGLGSVTMRAHQVRLLLDGYGLSRKQRGGFVDKLITFAVHDAAEQAKEAAVTPESNDAEPLWAIAWRTRSASWMLHHRQTLEAALA

SEQ ID NO: 509 - DNA, 786 bp, Rhizobium leguminosarum
  source 1..786: mol_type = genomic DNA; note = bv. trifolii
atgaatgatgagatttgcctgacaggtggcggacgaacgactgtcacgcggcgcggcggagtcgtgtatcgcgaaggcggcccgtggtcatcaaccgtcatttcgctcctgcggcatctggaagcctctggcttcgctgaagctccttccgttgtcggcaccggtttcgatgagcgcggccgggagacattatcgtttatcgagggtgagtttgttcacccaggcccttggtcggaggaggcttttccgcaatttggaatgatgttgcggcgactgcacgatgccaccgcctcgttcaaacctcccgaaaactcgatgtggcgcgattggttcgggcgtaacctcggtgagggtcaacacgtaataggacactgcgacacaggcccatggaacattgtttgccggtcaggattgcctgtcgggttgatagattgggaggtggctgggcctgtcagggcggatatcgaattggcccaggcttgttggctgaatgcccagctctacgatgacgacattgcggagagggtcggattaggctctgtgaccatgagagcgcatcaagttcgcctgctgcttgacggctatggtctgtctcggaagcaacgcggcggcttcgtcgacaagctaatcacgttcgcagttcacgatgcggccgagcaggcgaaagaggcggctgtcacgccagagtcgaacgatgcggaaccgctatgggcaattgcctggcgcactagaagtgcctcctggatgctccatcatcggcaaacactggaagcagcgctggcatag

SEQ ID NO: 510 - protein, 436 aa, Bacillus subtilis
MNNIIPIMSLLFKQLYSRQGKKDAIRIAAGLVILAVFEIGLIRQAGIDESVLRKTYIILALLLMNTYMVFLSVTSQWKESYMKLSCLLPISSRSFWLAQSVVLFVDTCLRRTLFFFILPLFLFGNGTLSGAQTLFWLGRFSFFTVYSIIFGVVLSNHFVKKKNLMFLLHAAIFACVCISAALMPAATIPLCAVHILWAVVIDFPVFLQAPPQQGKMHSFMRRSEFSFYKREWNRFISSKAMLLNYAVMAVFSGFFSFQMMNTGIFNQQVIYIVISALLLICSPIALLYSIEKNDRMLLITLPIKRKTMFWAKYRFYSGLLAGGFLLVVMIVGFISGRSISVLTFLQCIELLLAGAYIRLTADEKRPSFSWQTEQQLWSGFSKYRSYLFCLPLFLAILAGTAVSLAVIPIAGLVIVYYLQKQDGGFFDTSKRERLGS

SEQ ID NO: 511 - DNA, 1000 bp, Bacillus subtilis
  source 1..1000: mol_type = genomic DNA
atgaataacataatccctatcatgtctttgctgttcaaacagctttacagccggcaagggaaaaaggacgccatccgcattgccgcaggccttgtcattctggccgtgtttgaaatcgggctgatccgccaggccggcattgatgaatcggtgttgcgcaaaacgtatatcatactcgcgcttcttttgatgaacacatatatggtgtttctttccgtgacatcacaatggaaggaatcttatatgaagctgagctgcctgctgccgatttcttcacggagcttttggctcgcccagagtgtcgttttgtttgtcgatacctgtttgagaagaactttattcttttttattttaccgctgttcttatttggaaacggaacgctgtcaggggcgcaaacattgttttggctcggcaggttttcgttttttaccgtttactccattattttcggagttgtgctaagcaaccacttcgtcaaaaagaagaacttgatgtttctgctgcatgcggcgatattcgcctgtgtatgtatcagcgccgctttgatgccggccgccacgattccgctttgcgcggttcatatcctgtgggcggtggtcattgactttcctgtctttctgcaggcgcctccgcagcagggcaagatgcattcatttatgcggcgatctgaattttcgttttacaaaagagaatggaaccgatttatctcttctaaagcgatgctgttaaattacgcggtaatggcggtattcagcggcttcttttcgttccagatgatgaacaccggcatcttcaatcagcaagtgatttatatcgtgatttccgcgcttttgctcatctgctcgccgatcgcccttttgtattcgattgaaaaaaatgaccggatgctgctcatcacgcttccgatcaagcgaaaaacgatgttttgggcgaaatatcgcttttattcaggcctattggcaggcggatttctccttgtcgtgatgattgtgggtttca

SEQ ID NO: 512 - protein, 239 aa, Bacillus subtilis
MSILDIHDVSVWYERDNVILEQVDLHLEKGAVYGLLGVNGAGKTTLINTLTGVNRNFSGRFTLCGIEAEAGMPQKTSDQLKTHRYFAADYPLLFTEITAKDYVSFVHSLYQKDFSEQQFASLAEAFHFSKYINRRISELSLGNRQKVVLMTGLLLRAPLFILDEPLVGLDVESIEVFYQKMREYCEAGGTILFSSHLLDVVQRFCDYAAILHNKQIQKVIPIGEETDLRREFFEVIGHE

SEQ ID NO: 513 - DNA, 716 bp, Bacillus subtilis
  source 1..716: mol_type = genomic DNA
gcattttggatatacacgatgtatccgtttggtatgaacgggacaacgtcatcttagagcacgtggacttacacttagaaaaaggcgccgtttacggattgcttggggtaaacggtgccggcaaaacaacactgatcaatacgctgacaggagtgaaccgcaattacagcgggggctttacgctgtgcggcattgaagctgaggccggcatgccgcagaaaacatcagatcaactgaagattcaccgttacttcgccgctgattatccgctgctgtttacagaaattacggcgaaggactatgtgtctttcgtccattcgctttatcaaaaggatttttcagagcgacagtttgccagtttggctgaggcctttcatttttcaaaatacatcaacaggagaatctcggagctgtccttggggaacaggcaaaaggttgtgttgatgacaggattattgctgcgggctcccctgtttattttggatgagccgctcgtcggtttggatgtggaatcaatagaggtcttttatcagaaaatgcgggagtactgtgaggaaggcggaaccattttgttttcttcccatctgctcgatgtcgtgcagagattttgtgattttgcggccattctgcacaacaaacagatccaaaaggtcattccgattggggaggagaccgatctgcggcgggaattttttgaggttatcggccatgaataa

SEQ ID NO: 514 - protein, 53 aa, Bacillus subtilis
MSPAQRRILLYILSFIFVIGAVVYFVKSDYLFTLIFIAIAILFGMRARKADSR

SEQ ID NO: 515 - DNA, 162 bp, Bacillus subtilis
  source 1..162: mol_type = genomic DNA
ttgtcaccagcacaaagaagaattttactgtatatcctttcatttatctttgtcatcggcgcagtcgtctattttgtcaaaagcgattatctgtttacgctgattttcattgccattgccattctgttcgggatgcgcgcgcggaaggctgactcgcgatga

SEQ ID NO: 516 - protein, 87 aa, Escherichia coli
MELKNSISDYTEAEFVQLLKEIEKENVAATDDVLDVLLEHFVKITEHPDGTDLIYYPSDNRDDSPEGIVKEIKEWRAANGKPGFKQG

SEQ ID NO: 517 - DNA, 264 bp, Escherichia coli
  source 1..264: mol_type = genomic DNA
atggaactgaaaaatagtattagtgattacacagaggctgagtttgttcaacttcttaaggaaattgaaaaagagaatgttgctgcaactgatgatgtgttagatgtgttactcgaacactttgtaaaaattactgagcatccagatggaacggatctgatttattatcctagtgataatagagacgatagccccgaagggattgtcaaggaaattaaagaatggcgagctgctaacggtaagccaggatttaaacagggctga

SEQ ID NO: 518 - protein, 87 aa, Pseudomonas aeruginosa
MKSKISEYTEKEFLEFVEDIYTNNKKKFPTEESHIQAVLEFKKLTEHPSGSDLLYYPNENREDSPAGVVKEVKEWRASKGLPGFKAG

SEQ ID NO: 519 - DNA, 264 bp, Pseudomonas aeruginosa
  source 1..264: mol_type = genomic DNA
atgaagtccaagatttccgaatatacggaaaaagagtttcttgagtttgttgaagacatatacacaaacaataagaaaaagttccctaccgaggagtctcatattcaagccgtgcttgaatttaaaaaactaacggaacacccaagcggctcagaccttctttactaccccaacgaaaatagagaagatagcccagctggagttgtaaaggaagttaaagaatggcgtgcttccaaggggcttcctggctttaaggccggttag

SEQ ID NO: 520 - protein, 87 aa, Pseudomonas aeruginosa
MKSKISEYTEKEFLEFVKDIYTNNKKKFPTEESHIQAVLEFKKLTEHPSGSDLLYYPNENREDSPAGVVKEVKEWRASKGLPGFKAG

SEQ ID NO: 521 - DNA, 264 bp, Pseudomonas aeruginosa
  source 1..264: mol_type = genomic DNA
atgaagtccaagatttccgaatatacggaaaaagagtttcttgagtttgttaaagacatatacacaaacaataagaaaaagttccctaccgaggagtctcatattcaagccgtgcttgaatttaaaaaactaacggaacacccaagcggctcagaccttctttactaccccaacgaaaatagagaagatagcccagctggagttgtaaaggaagttaaagaatggcgtgcttccaaggggcttcctggctttaaggccggttag

SEQ ID NO: 522 - protein, 95 aa, Enterococcus hirae
MDFTKEEKLLNAISKVYNEATIDDYPDLKEKLFLYSKEISEGKSVGEVSMKLSSFLGRYILKHKFGLPKSLIELQEIVSKESQVYRGWASIGIWS

SEQ ID NO: 523 - DNA, 288 bp, Enterococcus hirae
  source 1..288: mol_type = genomic DNA
atggattttactaaagaagaaaaacttttaaatgcaattagtaaagtatacaatgaagcaactatagatgactatcctgacttaaaagaaaagctctttctttattctaaagaaatcagtgagggaaaaagtgttggtgaagttagtatgaaattaagtagttttcttggaagatatattttaaaacataaatttggattacctaaatctttaatagaattacaagaaattgttagtaaggaatctcaagtatatagaggatgggcttctattggtatttggagttaa

SEQ ID NO: 524 - protein, 113 aa, Leuconostoc mesenteroides
MKKKYRYLEDSKNYTSTLYSLLVDNVDKPGYSDICDVLLQVSKKLDNTQSVEALINRLVNYIRITASTYKIIFSKKEEELIIKLGVIGQKAGLNGQYMADFSDKSQFYSVFDQ

SEQ ID NO: 525 - DNA, 342 bp, Leuconostoc mesenteroides
  source 1..342: mol_type = genomic DNA
ttgaaaaaaaagtatcggtatttagaagatagcaaaaattacactagtacactctattctctgttagttgataatgttgacaaacctggatactcagatatttgcgatgttttgcttcaagtttctaagaagttggataatactcaaagtgttgaagcgctaattaatcgattggttaattatattcgtattactgcttcaacatacaaaattattttttcaaaaaaagaagaggaattgattataaaacttggtgttattggacaaaaagctggacttaatggtcagtatatggctgatttttcagacaagtctcagttttacagcgttttcgatcagtaa

SEQ ID NO: 526 - protein, 93 aa, Escherichia coli
MSFLNFAFSPVFFSIMACYFIVWRNKRNEFVCNRLLSIIIISFLICFIYPWLNYKIEVKYYIFEQFYLFCFLSSLVAVVINLIVYFILYRRCI

SEQ ID NO: 527 - DNA, 282 bp, Escherichia coli
  source 1..282: mol_type = genomic DNA
atgagttttcttaattttgcattttctcctgtattcttctccattatggcgtgttatttcattgtatggagaaataaacgaaacgaatttgtctgcaatagattgctatcaattataataatatcttttttgatatgcttcatatatccatggctaaattacaaaatcgaagttaaatattatatatttgaacagttttatcttttttgttttttatcgtcactcgtggctgttgtaataaacctaattgtatactttatattatacaggagatgtatatga

SEQ ID NO: 528 - protein, 96 aa, Escherichia coli
MHLKYYLHNLPESLIPWILILIFNDNDNTPLLFIFISSIHVLLYPYSKLTISRYIKENTKLKKEPWYLCKLSALFYLLMAIPVGLPSFIYYTLKRN

SEQ ID NO: 529 - DNA, 291 bp, Escherichia coli
  source 1..291: mol_type = genomic DNA
atgcatttaaaatactacctacataatttacctgaatcacttataccatggattcttattttaatatttaacgacaatgataacactcctttgttatttatatttatatcatcaatacatgtattgctatatccatactctaaattaaccatatctagatatatcaaagaaaatacaaagttaaaaaaagaaccctggtacttatgcaagttatctgcattgttttatttattaatggcaatcccagtaggattgccaagtttcatatattacactctaaagagaaattaa

SEQ ID NO: 530 - protein, 344 aa, Escherichia coli
MMIQSHPLLAAPLAVGDTIGFFSSSAPATVTAKNRFFRGVEFLQRKGFKLVSGKLTGKTDFYRSGTIKERAQEFNELVYNPDITCIMSTIGGDNSNSLLPFLDYDAIIANPKIIIGYSDTTALLAGIYAKTGLITFYGPALIPSFGEHPPLVDITYESFIKILTRKQSGIYTYTLPEKWSDESINWNENKILRPKKLYKNNCAFYGSGKVEGRVIGGNLNTLTGIWGSEWMPEILNGDILFIEDSRKSIATIERLFSMLKLNRVFDKVSAIILGKHELFDCAGSKRRPYEVLTEVLDGKQIPVLDGFDCSHTHPMLTLPLGVKLAIDFDNKNISITEQYLSTEK

SEQ ID NO: 531 - DNA, 1000 bp, Escherichia coli
  source 1..1000: mol_type = genomic DNA
atgatgatacaatctcatccactactggccgctcccctggcagtaggagatacaattggtttcttttcatcatctgctccggcaacagttactgcaaaaaatcgtttttttcggggagttgagtttcttcagagaaagggatttaagctggtatcagggaagcttaccggtaaaacagatttttatcgttcaggtactattaaagaaagagctcaagaatttaatgagttagtctacaatcctgatattacctgtataatgtcaacgatcggtggagataacagtaattcactactaccgtttctggactatgatgctatcattgcaaaccccaaaattatcataggttactcagatacaactgctttattagcaggaatatatgcaaaaacagggttaataacattctatggaccagctcttattccttcgtttggtgaacatccacctcttgtggatataacatatgaatcatttattaaaatactaacaagaaaacaatcaggaatatatacctacacattacctgaaaagtggagtgatgagagcataaactggaatgaaaacaagatattaaggcctaagaagctatataaaaacaactgtgccttttatggttccggaaaagttgaggggcgtgtaattggaggaaatctaaatactttgacaggtatatgggggagtgaatggatgcctgaaattcttaatggagatatattgtttattgaggacagtcggaaaagcattgcaacaattgaacgattattctctatgctaaagcttaatcgcgtgtttgataaagttagtgcaataatactcgggaaacatgagctttttgattgtgcaggaagtaaacgcagaccatatgaagtattaacagaggtattagatgggaaacagattcctgtactggatggatttgattgttcacatacacatccaatgctaactcttccacttggtgtaaaattagctattgactttgacaacaaaaatatat

SEQ ID NO: 532 - protein, 90 aa, Lactobacillus sakei
MKADYKKINSILTYTSTALKNPKIIKDKDLVVLLTIIQEEAKQNRIFYDYKRKFRPAVTRFTIDNNFEIPDCLVKLLSAVETPKAWSGFS

SEQ ID NO: 533 - DNA, 268 bp, Lactobacillus sakei
  source 1..268: mol_type = genomic DNA
ggcagattataaaaaaataaattcaatactaacttacacatctactgctttaaaaaaccctaaaattataaaagataaagatttagtagtccttctaactattattcaagaagaagccaaacaaaatagaatcttttatgattataaaagaaaatttcgtccagcggttactcgctttacaattgataataattttgagattcctgattgtttggttaaactactgtcagctgttgaaacacctaaggcgtggtctggatttagttag

SEQ ID NO: 534 - protein, 83 aa, Escherichia coli
MKLSPKAAIEVCNEAAKKGLWILGIDGGHWLNPGFRIDSSASWTYDMPEEYKSKTPENNRLAIENIKDDIENGYTAFIITLKM

SEQ ID NO: 535 - DNA, 251 bp, Escherichia coli
  source 1..251: mol_type = genomic DNA
tgaagttatcaccaaaagctgcaatagaagtttgtaatgaagcagcgaaaaaaggcttatggattttgggcattgatggtgggcattggctgaatcctggattcaggatagatagttcagcatcatggacatatgatatgccggaggaatacaaatcaaaaacccctgaaaataatagattggctattgaaaatattaaagatgatattgagaatggatacactgctttcattatcacgttaaagatgtaa

SEQ ID NO: 536 - protein, 436 aa, Bacillus subtilis
MNNIFPIMSLLFKQLYSRQGKKDAIRIAAGLVILAVFEIGLIRQAGIDESVLGKTYIILALLLMNTYMVFLSVTSQWKESYMKLSCLLPISSRSFWLAQSVVLFVDTCLRRTLFFFILPLFLFGNGTLSGAQTLFWLGRFSFFTVYSILFGVMLSNHFVKKKNSMFLLHAAVFAFVCLSAAFMPAVTIPLCAVHMLWAVIIDFPVFLQAPPHQSKMHFFMRRSEFSFYKREWNRFISSKAMLLNYVVMAAFSGFFSFQMMNTGIFNQQVIYIVISALLLICSPIALLYSIEKNDRMLLITLPIKRRTMFWAKYRFYSGLLAGGFLLVAIIVGFISGRPISALTFVQCMELLLAGAFIRLTADEKRPSFGWQTEQQLWSGFSKYRSYLFCLPLFLATLAGTAVSLAVIPIAALIIVYYLQKQDGGFFDTSKRERIGS

SEQ ID NO: 537 - DNA, 1000 bp, Bacillus subtilis
  source 1..1000: mol_type = genomic DNA
ttggggaggagaccgatctgcggcgggaattttttgaggttatcggccatgaataacatattccccatcatgtcgttgctgttcaaacagctgtacagccggcaagggaaaaaggacgctatccgcattgctgcagggcttgtgattctcgccgtgtttgaaatcgggctgatccgacaagccggcattgacgaatcggtgttgggaaaaacgtatatcatattggcgcttctcttaatgaacacgtatatggtgtttctttccgtgacatcacaatggaaggaatcttatatgaagctgagctgtctgctgccgatttcatcacggagcttttggctcgcccagagtgtcgttctgtttgtcgatacctgtttgagaagaacgttattcttttttattttaccgctgttcttatttggaaacggaacgctgtcaggggcgcaaacattgttttggcttggcagattttcgttttttaccgtttactcgattctattcggagttatgctaagcaaccatttcgtcaaaaagaagaactcgatgtttctgctgcatgcggcggtattcgcctttgtatgcctcagtgccgcttttatgccggccgtcacgatcccgctatgcgcggttcacatgctatgggcggtgatcattgactttccggtctttctgcaggcgcctccgcatcagagcaagatgcatttttttatgcggcgatctgaattttcgttttacaaaagagaatggaaccgatttatttcttctaaagcgatgctgttaaattacgtggtgatggcggcgttcagcggattcttttcgttccagatgatgaacactggcatcttcaatcagcaagtgatttatattgtgatttccgctctattgctgatttgctcgccgatcgcccttttgtactctattgaaaaaaacgatcgcatgctgctcatcacgcttccaattaaaagaagaacgatgttttgggcgaaatatcgcttttattcag

SEQ ID NO: 538 - protein, 580 aa, Escherichia coli
MERKQKNSLFNYIYSLMDVRGKFLFFSMLFITSLSSIIISISPLILAKITDLLSGSLSNFSYEYLVLLACLYMFCVISNKASVFLFMILQSSLRINMQKKMSLKYLRELYNENITNLSKNNAGYTTQSLNQASNDIYILVRNVSQNILSPVIQLISTIVVVLSTKDWFSAGVFFLYILVFVIFNTRLTGSLASLRKHSMDITLNSYSLLSDTVDNMIAAKKNNALRLISERYEDALTQENNAQKKYWLLSSKVLLLNSLLAVILFGSVFIYNILGVLNGVVSIGHFIMITSYIILLSTPVENIGALLSEIRQSMSSLAGFIQRHAENKATSPSIPFLNMERKLNLSIRELSFSYSDDKKILNSVSLDLFTGKMYSLTGPSGSGKSTLVKIISGYYKNYFGDIYLNDISLRNISDEDLNDAIYYLTQDDYIFMDTLRFNLRLANYDASENEIFKVLKLANLSVVNNEPVSLDTHLINRGNNYSGGQKQRISLARLFLRKPAIIIIDEATSALDYINESEILSSIRTHFPDALIINISHRINLLECSDCVYVLNEGNIVASGHFRDLMVSNEYISGLASVTE

SEQ ID NO: 539 - DNA, 1000 bp, Escherichia coli
  source 1..1000: mol_type = genomic DNA
atggaaagaaaacagaaaaactcattatttaattatatttattcattaatggatgtaagaggtaaatttttattcttttccatgttattcattacatcattatcatcgataatcatatctatttcaccattgattcttgcaaagattacagatttactgtctggctcattgtcaaattttagttatgaatatctggttttacttgcctgtttatacatgttttgcgttatatctaataaagcaagtgtttttttatttatgatactgcaaagtagtctacgtattaacatgcagaaaaaaatgtcgctaaagtatttgagagaattgtataacgaaaatataactaacttgagtaaaaataatgctggatatacaacgcaaagtcttaaccaggcttcaaatgacatttatattcttgtgagaaatgtttcccagaatatcctgtcacctgttatacaacttatttccactattgttgttgttttatctacgaaggactggttttctgccggtgtgttttttctctatattctggtatttgtaatttttaataccagactgactggcagtttagcgtctctcagaaaacacagcatggatatcactcttaactcttatagtctgttatctgatactgttgataacatgatagcagctaaaaagaataatgcattaagacttatttctgaacgttatgaagatgctctcactcaggaaaacaatgctcagaaaaaatactggttactcagttctaaagttcttttattgaactctttacttgctgtaatattatttggttctgtattcatatataatattttaggtgtgctgaatggtgtagttagtatcggccacttcattatgattacatcatatatcattcttctttcaacgccagtggaaaatataggggcattgctaagtgagatcaggcagtcaatgtctagcctggcaggttttattcaacgtcatgccgagaataaagccacatctccttcaa

SEQ ID NO: 540 - protein, 95 aa, Klebsiella pneumoniae
MTLLSFGFSPVFFSVMAFCIISRSKFYPQRTRNKVIVLILLTFFICFLYPLTKVYLVGSYGIFDKFYLFCFISTLIAIAINVVILTINGAKNERN

SEQ ID NO: 541 - DNA, 288 bp, Klebsiella pneumoniae
  source 1..288: mol_type = genomic DNA
atgacattactttcatttggattttctcctgttttcttttcagtcatggcgttctgtatcatttcacgtagtaaattctatccgcagagaacgcgaaacaaagttattgttctgattttactaactttttttatttgttttttatatccattaacaaaagtgtatctggtgggaagttacggtatatttgacaaattctacctcttttgctttatttctacgttaattgcaatagcaattaacgtagtgatacttacaataaatggagctaagaatgagagaaattag

SEQ ID NO: 542 - RNA, 13 nt, synthetic construct
  misc_feature 1..13: note = Kozak sequence
  source 1..13: mol_type = other RNA
gccgccrccatgg

SEQ ID NO: 543 - skipped

SEQ ID NO: 544 - DNA, 30 bp, synthetic construct
  misc_feature 1..30: note = Lead promoter
  source 1..30: mol_type = other DNA
gaaaaccttgtcaatgaagagcgatctatg

SEQ ID NO: 545 - DNA, 30 bp, synthetic construct
  misc_feature 1..30: note = FecA promoter
  source 1..30: mol_type = other DNA
ttctcgttcgactcatagctgaacacaaca

SEQ ID NO: 546 - DNA, 16 bp, synthetic construct
  misc_feature 1..16: note = Cu-sensitive promoter
  source 1..16: mol_type = other DNA
atgacaaaattgtcat

SEQ ID NO: 547 - DNA, 30 bp, synthetic construct
  misc_feature 1..30: note = Fe promoter
  source 1..30: mol_type = other DNA
accaatgctgggaacggccagggcacctaa

SEQ ID NO: 548 - DNA, 30 bp, synthetic construct
  misc_feature 1..30: note = Fe and UV promoters
  source 1..30: mol_type = other DNA
ctgaaagcgcataccgctatggagggggtt

SEQ ID NO: 549 - DNA, 30 bp, synthetic construct
  misc_feature 1..30: note = PrFe (PI + PII rus operon)
  source 1..30: mol_type = other DNA
tagatatgcctgaaagcgcataccgctatg

SEQ ID NO: 550 - DNA, 30 bp, synthetic construct
  misc_feature 1..30: note = Lux cassette right promoter
  source 1..30: mol_type = other DNA
tgttatagtcgaatacctctggcggtgata

SEQ ID NO: 551 - DNA, 30 bp, synthetic construct
  misc_feature 1..30: note = P(Las) TetO
  source 1..30: mol_type = other DNA
ttttggtacactccctatcagtgatagaga

SEQ ID NO: 552 - DNA, 30 bp, synthetic construct
  misc_feature 1..30: note = P(Las) CIO
  source 1..30: mol_type = other DNA
ctttttggtacactacctctggcggtgata

SEQ ID NO: 553 - DNA, 30 bp, synthetic construct
  misc_feature 1..30: note = P(Rhl)
  source 1..30: mol_type = other DNA
tacgcaagaaaatggtttgttatagtcgaa

SEQ ID NO: 554 - DNA, 30 bp, synthetic construct
  misc_feature 1..30: note = Double Promoter (LuxR/HSL, positive / cI,negative)
  source 1..30: mol_type = other DNA
cgtgcgtgttgataacaccgtgcgtgttga

SEQ ID NO: 555 - DNA, 30 bp, synthetic construct
  misc_feature 1..30: note = P2 promoter in agr operon from S. aureus
  source 1..30: mol_type = other DNA
agattgtactaaatcgtataatgacagtga

SEQ ID NO: 556 - DNA, 30 bp, synthetic construct
  misc_feature 1..30: note = plux-cI hybrid promoter
  source 1..30: mol_type = other DNA
gtgttgatgcttttatcaccgccagtggta

SEQ ID NO: 557 - DNA, 30 bp, synthetic construct
  misc_feature 1..30: note = plux-lac hybrid promoter
  source 1..30: mol_type = other DNA
agtgtgtggaattgtgagcggataacaatt

SEQ ID NO: 558 - DNA, 30 bp, synthetic construct
  misc_feature 1..30: note = CinR, CinL and glucose controlled promotor
  source 1..30: mol_type = other DNA
acatcttaaaagttttagtatcatattcgt

SEQ ID NO: 559 - DNA, 30 bp, synthetic construct; identical to SEQ ID NO: 553
  misc_feature 1..30: note = RhIR promoter repressible by CI
  source 1..30: mol_type = other DNA

SEQ ID NO: 560 - DNA, 30 bp, synthetic construct
  misc_feature 1..30: note = Reverse Lux Promoter
  source 1..30: mol_type = other DNA
tcttgcgtaaacctgtacgatcctacaggt

SEQ ID NO: 561 - DNA, 30 bp, synthetic construct
  misc_feature 1..30: note = rhlI promoter
  source 1..30: mol_type = other DNA
atcctcctttagtcttccccctcatgtgtg

SEQ ID NO: 562 - DNA, 30 bp, synthetic construct
  misc_feature 1..30: note = lasI promoter
  source 1..30: mol_type = other DNA
taaaattatgaaatttgcataaattcttca

SEQ ID NO: 563 - DNA, 30 bp, synthetic construct
  misc_feature 1..30: note = LuxR+3OC6HSL independent R0065
  source 1..30: mol_type = other DNA
gtgttgactattttacctctggcggtgata

SEQ ID NO: 564 - DNA, 30 bp, synthetic construct
  misc_feature 1..30: note = LasR/LasI Inducible & RHLR/RHLI repressiblePromoter
  source 1..30: mol_type = other DNA
gaaatctggcagtttttggtacacgaaagc

SEQ ID NO: 565 - DNA, 30 bp, synthetic construct
  misc_feature 1..30: note = pLux/cI Hybrid Promoter
  source 1..30: mol_type = other DNA
acaccgtgcgtgttgatatagtcgaataaa

SEQ ID NO: 566 - DNA, 30 bp, synthetic construct
  misc_feature 1..30: note = pLas promoter
  source 1..30: mol_type = other DNA
aaaattatgaaatttgtataaattcttcag

SEQ ID NO: 567 - DNA, 30 bp, synthetic construct
  misc_feature 1..30: note = pLas/cI Hybrid Promoter
  source 1..30: mol_type = other DNA
ggttctttttggtacctctggcggtgataa

SEQ ID NO: 568 - DNA, 30 bp, synthetic construct
  misc_feature 1..30: note = pLas/Lux Hybrid Promoter
  source 1..30: mol_type = other DNA
tgtaggatcgtacaggtataaattcttcag

SEQ ID NO: 569 - DNA, 30 bp, synthetic construct
  misc_feature 1..30: note = pLux
  source 1..30: mol_type = other DNA
caagaaaatggtttgttatagtcgaataaa

SEQ ID NO: 570 - DNA, 30 bp, synthetic construct
  misc_feature 1..30: note = pLux/Las Hybrid Promoter
  source 1..30: mol_type = other DNA
ctatctcatttgctagtatagtcgaataaa

SEQ ID NO: 571 - DNA, 30 bp, synthetic construct
  misc_feature 1..30: note = Hybrid promoter: HSL-LuxR activated, P22 C2repressed
  source 1..30: mol_type = other DNA
tagtttataatttaagtgttctttaatttc

SEQ ID NO: 572 - DNA, 30 bp, synthetic construct
  misc_feature 1..30: note = PAI+LasR -> LuxI (AI)
  source 1..30: mol_type = other DNA
caccttcgggtgggcctttctgcgtttata

SEQ ID NO: 573 - DNA, 30 bp, synthetic construct
  misc_feature 1..30: note = PAI+LasR -> LasI & AI+LuxR --| LasI
  source 1..30: mol_type = other DNA
aataactctgatagtgctagtgtagatctc

SEQ ID NO: 574 - DNA, 30 bp, synthetic construct; identical to SEQ ID NO: 572
  misc_feature 1..30: note = PAI+LasR -> LasI+GFP & AI+LuxR --| LasI+GFP
  source 1..30: mol_type = other DNA

SEQ ID NO: 575 - DNA, 30 bp, synthetic construct; identical to SEQ ID NO: 572
  misc_feature 1..30: note = Complex QS -> LuxI & LasI circuit
  source 1..30: mol_type = other DNA

SEQ ID NO: 576 - DNA, 30 bp, synthetic construct; identical to SEQ ID NO: 569
  misc_feature 1..30: note = position 3 mutated promoter lux pR-3 (luxR & HSLregulated)
  source 1..30: mol_type = other DNA

SEQ ID NO: 577 - DNA, 30 bp, synthetic construct; identical to SEQ ID NO: 569
  misc_feature 1..30: note = position 5 mutated promoter lux pR-5 (luxR & HSLregulated)
  source 1..30: mol_type = other DNA

SEQ ID NO: 578 - DNA, 30 bp, synthetic construct; identical to SEQ ID NO: 569
  misc_feature 1..30: note = position 3&5 mutated promoter lux pR-3/5 (luxR &HSL regulated)
  source 1..30: mol_type = other DNA

SEQ ID NO: 579 - DNA, 30 bp, synthetic construct
  misc_feature 1..30: note = Promoter (HSL-mediated luxR repressor)
  source 1..30: mol_type = other DNA
ttgacacctgtaggatcgtacaggtataat

SEQ ID NO: 580 - DNA, 30 bp, synthetic construct; identical to SEQ ID NO: 569
  misc_feature 1..30: note = Promoter (luxR & HSL regulated -- lux pR)
  source 1..30: mol_type = other DNA

SEQ ID NO: 581 - DNA, 30 bp, synthetic construct
  misc_feature 1..30: note = Promoter (luxR & HSL regulated -- lux pL)
  source 1..30: mol_type = other DNA
cacgcaaaacttgcgacaaacaataggtaa

SEQ ID NO: 582 - DNA, 30 bp, synthetic construct
  misc_feature 1..30: note = Promoter (RhlR & C4-HSL regulated)
  source 1..30: mol_type = other DNA
gttagctttcgaattggctaaaaagtgttc

SEQ ID NO: 583 - DNA, 30 bp, synthetic construct
  misc_feature 1..30: note = Promoter (cinR and HSL regulated)
  source 1..30: mol_type = other DNA
ccattctgctttccacgaacttgaaaacgc

SEQ ID NO: 584 - DNA, 30 bp, synthetic construct
  misc_feature 1..30: note = Promoter (LasR & PAI regulated)
  source 1..30: mol_type = other DNA
ggccgcgggttctttttggtacacgaaagc

SEQ ID NO: 585 - DNA, 30 bp, synthetic construct
  misc_feature 1..30: note = Promoter, Standard (luxR and HSL regulated -- luxpR)
  source 1..30: mol_type = other DNA
aagaaaatggtttgttgatactcgaataaa

SEQ ID NO: 586 - DNA, 30 bp, synthetic construct
  misc_feature 1..30: note = P(Bla)
  source 1..30: mol_type = other DNA
gtttatacataggcgagtactctgttatgg

SEQ ID NO: 587 - DNA, 30 bp, synthetic construct
  misc_feature 1..30: note = P(Cat)
  source 1..30: mol_type = other DNA
agaggttccaactttcaccataatgaaaca

SEQ ID NO: 588 - DNA, 30 bp, synthetic construct
  misc_feature 1..30: note = P(Kat)
  source 1..30: mol_type = other DNA
taaacaactaacggacaattctacctaaca

SEQ ID NO: 589 - DNA, 30 bp, synthetic construct
  misc_feature 1..30: note = Template for Building Primer Family Member
  source 1..30: mol_type = other DNA
acatcaagccaaattaaacaggattaacac

SEQ ID NO: 590 - DNA, 30 bp, synthetic construct
  misc_feature 1..30: note = Reverse lambda cI-regulated promoter
  source 1..30: mol_type = other DNA
gaggtaaaatagtcaacacgcacggtgtta

SEQ ID NO: 591 - DNA, 30 bp, synthetic construct
  misc_feature 1..30: note = Key Promoter absorbs 3
  source 1..30: mol_type = other DNA
caggccggaataactccctataatgcgcca

SEQ ID NO: 592 - DNA, 30 bp, synthetic construct
  misc_feature 1..30: note = constitutive promoter family member
  source 1..30: mol_type = other DNA
ggctagctcagtcctaggtacagtgctagc

SEQ ID NO: 593 - DNA, 30 bp, synthetic construct
  misc_feature 1..30: note = constitutive promoter family member
  source 1..30: mol_type = other DNA
agctagctcagtcctaggtattatgctagc

SEQ ID NO: 594 - DNA, 30 bp, synthetic construct
  misc_feature 1..30: note = constitutive promoter family member
  source 1..30: mol_type = other DNA
agctagctcagtcctaggtactgtgctagc

SEQ ID NO: 595 - DNA, 30 bp, synthetic construct
  misc_feature 1..30: note = constitutive promoter family member
  source 1..30: mol_type = other DNA
agctagctcagtcctagggattatgctagc

SEQ ID NO: 596 - DNA, 30 bp, synthetic construct
  misc_feature 1..30: note = constitutive promoter family member
  source 1..30: mol_type = other DNA
agctagctcagtcctaggtattgtgctagc

SEQ ID NO: 597 - DNA, 30 bp, synthetic construct
  misc_feature 1..30: note = constitutive promoter family member
  source 1..30: mol_type = other DNA
ggctagctcagtcctaggtactatgctagc

SEQ ID NO: 598 - DNA, 30 bp, synthetic construct
  misc_feature 1..30: note = constitutive promoter family member
  source 1..30: mol_type = other DNA
ggctagctcagtcctaggtatagtgctagc

SEQ ID NO: 599 - DNA, 30 bp, synthetic construct
  misc_feature 1..30: note = constitutive promoter family member
  source 1..30: mol_type = other DNA
ggctagctcagccctaggtattatgctagc

SEQ ID NO: 600 - DNA, 30 bp, synthetic construct
  misc_feature 1..30: note = constitutive promoter family member
  source 1..30: mol_type = other DNA
agctagctcagtcctaggtataatgctagc

SEQ ID NO: 601 - DNA, 30 bp, synthetic construct
  misc_feature 1..30: note = constitutive promoter family member
  source 1..30: mol_type = other DNA
agctagctcagtcctagggactgtgctagc

SEQ ID NO: 602 - DNA, 30 bp, synthetic construct
  misc_feature 1..30: note = constitutive promoter family member
  source 1..30: mol_type = other DNA
ggctagctcagtcctaggtacaatgctagc

SEQ ID NO: 603 - DNA, 30 bp, synthetic construct; identical to SEQ ID NO: 598
  misc_feature 1..30: note = constitutive promoter family member
  source 1..30: mol_type = other DNA

SEQ ID NO: 604 - DNA, 30 bp, synthetic construct; identical to SEQ ID NO: 595
  misc_feature 1..30: note = constitutive promoter family member
  source 1..30: mol_type = other DNA

SEQ ID NO: 605 - DNA, 30 bp, synthetic construct
  misc_feature 1..30: note = constitutive promoter family member
  source 1..30: mol_type = other DNA
ggctagctcagtcctagggattatgctagc

SEQ ID NO: 606 - DNA, 30 bp, synthetic construct; identical to SEQ ID NO: 602
  misc_feature 1..30: note = constitutive promoter family member
  source 1..30: mol_type = other DNA

SEQ ID NO: 607 - DNA, 30 bp, synthetic construct
  misc_feature 1..30: note = constitutive promoter family member
  source 1..30: mol_type = other DNA
agctagctcagcccttggtacaatgctagc

SEQ ID NO: 608 - DNA, 30 bp, synthetic construct
  misc_feature 1..30: note = constitutive promoter family member
  source 1..30: mol_type = other DNA
agctagctcagtcctagggactatgctagc

SEQ ID NO: 609 - DNA, 30 bp, synthetic construct
  misc_feature 1..30: note = constitutive promoter family member
  source 1..30: mol_type = other DNA
agctagctcagtcctagggattgtgctagc

SEQ ID NO: 610 - DNA, 30 bp, synthetic construct
  misc_feature 1..30: note = constitutive promoter family member
  source 1..30: mol_type = other DNA
ggctagctcagtcctaggtattgtgctagc

SEQ ID NO: 611 - DNA, 30 bp, synthetic construct; identical to SEQ ID NO: 600
  misc_feature 1..30: note = constitutive promoter family member
  source 1..30: mol_type = other DNA

SEQ ID NO: 612 - DNA, 30 bp, synthetic construct
  misc_feature 1..30: note = 1bp mutant from J23107
  source 1..30: mol_type = other DNA
ggctagctcagtcctaggtattatgctagc

SEQ ID NO: 613 - DNA, 30 bp, synthetic construct; identical to SEQ ID NO: 602
  misc_feature 1..30: note = 1bp mutant from J23114
  source 1..30: mol_type = other DNA

SEQ ID NO: 614 - DNA, 30 bp, synthetic construct
  misc_feature 1..30: note = pBAD reverse
  source 1..30: mol_type = other DNA
aaagtgtgacgccgtgcaaataatcaatgt

SEQ ID NO: 615 - DNA, 30 bp, synthetic construct
  misc_feature 1..30: note = NikR promoter, a protein of the ribbon helix-helixfamily of trancription factors that repress expre
  source 1..30: mol_type = other DNA
gacgaatacttaaaatcgtcatacttattt

SEQ ID NO: 616 - DNA, 30 bp, synthetic construct
  misc_feature 1..30: note = lacq_Promoter
  source 1..30: mol_type = other DNA
aaacctttcgcggtatggcatgatagcgcc

SEQ ID NO: 617 - DNA, 30 bp, synthetic construct
  misc_feature 1..30: note = lacIQ - promoter sequence
  source 1..30: mol_type = other DNA
tgatagcgcccggaagagagtcaattcagg

SEQ ID NO: 618 - DNA, 30 bp, synthetic construct
  misc_feature 1..30: note = E. Coli CreABCD phosphate sensing operon promoter
  source 1..30: mol_type = other DNA
ttatttaccgtgacgaactaattgctcgtg

SEQ ID NO: 619 - DNA, 30 bp, synthetic construct
  misc_feature 1..30: note = GlnRS promoter
  source 1..30: mol_type = other DNA
catacgccgttatacgttgtttacgctttg

SEQ ID NO: 620 - DNA, 30 bp, synthetic construct
  misc_feature 1..30: note = Constitutive weak promoter of lacZ
  source 1..30: mol_type = other DNA
ttatgcttccggctcgtatgttgtgtggac

SEQ ID NO: 621 - DNA, 30 bp, synthetic construct
  misc_feature 1..30: note = Mutated LacZ promoter
  source 1..30: mol_type = other DNA
ttatgcttccggctcgtatggtgtgtggac

SEQ ID NO: 622 - DNA, 30 bp, synthetic construct
  misc_feature 1..30: note = constitutive promoter with (TA)10 between -10 and-35 elements
  source 1..30: mol_type = other DNA
atatatatatatatataatggaagcgtttt

SEQ ID NO: 623 - DNA, 30 bp, synthetic construct; identical to SEQ ID NO: 622
  misc_feature 1..30: note = constitutive promoter with (TA)9 between -10 and-35 elements
  source 1..30: mol_type = other DNA

SEQ ID NO: 624 - DNA, 30 bp, synthetic construct
  misc_feature 1..30: note = constitutive promoter with (C)10 between -10 and-35 elements
  source 1..30: mol_type = other DNA
ccccgaaagcttaagaatataattgtaagc

SEQ ID NO: 625 - DNA, 30 bp, synthetic construct; identical to SEQ ID NO: 624
  misc_feature 1..30: note = constitutive promoter with (C)12 between -10 and-35 elements
  source 1..30: mol_type = other DNA

SEQ ID NO: 626 - DNA, 30 bp, synthetic construct
  misc_feature 1..30: note = optimized (TA) repeat constitutive promoter with13 bp between -10 and -35 elements
  source 1..30: mol_type = other DNA
tgacaatatatatatatatataatgctagc

SEQ ID NO: 627 - DNA, 30 bp, synthetic construct
  misc_feature 1..30: note = optimized (TA) repeat constitutive promoter with15 bp between -10 and -35 elements
  source 1..30: mol_type = other DNA
acaatatatatatatatatataatgctagc

SEQ ID NO: 628 - DNA, 30 bp, synthetic construct
  misc_feature 1..30: note = optimized (TA) repeat constitutive promoter with17 bp between -10 and -35 elements
  source 1..30: mol_type = other DNA
aatatatatatatatatatataatgctagc

SEQ ID NO: 629 - DNA, 30 bp, synthetic construct
  misc_feature 1..30: note = optimized (TA) repeat constitutive promoter with19 bp between -10 and -35 elements
  source 1..30: mol_type = other DNA
tatatatatatatatatatataatgctagc

SEQ ID NO: 630 - DNA, 30 bp, synthetic construct; identical to SEQ ID NO: 629
  misc_feature 1..30: note = optimized (TA) repeat constitutive promoter with21 bp between -10 and -35 elements
  source 1..30: mol_type = other DNA

SEQ ID NO: 631 - DNA, 30 bp, synthetic construct
  misc_feature 1..30: note = optimized (A) repeat constitutive promoter with 17bp between -10 and -35 elements
  source 1..30: mol_type = other DNA
aaaaaaaaaaaaaaaaaatataatgctagc

SEQ ID NO: 632 - DNA, 30 bp, synthetic construct; identical to SEQ ID NO: 631
  misc_feature 1..30: note = optimized (A) repeat constitutive promoter with 18bp between -10 and -35 elements
  source 1..30: mol_type = other DNA

SEQ ID NO: 633 - DNA, 30 bp, synthetic construct; identical to SEQ ID NO: 572
  misc_feature 1..30: note = J23101:GFP
  source 1..30: mol_type = other DNA

SEQ ID NO: 634 - DNA, 30 bp, synthetic construct; identical to SEQ ID NO: 572
  misc_feature 1..30: note = J23119:IFP
  source 1..30: mol_type = other DNA

SEQ ID NO: 635 - DNA, 30 bp, synthetic construct; identical to SEQ ID NO: 572
  misc_feature 1..30: note = J23119:HO1
  source 1..30: mol_type = other DNA

SEQ ID NO: 636 - DNA, 30 bp, synthetic construct; identical to SEQ ID NO: 572
  misc_feature 1..30: note = Infrared signal reporter (J23119:IFP:J23119:HO1)
  source 1..30: mol_type = other DNA

SEQ ID NO: 637 - DNA, 30 bp, synthetic construct; identical to SEQ ID NO: 592
  misc_feature 1..30: note = Double terminator + constitutive promoter
  source 1..30: mol_type = other DNA

SEQ ID NO: 638 - DNA, 30 bp, synthetic construct
  misc_feature 1..30: note = Double terminator + Constitutive promoter + StrongRBS
  source 1..30: mol_type = other DNA
tgctagctactagagattaaagaggagaaa

SEQ ID NO: 639 - DNA, 30 bp, synthetic construct
  misc_feature 1..30: note = IPTG inducible Lac promoter cassette
  source 1..30: mol_type = other DNA
ttgtgagcggataacaagatactgagcaca

SEQ ID NO: 640 - DNA, 30 bp, synthetic construct; identical to SEQ ID NO: 639
  misc_feature 1..30: note = IPTG inducible Lac promoter cassette
  source 1..30: mol_type = other DNA

SEQ ID NO: 641 - DNA, 30 bp, synthetic construct; identical to SEQ ID NO: 639
  misc_feature 1..30: note = IPTG inducible Lac promoter cassette
  source 1..30: mol_type = other DNA

SEQ ID NO: 642 - DNA, 30 bp, synthetic construct
  misc_feature 1..30: note = M13K07 gene I promoter
  source 1..30: mol_type = other DNA
cctgtttttatgttattctctctgtaaagg

SEQ ID NO: 643 - DNA, 30 bp, synthetic construct
  misc_feature 1..30: note = M13K07 gene II promoter
  source 1..30: mol_type = other DNA
aaatatttgcttatacaatcttcctgtttt

SEQ ID NO: 644 - DNA, 30 bp, synthetic construct
  misc_feature 1..30: note = M13K07 gene III promoter
  source 1..30: mol_type = other DNA
gctgataaaccgatacaattaaaggctcct

SEQ ID NO: 645 - DNA, 30 bp, synthetic construct
  misc_feature 1..30: note = M13K07 gene IV promoter
  source 1..30: mol_type = other DNA
ctcttctcagcgtcttaatctaagctatcg

SEQ ID NO: 646 - DNA, 30 bp, synthetic construct
  misc_feature 1..30: note = M13K07 gene V promoter
  source 1..30: mol_type = other DNA
atgagccagttcttaaaatcgcataaggta

SEQ ID NO: 647 - DNA, 30 bp, synthetic construct
  misc_feature 1..30: note = M13K07 gene VI promoter
  source 1..30: mol_type = other DNA
ctattgattgtgacaaaataaacttattcc

SEQ ID NO: 648 - DNA, 30 bp, synthetic construct
  misc_feature 1..30: note = M13K07 gene VIII promoter
  source 1..30: mol_type = other DNA
gtttcgcgcttggtataatcgctgggggtc

SEQ ID NO: 649 - DNA, 30 bp, synthetic construct
  misc_feature 1..30: note = M13110
  source 1..30: mol_type = other DNA
ctttgcttctgactataatagtcagggtaa

SEQ ID NO: 650 - DNA, 30 bp, synthetic construct
  misc_feature 1..30: note = Modified promoter sequence of g3.
  source 1..30: mol_type = other DNA
aaaccgatacaattaaaggctcctgctagc

SEQ ID NO: 651 - DNA, 30 bp, synthetic construct
  misc_feature 1..30: note = Constitutive Promoter I
  source 1..30: mol_type = other DNA
caccacactgatagtgctagtgtagatcac

SEQ ID NO: 652 - DNA, 30 bp, synthetic construct
  misc_feature 1..30: note = Constitutive Promoter II
  source 1..30: mol_type = other DNA
gccggaataactccctataatgcgccacca

SEQ ID NO: 653 - DNA, 30 bp, synthetic construct
  misc_feature 1..30: note = --Specify Parts List--
  source 1..30: mol_type = other DNA
ttgacaagcttttcctcagctccgtaaact

SEQ ID NO: 654 - DNA, 30 bp, synthetic construct
  misc_feature 1..30: note = Full-length stationary phase osmY promoter
  source 1..30: mol_type = other DNA
ggtttcaaaattgtgatctatatttaacaa

SEQ ID NO: 655 - DNA, 30 bp, synthetic construct; identical to SEQ ID NO: 654
  misc_feature 1..30: note = Minimal stationary phase osmY promoter
  source 1..30: mol_type = other DNA

SEQ ID NO: 656 - DNA, 30 bp, synthetic construct
  misc_feature 1..30: note = htpG Heat Shock Promoter
  source 1..30: mol_type = other DNA
tctattccaataaagaaatcttcctgcgtg

SEQ ID NO: 657 - DNA, 30 bp, synthetic construct
  misc_feature 1..30: note = Promoter veg a constitutive promoter for B.subtilis
  source 1..30: mol_type = other DNA
aaaaatgggctcgtgttgtacaataaatgt

SEQ ID NO: 658 - DNA, 30 bp, synthetic construct
  misc_feature 1..30: note = Promoter 43 a constitutive promoter for B.subtilis
  source 1..30: mol_type = other DNA
aaaaaaagcgcgcgattatgtaaaatataa

SEQ ID NO: 659 - DNA, 30 bp, synthetic construct
  misc_feature 1..30: note = Strong constitutive promoter for Bacillus subtilis
  source 1..30: mol_type = other DNA
aattgcagtaggcatgacaaaatggactca

SEQ ID NO: 660 - DNA, 30 bp, synthetic construct
  misc_feature 1..30: note = PliaG
  source 1..30: mol_type = other DNA
caagcttttcctttataatagaatgaatga

SEQ ID NO: 661 - DNA, 30 bp, synthetic construct
  misc_feature 1..30: note = PlepA
  source 1..30: mol_type = other DNA
tctaagctagtgtattttgcgtttaatagt

SEQ ID NO: 662 - DNA, 30 bp, synthetic construct
  misc_feature 1..30: note = Pveg
  source 1..30: mol_type = other DNA
aatgggctcgtgttgtacaataaatgtagt

SEQ ID NO: 663 - DNA, 30 bp, synthetic construct
  misc_feature 1..30: note = Promoter ctc for B. subtilis
  source 1..30: mol_type = other DNA
atccttatcgttatgggtattgtttgtaat

SEQ ID NO: 664 - DNA, 30 bp, synthetic construct
  misc_feature 1..30: note = Promoter gsiB for B. subtilis
  source 1..30: mol_type = other DNA
taaaagaattgtgagcgggaatacaacaac

SEQ ID NO: 665 - DNA, 30 bp, synthetic construct; identical to SEQ ID NO: 658
  misc_feature 1..30: note = Promoter 43 a constitutive promoter for B.subtilis
  source 1..30: mol_type = other DNA

SEQ ID NO: 666 - DNA, 30 bp, synthetic construct
  misc_feature 1..30: note = Pspv2 from Salmonella
  source 1..30: mol_type = other DNA
tacaaaataattcccctgcaaacattatca

SEQ ID NO: 667 - DNA, 30 bp, synthetic construct
  misc_feature 1..30: note = Pspv from Salmonella
  source 1..30: mol_type = other DNA
tacaaaataattcccctgcaaacattatcg

SEQ ID NO: 668 - DNA, 30 bp, synthetic construct
  misc_feature 1..30: note = T7 promoter (strong promoter from T7bacteriophage)
  source 1..30: mol_type = other DNA
agggaatacaagctacttgttctttttgca

SEQ ID NO: 669 - DNA, 23 bp, synthetic construct
  misc_feature 1..23: note = T7 Promoter
  source 1..23: mol_type = other DNA
taatacgactcactatagggaga

SEQ ID NO: 670 - DNA, 28 bp, synthetic construct
  misc_feature 1..28: note = T7 Promoter
  source 1..28: mol_type = other DNA
gaatttaatacgactcactatagggaga

SEQ ID NO: 671 - DNA, 19 bp, synthetic construct
  misc_feature 1..19: note = T7 consensus -10 and rest
  source 1..19: mol_type = other DNA
taatacgactcactatagg

SEQ ID NO: 672 - DNA, 30 bp, synthetic construct
  misc_feature 1..30: note = overlapping T7 promoter
  source 1..30: mol_type = other DNA
gagtcgtattaatacgactcactatagggg

SEQ ID NO: 673 - DNA, 30 bp, synthetic construct
  misc_feature 1..30: note = more overlapping T7 promoter
  source 1..30: mol_type = other DNA
agtgagtcgtactacgactcactatagggg

SEQ ID NO: 674 - DNA, 30 bp, synthetic construct
  misc_feature 1..30: note = weaken overlapping T7 promoter
  source 1..30: mol_type = other DNA
gagtcgtattaatacgactctctatagggg

SEQ ID NO: 675 - DNA, 23 bp, synthetic construct
  misc_feature 1..23: note = T7 Consensus Promoter Sequence
  source 1..23: mol_type = other DNA
taatacgactcactatagggaga

SEQ ID NO: 676 - DNA, 23 bp, synthetic construct
  misc_feature 1..23: note = T7 RNAP promoter
  source 1..23: mol_type = other DNA
ttatacgactcactatagggaga

SEQ ID NO: 677 - DNA, 23 bp, synthetic construct
  misc_feature 1..23: note = T7 RNAP promoter
  source 1..23: mol_type = other DNA
gaatacgactcactatagggaga

SEQ ID NO: 678 - DNA, 23 bp, synthetic construct
  misc_feature 1..23: note = T7 RNAP promoter
  source 1..23: mol_type = other DNA
taatacgtctcactatagggaga

SEQ ID NO: 679 - DNA, 23 bp, synthetic construct
  misc_feature 1..23: note = T7 RNAP promoter
  source 1..23: mol_type = other DNA
tcatacgactcactatagggaga

SEQ ID NO: 680 - DNA, 30 bp, synthetic construct
  misc_feature 1..30: note = T7 strong promoter
  source 1..30: mol_type = other DNA
taatacgactcactatagggagaccacaac

SEQ ID NO: 681 - DNA, 30 bp, synthetic construct
  misc_feature 1..30: note = T7 weak binding and processivity
  source 1..30: mol_type = other DNA
taattgaactcactaaagggagaccacagc

SEQ ID NO: 682 - DNA, 30 bp, synthetic construct
  misc_feature 1..30: note = T7 weak binding promoter
  source 1..30: mol_type = other DNA
cgaagtaatacgactcactattagggaaga

SEQ ID NO: 683 - DNA, 30 bp, synthetic construct
  misc_feature 1..30: note = pCyc (Medium) Promoter
  source 1..30: mol_type = other DNA
acaaacacaaatacacacactaaattaata

SEQ ID NO: 684 - DNA, 30 bp, synthetic construct
  misc_feature 1..30: note = pAdh (Strong) Promoter
  source 1..30: mol_type = other DNA
ccaagcatacaatcaactatctcatataca

SEQ ID NO: 685 - DNA, 30 bp, synthetic construct
  misc_feature 1..30: note = pSte5 (Weak) Promoter
  source 1..30: mol_type = other DNA
gatacaggatacagcggaaacaacttttaa

SEQ ID NO: 686 - DNA, 30 bp, synthetic construct
  misc_feature 1..30: note = yeast ADH1 promoter
  source 1..30: mol_type = other DNA
tttcaagctataccaagcatacaatcaact

SEQ ID NO: 687 - DNA, 30 bp, synthetic construct
  misc_feature 1..30: note = cyc100 minimal promoter
  source 1..30: mol_type = other DNA
cctttgcagcataaattactatacttctat

SEQ ID NO: 688 - DNA, 30 bp, synthetic construct; identical to SEQ ID NO: 687
  misc_feature 1..30: note = cyc70 minimal promoter
  source 1..30: mol_type = other DNA

SEQ ID NO: 689 - DNA, 30 bp, synthetic construct; identical to SEQ ID NO: 687
  misc_feature 1..30: note = cyc43 minimal promoter
  source 1..30: mol_type = other DNA

SEQ ID NO: 690 - DNA, 30 bp, synthetic construct; identical to SEQ ID NO: 687
  misc_feature 1..30: note = cyc28 minimal promoter
  source 1..30: mol_type = other DNA

SEQ ID NO: 691 - DNA, 30 bp, synthetic construct; identical to SEQ ID NO: 687
  misc_feature 1..30: note = cyc16 minimal promoter
  source 1..30: mol_type = other DNA

SEQ ID NO: 692 - DNA, 30 bp, synthetic construct
  misc_feature 1..30: note = pPGK1
  source 1..30: mol_type = other DNA
ttatctactttttacaacaaatataaaaca

SEQ ID NO: 693 - DNA, 30 bp, synthetic construct; identical to SEQ ID NO: 683
  misc_feature 1..30: note = pCYC Yeast Promoter
  source 1..30: mol_type = other DNA

SEQ ID NO: 694 - DNA, 30 bp, synthetic construct
  misc_feature 1..30: note = Yeast GPD (TDH3) Promoter
  source 1..30: mol_type = other DNA
gtttcgaataaacacacataaacaaacaaa

SEQ ID NO: 695 - DNA, 30 bp, synthetic construct; identical to SEQ ID NO: 684
  misc_feature 1..30: note = yeast mid-length ADH1 promoter
  source 1..30: mol_type = other DNA

SEQ ID NO: 696 - DNA, 30 bp, synthetic construct
  misc_feature 1..30: note = Yeast CLB1 promoter region, G2/M cell cyclespecific
  source 1..30: mol_type = other DNA
accatcaaaggaagctttaatcttctcata

SEQ ID NO: 697 - DNA, 30 bp, synthetic construct
  misc_feature 1..30: note = CMV promoter
  source 1..30: mol_type = other DNA
agaacccactgcttactggcttatcgaaat

SEQ ID NO: 698 - DNA, 30 bp, synthetic construct
  misc_feature 1..30: note = Ubc Promoter
  source 1..30: mol_type = other DNA
ggccgtttttggcttttttgttagacgaag

(19) United States
(12) Patent Application Publication
(10) Pub. No.: US 2026/0176574 A1
(43) Pub. Date: Jun. 25, 2026

(54) CONTROLLED GROWTH OF MICROORGANISMS

(71) Applicant: Syngulon SRL, Charleroi (BE)

(72) Inventor: Philippe Gabant, Ottignies Louvain-La-Neuve (BE)

(21) Appl. No.: 19/205,894

(22) Filed: May 12, 2025

Related U.S. Application Data
(63) Continuation of application No. 17/822,663, filed on Aug. 26, 2022, now Pat. No. 12,297,422.
Application No. 17/822,663 is a continuation of application No. 16/227,371, filed on Dec. 20, 2018, now Pat. No. 11,427,800.
Application No. 16/227,371 is a continuation of application No. 15/087,706, filed on Mar. 31, 2016, now Pat. No. 10,188,114.
Application No. 15/087,706 is a division of application No. 14/459,810, filed on Aug. 14, 2014, now Pat. No. 9,333,227.
(60) Provisional application No. 61/867,510, filed on Aug. 19, 2013.

(51) Int. Cl. C12N 1/12; A01N 63/50; A61K 35/74; A61K 36/02; A61K 36/06; C07K 14/195; C12N 1/16; C12N 1/20; C12N 1/38

(52) U.S. Cl. CPC C12N 1/12; A01N 63/50; A61K 35/74; A61K 36/02; A61K 36/06; C07K 14/195; C12N 1/16; C12N 1/20; C12N 1/38

(57) ABSTRACT

It can be useful to regulate the growth of microbial cells. Some embodiments herein provide genetically engineered microbial cells that can produce bacteriocins to control the growth of microbial cells. In some embodiments, microbial cells are contained within a desired environment. In some embodiments, contaminating microbial cells are neutralized. In some embodiments, a first microbial cell type regulates the growth of a second microbial cell type so as to maintain a desired ratio of the two cell types.

### RELATED APPLICATIONS

This application is a continuation of U.S. application Ser. No. 16/227,371, filed Dec. 20, 2018, which is a continuation of U.S. application Ser. No. 15/087,706, filed Mar. 31, 2016, issued Jan. 19, 2019 as U.S. Pat. No. 10,188,114, which is a divisional of U.S. application Ser. No. 14/459,810, filed Aug. 14, 2014, issued May 10, 2016 as U.S. Pat. No. 9,333,227, which claims the benefit of U.S. Provisional Application No. 61/867,510, filed on Aug. 19, 2013, each of which is hereby incorporated by reference in its entirety.

### REFERENCE TO SEQUENCE LISTING

The present application is being filed along with a Sequence Listing in electronic format. The Sequence Listing is provided as a file entitled SYNG001C3_SEQLIST.xml, created and last saved on May 12, 2025, which is 811,716 bytes in size. The information in the electronic format of the Sequence Listing is incorporated herein by reference in its entirety.

### BACKGROUND

Humans have used microbial organisms to generate products since the beginning of human history, for example in processing foods such as cheese, beer, and wine. During the centuries, microbial organism-mediated processes have been studied and scaled-up, often by controlling fermentation conditions or identification of phenotypic characteristics of microbial organisms.

Presently, many products are produced using a process that involves microbial organisms. In laboratories, and in some pharmaceutical manufacturing processes, microbial organisms, including genetically engineered microbial organisms, can be cultured in sterile, controlled environments. On the other hand, feedstocks used for various industrial processes involving microorganisms are not sterile, and may contain a variety of strains and species of microorganisms. As such, genetically engineered microorganisms for laboratory and pharmaceutical processes are not necessarily suited for processes, such as industrial processes, which involve using feedstocks or which are exposed to other microorganisms in the environment which could potentially contaminate the culture and which may also involve, changing environmental conditions. Herein microorganisms which have been engineered to control their own growth and the growth of other microorganisms and/or to respond to changes in their environment are described. Such microorganisms are suitable for growth in non-sterile, less rigidly controlled feedstocks. Such microorganisms can be useful for robust, consistent production of a desired product across a range of different feedstocks and environments.

### FIELD

Embodiments herein relate generally to the control of growth of microorganisms. More particularly, some embodiments herein relate to microorganisms engineered for regulated growth in response to other microorganisms and/or conditions of the culture environment, and methods of making and using such engineered microorganisms.

### SUMMARY

One embodiment disclosed herein includes a first microbial cell comprising a nucleic acid encoding a secreted bacteriocin which controls the growth of a second microbial cell and a nucleic acid which confers resistance to the secreted bacteriocin is provided, in which the first microbial cell has been genetically engineered to allow the expression or activity of the nucleic acid which confers resistance to the bacteriocin to be regulated. According to some aspects of this embodiment, the expression or activity of the nucleic acid which confers resistance to the bacteriocin is reduced to a level which causes the first microbial cell to be neutralized by the bacteriocin if the first microbial cell is released from a desired growth environment. According to some aspects of this embodiment, the first microbial cell has been genetically engineered to make a desired product. According to some aspects of this embodiment, the secreted bacteriocin further has been selected to maintain at least one condition within a culture in which the first microbial cell is producing the desired product. According to some aspects of this embodiment, the culture comprises at least one invading microbial organism. According to some aspects of this embodiment, the at least one condition of the culture comprises controlling the growth of the second microbial cell, wherein the second microbial cell is a common contaminate of the culture. According to some aspects of this embodiment, the second microbial cell is a different strain, species or genus than the first microbial cell. According to some aspects of this embodiment, the microbial cell further comprises a nucleic acid encoding a second secreted bacteriocin which controls the growth of a third microbial cell and a nucleic acid which confers resistance to the secreted second bacteriocin, and also the first microbial cell has been genetically engineered to allow the expression or activity of the nucleic acid which confers resistance to the bacteriocin to be regulated. According to some aspects of this embodiment, the bacteriocin kills the second microbial cell. According to some aspects of this embodiment, the bacteriocin reduces the growth rate of the second microbial cell. According to some aspects of this embodiment, the bacteriocin arrests the growth of the second microbial cell. According to some aspects of this embodiment, the transcription of the nucleic acid conferring resistance to the bacteriocin is under the control of a regulatable promoter. According to some aspects of this embodiment, the activity of a polypeptide encoded by the nucleic acid conferring resistance to the bacteriocin is regulatable. According to some aspects of this embodiment, the nucleic acid encoding the bacteriocin is on a chromosome of the microbial cell. According to some aspects of this embodiment, the nucleic acid conferring resistance to the bacteriocin is on a plasmid. According to some aspects of this embodiment, the nucleic acid encoding the bacteriocin is on a chromosome of the microbial cell, and the nucleic acid conferring resistance to the bacteriocin is on a plasmid. According to some aspects of this embodiment, the nucleic acid encoding the bacteriocin and the nucleic acid conferring resistance to the bacteriocin are on one or more plasmids. According to some aspects of this embodiment, the first microbial cell is selected from the group consisting of bacteria, yeast, and algae, for example photosynthetic microalgae.

Another embodiment disclosed herein includes a method of controlling the growth of a second microbial cell in a culture medium, in which the method includes comprising culturing a first microbial cell as described herein in a culture medium comprising the second microbial cell under conditions in which the first microbial cell produces a bacteriocin at a level sufficient to control the growth of the second microbial cell. According to some aspects of this embodiment, the culture is maintained continually for at least 30 days, for example at least 30, 35, 40, 45, 50, 55, 60, 65, 70, 75, 80, 85, 90, 95, 100, 110, 120, 130, 140, 150, 160, 170, 180, 190, 200, 250, 300, 350, 400, 450, or 500 days. According to some aspects of this embodiment, the method further includes detecting at least one change in the culture medium, the change comprising a presence or increase in the levels or activity of a third microbial cell, and reengineering the first microbial cell in response to the change to produce a second bacteriocin at a level sufficient to control the growth of the third microbial cell.

Another embodiment disclosed herein includes a method of detecting a presence, absence, or amount of a molecule in a culture is provided. The method can comprise culturing a first genetically engineered microbial cell comprising a bacteriocin under the control of a genetically regulatable promoter, such that the regulatable promoter is regulated by the molecule so that either (a) the regulatable promoter drives transcription in the presence of the molecule, but not in the absence of the molecule; or (b) the regulatable promoter drives transcription in the absence of the molecule, but not in the presence of the molecule. The method can comprise isolating an amount of genomic nucleic acid of the first microbial cell from the culture. The method can comprise detecting from the amount of genomic nucleic acid, a presence, absence, or quantity of a nucleic acid sequence characteristic of the first microbial cell. According to some aspects of this embodiment, the method further includes comparing the quantity of the nucleic acid sequence characteristic of the first microbial cell to a quantity of a reference nucleic acid sequence.

Another embodiment disclosed herein includes a genetically engineered vector comprising a nucleic acid conferring resistance to a bacteriocin, in which the expression or activity of the nucleic acid is configured to change in response to the presence, level or absence of a component of a feedstock. According to some aspects of this embodiment, the vector further comprises a nucleic acid encoding the bacteriocin. According to some aspects of this embodiment, the vector further comprises a nucleic acid which encodes a desired product.

Another embodiment disclosed herein includes a kit, which can includes a plurality of strains of a genetically engineered microbial organism, in which each strain has been genetically engineered to allow the expression or activity of a nucleic acid which confers resistance to a different bacteriocin to be regulated.

Another embodiment disclosed herein includes a method of identifying at least one bacteriocin which modulates the growth of at least one microbial cell in an industrial culture medium, in which the method includes contacting the industrial culture medium with a medium or composition comprising the at least one bacteriocin; and determining whether the at least one bacteriocin has a desired effect on the growth of the at least one microbial cell. According to some aspects of this embodiment, the method includes contacting the industrial culture medium with at least one bacteriocin produced by a first microbial cell as described herein. According to some aspects of this embodiment, the at least one bacteriocin produced by the first microbial cell is in a supernatant obtained from a culture comprising the first microbial cell. According to some aspects of this embodiment, the method further includes constructing a genetically engineered microbial cell to produce at least one bacteriocin which has been determined to have a desired effect on the growth of the at least one microbial cell. According to some aspects of this embodiment, the at least one microbial cell is an organism which is a common invader of the industrial culture medium. According to some aspects of this embodiment, the at least one microbial cell is an organism which has newly invaded an existing industrial culture.

Another embodiment disclosed herein includes a system for neutralizing undesired microbial organisms in a culture medium. The system can comprise a first environment comprising a culture medium, and a second environment comprising a second microbial organism that secretes two or more different bacteriocins, in which the second microbial organism comprises immunity modulators for each of the two or more different bacteriocins, in which the second environment is in fluid communication with the first environment, in which the second environment is physically separated from the first environment so that the second microbial organism cannot move from the second environment to the first environment, and in which the secreted two or more different bacteriocins enter the culture medium of the first environment. According to some aspects of this embodiment, the system further comprises a first microbial organism in the the culture medium, in which the first microbial organism does not secrete the two or more different bacteriocins, and in which the first microbial organism is not neutralized by any of the two or more different bacteriocins. According to some aspects of this embodiment, the first microbial organism is non-GMO. According to some aspects of this embodiment, the first microbial organism ferments a component of the culture medium. According to some aspects of this embodiment, the first microbial organism decontaminates the culture medium. According to some aspects of this embodiment, the first microbial organism conducts photosynthesis, and the photosynthesis comprises a substrate comprised by the culture medium. According to some aspects of this embodiment, the second environment is separated from the first environment by at least one of a membrane, a mesh, a filter, or a valve that is permeable to the two or more different bacteriocins, but is not permeable to the second microbial organisms. According to some aspects of this embodiment, the second microbial organism secretes at least 3 bacteriocins, for example at least 3, 4, 5, 6, 7, 8, 9, 10, 11, 12, 13, 14, 15, 16, 17, 18, 19, or 20 bacteriocins. According to some aspects of this embodiment, the second environment comprises at least a third microbial organism that is different from the second microbial organism, and also secretes bacteriocins. According to some aspects of this embodiment, the third microbial organism secretes at least 2 bacteriocins, for example at least 2, 3, 4, 5, 6, 7, 8, 9, 10, 11, 12, 13, 14, 15, 16, 17, 18, 19, or 20 bacteriocins. Another embodiment disclosed herein includes a method of storing a feedstock. The method can comprise providing a feedstock, providing a first microbial organism, in which the first microbial organism secretes two or more different bacteriocins, contacting the feedstock with the bacteriocins, and storing the feedstock for a desired period of time. According to some aspects of this embodiment, contacting the feedstock with the bacteriocins comprises contacting the feedstock with the microbial organism. According to some aspects of this embodiment, contacting the feedstock with the bacteriocins comprises placing the microbial organism in fluid communication with the feedstock, while maintaining physical separation between the microbial organism and the feedstock, so that the bacteriocins contact the feedstock, but the microbial organism does not directly contact the feedstock. According to some aspects of this embodiment, the separation is maintained by at least one or more of a membrane, a mesh, a filter, or a valve that is permeable to the two or more different bacteriocins, but is not permeable to the first microbial organism. According to some aspects of this embodiment, the method further comprises fermenting the feedstock with a second microbial organism prior to or concurrently with contacting the feedstock with the bacteriocins. According to some aspects of this embodiment, the fermentation comprises at least one of producing a desired component in the feedstock or removing an undesired component from the feedstock. According to some aspects of this embodiment, the desired period of time comprises at least one month, for example at least one, two, three, four, five, six, seven, eight, nine, ten, eleven, or twelve months. According to some aspects of this embodiment, the desired period of time comprises at least six months, for example six, seven, eight nine, ten, eleven, or tweleve months. According to some aspects of this embodiment, the first microbial organism secretes at least 3 bacteriocins, for example at least 3, 4, 5, 6, 7, 8, 9, 10, 11, 12, 13, 14, 15, 16, 17, 18, 19, or 20 bacteriocins.

### BRIEF DESCRIPTION OF THE DRAWINGS

FIG. 1 is a flow diagram depicting options for configuring a microbial cell to control the growth of a second microbial cell according to some of the embodiments herein.

FIG. 2A is a schematic diagram illustrating a first microbial cell controlling the growth of other microbial cells according to some of the embodiments herein.

FIG. 2B is a schematic diagram illustrating control of the growth of a first microbial cell when the first microbial cell is no longer in a desired growth environment according to some of the embodiments herein.

FIG. 3 is a schematic diagram illustrating a first microbial cell controlling growth of a second microbial cell and neutralizing an invading cell in a desired environment according to some of the embodiments herein.

FIG. 4 is a schematic diagram illustrating a first microbial cell neutralizing a first invading cell with a first bacteriocin and second invading cells with a second bacteriocin in a desired environment according to some of the embodiments herein.

FIG. 5 is a flow diagram illustrating methods of controlling the growth of at least a second microbial cell in culture according to some embodiments herein.

FIG. 6 is a schematic diagram illustrating a system comprising a genetic guard in accordance with some embodiments herein.

FIG. 7 is a schematic diagram illustrating a genetic guard system that can be useful for photosynthetic production in accordance with some embodiments herein.

FIG. 8 is a flow diagram illustrating methods of producing and using bacteriocins in accordance with some embodiments herein.

### DETAILED DESCRIPTION

According to some of the embodiments herein, genetically engineered microbial organisms are provided. In some embodiments, the microbial organisms are engineered to control the growth of the microbial population in an environment such as those employing a feedstock. As used herein, “neutralizing” activity (and variations of the same root word) of bacteriocins can refer to either arrest of microbial reproduction, or cytotoxicity. Microbial organisms can be engineered to produce bacteriocins, which are secreted polypeptides that can neutralize microorganisms. However, microbial organisms that produce bacteriocin immunity modulators can resist certain bacteriocins. Thus, in some embodiments, a first microbial organism is engineered to secrete bacteriocins. In some embodiments, the particular bacteriocins are selected based on the type of microbial cell, the types of microbial cells being regulated, the composition of the culture medium, or geographic location (for example, to target particular contaminating microbial organisms associated with a particular type of culture medium and/or geographical location). Other microbial organisms that possess desired characteristics for a particular environment can produce bacteriocin immunity modulators (and thus survive in the presence of bacteriocins), while undesired other microbial organisms (for example contaminants, microbial organisms that have lost a desired characteristic or organisms which are involved in an industrial process but whose growth or production of a particular product is not desired under the prevailing conditions) fail to produce bacteriocin immunity modulators, and are thus neutralized by the bacteriocins.

### Microbial Organisms

According to some aspects, genetically engineered microorganisms are provided. As used herein, genetically engineered “microbial organism,” “microorganism,” and variations of these root terms (such as pluralizations and the like), encompasses genetic modification of any naturally-occurring species or fully synthetic prokaryotic or eukaryotic unicellular organism, as well as Archae species. Thus, this expression can refer to cells of bacterial species, fungal species, and algae.

Exemplary microorganisms that can be used in accordance with embodiments herein include, but are not limited to, bacteria, yeast, and algae, for example photosynthetic microalgae. Furthermore, fully synthetic microorganism genomes can be synthesized and transplanted into single microbial cells, to produce synthetic microorganisms capable of continuous self-replication (see Gibson et al. (2010), “Creation of a Bacterial Cell Controlled by a Chemically Synthesized Genome,” Science 329: 52-56, hereby incorporated by reference in its entirety). As such, in some embodiments, the microorganism is fully synthetic. A desired combination of genetic elements, including elements that regulate gene expression, and elements encoding gene products (for example bacteriocins, immunity modulators, poison, antidote, and industrially useful molecules) can be assembled on a desired chassis into a partially or fully synthetic microorganism. Description of genetically engineered microbial organisms for industrial applications can also be found in Wright, et al. (2013) “Building-in biosafety for synthetic biology” Microbiology 159: 1221-1235.

A variety of bacterial species and strains can be used in accordance with embodiments herein, and genetically modified variants, or synthetic bacteria based on a “chassis” of a known species can be provided. Exemplary bacteria with industrially applicable characteristics, which can be used in accordance with embodiments herein include, but are not limited to, Bacillus species (for example Bacillus coagulans, Bacillus subtilis, and Bacillus licheniformis), Paenibacillus species, Streptomyces species, Micrococcus species, Corynebacterium species, Acetobacter species, Cyanobacteria species, Salmonella species, Rhodococcus species, Pseudomonas species, Lactobacillus species, Enterococcus species, Alcaligenes species, Klebsiella species, Paenibacillus species, Arthrobacter species, Corynebacterium species, Brevibacterium species, Thermus aquaticus, Pseudomonas stutzeri, Clostridium thermocellus, and Escherichia coli.

A variety of yeast species and strains can be used in accordance with embodiments herein, and genetically modified variants, or synthetic yeast based on a “chassis” of a known species can be provided. Exemplary yeast with industrially applicable characteristics, which can be used in accordance with embodiments herein include, but are not limited to Saccharomyces species (for example, Saccharomyces cerevisiae, Saccharomyces bayanus, Saccharomyces boulardii), Candida species (for example, Candida utilis, Candida krusei), Schizosaccharomyces species (for example Schizosaccharomyces pombe, Schizosaccharomyces japonicas), Pichia or Hansenula species (for example, Pichia pastoris or Hansenula polymorpha) species, and Brettanomyces species (for example, Brettanomyces claussenii).

A variety of algae species and strains can be used in accordance with embodiments herein, and genetically modified variants, or synthetic algae based on a “chassis” of a known species can be created. In some embodiments, the algae comprises photosynthetic microalgae. Exemplary algae species that can be useful for biofuels, and can be used in accordance with embodiments herein, include Botryococcus braunii, Chlorella species, Dunaliella tertiolecta, Gracilaria species, Pleurochrysis carterae, and Sargassum species. Additionally, many algaes can be useful for food products, fertilizer products, waste neutralization, environmental remediation, and carbohydrate manufacturing (for example, biofuels).

### Bacteriocins

As used herein, “bacteriocin,” and variations of this root term, refers to a polypeptide that is secreted by a host cell and can neutralize at least one cell other than the individual host cell in which the polypeptide is made, including cells clonally related to the host cell and other microbial cells. As used herein, “bacteriocin” also encompasses a cell-free or chemically synthesized version of such a polypeptide. A cell that expresses a particular “immunity modulator” (discussed in more detail herein) is immune to the neutralizing effects of a particular bacteriocin or group of bacteriocins. As such, bacteriocins can neutralize a cell producing the bacteriocin and/or other microbial cells, so long as these cells do not produce an appropriate immunity modulator. As such, a host cell can exert cytotoxic or growth-inhibiting effects on a plurality of other microbial organisms by secreting bacteriocins. In some embodiments, a bacteriocin is produced by the translational machinery (e.g. a ribosome, etc.) of a microbial cell. In some embodiments, a bacteriocin is chemically synthesized. Some bacteriocins can be derived from a polypeptide precursor. The polypeptide precursor can undergo cleavage (for example processing by a protease) to yield the polypeptide of the bacteriocin itself. As such, in some embodiments, a bacteriocin is produced from a precursor polypeptide. In some embodiments, a bacteriocin comprises a polypeptide that has undergone post-translational modifications, for example cleavage, or the addition of one or more functional groups.

“Antibiotic,” and variations of this root term, refers to a metabolite, or an intermediate of a metabolic pathway which can kill or arrest the growth of at least one microbial cell. Some antibiotics can be produced by microbial cells, for example bacteria. Some antibiotics can be synthesized chemically. It is understood that bacteriocins are distinct from antibiotics, at least in that bacteriocins refer to gene products (which, in some embodiments, undergo additional post-translational processing) or synthetic analogs of the same, while antibiotics refer to intermediates or products of metabolic pathways or synthetic analogs of the same.

Neutralizing activity of bacteriocins can include arrest of microbial reproduction, or cytotoxicity. Some bacteriocins have cytotoxic activity (e.g. “bacteriocide” effects), and thus can kill microbial organisms, for example bacteria, yeast, algae, synthetic micoorganisms, and the like. Some bacteriocins can inhibit the reproduction of microbial organisms (e.g. “bacteriostatic” effects), for example bacteria, yeast, algae, synthetic micoorganisms, and the like, for example by arresting the cell cycle.

It is noted that non-bacteriocin approaches have been proposed to target various microbial organisms. For example, KAMORAN™ chemical has been proposed to target Lactic Acid Bacteria (LAB) family bacteria (see Union Nationale des Groupements de Distillateurs D'Alcool, (2005) “Kamoran”). It is noted that phage has also been proposed to target LAB family bacteria (see U.S. Pub. No. 2010/0330041). It is noted that pesticides have been proposed to target various contaminating microbial organsims (see McBride et al., “Contamination Management in Low Cost Open Algae Ponds for Biofuels Production” Industrial Biotechnology 10: 221-227 (2014)). However, bacteriocins can provide numerous advantages over chemicals, pesticides, or phages. For example, bacteriocins can avoid potentially toxic runoff or byproduct in a feedstock. For example, bacteriocins can have higher efficacy against particular undesired microbial organisms than phages, chemicals, or pesticides. For example, bacteriocins can be produced by microbial organisms that undergo logarithmic growth, and thus can readily be scaled-up or scaled down, whereas the scalability of phages or chemical/pesticide systems can be more limited. For example, bacteriocins can allow for precise control over which organsims are neutralized and which are not, for example to avoid neutralization of industrially useful microbial organisms in the culture medium. For example, phages can be difficult to produce at an industrial scale, and also can be difficult to control, in that phages can be infectious, can raise questions of gene control, and in that conservation of phages can be difficult. On the other hand, bacteriocins in accordance with some embodiments herein can comprise part of an industrial process and thus can be involved in gene containment and/or control a fermentation process via bacteriostatic activity. Additionally, the susceptibility of the microbial organisms involved in the industrial process can be tuned via immunity control. Additionally, bacteriocins typically have a low level of toxicity for industrial applications such as human or animal food, and it is contemplated that bacteriocins in accordance with some embodiments herein are suitable for use as a food preservative, such as an additive.

In some embodiments, a particular neutralizing activity (e.g. cytoxicity or arrest of microbial reproduction) is selected based on the type of microbial regulation that is desired. As such in some embodiments, microbial cells are engineered to express particular bacteriocins or combination of bacteriocins. For example, in some embodiments, microbial cells are engineered to express particular bacteriocins based on the cells being regulated. In some embodiments, for example if contaminating cells are to be killed at least one cytotoxic bacteriocin is provided. In some embodiments, a bacteriocin or combination of bacteriocins which is effective against contaminants which commonly occur in a particular culture, or a particular geographic location, or a particular type of culture grown in a particular geographic location are selected. In some embodiments, for example embodiments in which reversible regulation of microbial cell ratios is desired, a bacteriocin that inhibits microbial reproduction is provided. Without being limited by any particular theory, many bacteriocins can have neutralizing activity against microbial organisms that typically occupy the same ecological niche as the species that produces the bacteriocin. As such, in some embodiments, when a particular spectrum of bacteriocin activity is desired, a bacteriocin is selected from a host species that occupies the same (or similar) ecological niche as the microbial organism or organisms targeted by the bacteriocin.

In some embodiments, one or more bacteriocin activities are selected in advance of culture growth, and one or more microbial organisms are engineered to generate a desired culture environment. In some embodiments, bacteriocins may be selected based on their ability to neutralize one or more invading organisms which are likely to attempt to grow in a particular culture. In another embodiment, in an industrial environment in which strain A makes intermediate A, and strain B converts intermediate A into intermediate B, strains A and B can be engineered so that an abundance of intermediate A shifts the equilibrium to favor strain B by generating a bacteriocin activity profile such that growth of strain A is inhibited or prevented under these conditions, while a lack of intermediate A shifts the equilibrium to favor strain A by generating a bacteriocin activity profile such that growth of strain B is inhibited or prevented. In some embodiments, one or more bacteriocin activities are selected based on one or more conditions of an existing culture environment. For example, if particular invaders are identified in a culture environment, “neutralizer” microrganisms can be engineered to produce bacteriocins to neutralize the identified invaders. In some embodiments, genetically engineered cells that produce bacteriocins are added to an existing culture to re-equilibrate the culture, for example if a growth of a particular microbial cell type in the microbial cell culture is too high. In some embodiments, genetically engineered cells that produce bacteriocins are added to an existing culture to neutralize all or substantially all of the microbial cells in a culture, for example to eliminate an industrial culture in a culture environment so that a new industrial culture can be introduced to the culture environment.

For example, in some embodiments, an anti-fungal activity (such as anti-yeast activity) is desired. A number of bacteriocins with anti-fungal activity have been identified. For example, bacteriocins from Bacillus have been shown to have neutralizing activity against yeast strains (see Adetunji and Olaoye (2013) Malaysian Journal of Microbiology 9: 130-13, hereby incorporated by reference in its entirety), an Enterococcus faecalis peptide (WLPPAGLLGRCGRWFRPWLLWLQ SGAQY KWLGNLFGLGPK, SEQ ID NO: 1) has been shown to have neutralizing activity against Candida species (see Shekh and Roy (2012) BMC Microbiology 12: 132, hereby incorporated by reference in its entirety), and bacteriocins from Pseudomonas have been shown to have neutralizing activity against fungi such as Curvularia lunata, Fusarium species, Helminthosporium species, and Biopolaris species (Shalani and Srivastava (2008) The Internet Journal of Microbiology. Volume 5 Number 2. DOI: 10.5580/27dd—accessible on the worldwide web at archive.ispub.com/journal/the-internet-journal-of-microbiology/volume-5-number-2/screening-for-antifungal-activity-of-pseudomonas-fluorescens-against-phytopathogenic-fungi.html#sthash.d0Ys03UO.1DKuTlUS.dpuf, hereby incorporated by reference in its entirety). By way of example, botrycidin AJ1316 (see Zuber, P et al. (1993) Peptide Antibiotics. In Bacillus subtilis and Other Gram-Positive Bacteria: Biochemistry, Physiology, and Molecular Genetics ed Sonenshein et al., pp. 897-916, American Society for Microbiology, hereby incorporated by reference in its entirety) and alirin B1 (see Shenin et al. (1995) Antibiot Khimioter 50: 3-7, hereby incorporated by reference in its entirity) from B. subtilis have been shown to have antifungal activities. As such, in some embodiments, for example embodiments in which neutralization of a fungal microbial organism is desired, a bacteriocin comprises at least one of botrycidin AJ1316 or alirin B1.

For example, in some embodiments, bacteriocin activity in a culture of cyanobacteria is desirable. In some embodiments, bacteriocins are provided to neutralize cyanobacteria. In some embodiments, bacteriocins are provided to neutralize invading microbial organisms typically found in a cyanobacteria culture environment. Clusters of conserved bacteriocin polypetides have been identified in a wide variety of cyanobacteria species. For example, at least 145 putative bacteriocin gene clusters have been identified in at least 43 cyanobacteria species, as reported in Wang et al. (2011), Genome Mining Demonstrates the Widespread Occurrence of Gene Clusters Encoding Bacteriocins in Cyanobacteria. PLoS ONE 6(7): e22384, hereby incorporated by reference in its entirety. Exemplary cyanobacteria bacteriocins are shown in Table 1.2, as SEQ ID NO's 420, 422, 424, 426, 428, 30, 432, 434, 436, 438, 440, 442, 444, 446, 448, and 450.

In some embodiments, the host cell itself is a microbial cell. In some embodiments, bacteriocins neutralize cells of a different species or strain from the host cell. In some embodiments, bacteriocins neutralize cells of the same species or strain as the host cell if these cells lack an appropriate immunity modulator. As bacteriocins can mediate neutralization of both host and non-host microbial organisms, the skilled artisan will readily appreciate that a bacteriocin is distinct from poison-antidote systems (described in more detail herein), which involve an endogenous mechanism by which a host microorganism can neutralize only itself. In other words, bacteriocins can neutralize cells other than the cell in which they are produced (for example, bacteriocins can be selected and/or engineered to act as an ecological niche protector), while poison molecules kill only the individual cell in which they are produced (for example, to act as suicidal systems).

A number of bacteriocins have been identified and characterized. Without being limited by any particular theory, exemplary bacteriocins can be classified as “class I” bacteriocins, which typically undergo post-translational modification, and “class II” bacteriocins, which are typically unmodified. Additionally, exemplary bacteriocins in each class can be categorized into various subgroups, as summarized in Table 1.1, which is adapted from Cotter, P. D. et al. “Bacteriocins—a viable alternative to antibiotics” Nature Reviews Microbiology 11: 95-105, hereby incorporated by reference in its entirety.

Without being limited by any particular theory, bacteriocins can effect neutralization of a target microbial cell in a variety of ways. For example, a bacteriocin can permeablize a cell wall, thus depolarizing the cell wall and interfering with respiration.

TABLE 1.1
Classification of Exemplary Bacteriocins

| | | |
|---|---|---|
| Group | Distinctive feature | Examples |

Class I (typically modified)

| | | |
|---|---|---|
| MccC7-C51-type | Is covalently attached to a carboxy- | MccC7-C51 |
| bacteriocins | terminal aspartic acid | |
| Lasso peptides | Have a lasso structure | MccJ25 |
| Linear azole- or | Possess heterocycles but not other | MccB17 |
| azoline-containing | modifications | |
| peptides | | |
| Lantibiotics | Possess lanthionine bridges | Nisin, planosporicin, |
| | | mersacidin, actagardine, |
| | | mutacin 1140 |
| Linaridins | Have a linear structure and contain | Cypemycin |
| | dehydrated amino acids | |
| Proteusins | Contain multiple hydroxylations, | Polytheonamide A |
| | epimerizations and methylations | |
| Sactibiotics | Contain sulphur-α-carbon linkages | Subtilosin A, thuricin CD |
| Group | Distinctive feature | Examples |
| Patellamide-like | Possess heterocycles and undergo | Patellamide A |
| cyanobactins | macrocyclization | |
| Anacyclamide-like | Cyclic peptides consisting of | Anacyclamide A10 |
| cyanobactins | proteinogenic amino acids with prenyl | |
| | attachments | |
| Thiopeptides | Contain a central pyridine, | Thiostrepton, nocathiacin |
| | dihydropyridine or piperidine ring as | I, GE2270 A, philipimycin |
| | well as heterocycles | |
| Bottromycins | Contain macrocyclic amidine, a | Bottromycin A2 |
| | decarboxylated carboxy-terminal | |
| | thiazole and carbon-methylated amino | |
| | acids | |
| Glycocins | Contain S-linked glycopeptides | Sublancin 168 |

Class II (typically unmodified or cyclic)

| | | |
|---|---|---|
| IIa peptides (pediocin | Possess a conserved YGNGV motif | Pediocin PA-1, enterocin |
| PA-1-like | (in which N represents any amino | CRL35, carnobacteriocin |
| bacteriocins) | acid) | BM1 |
| IIb peptides | Two unmodified peptides are required | ABP118, lactacin F |
| | for activity | |
| IIc peptides | Cyclic peptides | Enterocin AS-48 |
| IId peptides | Unmodified, linear, non-pediocin-like, | MccV, MccS, epidermicin |
| | single-peptide bacteriocins | NI01, lactococcin A |
| IIe peptides | Contain a serine-rich carboxy-terminal | MccE492, MccM |
| | region with a non-ribosomal | |
| | siderophore-type modification | |

A number of bacteriocins can be used in accordance with embodiments herein. Exemplary bacteriocins are shown in Table 1.2. In some embodiments, at least one bacteriocin comprising a polypeptide sequence of Table 1.2 is provided. As shown in Table 1.2, some bacteriocins function as pairs of molecules. As such, it will be understood that unless explicity stated otherwise, when a functional “bacteriocin” or “providing a bacteriocin,” or the like is discussed herein, functional bacteriocin pairs are included along with bacteriocins that function individually. With reference to Table 1.2, “organisms of origin” listed in parentheses indicate alternative names and/or strain information for organisms known the produce the indicated bacteriocin.

Embodiments herein also include peptides and proteins with identity to bacteriocins described in Table 1.2. The term “identity” is meant to include nucleic acid or protein sequence homology or three-dimensional homology. Several techniques exist to determine nucleic acid or polypeptide sequence homology and/or three-dimensional homology to polypeptides. These methods are routinely employed to discover the extent of identity that one sequence, domain, or model has to a target sequence, domain, or model. A vast range of functional bacteriocins can incorporate features of bacteriocins disclosed herein, thus providing for a vast degree of identity to the bacteriocins in Table 1.2. In some embodiments, a bacteriocin has at least about 50% identity, for example, at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the polypeptides of Table 1.2. Percent identity may be determined using the BLAST software (Altschul, S. F., et al. (1990) “Basic local alignment search tool.” J. Mol. Biol. 215:403-410, accessible on the world wide web at blast.ncbi.nlm.nih.gov) with the default parameters.

In some embodiments, a polynucleotide encoding a bacteriocin as described herein is provided. In some embodiments, the polynucleotide is comprised within an expression vector. In some embodiments, the polynucleotide or expression vector is in a microbial cell. Exemplary polynucleotide sequences encoding the polypeptides of table 1.2 are indicated in table 1.2. SEQ ID NOs: 341 to 419 (odd SEQ ID numbers) represent exemplary polynucleotides based on the reverse translation of the respective polypeptide. The skilled artisan will readily understand that more than one polynucleotide can encode a particular polypeptide. For example, the genetic code is degenerate, and moreover, codon usage can vary based on the particular organism in which the gene product is being expressed. In some embodiments, a polynucleotide encoding a bacteriocin is selected based on the codon usage of the organism expressing the bacteriocin. In some embodiments, a polynucleotide encoding a bacteriocin is codon optimized based on the particular organism expressing the bacteriocin.

While the bacteriocins in Table 1.2 are naturally-occuring, the skilled artisan will appreciate that variants of the bacteriocins of Table 1.2, naturally-occuring bacteriocins other than the bacteriocins of Table 1.2 or variants thereof, or synthetic bacteriocins can be used according to some embodiments herein. In some embodiments, such variants have enhanced or decreased levels of cytotoxic or growth inhibition activity on the same or a different microorganism or species of microorganism relative to the wild type protein. Several motifs have been recognized as characteristic of bacteriocins. For example, the motif YGXGV (SEQ ID NO: 2), wherein X is any amino acid residue, is a N-terminal consensus sequence characteristic of class IIa bacteriocins. Accordingly, in some embodiments, a synthetic bacteriocin comprises an N-terminal sequence with at least about 50% identity to SEQ ID NO: 2, for example at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 2. In some embodiments, a synthetic bacteriocin comprises a N-terminal sequence comprising SEQ ID NO: 2. Additionally, some class IIb bacteriocins comprise a GxxxG motif. Without being limited by any particular theory, it is believed that the GxxxG motif can mediate association between helical proteins in the cell membrane, for example to facilitate bacterioncin-mediated neutralization through cell membrane interactions. As such, in some embodiments, the bacteriocin comprises a motif that facilitates interactions with the cell membrane. In some embodiments, the bacteriocin comprises a GxxxG motif. Optionally, the bacteriocin comprising a GxxxG motif can comprise a helical structure. In addition to structures described herein, “bacteriocin” as used herein also encompasses structures that have substantially the same effect on microbial cells as any of the bacteriocins explicitly provided herein.

It has been shown that fusion polypeptides comprising two or more bacteriocins or portions thereof can have neutralizing activity against a broader range of microbial organisms than either individual bacteriocin. For example, it has been shown that a hybrid bacteriocin, Ent35-MccV (GKYYGNGVSCNKKGCSVDWGRAIGIIGNNSAANLATGGAAGWKSGGGASGRDIAM AIGTLSGQFVAGGIGAAAGGVAGGAIYDYASTHKPNPAMSPSGLGGTIKQKPEGIPSE AWNYAAGRLCNWSPNNLSDVCL, SEQ ID NO: 3), displays antimicrobial activity against pathogenic Gram-positive and Gram-negative bacteria (Acuna et al. (2012), FEBS Open Bio, 2: 12-19). It is noted that that Ent35-MccV fusion bacteriocin comprises, from N-terminus to C-terminus, an N-terminal glycine, Enterocin CRL35, a linker comprising three glycines, and a C-terminal Microcin V. It is contemplated herein that bacteriocins can comprise fusions of two or more polypeptides having bacteriocin activity. In some embodiments, a fusion polypeptide of two or more bacteriocins is provided. In some embodiments, the two or more bacteriocins comprise polypeptides from Table 1.2, or modifications thereof. In some embodiments, the fusion polypeptide comprising of two or more bacteriocins has a broader spectrum of activity than either individual bacteriocin, for example having neutralizing activity against more microbial organisms, neutralizing activity under a broader range of environmental conditions, and/or a higher efficiency of neutralization activity. In some embodiments, a fusion of two or more bacteriocins is provided, for example two, three, four, five, six, seven, eight, nine, or ten bacteriocins. In some embodiments, two or more bacteriocin polypeptides are fused to each other via a covalent bond, for example a peptide linkage. In some embodiments, a linker is positioned between the two bacteriocin polypeptides. In some embodiments, the linker comprises one or glycines, for example about 1, 2, 3, 4, 5, 6, 7, 8, 9, 10, 11, 12, 13, 14, 15, 16, 17, 18, 19, or 20 glycines. In some embodiments, the linker is cleaved within the cell to produce the individual bacteriocins included in the fusion protein. In some embodiments, a bacteriocin as provided herein is modified to provide a desired spectrum of activity relative to the unmodified bacteriocin. For example, the modified bacteriocin may have enhanced or decreased activity agains the same organisms as the unmodified bacteriocin. Alternatively, the modified bacteriocin may have enhanced activity against an organism against which the unmodified bacteriocin has less activity or no activity.

TABLE 1.2
Exemplary Bacteriocins

| Poly- | | | | | Poly- | |
|---|---|---|---|---|---|---|
| pep- | | | | | nucleo- | |
| tide | | | | | tide | Poly- |
| SEQ | | | Poly- | | SEQ | nucleo- |
| ID | | | peptide | Organism of | ID | tide |
| NO: | Name | Class | Sequence | origin | NO: | Sequence |
| 4 | Acidocin | Unclassified | MISSHQKTL | Lactobacillus | 5 | ATGATTTCATC |
| | 8912 | | TDKELALISG | acidophilus | | TCATCAAAAA |
| | | | GKTHYPTNA | | | ACGTTAACTG |
| | | | WKSLWKGF | | | ATAAAGAATT |
| | | | WESLRYTDG | | | AGCATTAATTT |
| | | | F | | | CTGGGGGGAA |
| | | | | | | AACGCACTAC |
| | | | | | | CCGACTAATG |
| | | | | | | CATGGAAAAG |
| | | | | | | TCTTTGGAAA |
| | | | | | | GGTTTCTGGG |
| | | | | | | AAAGCCTTCG |
| | | | | | | TTATACTGAC |
| | | | | | | GGTTTTTAG |
| 6 | Acidocin | class | MISMISSHQ | Lactobacillus | 7 | ATGATTTCAAT |
| | A | IIA/YG | KTLTDKELA | acidophilus | | GATTTCATCTC |
| | | NGV | LISGGKTYY | | | ATCAAAAAAC |
| | | | GTNGVHCTK | | | GTTAACTGAT |
| | | | KSLWGKVRL | | | AAAGAATTAG |
| | | | KNVIPGTLC | | | CATTAATTTCT |
| | | | RKQSLPIKQ | | | GGGGGGAAAA |
| | | | DLKILLGWA | | | CGTACTATGG |
| | | | TGAFGKTFH | | | TACTAATGGT |
| | | | | | | GTGCATTGTA |
| | | | | | | CTAAAAAGAG |
| | | | | | | TCTTTGGGGT |
| | | | | | | AAAGTACGCT |
| | | | | | | TAAAAAACGT |
| | | | | | | GATTCCTGGA |
| | | | | | | ACTCTTTGTCG |
| | | | | | | TAAGCAATCG |
| | | | | | | TTGCCGATCA |
| | | | | | | AACAGGATTT |
| | | | | | | AAAAATTTTA |
| | | | | | | CTGGGCTGGG |
| | | | | | | CTACAGGTGC |
| | | | | | | TTTTGGCAAG |
| | | | | | | ACATTTCATTA |
| | | | | | | A |
| 8 | Acidocin | Unclassified | MDKKTKILF | Lactobacillus | 9 | ATGGATAAGA |
| | B (AcdB) | | EVLYIICIIGP | acidophilus | | AAACAAAAAT |
| | | | QFILFVTAKN | | | ATTATTTGAA |
| | | | NMYQLVGSF | | | GTATTATACAT |
| | | | VGIVWFSYIF | | | CATCTGTATA |
| | | | WYIFFKQHK | | | ATAGGCCCTC |
| | | | KM | | | AATTTATATTA |
| | | | | | | TTTGTGACTGC |
| | | | | | | AAAAAACAAT |
| | | | | | | ATGTATCAGT |
| | | | | | | TGGTGGGTTC |
| | | | | | | GTTTGTTGGA |
| | | | | | | ATAGTATGGT |
| | | | | | | TTTCGTATATT |
| | | | | | | TTTTGGTATAT |
| | | | | | | TTTTTTCAAAC |
| | | | | | | AACATAAAAA |
| | | | | | | AATGTAG |
| 10 | Acidocin | Unclassified | MALKTLEKH | Lactobacillus | 11 | ATGGCTTTAA |
| | LF221B | | ELRNVMGG | gasseri | | AAACATTAGA |
| | (Gassericin | | NKWGNAVI | | | AAAACATGAA |
| | K7 B) | | GAATGATRG | | | TTAAGAAATG |
| | | | VSWCRGFGP | | | TAATGGGTGG |
| | | | WGMTACAL | | | AAACAAGTGG |
| | | | GGAAIGGYL | | | GGGAATGCTG |
| | | | GYKSN | | | TAATAGGAGC |
| | | | | | | TGCTACGGGA |
| | | | | | | GCTACTCGCG |
| | | | | | | GAGTAAGTTG |
| | | | | | | GTGCAGAGGA |
| | | | | | | TTCGGACCAT |
| | | | | | | GGGGAATGAC |
| | | | | | | TGCCTGTGCG |
| | | | | | | TTAGGAGGTG |
| | | | | | | CTGCAATTGG |
| | | | | | | AGGATATCTG |
| | | | | | | GGATATAAGA |
| | | | | | | GTAATTAA |
| 12 | Aureocin | Unclassified | MSWLNFLK | Staphylococcus | 13 | ATGAGTTGGT |
| | A53 | | YIAKYGKKA | aureus | | TAAATTTTTTA |
| | | | VSAAWKYK | | | AAATACATCG |
| | | | GKVLEWLN | | | CTAAATATGG |
| | | | VGPTLEWV | | | CAAAAAAGCG |
| | | | WQKLKKIAG | | | GTATCTGCTG |
| | | | L | | | CTTGGAAGTA |
| | | | | | | CAAAGGTAAA |
| | | | | | | GTATTAGAAT |
| | | | | | | GGCTTAATGT |
| | | | | | | TGGTCCTACTC |
| | | | | | | TTGAATGGGT |
| | | | | | | ATGGCAAAAA |
| | | | | | | TTAAAGAAAA |
| | | | | | | TTGCTGGATT |
| | | | | | | ATAA |
| 14 | Avicin A | class | MTRSKKLNL | Enterococcusavium | 15 | ATGACAAGAT |
| | | IIA/YG | REMKNVVG | (Streptococcus | | CAAAAAAATT |
| | | NGV | GTYYGNGVS | avium) | | AAATTTACGC |
| | | | CNKKGCSVD | | | GAAATGAAGA |
| | | | WGKAISIIGN | | | ATGTTGTTGG |
| | | | NSAANLATG | | | TGGTACCTAC |
| | | | GAAGWKS | | | TATGGAAATG |
| | | | | | | GTGTATCTTGT |
| | | | | | | AACAAGAAAG |
| | | | | | | GCTGTTCAGTT |
| | | | | | | GACTGGGGCA |
| | | | | | | AAGCCATCAG |
| | | | | | | TATTATAGGA |
| | | | | | | AATAATTCCG |
| | | | | | | CAGCAAACTT |
| | | | | | | AGCAACTGGT |
| | | | | | | GGTGCTGCTG |
| | | | | | | GTTGGAAGTC |
| | | | | | | ATAA |
| 16 | Bacteriocin | Unclassified | MKKKLVICG | Enterococcus | 17 | ATGAAAAAGA |
| | 31 | | IIGIGFTALG | faecalis | | AATTAGTTATT |
| | | | TNVEAATYY | (Streptococcus | | TGTGGCATTA |
| | | | GNGLYCNK | faecalis) | | TTGGGATTGG |
| | | | QKCWVDWN | | | TTTTACAGCAT |
| | | | KASREIGKII | | | TAGGAACAAA |
| | | | VNGWVQHG | | | TGTAGAAGCT |
| | | | PWAPR | | | GCTACGTATT |
| | | | | | | ACGGAAATGG |
| | | | | | | TTTATATTGTA |
| | | | | | | ATAAGCAAAA |
| | | | | | | ATGTTGGGTA |
| | | | | | | GACTGGAATA |
| | | | | | | AAGCTTCAAG |
| | | | | | | GGAAATTGGA |
| | | | | | | AAAATTATTG |
| | | | | | | TTAATGGTTG |
| | | | | | | GGTACAACAT |
| | | | | | | GGCCCTTGGG |
| | | | | | | CTCCTAGATA |
| | | | | | | G |
| 18 | Bacteriocin | Unclassified | MKEQNSFNL | Lactococcus | 19 | ATGAAAGAAC |
| | J46 | | LQEVTESEL | lactis | | AAAACTCTTTT |
| | | | DLILGAKGG | | | AATCTTCTTCA |
| | | | SGVIHTISHE | | | AGAAGTGACA |
| | | | VIYNSWNFV | | | GAAAGTGAAT |
| | | | FTCCS | | | TGGACCTTATT |
| | | | | | | TTAGGTGCAA |
| | | | | | | AAGGCGGCAG |
| | | | | | | TGGAGTTATT |
| | | | | | | CATACAATTTC |
| | | | | | | TCATGAAGTA |
| | | | | | | ATATATAATA |
| | | | | | | GCTGGAACTT |
| | | | | | | TGTATTTACTT |
| | | | | | | GCTGCTCTTA |
| | | | | | | A |
| 20 | Bacteriocin | class IIa | MKKKVLKH | Enterococcus | 21 | ATGAAAAAGA |
| | T8 | | CVILGILGTC | faecium | | AAGTATTAAA |
| | | | LAGIGTGIKV | (Streptococcus | | ACATTGTGTT |
| | | | DAATYYGN | faecium) | | ATTCTAGGAA |
| | | | GLYCNKEKC | | | TATTAGGAAC |
| | | | WVDWNQAKG | | | TTGTCTAGCTG |
| | | | EIGKIIVNG | | | GCATCGGTAC |
| | | | WVNHGPWA | | | AGGAATAAAA |
| | | | PRR | | | GTTGATGCAG |
| | | | | | | CTACTTACTAT |
| | | | | | | GGAAATGGTC |
| | | | | | | TTTATTGTAAC |
| | | | | | | AAAGAAAAAT |
| | | | | | | GTTGGGTAGA |
| | | | | | | TTGGAATCAA |
| | | | | | | GCTAAAGGAG |
| | | | | | | AAATTGGAAA |
| | | | | | | AATTATTGTTA |
| | | | | | | ATGGTTGGGT |
| | | | | | | TAATCATGGT |
| | | | | | | CCATGGGCAC |
| | | | | | | CTAGAAGGTA |
| | | | | | | G |
| 22 | Boticin B | Unclassified | MQKPEIISAD | Clostridium | 23 | ATGCAAAAAC |
| | | | LGLCAVNEF | botulinum | | CAGAAATTAT |
| | | | VALAAIPGG | | | TAGTGCTGAT |
| | | | AATFAVCQ | | | TTAGGGCTTT |
| | | | MPNLDEIVS | | | GTGCAGTTAA |
| | | | NAAYV | | | TGAATTTGTA |
| | | | | | | GCTCTTGCTGC |
| | | | | | | CATTCCTGGT |
| | | | | | | GGTGCTGCTA |
| | | | | | | CATTTGCAGT |
| | | | | | | ATGCCAAATG |
| | | | | | | CCAAACTTGG |
| | | | | | | ATGAGATTGT |
| | | | | | | TAGTAATGCA |
| | | | | | | GCATATGTTT |
| | | | | | | AA |
| 24 | Bovicin | Lantibiotic | MMNATENQI | Streptococcus | 25 | ATGATGAATG |
| | HJ50 | | FVETVSDQE | equinus | | CTACTGAAAA |
| | | | LEMLIGGAD | (Streptococcus | | CCAAATTTTTG |
| | | | RGWIKTLTK | bovis) | | TTGAGACTGT |
| | | | DCPNVISSIC | | | GAGTGACCAA |
| | | | AGTIITACKN | | | GAATTAGAAA |
| | | | CA | | | TGTTAATTGGT |
| | | | | | | GGTGCAGATC |
| | | | | | | GTGGATGGAT |
| | | | | | | TAAGACTTTA |
| | | | | | | ACAAAAGATT |
| | | | | | | GTCCAAATGT |
| | | | | | | AATTTCTTCAA |
| | | | | | | TTTGTGCAGG |
| | | | | | | TACAATTATTA |
| | | | | | | CAGCTTGTAA |
| | | | | | | AAATTGTGCT |
| | | | | | | TAA |
| 26 | Brochocin- | Unclassified | MHKVKKLN | Brochothrix | 27 | ATGCACAAGG |
| | c | | NQELQQIVG | campestris | | TAAAAAAATT |
| | | | GYSSKDCLK | | | AAACAATCAA |
| | | | DIGKGIGAG | | | GAGTTACAAC |
| | | | TVAGAAGG | | | AGATCGTGGG |
| | | | GLAAGLGAI | | | AGGTTACAGT |
| | | | PGAFVGAHF | | | TCAAAAGATT |
| | | | GVIGGSAACI | | | GTCTAAAAGA |
| | | | GGLLGN | | | TATTGGTAAA |
| | | | | | | GGAATTGGTG |
| | | | | | | CTGGTACAGT |
| | | | | | | AGCTGGGGCA |
| | | | | | | GCCGGCGGTG |
| | | | | | | GCCTAGCTGC |
| | | | | | | AGGATTAGGT |
| | | | | | | GCTATCCCAG |
| | | | | | | GAGCATTCGT |
| | | | | | | TGGAGCACAT |
| | | | | | | TTTGGAGTAA |
| | | | | | | TCGGCGGATC |
| | | | | | | TGCCGCATGC |
| | | | | | | ATTGGTGGAT |
| | | | | | | TATTAGGTAA |
| | | | | | | CTAG |
| 28 | Butyrivibriocin | Unclassified | MSKKQIMSN | Butyrivibrio | 29 | ATGAGTAAAA |
| | AR10 | | CISIALLIALI | fibrisolvens | | AACAAATTAT |
| | | | PNIYFIADKM | | | GAGTAACTGT |
| | | | GIQLAPAWY | | | ATATCAATTG |
| | | | QDIVNWVSA | | | CATTATTAATA |
| | | | GGTLTTGFAI | | | GCACTAATTC |
| | | | IVGVTVPAW | | | CTAATATCTAT |
| | | | IAEAAAAFGI | | | TTTATTGCAG |
| | | | ASA | | | ATAAAATGGG |
| | | | | | | AATTCAGTTA |
| | | | | | | GCACCTGCTT |
| | | | | | | GGTATCAAGA |
| | | | | | | TATTGTGAATT |
| | | | | | | GGGTATCTGC |
| | | | | | | TGGTGGAACA |
| | | | | | | CTTACTACTG |
| | | | | | | GTTTTGCGATT |
| | | | | | | ATTGTAGGAG |
| | | | | | | TTACAGTACC |
| | | | | | | GGCATGGATA |
| | | | | | | GCAGAAGCAG |
| | | | | | | CTGCAGCTTTT |
| | | | | | | GGTATAGCTT |
| | | | | | | CAGCATGA |
| 30 | Butyrivibriocin | Lantibiotic | MNKELNALT | Butyrivibrio | 31 | ATGAACAAAG |
| | OR79 | | NPIDEKELEQ | fibrisolvens | | AACTTAATGC |
| | | | ILGGGNGVI | | | ACTTACAAAT |
| | | | KTISHECHM | | | CCTATTGACG |
| | | | NTWQFIFTC | | | AGAAGGAGCT |
| | | | CS | | | TGAGCAGATC |
| | | | | | | CTCGGTGGTG |
| | | | | | | GCAATGGTGT |
| | | | | | | CATCAAGACA |
| | | | | | | ATCAGCCACG |
| | | | | | | AGTGCCACAT |
| | | | | | | GAACACATGG |
| | | | | | | CAGTTCATTTT |
| | | | | | | CACATGTTGC |
| | | | | | | TCTTAA |
| 32 | Carnobacteriocin | class | MNSVKELN | Carnobacterium | 33 | ATGAATAGCG |
| | B2 | IIA/YG | VKEMKQLH | maltaromaticum | | TAAAAGAATT |
| | (Carnocin | NGV | GGVNYGNG | (Carnobacterium | | AAACGTGAAA |
| | CP52) | | VSCSKTKCS | piscicola) | | GAAATGAAAC |
| | | | VNWGQAFQ | | | AATTACACGG |
| | | | ERYTAGINSF | | | TGGAGTAAAT |
| | | | VSGVASGAG | | | TATGGTAATG |
| | | | SIGRRP | | | GTGTTTCTTGC |
| | | | | | | AGTAAAACAA |
| | | | | | | AATGTTCAGT |
| | | | | | | TAACTGGGGA |
| | | | | | | CAAGCCTTTC |
| | | | | | | AAGAAAGATA |
| | | | | | | CACAGCTGGA |
| | | | | | | ATTAACTCATT |
| | | | | | | TGTAAGTGGA |
| | | | | | | GTCGCTTCTG |
| | | | | | | GGGCAGGATC |
| | | | | | | CATTGGTAGG |
| | | | | | | AGACCGTAA |
| 34 | Carnobacteriocin | class | MKSVKELNK | Carnobacterium | 35 | ATGAAAAGCG |
| | BM1 | IIA/YG | KEMQQINGG | maltaromaticum | | TTAAAGAACT |
| | (Carno | NGV | AISYGNGVY | (Carnobacterium | | AAATAAAAAA |
| | bacteriocin | | CNKEKCWV | piscicola) | | GAAATGCAAC |
| | B1) | | NKAENKQAI | | | AAATTAATGG |
| | | | TGIVIGGWA | | | TGGAGCTATC |
| | | | SSLAGMGH | | | TCTTATGGCA |
| | | | | | | ATGGTGTTTAT |
| | | | | | | TGTAACAAAG |
| | | | | | | AGAAATGTTG |
| | | | | | | GGTAAACAAG |
| | | | | | | GCAGAAAACA |
| | | | | | | AACAAGCTAT |
| | | | | | | TACTGGAATA |
| | | | | | | GTTATCGGTG |
| | | | | | | GATGGGCTTC |
| | | | | | | TAGTTTAGCA |
| | | | | | | GGAATGGGAC |
| | | | | | | ATTAA |
| 36 | Carno | class IIc, | MNNVKELSI | Carnobacterium | 37 | ATGAATAATG |
| | bacteriocin-A | non | KEMQQVTG | maltaromaticum | | TAAAAGAGTT |
| | (Piscicolin | subgrouped | GDQMSDGV | (Carnobacterium | | AAGTATTAAA |
| | -61) | bacteriocins | NYGKGSSLS | piscicola) | | GAAATGCAAC |
| | | (problematic) | KGGAKCGL | | | AAGTTACTGG |
| | | | GIVGGLATIP | | | TGGAGACCAA |
| | | | SGPLGWLAG | | | ATGTCAGATG |
| | | | AAGVINSCM | | | GTGTAAATTA |
| | | | K | | | TGGAAAAGGC |
| | | | | | | TCTAGCTTATC |
| | | | | | | AAAAGGTGGT |
| | | | | | | GCCAAATGTG |
| | | | | | | GTTTAGGGAT |
| | | | | | | CGTCGGCGGA |
| | | | | | | TTAGCTACTAT |
| | | | | | | CCCTTCAGGT |
| | | | | | | CCTTTAGGCT |
| | | | | | | GGTTAGCCGG |
| | | | | | | AGCAGCAGGT |
| | | | | | | GTAATTAATA |
| | | | | | | GCTGTATGAA |
| | | | | | | ATAA |
| 38 | Carnocyclin- | Unclassified | MLYELVAY | Carnobacterium | 39 | ATGTTATATG |
| | A | | GIAQGTAEK | maltaromaticum | | AATTAGTTGC |
| | | | VVSLINAGL | (Carnobacterium | | ATATGGTATC |
| | | | TVGSIISILG | piscicola) | | GCACAAGGTA |
| | | | GVTVGLSGV | | | CAGCTGAAAA |
| | | | FTAVKAAIA | | | GGTTGTAAGT |
| | | | KQGIKKAIQ | | | CTAATTAACG |
| | | | L | | | CAGGTTTAAC |
| | | | | | | AGTAGGGTCT |
| | | | | | | ATTATTTCAAT |
| | | | | | | TTTGGGTGGG |
| | | | | | | GTCACAGTCG |
| | | | | | | GTTTATCAGG |
| | | | | | | TGTCTTCACA |
| | | | | | | GCAGTTAAAG |
| | | | | | | CAGCAATTGC |
| | | | | | | TAAACAAGGA |
| | | | | | | ATAAAAAAAG |
| | | | | | | CAATTCAATT |
| | | | | | | ATAA |
| 40 | Carocin D | Unclassified | MIKYRLYAP | Pectobacterium | 41 | ATGATTAAAT |
| | | | NDGDTMTV | carotovorum | | ACCGTTTATAT |
| | | | SGGGGWVS | subsp. | | GCTCCAAATG |
| | | | NDDRKGGN | carotovorum | | ATGGAGACAC |
| | | | DRDNGKGG | (Erwinia | | CATGACAGTG |
| | | | SAVDFSKNP | carotovora | | AGTGGTGGTG |
| | | | EKQAIVNPY | subsp. | | GTGGTTGGGT |
| | | | LAIAIPMPVY | carotovora) | | TTCAAACGAT |
| | | | PLYGKLGFTI | | | GATCGCAAAG |
| | | | NTTAIETELA | | | GTGGTAATGA |
| | | | NVRAAINTK | | | CAGGGACAAT |
| | | | LATLSAVIGR | | | GGCAAAGGTG |
| | | | SLPVVGRVF | | | GTTCTGCCGTT |
| | | | GVTAAGMW | | | GATTTTAGTA |
| | | | PSSTAPSSLD | | | AAAATCCAGA |
| | | | SIYNQAHQQ | | | AAAGCAGGCT |
| | | | ALAQLAAQQ | | | ATCGTTAATCC |
| | | | GVLNKGYN | | | CTATTTGGCA |
| | | | VTAMPAGFV | | | ATCGCGATAC |
| | | | SSLPVSEIKS | | | CGATGCCGGT |
| | | | LPTAPASLLA | | | CTACCCTCTTT |
| | | | QSVINTELSQ | | | ATGGAAAGCT |
| | | | RQLALTQPT | | | AGGGTTCACA |
| | | | TNAPVANIP | | | ATAAATACGA |
| | | | VVKAEKTA | | | CGGCAATTGA |
| | | | MPGVYSAKI | | | GACTGAACTC |
| | | | IAGEPAFQIK | | | GCAAATGTCA |
| | | | VDNTKPALA | | | GAGCAGCAAT |
| | | | QNPPKVKDD | | | TAACACTAAA |
| | | | IQVSSFLSSP | | | CTTGCAACAC |
| | | | VADTHHAFI | | | TCAGTGCAGT |
| | | | DFGSDHEPV | | | GATTGGCAGA |
| | | | YVSLSKIVT | | | TCACTTCCGGT |
| | | | AEEEKKQVE | | | CGTTGGGCGG |
| | | | EAKRREQEW | | | GTATTTGGTG |
| | | | LLRHPITAAE | | | TTACTGCCGC |
| | | | RKLTEIRQVI | | | CGGAATGTGG |
| | | | SFAQQLKES | | | CCTTCTAGTAC |
| | | | SVATISEKTK | | | CGCTCCCAGT |
| | | | TVAVYQEQ | | | AGTCTCGATT |
| | | | VNTAAKNR | | | CTATATACAA |
| | | | DNFYNQNR | | | TCAAGCACAT |
| | | | GLLSAGITG | | | CAGCAGGCTT |
| | | | GPGYPIYLA | | | TAGCCCAGTT |
| | | | LWQTMNNF | | | AGCTGCTCAA |
| | | | HQAYFRANN | | | CAGGGAGTAT |
| | | | ALEQESHVL | | | TAAATAAAGG |
| | | | NLARSDLAK | | | GTATAACGTT |
| | | | AEQLLAENN | | | ACAGCAATGC |
| | | | RLQVETERT | | | CTGCAGGTTT |
| | | | LAEEKEIKR | | | CGTCAGCAGT |
| | | | NRVNVSTFG | | | TTGCCTGTTAG |
| | | | TVQTQLSKL | | | TGAAATCAAA |
| | | | LSDFYAVTS | | | TCATTGCCAA |
| | | | LSQSVPSGA | | | CAGCTCCCGC |
| | | | LASFSYNPQ | | | CAGTTTACTG |
| | | | GMIGSGKIV | | | GCACAAAGTG |
| | | | GKDVDVLFS | | | TGATTAATAC |
| | | | IPVKDIPGYK | | | CGAACTTTCCC |
| | | | SPINLDDLAK | | | AGCGTCAACT |
| | | | KNGSLDLPIR | | | GGCTCTTACTC |
| | | | LAFSDENGE | | | AGCCCACGAC |
| | | | RVLRAFKAD | | | GAATGCACCA |
| | | | SLRIPSSVRG | | | GTCGCGAATA |
| | | | VAGSYDKNT | | | TTCCCGTAGTT |
| | | | GIFSAEIDGV | | | AAAGCAGAGA |
| | | | SSRLVLENP | | | AAACAGCAAT |
| | | | AFPPTGNVG | | | GCCAGGTGTG |
| | | | NTGNTAPDY | | | TATTCAGCGA |
| | | | KALLNTGVD | | | AAATTATTGCT |
| | | | VKPVDKITV | | | GGTGAGCCTG |
| | | | TVTPVADPV | | | CATTCCAAAT |
| | | | DIDDYIIWLP | | | CAAGGTCGAT |
| | | | TASGSGVEPI | | | AATACCAAAC |
| | | | YVVFNSNPY | | | CTGCTTTGGC |
| | | | GGTEKGKYS | | | ACAGAATCCG |
| | | | KRYYNPDKA | | | CCGAAAGTAA |
| | | | GGPILELDW | | | AAGATGATAT |
| | | | KNVKIDHAG | | | TCAGGTATCTT |
| | | | VDNVKLHT | | | CTTTCCTTTCC |
| | | | GRFKASVEN | | | TCGCCAGTAG |
| | | | KVMIERLENI | | | CTGATACGCA |
| | | | LNGQITATD | | | CCATGCATTTA |
| | | | TDKRFYTHE | | | TTGATTTTGGC |
| | | | LRELNRYRN | | | AGCGATCATG |
| | | | LGIKDGEVP | | | AACCGGTATA |
| | | | SSIQEESAV | | | CGTGTCTCTTT |
| | | | WNDTHTAT | | | CAAAGATCGT |
| | | | LEDYKINEK | | | GACAGCCGAG |
| | | | EQPLYTDAA | | | GAGGAGAAAA |
| | | | LQAAYEQEL | | | AACAGGTTGA |
| | | | KDALGGKH | | | AGAGGCCAAG |
| | | | G | | | CGCCGTGAGC |
| | | | | | | AGGAGTGGTT |
| | | | | | | GTTGCGTCAT |
| | | | | | | CCAATTACAG |
| | | | | | | CTGCGGAGCG |
| | | | | | | AAAATTAACT |
| | | | | | | GAAATCCGCC |
| | | | | | | AAGTGATCTC |
| | | | | | | TTTTGCTCAAC |
| | | | | | | AGCTAAAAGA |
| | | | | | | AAGCTCTGTC |
| | | | | | | GCAACCATTT |
| | | | | | | CAGAAAAAAC |
| | | | | | | TAAAACTGTT |
| | | | | | | GCGGTTTACC |
| | | | | | | AAGAACAGGT |
| | | | | | | GAATACCGCT |
| | | | | | | GCAAAAAATC |
| | | | | | | GCGACAATTT |
| | | | | | | TTATAATCAA |
| | | | | | | AATAGAGGTC |
| | | | | | | TGTTAAGTGC |
| | | | | | | GGGTATAACT |
| | | | | | | GGGGGACCGG |
| | | | | | | GATATCCTATT |
| | | | | | | TATCTTGCTTT |
| | | | | | | ATGGCAAACG |
| | | | | | | ATGAATAACT |
| | | | | | | TTCATCAGGC |
| | | | | | | TTATTTCAGA |
| | | | | | | GCAAATAATG |
| | | | | | | CATTGGAACA |
| | | | | | | AGAGAGTCAT |
| | | | | | | GTTCTGAACC |
| | | | | | | TGGCTCGTTCT |
| | | | | | | GATCTGGCTA |
| | | | | | | AGGCTGAGCA |
| | | | | | | ATTGCTTGCTG |
| | | | | | | AGAATAATCG |
| | | | | | | ACTTCAGGTT |
| | | | | | | GAAACGGAGC |
| | | | | | | GAACGCTTGC |
| | | | | | | CGAAGAAAAA |
| | | | | | | GAGATAAAAC |
| | | | | | | GCAACAGGGT |
| | | | | | | TAATGTATCA |
| | | | | | | ACATTTGGCA |
| | | | | | | CAGTGCAAAC |
| | | | | | | TCAACTTAGT |
| | | | | | | AAATTGCTGT |
| | | | | | | CAGATTTTTAT |
| | | | | | | GCTGTTACAT |
| | | | | | | CACTTTCCCAA |
| | | | | | | AGTGTTCCTTC |
| | | | | | | GGGGGCATTA |
| | | | | | | GCCTCTTTTTC |
| | | | | | | ATATAATCCA |
| | | | | | | CAAGGGATGA |
| | | | | | | TTGGCAGCGG |
| | | | | | | TAAGATTGTT |
| | | | | | | GGGAAGGATG |
| | | | | | | TCGATGTTTTA |
| | | | | | | TTTTCCATCCC |
| | | | | | | AGTAAAAGAT |
| | | | | | | ATTCCGGGAT |
| | | | | | | ATAAATCTCCT |
| | | | | | | ATTAACTTGG |
| | | | | | | ACGATTTAGC |
| | | | | | | CAAGAAAAAT |
| | | | | | | GGAAGTCTGG |
| | | | | | | ATCTTCCCATT |
| | | | | | | CGTCTGGCAT |
| | | | | | | TTTCTGATGA |
| | | | | | | GAATGGAGAA |
| | | | | | | AGGGTTCTTC |
| | | | | | | GGGCATTCAA |
| | | | | | | AGCGGATAGT |
| | | | | | | CTGCGAATCC |
| | | | | | | CTTCGAGTGT |
| | | | | | | CAGAGGTGTA |
| | | | | | | GCGGGCAGTT |
| | | | | | | ATGACAAAAA |
| | | | | | | TACGGGTATT |
| | | | | | | TTTAGTGCAG |
| | | | | | | AAATTGATGG |
| | | | | | | TGTTTCATCTC |
| | | | | | | GCCTTGTACT |
| | | | | | | GGAAAACCCA |
| | | | | | | GCGTTTCCTCC |
| | | | | | | GACCGGAAAT |
| | | | | | | GTCGGTAATA |
| | | | | | | CGGGTAATAC |
| | | | | | | TGCACCTGAC |
| | | | | | | TATAAAGCAT |
| | | | | | | TACTGAATAC |
| | | | | | | TGGTGTTGAT |
| | | | | | | GTTAAACCTG |
| | | | | | | TTGATAAAAT |
| | | | | | | CACAGTTACG |
| | | | | | | GTAACACCAG |
| | | | | | | TTGCTGATCC |
| | | | | | | AGTGGATATT |
| | | | | | | GATGACTATA |
| | | | | | | TAATCTGGTT |
| | | | | | | GCCAACTGCG |
| | | | | | | TCTGGTTCTG |
| | | | | | | GCGTGGAACC |
| | | | | | | CATTTATGTCG |
| | | | | | | TGTTTAACAG |
| | | | | | | TAATCCGTAT |
| | | | | | | GGTGGGACGG |
| | | | | | | AAAAAGGAAA |
| | | | | | | ATATAGCAAA |
| | | | | | | CGTTATTATAA |
| | | | | | | TCCAGATAAG |
| | | | | | | GCAGGCGGTC |
| | | | | | | CGATCTTGGA |
| | | | | | | GCTGGATTGG |
| | | | | | | AAAAACGTTA |
| | | | | | | AGATTGACCA |
| | | | | | | TGCAGGTGTG |
| | | | | | | GACAATGTTA |
| | | | | | | AATTACACAC |
| | | | | | | AGGGCGTTTC |
| | | | | | | AAAGCGTCGG |
| | | | | | | TTGAAAACAA |
| | | | | | | AGTGATGATT |
| | | | | | | GAACGTTTGG |
| | | | | | | AAAACATACT |
| | | | | | | GAATGGTCAA |
| | | | | | | ATCACGGCCA |
| | | | | | | CGGATACTGA |
| | | | | | | CAAGCGATTC |
| | | | | | | TATACGCATG |
| | | | | | | AATTAAGAGA |
| | | | | | | GTTAAACCGC |
| | | | | | | TACAGAAATT |
| | | | | | | TAGGCATCAA |
| | | | | | | AGACGGTGAA |
| | | | | | | GTGCCTAGTA |
| | | | | | | GCATTCAAGA |
| | | | | | | AGAAAGCGCT |
| | | | | | | GTTTGGAACG |
| | | | | | | ACACACACAC |
| | | | | | | AGCGACGCTT |
| | | | | | | GAAGACTACA |
| | | | | | | AAATTAATGA |
| | | | | | | GAAAGAGCAA |
| | | | | | | CCGTTGTACA |
| | | | | | | CTGATGCTGC |
| | | | | | | TTTGCAGGCA |
| | | | | | | GCCTACGAAC |
| | | | | | | AGGAACTCAA |
| | | | | | | AGACGCATTA |
| | | | | | | GGAGGGAAAC |
| | | | | | | ATGGCTAA |
| 42 | Cerein 7B | Unclassified | MENLQMLT | Bacillus | 43 | ATGGAAAACT |
| | | | EEELMEIEG | cereus | | TACAAATGTT |
| | | | GGWWNSWG | | | AACTGAAGAA |
| | | | KCVAGTIGG | | | GAATTAATGG |
| | | | AGTGGLGGA | | | AAATTGAAGG |
| | | | AAGSAVPVI | | | TGGAGGCTGG |
| | | | GTGIGGAIG | | | TGGAATAGCT |
| | | | GVSGGLTGA | | | GGGGTAAATG |
| | | | ATFC | | | TGTTGCTGGA |
| | | | | | | ACTATCGGTG |
| | | | | | | GAGCTGGAAC |
| | | | | | | TGGTGGTTTA |
| | | | | | | GGTGGAGCTG |
| | | | | | | CTGCAGGTTC |
| | | | | | | AGCTGTTCCG |
| | | | | | | GTTATTGGTA |
| | | | | | | CTGGTATTGG |
| | | | | | | TGGCGCTATT |
| | | | | | | GGTGGAGTTA |
| | | | | | | GCGGTGGCCT |
| | | | | | | TACAGGTGCA |
| | | | | | | GCTACTTTTTG |
| | | | | | | CTAA |
| 44 | Cinnamycin | Lantibiotic | MTASILQQS | Strepto- | 45 | ATGACCGCTT |
| | (Lanthiopeptin) | | VVDADFRAA | verticiilium | | CCATTCTTCAG |
| | | | LLENPAAFG | griseo | | CAGTCCGTCG |
| | | | ASAAALPTP | verticillatum | | TGGACGCCGA |
| | | | VEAQDQASL | | | CTTCCGCGCG |
| | | | DFWTKDIAA | | | GCGCTGCTTG |
| | | | TEAFACRQS | | | AGAACCCCGC |
| | | | CSFGPFTFVC | | | CGCCTTCGGC |
| | | | DGNTK | | | GCTTCCGCCG |
| | | | | | | CGGCCCTGCC |
| | | | | | | CACGCCCGTC |
| | | | | | | GAGGCCCAGG |
| | | | | | | ACCAGGCGTC |
| | | | | | | CCTTGACTTCT |
| | | | | | | GGACCAAGGA |
| | | | | | | CATCGCCGCC |
| | | | | | | ACGGAAGCCT |
| | | | | | | TCGCCTGCCG |
| | | | | | | CCAGAGCTGC |
| | | | | | | AGCTTCGGCC |
| | | | | | | CGTTCACCTTC |
| | | | | | | GTGTGCGACG |
| | | | | | | GCAACACCAA |
| | | | | | | GTAA |
| 46 | Circularin | Unclassified | MSLLALVAG | Geobacillus | 47 | ATGAGTTTGC |
| | A | | TLGVSQSIAT | kaustophilus | | TGGCGCTTGT |
| | | | TVVSIVLTGS | (strain | | TGCCGGGACG |
| | | | TLISIILGIT | HTA426) | | CTCGGCGTGT |
| | | | AILSGGVDAIL | | | CACAGTCAAT |
| | | | EIGWSAFVA | | | CGCGACGACG |
| | | | TVKKIVAER | | | GTTGTTTCGAT |
| | | | GKAAAIAW | | | TGTGTTGACC |
| | | | | | | GGCTCCACTC |
| | | | | | | TCATTTCTATT |
| | | | | | | ATTCTTGGGA |
| | | | | | | TCACCGCTATT |
| | | | | | | TTGTCAGGTG |
| | | | | | | GAGTCGACGC |
| | | | | | | CATTTTGGAA |
| | | | | | | ATTGGGTGGT |
| | | | | | | CAGCTTTTGTC |
| | | | | | | GCGACGGTGA |
| | | | | | | AAAAAATAGT |
| | | | | | | GGCGGAACGA |
| | | | | | | GGAAAAGCGG |
| | | | | | | CAGCGATTGC |
| | | | | | | ATGGTAA |
| 48 | Closticin | Unclassified | MRKVFLRSII | Clostridium | 49 | TTGAGAAAAG |
| | 574 | | STLVMCAFV | tyrobutyricum | | TATTTTTAAGA |
| | | | SSSFSVNAD | | | TCAATAATTTC |
| | | | ESKPNDEKII | | | AACATTAGTT |
| | | | NNIENVTTT | | | ATGTGTGCAT |
| | | | KDIVKSNKN | | | TTGTTTCAAGC |
| | | | NIVYLDEGV | | | AGCTTTTCAGT |
| | | | MSIPLSGRKP | | | AAATGCGGAT |
| | | | IAIKDDNNK | | | GAAAGCAAAC |
| | | | EDLTVTLPIK | | | CAAATGATGA |
| | | | NTGDISKISS | | | AAAAATAATT |
| | | | NGTILYKNN | | | AATAACATAG |
| | | | SSNSSNIALQ | | | AAAACGTTAC |
| | | | PKNDGFKAL | | | TACTACTAAA |
| | | | ININDKLAN | | | GATATTGTAA |
| | | | KEYEFTFNL | | | AAAGTAATAA |
| | | | PKNSKLISAA | | | AAATAATATT |
| | | | TYLGKEYDT | | | GTATATTTAG |
| | | | KEVFVVDKN | | | ATGAAGGTGT |
| | | | NIITSIISPAW | | | AATGAGTATT |
| | | | AKDANGHN | | | CCATTGTCTG |
| | | | VSTYYKIVS | | | GGAGAAAACC |
| | | | NNKLVQVV | | | CATTGCTATTA |
| | | | EFTENTAFP | | | AAGATGATAA |
| | | | VVADPNWT | | | TAATAAAGAA |
| | | | KIGKCAGSIA | | | GATTTAACTG |
| | | | WAIGSGLFG | | | TTACATTACCT |
| | | | GAKLIKIKKY | | | ATTAAGAATA |
| | | | IAELGGLQK | | | CTGGAGATAT |
| | | | AAKLLVGAT | | | ATCTAAAATT |
| | | | TWEEKLHAG | | | AGTAGTAATG |
| | | | GYALINLAA | | | GTACTATTCTG |
| | | | ELTGVAGIQ | | | TATAAAAATA |
| | | | ANCF | | | ATAGTAGTAA |
| | | | | | | TTCATCTAATA |
| | | | | | | TAGCTTTACA |
| | | | | | | ACCTAAAAAT |
| | | | | | | GATGGATTTA |
| | | | | | | AGGCTTTAAT |
| | | | | | | AAATATTAAT |
| | | | | | | GATAAGTTAG |
| | | | | | | CTAATAAAGA |
| | | | | | | ATATGAATTT |
| | | | | | | ACATTTAATTT |
| | | | | | | ACCCAAAAAC |
| | | | | | | AGTAAATTAA |
| | | | | | | TTAGTGCTGC |
| | | | | | | CACATATTTG |
| | | | | | | GGTAAAGAAT |
| | | | | | | ATGATACAAA |
| | | | | | | AGAAGTATTT |
| | | | | | | GTAGTAGACA |
| | | | | | | AAAATAATAT |
| | | | | | | AATTACGAGT |
| | | | | | | ATTATTAGTCC |
| | | | | | | AGCTTGGGCT |
| | | | | | | AAAGATGCAA |
| | | | | | | ATGGACATAA |
| | | | | | | TGTTTCTACTT |
| | | | | | | ATTATAAGAT |
| | | | | | | AGTATCGAAT |
| | | | | | | AATAAATTAG |
| | | | | | | TACAAGTTGT |
| | | | | | | TGAATTCACA |
| | | | | | | GAAAATACTG |
| | | | | | | CATTCCCGGT |
| | | | | | | GGTAGCTGAT |
| | | | | | | CCTAATTGGA |
| | | | | | | CTAAAATTGG |
| | | | | | | GAAATGCGCT |
| | | | | | | GGGTCAATAG |
| | | | | | | CATGGGCTAT |
| | | | | | | AGGTTCTGGC |
| | | | | | | CTTTTTGGTGG |
| | | | | | | AGCAAAGCTA |
| | | | | | | ATTAAAATAA |
| | | | | | | AAAAATATAT |
| | | | | | | AGCAGAGCTT |
| | | | | | | GGAGGACTTC |
| | | | | | | AAAAAGCAGC |
| | | | | | | TAAATTATTA |
| | | | | | | GTTGGTGCAA |
| | | | | | | CCACTTGGGA |
| | | | | | | AGAAAAATTA |
| | | | | | | CACGCAGGCG |
| | | | | | | GTTATGCATT |
| | | | | | | AATTAACTTA |
| | | | | | | GCTGCTGAGC |
| | | | | | | TAACAGGTGT |
| | | | | | | AGCAGGTATA |
| | | | | | | CAAGCAAATT |
| | | | | | | GTTTTTAA |
| 50 | Coagulin | Unclassified | MKKIEKLTE | Bacillus | 51 | ATGAAAAAAA |
| | A | | KEMANIIGG | coagulans | | TTGAAAAATT |
| | | | KYYGNGVT | | | AACTGAAAAA |
| | | | CGKHSCSVD | | | GAAATGGCCA |
| | | | WGKATTCII | | | ATATCATTGG |
| | | | NNGAMAWA | | | TGGTAAATAC |
| | | | TGGHQGTH | | | TACGGTAATG |
| | | | KC | | | GGGTTACTTG |
| | | | | | | TGGCAAACAT |
| | | | | | | TCCTGCTCTGT |
| | | | | | | TGACTGGGGT |
| | | | | | | AAGGCTACCA |
| | | | | | | CCTGCATAAT |
| | | | | | | CAATAATGGA |
| | | | | | | GCTATGGCAT |
| | | | | | | GGGCTACTGG |
| | | | | | | TGGACATCAA |
| | | | | | | GGTACTCATA |
| | | | | | | AATGCTAG |
| 52 | Colicin-10 | Unclassified | MDKVTDNSP | Escherichia | 53 | ATGGATAAAG |
| | | | DVESTESTE | coli | | TCACTGATAA |
| | | | GSFPTVGVD | | | TTCTCCAGAT |
| | | | TGDTITATL | | | GTGGAGAGCA |
| | | | ATGTENVGG | | | CAGAATCTAC |
| | | | GGGAFGGAS | | | TGAGGGGTCA |
| | | | ESSAAIHATA | | | TTCCCAACTGT |
| | | | KWSTAQLKK | | | TGGGGTTGAT |
| | | | HQAEQAAR | | | ACTGGCGATA |
| | | | AAAAEAALA | | | CGATTACAGC |
| | | | KAKSQRDAL | | | GACGCTTGCA |
| | | | TQRLKDIVN | | | ACTGGAACTG |
| | | | DALRANAAR | | | AAAATGTTGG |
| | | | SPSVTDLAH | | | TGGAGGCGGT |
| | | | ANNMAMQA | | | GGAGCATTTG |
| | | | EAERLRLAK | | | GTGGGGCCAG |
| | | | AEQKAREEA | | | TGAAAGTTCT |
| | | | EAAEKALRE | | | GCTGCGATAC |
| | | | AERQRDEIA | | | ATGCAACCGC |
| | | | RQQAETAHL | | | TAAATGGTCT |
| | | | LAMAEAAEA | | | ACCGCGCAGT |
| | | | EKNRQDSLD | | | TGAAAAAACA |
| | | | EEHRAVEVA | | | TCAGGCTGAA |
| | | | EKKLAEAKA | | | CAGGCTGCCC |
| | | | ELAKAESDV | | | GTGCTGCTGC |
| | | | QSKQAIVSR | | | GGCTGAGGCA |
| | | | VAGELENAQ | | | GCATTGGCAA |
| | | | KSVDVKVTG | | | AAGCGAAATC |
| | | | FPGWRDVQ | | | TCAGCGTGAT |
| | | | KKLERQLQD | | | GCCCTGACTC |
| | | | KKNEYSSVT | | | AACGTCTCAA |
| | | | NALNSAVSI | | | GGATATTGTT |
| | | | RDAKKTEVQ | | | AATGACGCTT |
| | | | NAEIKLKEA | | | TACGTGCTAA |
| | | | KDALEKSQV | | | TGCCGCTCGT |
| | | | KDSVDTMV | | | AGTCCATCAG |
| | | | GFYQYITEQ | | | TAACTGACCTT |
| | | | YGEKYSRIA | | | GCTCATGCCA |
| | | | QDLAEKAKG | | | ATAATATGGC |
| | | | SKFNSVDEA | | | AATGCAGGCA |
| | | | LAAFEKYKN | | | GAGGCTGAGC |
| | | | VLDKKFSKV | | | GTTTGCGCCTT |
| | | | DRDDIFNAL | | | GCGAAGGCAG |
| | | | ESITYDEWA | | | AGCAAAAAGC |
| | | | KHLEKISRAL | | | CCGTGAAGAA |
| | | | KVTGYLSFG | | | GCTGAAGCAG |
| | | | YDVWDGTL | | | CAGAAAAAGC |
| | | | KGLKTGDW | | | GCTCCGGGAA |
| | | | KPLFVTLEKS | | | GCAGAACGCC |
| | | | AVDFGVAKI | | | AACGTGATGA |
| | | | VALMFSFIV | | | GATTGCCCGC |
| | | | GAPLGFWGI | | | CAACAGGCTG |
| | | | AIITGIVSSYI | | | AAACCGCGCA |
| | | | GDDELNKLN | | | TTTGTTAGCA |
| | | | ELLGI | | | ATGGCGGAGG |
| | | | | | | CAGCAGAGGC |
| | | | | | | TGAGAAAAAT |
| | | | | | | CGACAGGATT |
| | | | | | | CTCTTGATGA |
| | | | | | | AGAGCATCGG |
| | | | | | | GCTGTGGAAG |
| | | | | | | TGGCAGAGAA |
| | | | | | | GAAGCTGGCT |
| | | | | | | GAGGCTAAAG |
| | | | | | | CTGAACTGGC |
| | | | | | | GAAGGCCGAA |
| | | | | | | AGCGATGTAC |
| | | | | | | AGAGTAAGCA |
| | | | | | | AGCGATTGTT |
| | | | | | | TCCAGAGTTG |
| | | | | | | CAGGGGAGCT |
| | | | | | | TGAAAACGCT |
| | | | | | | CAAAAAAGTG |
| | | | | | | TTGATGTGAA |
| | | | | | | GGTTACCGGA |
| | | | | | | TTTCCTGGATG |
| | | | | | | GCGTGATGTT |
| | | | | | | CAGAAAAAAC |
| | | | | | | TGGAGAGACA |
| | | | | | | ATTGCAGGAT |
| | | | | | | AAGAAGAATG |
| | | | | | | AATATTCGTC |
| | | | | | | AGTGACGAAT |
| | | | | | | GCTCTTAATTC |
| | | | | | | TGCTGTTAGC |
| | | | | | | ATTAGAGATG |
| | | | | | | CTAAAAAAAC |
| | | | | | | AGAAGTTCAG |
| | | | | | | AATGCTGAGA |
| | | | | | | TAAAATTAAA |
| | | | | | | AGAAGCTAAG |
| | | | | | | GATGCTCTTG |
| | | | | | | AGAAGAGTCA |
| | | | | | | GGTAAAAGAC |
| | | | | | | TCTGTTGATAC |
| | | | | | | TATGGTTGGG |
| | | | | | | TTTTATCAATA |
| | | | | | | TATAACCGAA |
| | | | | | | CAATATGGGG |
| | | | | | | AAAAATATTC |
| | | | | | | CAGAATAGCT |
| | | | | | | CAGGATTTAG |
| | | | | | | CTGAAAAGGC |
| | | | | | | GAAGGGTAGT |
| | | | | | | AAATTTAATA |
| | | | | | | GTGTTGATGA |
| | | | | | | AGCACTTGCT |
| | | | | | | GCATTTGAAA |
| | | | | | | AGTATAAAAA |
| | | | | | | TGTACTGGAT |
| | | | | | | AAGAAATTCA |
| | | | | | | GTAAGGTTGA |
| | | | | | | TAGGGATGAT |
| | | | | | | ATTTTTAATGC |
| | | | | | | TTTAGAGTCT |
| | | | | | | ATTACTTATGA |
| | | | | | | TGAGTGGGCC |
| | | | | | | AAGCATCTAG |
| | | | | | | AAAAGATCTC |
| | | | | | | TAGGGCTCTT |
| | | | | | | AAGGTTACTG |
| | | | | | | GATATTTGTCT |
| | | | | | | TTCGGGTATG |
| | | | | | | ATGTATGGGA |
| | | | | | | TGGTACCCTA |
| | | | | | | AAGGGATTAA |
| | | | | | | AAACAGGAGA |
| | | | | | | CTGGAAGCCT |
| | | | | | | TTATTTGTCAC |
| | | | | | | TCTGGAGAAG |
| | | | | | | AGCGCGGTAG |
| | | | | | | ATTTCGGCGT |
| | | | | | | GGCAAAAATT |
| | | | | | | GTGGCATTAA |
| | | | | | | TGTTTAGTTTT |
| | | | | | | ATTGTTGGTG |
| | | | | | | CGCCTCTTGG |
| | | | | | | CTTCTGGGGA |
| | | | | | | ATTGCAATTAT |
| | | | | | | CACAGGTATT |
| | | | | | | GTTTCTTCTTA |
| | | | | | | CATAGGGGAT |
| | | | | | | GATGAGTTGA |
| | | | | | | ACAAGCTTAA |
| | | | | | | TGAATTACTA |
| | | | | | | GGTATTTAA |
| 54 | Colicin- | Unclassified | METAVAYY | Escherichia | 55 | ATGGAAACCG |
| | E1 | | KDGVPYDD | coli | | CGGTAGCGTA |
| | | | KGQVIITLLN | | | CTATAAAGAT |
| | | | GTPDGSGSG | | | GGTGTTCCTTA |
| | | | GGGGKGGS | | | TGATGATAAG |
| | | | KSESSAAIHA | | | GGACAGGTAA |
| | | | TAKWSTAQL | | | TTATTACTCTT |
| | | | KKTQAEQAA | | | TTGAATGGTA |
| | | | RAKAAAEAQ | | | CTCCTGACGG |
| | | | AKAKANRD | | | GAGTGGCTCT |
| | | | ALTQRLKDI | | | GGCGGCGGAG |
| | | | VNEALRHNA | | | GTGGAAAAGG |
| | | | SRTPSATELA | | | AGGCAGTAAA |
| | | | HANNAAMQ | | | AGTGAAAGTT |
| | | | AEDERLRLA | | | CTGCAGCTAT |
| | | | KAEEKARKE | | | TCATGCAACT |
| | | | AEAAEKAFQ | | | GCTAAATGGT |
| | | | EAEQRRKEI | | | CTACTGCTCA |
| | | | EREKAETER | | | ATTAAAGAAA |
| | | | QLKLAEAEE | | | ACACAGGCAG |
| | | | KRLAALSEE | | | AGCAGGCTGC |
| | | | AKAVEIAQK | | | CCGGGCAAAA |
| | | | KLSAAQSEV | | | GCTGCAGCGG |
| | | | VKMDGEIKT | | | AAGCACAGGC |
| | | | LNSRLSSSIH | | | GAAAGCAAAG |
| | | | ARDAEMKTL | | | GCAAACAGGG |
| | | | AGKRNELAQ | | | ATGCGCTGAC |
| | | | ASAKYKELD | | | TCAGCGCCTG |
| | | | ELVKKLSPR | | | AAGGATATCG |
| | | | ANDPLQNRP | | | TGAATGAGGC |
| | | | FFEATRRRV | | | TCTTCGTCACA |
| | | | GAGKIREEK | | | ATGCCTCACG |
| | | | QKQVTASET | | | TACGCCTTCA |
| | | | RINRINADIT | | | GCAACAGAGC |
| | | | QIQKAISQVS | | | TTGCTCATGCT |
| | | | NNRNAGIAR | | | AATAATGCAG |
| | | | VHEAEENLK | | | CTATGCAGGC |
| | | | KAQNNLLNS | | | GGAAGACGAG |
| | | | QIKDAVDAT | | | CGTTTGCGCCT |
| | | | VSFYQTLTE | | | TGCGAAAGCA |
| | | | KYGEKYSK | | | GAAGAAAAAG |
| | | | MAQELADKS | | | CCCGTAAAGA |
| | | | KGKKIGNVN | | | AGCGGAAGCA |
| | | | EALAAFEKY | | | GCAGAAAAGG |
| | | | KDVLNKKFS | | | CTTTTCAGGA |
| | | | KADRDAIFN | | | AGCAGAACAA |
| | | | ALASVKYDD | | | CGACGTAAAG |
| | | | WAKHLDQF | | | AGATTGAACG |
| | | | AKYLKITGH | | | GGAGAAGGCT |
| | | | VSFGYDVVS | | | GAAACAGAAC |
| | | | DILKIKDTGD | | | GCCAGTTGAA |
| | | | WKPLFLTLE | | | ACTGGCTGAA |
| | | | KKAADAGVS | | | GCTGAAGAGA |
| | | | YVVALLFSL | | | AACGACTGGC |
| | | | LAGTTLGIW | | | TGCATTGAGT |
| | | | GIAIVTGILC | | | GAAGAAGCTA |
| | | | SYIDKNKLN | | | AAGCTGTTGA |
| | | | TINEVLGI | | | GATCGCCCAA |
| | | | | | | AAAAAACTTT |
| | | | | | | CTGCTGCACA |
| | | | | | | ATCTGAAGTG |
| | | | | | | GTGAAAATGG |
| | | | | | | ATGGAGAGAT |
| | | | | | | TAAGACTCTC |
| | | | | | | AATTCTCGTTT |
| | | | | | | AAGCTCCAGT |
| | | | | | | ATCCATGCCC |
| | | | | | | GTGATGCAGA |
| | | | | | | AATGAAAACG |
| | | | | | | CTCGCTGGAA |
| | | | | | | AACGAAATGA |
| | | | | | | ACTGGCTCAG |
| | | | | | | GCATCCGCTA |
| | | | | | | AATATAAAGA |
| | | | | | | ACTGGATGAG |
| | | | | | | CTGGTCAAAA |
| | | | | | | AACTATCACC |
| | | | | | | AAGAGCCAAT |
| | | | | | | GATCCGCTTC |
| | | | | | | AGAACCGTCC |
| | | | | | | TTTTTTTGAAG |
| | | | | | | CAACCAGACG |
| | | | | | | ACGGGTTGGG |
| | | | | | | GCCGGTAAGA |
| | | | | | | TTAGAGAAGA |
| | | | | | | AAAACAAAAA |
| | | | | | | CAGGTAACAG |
| | | | | | | CATCAGAAAC |
| | | | | | | ACGTATTAAC |
| | | | | | | CGGATAAATG |
| | | | | | | CTGATATAAC |
| | | | | | | TCAGATCCAG |
| | | | | | | AAGGCTATTT |
| | | | | | | CTCAGGTCAG |
| | | | | | | TAATAATCGT |
| | | | | | | AATGCCGGTA |
| | | | | | | TCGCTCGTGTT |
| | | | | | | CATGAAGCTG |
| | | | | | | AAGAAAATTT |
| | | | | | | GAAAAAAGCA |
| | | | | | | CAGAATAATC |
| | | | | | | TCCTTAATTCA |
| | | | | | | CAGATTAAGG |
| | | | | | | ATGCTGTTGA |
| | | | | | | TGCAACAGTT |
| | | | | | | AGCTTTTATCA |
| | | | | | | AACGCTGACT |
| | | | | | | GAAAAATATG |
| | | | | | | GTGAAAAATA |
| | | | | | | TTCGAAAATG |
| | | | | | | GCACAGGAAC |
| | | | | | | TTGCTGATAA |
| | | | | | | GTCTAAAGGT |
| | | | | | | AAGAAAATCG |
| | | | | | | GCAATGTGAA |
| | | | | | | TGAAGCTCTC |
| | | | | | | GCTGCTTTTGA |
| | | | | | | AAAATACAAG |
| | | | | | | GATGTTTTAA |
| | | | | | | ATAAGAAATT |
| | | | | | | CAGCAAAGCC |
| | | | | | | GATCGTGATG |
| | | | | | | CTATTTTTAAT |
| | | | | | | GCGTTGGCAT |
| | | | | | | CGGTGAAGTA |
| | | | | | | TGATGACTGG |
| | | | | | | GCTAAACATT |
| | | | | | | TAGATCAGTT |
| | | | | | | TGCCAAGTAC |
| | | | | | | TTGAAGATTA |
| | | | | | | CGGGGCATGT |
| | | | | | | TTCTTTTGGAT |
| | | | | | | ATGATGTGGT |
| | | | | | | ATCTGATATCC |
| | | | | | | TAAAAATTAA |
| | | | | | | GGATACAGGT |
| | | | | | | GACTGGAAGC |
| | | | | | | CACTATTTCTT |
| | | | | | | ACATTAGAGA |
| | | | | | | AGAAAGCTGC |
| | | | | | | AGATGCAGGG |
| | | | | | | GTGAGTTATG |
| | | | | | | TTGTTGCTTTA |
| | | | | | | CTTTTTAGCTT |
| | | | | | | GCTTGCTGGA |
| | | | | | | ACTACATTAG |
| | | | | | | GTATTTGGGG |
| | | | | | | TATTGCTATTG |
| | | | | | | TTACAGGAAT |
| | | | | | | TCTATGCTCCT |
| | | | | | | ATATTGATAA |
| | | | | | | GAATAAACTT |
| | | | | | | AATACTATAA |
| | | | | | | ATGAGGTGTT |
| | | | | | | AGGGATTTAA |
| 56 | Colicin-Ia | Unclassified | MSDPVRITN | Escherichia | 57 | ATGTCTGACC |
| | | | PGAESLGYD | coli | | CTGTACGTATT |
| | | | SDGHEIMAV | | | ACAAATCCCG |
| | | | DIYVNPPRV | | | GTGCAGAATC |
| | | | DVFHGTPPA | | | GCTGGGGTAT |
| | | | WSSFGNKTI | | | GATTCAGATG |
| | | | WGGNEWVD | | | GCCATGAAAT |
| | | | DSPTRSDIEK | | | TATGGCCGTT |
| | | | RDKEITAYK | | | GATATTTATGT |
| | | | NTLSAQQKE | | | AAACCCTCCA |
| | | | NENKRTEAG | | | CGTGTCGATG |
| | | | KRLSAAIAA | | | TCTTTCATGGT |
| | | | REKDENTLK | | | ACCCCGCCTG |
| | | | TLRAGNADA | | | CATGGAGTTC |
| | | | ADITRQEFRL | | | CTTCGGGAAC |
| | | | LQAELREYG | | | AAAACCATCT |
| | | | FRTEIAGYD | | | GGGGCGGAAA |
| | | | ALRLHTESR | | | CGAGTGGGTT |
| | | | MLFADADSL | | | GATGATTCCC |
| | | | RISPREARSL | | | CAACCCGAAG |
| | | | IEQAEKRQK | | | TGATATCGAA |
| | | | DAQNADKK | | | AAAAGGGACA |
| | | | AADMLAEY | | | AGGAAATCAC |
| | | | ERRKGILDT | | | AGCGTACAAA |
| | | | RLSELEKNG | | | AACACGCTCA |
| | | | GAALAVLDA | | | GCGCGCAGCA |
| | | | QQARLLGQQ | | | GAAAGAGAAT |
| | | | TRNDRAISE | | | GAGAATAAGC |
| | | | ARNKLSSVT | | | GTACTGAAGC |
| | | | ESLNTARNA | | | CGGAAAACGC |
| | | | LTRAEQQLT | | | CTCTCTGCGG |
| | | | QQKNTPDGK | | | CGATTGCTGC |
| | | | TIVSPEKFPG | | | AAGGGAAAAA |
| | | | RSSTNHSIVV | | | GATGAAAACA |
| | | | SGDPRFAGTI | | | CACTGAAAAC |
| | | | KITTSAVIDN | | | ACTCCGTGCC |
| | | | RANLNYLLS | | | GGAAACGCAG |
| | | | HSGLDYKRN | | | ATGCCGCTGA |
| | | | ILNDRNPVV | | | TATTACACGA |
| | | | TEDVEGDKK | | | CAGGAGTTCA |
| | | | IYNAEVAEW | | | GACTCCTGCA |
| | | | DKLRQRLLD | | | GGCAGAGCTG |
| | | | ARNKITSAES | | | AGAGAATACG |
| | | | AVNSARNNL | | | GATTCCGTAC |
| | | | SARTNEQKH | | | TGAAATCGCC |
| | | | ANDALNALL | | | GGATATGACG |
| | | | KEKENIRNQ | | | CCCTCCGGCT |
| | | | LSGINQKIAE | | | GCATACAGAG |
| | | | EKRKQDELK | | | AGCCGGATGC |
| | | | ATKDAINFT | | | TGTTTGCTGAT |
| | | | TEFLKSVSE | | | GCTGATTCTCT |
| | | | KYGAKAEQL | | | TCGTATATCTC |
| | | | AREMAGQA | | | CCCGGGAGGC |
| | | | KGKKIRNVE | | | CAGGTCGTTA |
| | | | EALKTYEKY | | | ATCGAACAGG |
| | | | RADINKKIN | | | CTGAAAAACG |
| | | | AKDRAAIAA | | | GCAGAAGGAT |
| | | | ALESVKLSDI | | | GCGCAGAACG |
| | | | SSNLNRFSR | | | CAGACAAGAA |
| | | | GLGYAGKFT | | | GGCCGCTGAT |
| | | | SLADWITEF | | | ATGCTTGCTG |
| | | | GKAVRTEN | | | AATACGAGCG |
| | | | WRPLFVKTE | | | CAGAAAAGGT |
| | | | TIIAGNAATA | | | ATTCTGGACA |
| | | | LVALVFSILT | | | CCCGGTTGTC |
| | | | GSALGIIGYG | | | AGAGCTGGAA |
| | | | LLMAVTGAL | | | AAAAATGGCG |
| | | | IDESLVEKA | | | GGGCAGCCCT |
| | | | NKFWGI | | | TGCCGTTCTTG |
| | | | | | | ATGCACAACA |
| | | | | | | GGCCCGTCTG |
| | | | | | | CTCGGGCAGC |
| | | | | | | AGACACGGAA |
| | | | | | | TGACAGGGCC |
| | | | | | | ATTTCAGAGG |
| | | | | | | CCCGGAATAA |
| | | | | | | ACTCAGTTCA |
| | | | | | | GTGACGGAAT |
| | | | | | | CGCTTAACAC |
| | | | | | | GGCCCGTAAT |
| | | | | | | GCATTAACCA |
| | | | | | | GAGCTGAACA |
| | | | | | | ACAGCTGACG |
| | | | | | | CAACAGAAAA |
| | | | | | | ACACGCCTGA |
| | | | | | | CGGCAAAACG |
| | | | | | | ATAGTTTCCCC |
| | | | | | | TGAAAAATTC |
| | | | | | | CCGGGGCGTT |
| | | | | | | CATCAACAAA |
| | | | | | | TGATTCTATTG |
| | | | | | | TTGTGAGCGG |
| | | | | | | TGATCCGAGA |
| | | | | | | TTTGCCGGTA |
| | | | | | | CGATAAAAAT |
| | | | | | | CACAACCAGC |
| | | | | | | GCAGTCATCG |
| | | | | | | ATAACCGTGC |
| | | | | | | AAACCTGAAT |
| | | | | | | TATCTTCTGAG |
| | | | | | | CCATTCCGGT |
| | | | | | | CTGGACTATA |
| | | | | | | AACGCAATAT |
| | | | | | | TCTGAATGAC |
| | | | | | | CGGAATCCGG |
| | | | | | | TGGTGACAGA |
| | | | | | | GGATGTGGAA |
| | | | | | | GGTGACAAGA |
| | | | | | | AAATTTATAA |
| | | | | | | TGCTGAAGTT |
| | | | | | | GCTGAATGGG |
| | | | | | | ATAAGTTACG |
| | | | | | | GCAAAGATTG |
| | | | | | | CTTGATGCCA |
| | | | | | | GAAATAAAAT |
| | | | | | | CACCTCTGCT |
| | | | | | | GAATCTGCGG |
| | | | | | | TAAATTCGGC |
| | | | | | | GAGAAATAAC |
| | | | | | | CTCAGTGCCA |
| | | | | | | GAACAAATGA |
| | | | | | | GCAAAAGCAT |
| | | | | | | GCAAATGACG |
| | | | | | | CTCTTAATGCC |
| | | | | | | CTGTTGAAGG |
| | | | | | | AAAAAGAGAA |
| | | | | | | TATCCGTAAC |
| | | | | | | CAGCTTTCCG |
| | | | | | | GCATCAATCA |
| | | | | | | GAAGATAGCG |
| | | | | | | GAAGAGAAAA |
| | | | | | | GAAAACAGGA |
| | | | | | | TGAACTGAAG |
| | | | | | | GCAACGAAAG |
| | | | | | | ACGCAATTAA |
| | | | | | | TTTCACAACA |
| | | | | | | GAGTTCCTGA |
| | | | | | | AATCAGTTTC |
| | | | | | | AGAAAAATAT |
| | | | | | | GGTGCAAAAG |
| | | | | | | CTGAGCAGTT |
| | | | | | | AGCCAGAGAG |
| | | | | | | ATGGCCGGGC |
| | | | | | | AGGCTAAAGG |
| | | | | | | GAAGAAAATA |
| | | | | | | CGTAATGTTG |
| | | | | | | AAGAGGCATT |
| | | | | | | AAAAACGTAT |
| | | | | | | GAAAAGTACC |
| | | | | | | GGGCTGACAT |
| | | | | | | TAACAAAAAA |
| | | | | | | ATTAATGCAA |
| | | | | | | AAGATCGTGC |
| | | | | | | AGCGATTGCC |
| | | | | | | GCAGCCCTTG |
| | | | | | | AGTCTGTGAA |
| | | | | | | GCTGTCTGAT |
| | | | | | | ATATCGTCTA |
| | | | | | | ATCTGAACAG |
| | | | | | | ATTCAGTCGG |
| | | | | | | GGACTGGGAT |
| | | | | | | ATGCAGGAAA |
| | | | | | | ATTTACAAGT |
| | | | | | | CTTGCTGACT |
| | | | | | | GGATCACTGA |
| | | | | | | GTTTGGTAAG |
| | | | | | | GCTGTCCGGA |
| | | | | | | CAGAGAACTG |
| | | | | | | GCGTCCTCTTT |
| | | | | | | TTGTTAAAAC |
| | | | | | | AGAAACCATC |
| | | | | | | ATAGCAGGCA |
| | | | | | | ATGCCGCAAC |
| | | | | | | GGCTCTTGTG |
| | | | | | | GCACTGGTCT |
| | | | | | | TCAGTATTCTT |
| | | | | | | ACCGGAAGCG |
| | | | | | | CTTTAGGCATT |
| | | | | | | ATCGGGTATG |
| | | | | | | GTTTACTGAT |
| | | | | | | GGCTGTCACC |
| | | | | | | GGTGCGCTGA |
| | | | | | | TTGATGAATC |
| | | | | | | GCTTGTGGAA |
| | | | | | | AAAGCGAATA |
| | | | | | | AGTTCTGGGG |
| | | | | | | TATTTAA |
| 58 | Colicin-Ib | Unclassified | MSDPVRITN | Escherichia | 59 | ATGTCTGACC |
| | | | PGAESLGYD | coli | | CTGTACGTATT |
| | | | SDGHEIMAV | | | ACAAATCCCG |
| | | | DIYVNPPRV | | | GTGCAGAATC |
| | | | DVFHGTPPA | | | GCTGGGATAT |
| | | | WSSFGNKTI | | | GATTCAGATG |
| | | | WGGNEWVD | | | GCCATGAAAT |
| | | | DSPTRSDIEK | | | TATGGCCGTT |
| | | | RDKEITAYK | | | GATATTTATGT |
| | | | NTLSAQQKE | | | AAACCCTCCA |
| | | | NENKRTEAG | | | CGTGTCGATG |
| | | | KRLSAAIAA | | | TCTTTCATGGT |
| | | | REKDENTLK | | | ACCCCGCCTG |
| | | | TLRAGNADA | | | CATGGAGTTC |
| | | | ADITRQEFRL | | | CTTCGGGAAC |
| | | | LQAELREYG | | | AAAACCATCT |
| | | | FRTEIAGYD | | | GGGGTGGAAA |
| | | | ALRLHTESR | | | CGAGTGGGTC |
| | | | MLFADADSL | | | GATGATTCCC |
| | | | RISPREARSL | | | CAACCCGAAG |
| | | | IEQAEKRQK | | | TGATATCGAA |
| | | | DAQNADKK | | | AAAAGGGACA |
| | | | AADMLAEY | | | AGGAAATCAC |
| | | | ERRKGILDT | | | AGCGTACAAA |
| | | | RLSELEKNG | | | AACACGCTCA |
| | | | GAALAVLDA | | | GCGCGCAGCA |
| | | | QQARLLGQQ | | | GAAAGAGAAT |
| | | | TRNDRAISE | | | GAGAATAAGC |
| | | | ARNKLSSVT | | | GTACTGAAGC |
| | | | ESLKTARNA | | | TGGAAAACGC |
| | | | LTRAEQQLT | | | CTTTCTGCGGC |
| | | | QQKNTPDGK | | | AATTGCTGCA |
| | | | TIVSPEKFPG | | | AGGGAAAAAG |
| | | | RSSTNHSIVV | | | ATGAAAACAC |
| | | | SGDPRFAGTI | | | ACTGAAAACA |
| | | | KITTSAVIDN | | | CTCCGTGCCG |
| | | | RANLNYLLT | | | GAAACGCAGA |
| | | | HSGLDYKRN | | | TGCCGCTGAT |
| | | | ILNDRNPVV | | | ATTACACGAC |
| | | | TEDVEGDKK | | | AGGAGTTCAG |
| | | | IYNAEVAEW | | | ACTCCTGCAG |
| | | | DKLRQRLLD | | | GCAGAGCTGA |
| | | | ARNKITSAES | | | GAGAATACGG |
| | | | AINSARNNV | | | ATTCCGTACT |
| | | | SARTNEQKH | | | GAAATCGCCG |
| | | | ANDALNALL | | | GATATGATGC |
| | | | KEKENIRSQ | | | CCTCCGGCTG |
| | | | LADINQKIAE | | | CATACAGAGA |
| | | | EKRKRDEIN | | | GCCGGATGCT |
| | | | MVKDAIKLT | | | GTTTGCTGAT |
| | | | SDFYRTIYDE | | | GCTGATTCTCT |
| | | | FGKQASELA | | | TCGTATATCTC |
| | | | KELASVSQG | | | CCCGCGAGGC |
| | | | KQIKSVDDA | | | CAGGTCGTTA |
| | | | LNAFDKFRN | | | ATCGAACAGG |
| | | | NLNKKYNIQ | | | CTGAAAAACG |
| | | | DRMAISKAL | | | GCAGAAGGAT |
| | | | EAINQVHMA | | | GCGCAGAACG |
| | | | ENFKLFSKAF | | | CAGACAAGAA |
| | | | GFTGKVIER | | | GGCCGCTGAT |
| | | | YDVAVELQK | | | ATGCTTGCTG |
| | | | AVKTDNWR | | | AATACGAGCG |
| | | | PFFVKLESLA | | | CAGAAAAGGT |
| | | | AGRAASAVT | | | ATTCTGGACA |
| | | | AWAFSVML | | | CGCGGTTGTC |
| | | | GTPVGILGF | | | AGAGCTGGAA |
| | | | AIIMAAVSA | | | AAAAATGGCG |
| | | | LVNDKFIEQ | | | GGGCAGCCCT |
| | | | VNKLIGI | | | TGCCGTTCTTG |
| | | | | | | ATGCACAACA |
| | | | | | | GGCCCGTCTG |
| | | | | | | CTCGGGCAGC |
| | | | | | | AGACACGGAA |
| | | | | | | TGACAGGGCC |
| | | | | | | ATTTCAGAGG |
| | | | | | | CCCGGAATAA |
| | | | | | | ACTCAGTTCG |
| | | | | | | GTGACGGAAT |
| | | | | | | CGCTTAAGAC |
| | | | | | | GGCCCGTAAT |
| | | | | | | GCATTAACCA |
| | | | | | | GAGCTGAACA |
| | | | | | | ACAGCTGACG |
| | | | | | | CAACAGAAAA |
| | | | | | | ACACGCCTGA |
| | | | | | | CGGCAAAACG |
| | | | | | | ATAGTTTCCCC |
| | | | | | | TGAAAAATTC |
| | | | | | | CCGGGGCGTT |
| | | | | | | CATCAACAAA |
| | | | | | | TCATTCTATTG |
| | | | | | | TTGTGAGTGG |
| | | | | | | TGATCCGAGG |
| | | | | | | TTTGCCGGTA |
| | | | | | | CGATAAAAAT |
| | | | | | | CACAACCAGC |
| | | | | | | GCGGTCATCG |
| | | | | | | ATAACCGTGC |
| | | | | | | AAACCTGAAT |
| | | | | | | TATCTTCTGAC |
| | | | | | | CCATTCCGGT |
| | | | | | | CTGGACTATA |
| | | | | | | AACGCAATAT |
| | | | | | | TCTGAATGAC |
| | | | | | | CGGAATCCGG |
| | | | | | | TGGTGACAGA |
| | | | | | | GGATGTGGAA |
| | | | | | | GGTGACAAGA |
| | | | | | | AAATTTATAA |
| | | | | | | TGCTGAAGTT |
| | | | | | | GCTGAATGGG |
| | | | | | | ATAAGTTACG |
| | | | | | | GCAACGATTG |
| | | | | | | CTTGATGCCA |
| | | | | | | GAAATAAAAT |
| | | | | | | CACCTCTGCT |
| | | | | | | GAATCTGCGA |
| | | | | | | TAAATTCGGC |
| | | | | | | GAGAAATAAC |
| | | | | | | GTCAGTGCCA |
| | | | | | | GAACAAATGA |
| | | | | | | ACAAAAGCAT |
| | | | | | | GCAAATGACG |
| | | | | | | CTCTTAATGCC |
| | | | | | | CTGTTGAAGG |
| | | | | | | AAAAAGAGAA |
| | | | | | | TATCCGTAGC |
| | | | | | | CAGCTTGCTG |
| | | | | | | ACATCAATCA |
| | | | | | | GAAAATAGCT |
| | | | | | | GAAGAGAAAA |
| | | | | | | GAAAAAGGGA |
| | | | | | | TGAAATAAAT |
| | | | | | | ATGGTAAAGG |
| | | | | | | ATGCCATAAA |
| | | | | | | ACTCACCTCTG |
| | | | | | | ATTTCTACAG |
| | | | | | | AACGATATAT |
| | | | | | | GATGAGTTCG |
| | | | | | | GTAAACAAGC |
| | | | | | | ATCCGAACTT |
| | | | | | | GCTAAGGAGC |
| | | | | | | TGGCTTCTGTA |
| | | | | | | TCTCAAGGGA |
| | | | | | | AACAGATTAA |
| | | | | | | GAGTGTGGAT |
| | | | | | | GATGCACTGA |
| | | | | | | ACGCTTTTGAT |
| | | | | | | AAATTCCGTA |
| | | | | | | ATAATCTGAA |
| | | | | | | CAAGAAATAT |
| | | | | | | AACATACAAG |
| | | | | | | ATCGCATGGC |
| | | | | | | CATTTCTAAA |
| | | | | | | GCCCTGGAAG |
| | | | | | | CTATTAATCA |
| | | | | | | GGTCCATATG |
| | | | | | | GCGGAGAATT |
| | | | | | | TTAAGCTGTTC |
| | | | | | | AGTAAGGCAT |
| | | | | | | TTGGTTTTACC |
| | | | | | | GGAAAAGTTA |
| | | | | | | TTGAACGTTA |
| | | | | | | TGATGTTGCT |
| | | | | | | GTGGAGTTAC |
| | | | | | | AAAAGGCTGT |
| | | | | | | AAAAACGGAC |
| | | | | | | AACTGGCGTC |
| | | | | | | CATTTTTTGTA |
| | | | | | | AAACTTGAAT |
| | | | | | | CACTGGCAGC |
| | | | | | | AGGAAGAGCT |
| | | | | | | GCTTCAGCAG |
| | | | | | | TTACAGCATG |
| | | | | | | GGCGTTTTCC |
| | | | | | | GTCATGCTGG |
| | | | | | | GAACCCCTGT |
| | | | | | | AGGTATTCTG |
| | | | | | | GGTTTTGCAA |
| | | | | | | TTATTATGGC |
| | | | | | | GGCTGTGAGT |
| | | | | | | GCGCTTGTTA |
| | | | | | | ATGATAAGTT |
| | | | | | | TATTGAGCAG |
| | | | | | | GTCAATAAAC |
| | | | | | | TTATTGGTATC |
| | | | | | | TGA |
| 60 | Colicin-M | Unclassified | METLTVHAP | Escherichia | 61 | ATGGAAACCT |
| | | | SPSTNLPSYG | coli | | TAACTGTTCAT |
| | | | NGAFSLSAP | | | GCACCATCAC |
| | | | HVPGAGPLL | | | CATCAACTAA |
| | | | VQVVYSFFQ | | | CTTACCAAGTT |
| | | | SPNMCLQAL | | | ATGGCAATGG |
| | | | TQLEDYIKK | | | TGCATTTTCTC |
| | | | HGASNPLTL | | | TTTCAGCACC |
| | | | QIISTNIGYF | | | ACATGTGCCT |
| | | | CNADRNLVL | | | GGTGCTGGCC |
| | | | HPGISVYDA | | | CTCTTTTAGTC |
| | | | YHFAKPAPS | | | CAGGTTGTTT |
| | | | QYDYRSMN | | | ATAGTTTTTTC |
| | | | MKQMSGNV | | | CAGAGTCCAA |
| | | | TTPIVALAH | | | ACATGTGTCTT |
| | | | YLWGNGAE | | | CAGGCTTTAA |
| | | | RSVNIANIGL | | | CTCAACTTGA |
| | | | KISPMKINQI | | | GGATTACATC |
| | | | KDIIKSGVV | | | AAAAAACATG |
| | | | GTFPVSTKFT | | | GGGCCAGCAA |
| | | | HATGDYNVI | | | CCCTCTCACAT |
| | | | TGAYLGNIT | | | TGCAGATCAT |
| | | | LKTEGTLTIS | | | ATCGACAAAT |
| | | | ANGSWTYN | | | ATTGGTTACTT |
| | | | GVVRSYDD | | | CTGTAACGCC |
| | | | KYDFNASTH | | | GACCGAAATC |
| | | | RGIIGESLTR | | | TGGTTCTTCAC |
| | | | LGAMFSGKE | | | CCTGGAATAA |
| | | | YQILLPGEIH | | | GCGTTTATGA |
| | | | IKESGKR | | | CGCTTACCACT |
| | | | | | | TCGCAAAACC |
| | | | | | | AGCGCCAAGT |
| | | | | | | CAATATGACT |
| | | | | | | ATCGCTCAAT |
| | | | | | | GAATATGAAA |
| | | | | | | CAAATGAGCG |
| | | | | | | GTAATGTCAC |
| | | | | | | TACACCAATT |
| | | | | | | GTGGCGCTTG |
| | | | | | | CTCACTATTTA |
| | | | | | | TGGGGTAATG |
| | | | | | | GCGCTGAAAG |
| | | | | | | GAGCGTTAAT |
| | | | | | | ATCGCCAACA |
| | | | | | | TTGGTCTTAA |
| | | | | | | AATTTCCCCTA |
| | | | | | | TGAAAATTAA |
| | | | | | | TCAGATAAAA |
| | | | | | | GACATTATAA |
| | | | | | | AATCTGGTGT |
| | | | | | | AGTAGGCACA |
| | | | | | | TTCCCTGTTTC |
| | | | | | | TACAAAGTTC |
| | | | | | | ACACATGCCA |
| | | | | | | CTGGTGATTA |
| | | | | | | TAATGTTATTA |
| | | | | | | CCGGTGCATA |
| | | | | | | TCTTGGTAAT |
| | | | | | | ATCACACTGA |
| | | | | | | AAACAGAAGG |
| | | | | | | TACTTTAACTA |
| | | | | | | TCTCTGCCAAT |
| | | | | | | GGCTCCTGGA |
| | | | | | | CTTACAATGG |
| | | | | | | CGTTGTTCGTT |
| | | | | | | CATATGATGA |
| | | | | | | TAAATACGAT |
| | | | | | | TTTAACGCCA |
| | | | | | | GCACTCACCG |
| | | | | | | TGGCATTATC |
| | | | | | | GGAGAGTCGC |
| | | | | | | TCACAAGGCT |
| | | | | | | CGGGGCGATG |
| | | | | | | TTTTCTGGTAA |
| | | | | | | AGAGTACCAG |
| | | | | | | ATACTGCTTCC |
| | | | | | | TGGTGAAATT |
| | | | | | | CACATTAAAG |
| | | | | | | AAAGTGGTAA |
| | | | | | | GCGATAA |
| 62 | Colicin-N | Unclassified | MGSNGADN | Escherichia | 63 | GCAAATCGAG |
| | | | AHNNAFGG | coli | | TTTCGAATATA |
| | | | GKNPGIGNT | | | AATAACATTA |
| | | | SGAGSNGSA | | | TATCTAGTGTT |
| | | | SSNRGNSNG | | | ATTCGATGA |
| | | | WSWSNKPH | | | |
| | | | KNDGFHSDG | | | |
| | | | SYHITFHGD | | | |
| | | | NNSKPKPGG | | | |
| | | | NSGNRGNN | | | |
| | | | GDGASAKV | | | |
| | | | GEITITPDNS | | | |
| | | | KPGRYISSNP | | | |
| | | | EYSLLAKLID | | | |
| | | | AESIKGTEV | | | |
| | | | YTFHTRKGQ | | | |
| | | | YVKVTVPDS | | | |
| | | | NIDKMRVDY | | | |
| | | | VNWKGPKY | | | |
| | | | NNKLVKRFV | | | |
| | | | SQFLLFRKEE | | | |
| | | | KEKNEKEAL | | | |
| | | | LKASELVSG | | | |
| | | | MGDKLGEY | | | |
| | | | LGVKYKNV | | | |
| | | | AKEVANDIK | | | |
| | | | NFHGRNIRS | | | |
| | | | YNEAMASLN | | | |
| | | | KVLANPKM | | | |
| | | | KVNKSDKD | | | |
| | | | AIVNAWKQ | | | |
| | | | VNAKDMAN | | | |
| | | | KIGNLGKAF | | | |
| | | | KVADLAIKV | | | |
| | | | EKIREKSIEG | | | |
| | | | YNTGNWGP | | | |
| | | | LLLEVESWII | | | |
| | | | GGVVAGVAI | | | |
| | | | SLFGAVLSFL | | | |
| | | | PISGLAVTAL | | | |
| | | | GVIGIMTISY | | | |
| | | | LSSFIDANRV | | | |
| | | | SNINNIISSVI | | | |
| | | | R | | | |
| 64 | Colicin-V | Unclassified | MRTLTLNEL | Escherichia | 65 | ATGAGAACTC |
| | (Microcin | | DSVSGGASG | coli | | TGACTCTAAA |
| | -v) | | RDIAMAIGT | | | TGAATTAGAT |
| | | | LSGQFVAGG | | | TCTGTTTCTGG |
| | | | IGAAAGGVA | | | TGGTGCTTCA |
| | | | GGAIYDYAS | | | GGGCGTGATA |
| | | | THKPNPAMS | | | TTGCGATGGC |
| | | | PSGLGGTIK | | | TATAGGAACA |
| | | | QKPEGIPSEA | | | CTATCCGGAC |
| | | | WNYAAGRL | | | AATTTGTTGC |
| | | | CNWSPNNLS | | | AGGAGGAATT |
| | | | DVCL | | | GGAGCAGCTG |
| | | | | | | CTGGGGGTGT |
| | | | | | | GGCTGGAGGT |
| | | | | | | GCAATATATG |
| | | | | | | ACTATGCATC |
| | | | | | | CACTCACAAA |
| | | | | | | CCTAATCCTGC |
| | | | | | | AATGTCTCCAT |
| | | | | | | CCGGTTTAGG |
| | | | | | | AGGAACAATT |
| | | | | | | AAGCAAAAAC |
| | | | | | | CCGAAGGGAT |
| | | | | | | ACCTTCAGAA |
| | | | | | | GCATGGAACT |
| | | | | | | ATGCTGCGGG |
| | | | | | | AAGATTGTGT |
| | | | | | | AATTGGAGTC |
| | | | | | | CAAATAATCT |
| | | | | | | TAGTGATGTTT |
| | | | | | | GTTTATAA |
| 66 | Columbicin | Lantibiotic | MMNATENQI | Enterococcus | 67 | ATGATGAATG |
| | A | | FVETVSDQE | columbae | | CTACTGAAAA |
| | | | LEMLIGGAG | | | CCAAATTTTTG |
| | | | RGWIKTLTK | | | TTGAGACTGT |
| | | | DCPNVISSIC | | | GAGTGACCAA |
| | | | AGTIITACKN | | | GAATTAGAAA |
| | | | CA | | | TGTTAATTGGT |
| | | | | | | GGTGCAGGTC |
| | | | | | | GTGGATGGAT |
| | | | | | | TAAGACTTTA |
| | | | | | | ACAAAAGATT |
| | | | | | | GTCCAAATGT |
| | | | | | | GATTTCTTCAA |
| | | | | | | TTTGTGCAGG |
| | | | | | | TACAATTATTA |
| | | | | | | CAGCTTGTAA |
| | | | | | | AAATTGTGCT |
| | | | | | | TAA |
| 68 | Curvacin- | class | MNNVKELS | Lactobacillus | 69 | ATGAATAATG |
| | A | IIA/YG | MTELQTITG | curvatus | | TAAAAGAATT |
| | | NGV | GARSYGNG | | | AAGTATGACA |
| | | | VYCNNKKC | | | GAATTACAAA |
| | | | WVNRGEAT | | | CAATTACCGG |
| | | | QSIIGGMISG | | | CGGTGCTAGA |
| | | | WASGLAGM | | | TCATATGGCA |
| | | | | | | ACGGTGTTTA |
| | | | | | | CTGTAATAAT |
| | | | | | | AAAAAATGTT |
| | | | | | | GGGTAAATCG |
| | | | | | | GGGTGAAGCA |
| | | | | | | ACGCAAAGTA |
| | | | | | | TTATTGGTGG |
| | | | | | | TATGATTAGC |
| | | | | | | GGCTGGGCTA |
| | | | | | | GTGGTTTAGC |
| | | | | | | TGGAATGTAA |
| 70 | Cypemycin | Unclassified | MRSEMTLTS | Streptomyces | 71 | GTGCGATCTG |
| | | tied | TNSAEALAA | sp. | | AGATGACTCT |
| | | | QDFANTVLS | | | TACGAGCACG |
| | | | AAAPGFHAD | | | AATTCCGCTG |
| | | | CETPAMATP | | | AGGCTCTGGC |
| | | | ATPTVAQFV | | | GGCGCAGGAC |
| | | | IQGSTICLVC | | | TTTGCGAACA |
| | | | | | | CCGTTCTCAG |
| | | | | | | CGCGGCGGCC |
| | | | | | | CCGGGCTTCC |
| | | | | | | ACGCGGACTG |
| | | | | | | CGAGACGCCG |
| | | | | | | GCCATGGCCA |
| | | | | | | CCCCGGCCAC |
| | | | | | | GCCGACCGTC |
| | | | | | | GCCCAGTTCG |
| | | | | | | TGATCCAGGG |
| | | | | | | CAGCACGATC |
| | | | | | | TGCCTGGTCT |
| | | | | | | GCTGA |
| 72 | Cytolysin | Lantibiotic | MVNSKDLR | Bacillus | 73 | ATGGTAAATT |
| | | | NPEFRKAQG | halodurans | | CAAAAGATTT |
| | | | LQFVDEVNE | (strain ATCC | | GCGTAATCCT |
| | | | KELSSLAGS | BAA-125/ | | GAATTCCGCA |
| | | | GDVHAQTT | DSM 18197/ | | AAGCCCAAGG |
| | | | WPCATVGVS | FERM 7344 | | TCTACAATTCG |
| | | | VALCPTTKC | /JCM9153/ | | TTGACGAGGT |
| | | | TSQC | C-125) | | GAACGAGAAG |
| | | | | | | GAACTTTCGT |
| | | | | | | CTCTAGCTGG |
| | | | | | | TTCAGGAGAT |
| | | | | | | GTGCATGCAC |
| | | | | | | AAACAACTTG |
| | | | | | | GCCTTGCGCT |
| | | | | | | ACAGTTGGTG |
| | | | | | | TCTCCGTAGC |
| | | | | | | CTTGTGCCCA |
| | | | | | | ACTACAAAGT |
| | | | | | | GTACAAGCCA |
| | | | | | | GTGCTAA |
| 74 | Divercin | class | MKNLKEGSY | Carnobacterium | 75 | ATGAAAAACT |
| | V41 | IIa/YGN | TAVNTDELK | divergens | | TAAAAGAAGG |
| | | GV | SINGGTKYY | (Lactobacillus | | TTCATACACTG |
| | | | GNGVYCNS | divergens) | | CTGTTAATACT |
| | | | KKCWVDWG | | | GATGAATTAA |
| | | | QASGCIGQT | | | AAAGTATCAA |
| | | | VVGGWLGG | | | TGGTGGAACA |
| | | | AIPGKC | | | AAATATTATG |
| | | | | | | GGAATGGCGT |
| | | | | | | TTATTGCAATT |
| | | | | | | CTAAAAAATG |
| | | | | | | TTGGGTAGAT |
| | | | | | | TGGGGACAAG |
| | | | | | | CTTCAGGTTGT |
| | | | | | | ATCGGTCAAA |
| | | | | | | CTGTTGTTGG |
| | | | | | | CGGATGGCTA |
| | | | | | | GGCGGAGCTA |
| | | | | | | TACCAGGTAA |
| | | | | | | ATGCTAA |
| 76 | Divergicin | Unclassified | MIKREKNRT | Carnobacterium | 77 | ATGATTAAAA |
| | 750 | | ISSLGYEEIS | divergens | | GAGAAAAGAA |
| | | | NHKLQEIQG | (Lactobacillus | | CAGAACAATT |
| | | | GKGILGKLG | divergens) | | TCTTCCCTTGG |
| | | | VVQAGVDF | | | TTATGAAGAA |
| | | | VSGVWAGIK | | | ATTTCTAATCA |
| | | | QSAKDHPNA | | | TAAATTGCAA |
| | | | | | | GAAATACAAG |
| | | | | | | GTGGAAAAGG |
| | | | | | | AATTCTTGGT |
| | | | | | | AAACTAGGAG |
| | | | | | | TAGTACAGGC |
| | | | | | | AGGAGTGGAT |
| | | | | | | TTTGTATCAG |
| | | | | | | GAGTGTGGGC |
| | | | | | | TGGAATAAAA |
| | | | | | | CAGTCTGCCA |
| | | | | | | AAGATCATCC |
| | | | | | | TAATGCGTAA |
| 78 | Divergicin | Class IIc | MKKQILKGL | Carnobacterium | 79 | ATGAAAAAAC |
| | A | | VIVVCLSGA | divergens | | AAATTTTAAA |
| | | | TFFSTPQQAS | (Lactobacillus | | AGGGTTGGTT |
| | | | AAAPKITQK | divergens) | | ATAGTTGTTTG |
| | | | QKNCVNGQ | | | TTTATCTGGG |
| | | | LGGMLAGA | | | GCAACATTTTT |
| | | | LGGPGGVVL | | | CTCAACACCA |
| | | | GGIGGAIAG | | | CAACAAGCTT |
| | | | GCFN | | | CTGCTGCTGC |
| | | | | | | ACCGAAAATT |
| | | | | | | ACTCAAAAAC |
| | | | | | | AAAAAAATTG |
| | | | | | | TGTTAATGGA |
| | | | | | | CAATTAGGTG |
| | | | | | | GAATGCTTGC |
| | | | | | | TGGAGCTTTG |
| | | | | | | GGTGGACCTG |
| | | | | | | GCGGAGTTGT |
| | | | | | | GTTAGGTGGT |
| | | | | | | ATAGGTGGTG |
| | | | | | | CAATAGCAGG |
| | | | | | | AGGTTGTTTTA |
| | | | | | | ATTAA |
| 80 | Durancin | Unclassified | MQTIKELNT | Enterococcus | 81 | ATGCAAACGA |
| | Q | | MELQEIIGGE | durans | | TCAAAGAATT |
| | | | NDHRMPYEL | | | GAACACGATG |
| | | | NRPNNLSKG | | | GAATTACAAG |
| | | | GAKCAAGIL | | | AAATAATTGG |
| | | | GAGLGAVG | | | AGGTGAAAAT |
| | | | GGPGGFISA | | | GACCATCGGA |
| | | | GISAVLGCM | | | TGCCTTACGA |
| | | | | | | ATTGAACCGT |
| | | | | | | CCAAATAATT |
| | | | | | | TATCCAAAGG |
| | | | | | | TGGGGCTAAG |
| | | | | | | TGTGCTGCTG |
| | | | | | | GAATACTTGG |
| | | | | | | CGCTGGACTA |
| | | | | | | GGCGCAGTAG |
| | | | | | | GCGGTGGACC |
| | | | | | | TGGCGGATTT |
| | | | | | | ATTAGTGCCG |
| | | | | | | GAATCAGTGC |
| | | | | | | TGTTCTTGGTT |
| | | | | | | GTATGTAA |
| 82 | Durancin | Unclassified | MQTIKELNT | Enterococcus | 83 | ATGCAAACGA |
| | TW-49M | | MELQKIIGG | durans | | TCAAAGAATT |
| | | | ENDHRMPYE | | | GAACACGATG |
| | | | LNRPNNLSK | | | GAATTACAAA |
| | | | GGAKCAAGI | | | AAATAATTGG |
| | | | LGAGLGAVG | | | AGGTGAAAAT |
| | | | GGPGGFISA | | | GACCATCGGA |
| | | | GISAVLGCM | | | TGCCTTACGA |
| | | | | | | ATTGAACCGT |
| | | | | | | CCAAATAATT |
| | | | | | | TATCCAAAGG |
| | | | | | | TGGAGCTAAG |
| | | | | | | TGCGCTGCCG |
| | | | | | | GAATACTTGG |
| | | | | | | TGCTGGATTA |
| | | | | | | GGCGCAGTAG |
| | | | | | | GCGGTGGACC |
| | | | | | | TGGCGGATTT |
| | | | | | | ATTAGTGCCG |
| | | | | | | GAATCAGTGC |
| | | | | | | TGTTCTTGGTT |
| | | | | | | GTATGTAA |
| 84 | Dysgalacticin | Unclassified | MKKLKRLVI | Streptococcus | 85 | ATGAAAAAAT |
| | | | SLVTSLLVIS | dysgalactiae | | TAAAACGTCT |
| | | | STVPALVYA | subsp. | | TGTTATCTCTC |
| | | | NETNNFAET | equisimilis | | TTGTTACTTCA |
| | | | QKEITTNSEA | (Streptococcus | | TTACTAGTAAT |
| | | | TLTNEDYTK | equisimilis) | | TTCAAGTACA |
| | | | LTSEVKTIYT | | | GTTCCAGCAC |
| | | | NLIQYDQTK | | | TTGTTTACGCT |
| | | | NKFYVDEDK | | | AATGAAACAA |
| | | | TEQYYNYD | | | ATAACTTTGC |
| | | | DESIKGVYL | | | AGAAACTCAA |
| | | | MKDSLNDEL | | | AAAGAAATTA |
| | | | NNNNSSNYS | | | CAACAAATTC |
| | | | EIINQKISEID | | | AGAAGCAACA |
| | | | YVLQGNDIN | | | TTAACCAATG |
| | | | NLIPSNTRVK | | | AAGACTACAC |
| | | | RSADFSWIQ | | | TAAATTAACTT |
| | | | RCLEEAWGY | | | CCGAAGTAAA |
| | | | AISLVTLKGI | | | AACAATTTAT |
| | | | INLFKAGKFE | | | ACAAATCTGA |
| | | | AAAAKLASA | | | TTCAATACGA |
| | | | TAGRIAGMA | | | CCAAACAAAA |
| | | | ALFAFVATC | | | AACAAATTTT |
| | | | GATTVS | | | ACGTCGATGA |
| | | | | | | AGACAAAACT |
| | | | | | | GAACAATATT |
| | | | | | | ATAACTACGA |
| | | | | | | TGATGAAAGT |
| | | | | | | ATAAAAGGGG |
| | | | | | | TTTATCTCATG |
| | | | | | | AAAGATAGTT |
| | | | | | | TGAACGATGA |
| | | | | | | GTTAAACAAT |
| | | | | | | AATAACTCTTC |
| | | | | | | AAACTATTCT |
| | | | | | | GAAATAATTA |
| | | | | | | ATCAAAAAAT |
| | | | | | | CTCTGAAATT |
| | | | | | | GACTATGTCC |
| | | | | | | TTCAAGGAAA |
| | | | | | | CGATATAAAT |
| | | | | | | AATTTAATTCC |
| | | | | | | TAGCAATACC |
| | | | | | | AGAGTAAAAA |
| | | | | | | GATCAGCAGA |
| | | | | | | TTTTTCTTGGA |
| | | | | | | TTCAAAGATG |
| | | | | | | TCTAGAAGAA |
| | | | | | | GCATGGGGAT |
| | | | | | | ATGCTATTAG |
| | | | | | | TCTAGTTACTC |
| | | | | | | TAAAAGGAAT |
| | | | | | | AATCAATCTA |
| | | | | | | TTTAAAGCAG |
| | | | | | | GAAAATTTGA |
| | | | | | | AGCTGCTGCT |
| | | | | | | GCTAAATTAG |
| | | | | | | CTTCTGCTACA |
| | | | | | | GCAGGTAGAA |
| | | | | | | TCGCTGGAAT |
| | | | | | | GGCTGCCTTA |
| | | | | | | TTTGCTTTCGT |
| | | | | | | AGCAACTTGC |
| | | | | | | GGTGCGACAA |
| | | | | | | CTGTATCATA |
| | | | | | | A |
| 86 | Enterocin | Unclassified | MKQYKVLN | Enterococcus | 87 | ATGAAGCAAT |
| | 1071A | | EKEMKKPIG | faecalis | | ATAAAGTATT |
| | | | GESVFSKIGN | (Streptococcus | | GAATGAAAAA |
| | | | AVGPAAYWI | faecalis) | | GAAATGAAAA |
| | | | LKGLGNMSD | | | AACCTATTGG |
| | | | VNQADRINR | | | GGGAGAGTCG |
| | | | KKH | | | GTTTTTAGTAA |
| | | | | | | AATAGGTAAT |
| | | | | | | GCTGTAGGTC |
| | | | | | | CAGCTGCTTA |
| | | | | | | TTGGATTTTAA |
| | | | | | | AAGGATTAGG |
| | | | | | | TAATATGAGT |
| | | | | | | GATGTAAACC |
| | | | | | | AAGCTGATAG |
| | | | | | | AATTAATAGA |
| | | | | | | AAGAAACATT |
| | | | | | | AA |
| 88 | Enterocin | bacteriocins | MGAIAKLVA | Enterococcus | 89 | ATGGGAGCAA |
| | 7A | without | KFGWPIVKK | faecalis | | TCGCAAAATT |
| | (Enterocin | sequence | YYKQIMQFI | (Streptococcus | | AGTAGCAAAG |
| | L50A) | leader | GEGWAINKII | faecalis) | | TTTGGATGGC |
| | | | DWIKKHI | | | CAATTGTTAA |
| | | | | | | AAAGTATTAC |
| | | | | | | AAACAAATTA |
| | | | | | | TGCAATTTATT |
| | | | | | | GGAGAAGGAT |
| | | | | | | GGGCAATTAA |
| | | | | | | CAAAATTATT |
| | | | | | | GATTGGATCA |
| | | | | | | AAAAACATAT |
| | | | | | | TTAA |
| 90 | Enterocin | Unclassified | MGAIAKLVA | Enterococcus | 91 | ATGGGAGCAA |
| | 7B | | KFGWPFIKK | faecalis | | TCGCAAAATT |
| | | | FYKQIMQFIG | (Streptococcus | | AGTAGCAAAG |
| | | | QGWTIDQIE | faecalis) | | TTTGGATGGC |
| | | | KWLKRH | | | CATTTATTAAA |
| | | | | | | AAATTCTACA |
| | | | | | | AACAAATTAT |
| | | | | | | GCAGTTTATC |
| | | | | | | GGACAAGGAT |
| | | | | | | GGACAATAGA |
| | | | | | | TCAAATTGAA |
| | | | | | | AAATGGTTAA |
| | | | | | | AAAGACATTG |
| | | | | | | A |
| 92 | Enterocin | Class II | MLNKKLLEN | Enterococcus | 93 | ATGTTAAATA |
| | 96 | | GVVNAVTID | faecalis | | AAAAATTATT |
| | | | ELDAQFGGM | (strain ATCC | | AGAAAATGGT |
| | | | SKRDCNLMK | 700802/ | | GTAGTAAATG |
| | | | ACCAGQAVT | V583) | | CTGTAACAAT |
| | | | YAIHSLLNRL | | | TGATGAACTT |
| | | | GGDSSDPAG | | | GATGCTCAAT |
| | | | CNDIVRKYC | | | TTGGTGGAAT |
| | | | K | | | GAGCAAACGT |
| | | | | | | GATTGTAACT |
| | | | | | | TGATGAAGGC |
| | | | | | | GTGTTGTGCT |
| | | | | | | GGACAAGCAG |
| | | | | | | TAACATATGC |
| | | | | | | TATTCATAGTC |
| | | | | | | TTTTAAATCGA |
| | | | | | | TTAGGTGGAG |
| | | | | | | ACTCTAGTGA |
| | | | | | | TCCAGCTGGT |
| | | | | | | TGTAATGATA |
| | | | | | | TTGTAAGAAA |
| | | | | | | ATATTGTAAA |
| | | | | | | TAA |
| 94 | Enterocin | Class | MKHLKILSIK | Enterococcus | 95 | ATGAAACATT |
| | A | IIa, IIc | ETQLIYGGT | faecium | | TAAAAATTTT |
| | | (problem | THSGKYYGN | (Streptococcus | | GTCTATTAAA |
| | | atic) | GVYCTKNKC | faecium) | | GAGACACAAC |
| | | | TVDWAKAT | | | TTATCTATGG |
| | | | TCIAGMSIG | | | GGGTACCACT |
| | | | GFLGGAIPG | | | CATAGTGGAA |
| | | | KC | | | AATATTATGG |
| | | | | | | AAATGGAGTG |
| | | | | | | TATTGCACTA |
| | | | | | | AAAATAAATG |
| | | | | | | TACGGTCGAT |
| | | | | | | TGGGCCAAGG |
| | | | | | | CAACTACTTGT |
| | | | | | | ATTGCAGGAA |
| | | | | | | TGTCTATAGG |
| | | | | | | TGGTTTTTTAG |
| | | | | | | GTGGAGCAAT |
| | | | | | | TCCAGGGAAG |
| | | | | | | TGC |
| 96 | Enterocin | Unclassified | MVKENKFSK | Enterococcus | 97 | ATGGTTAAAG |
| | AS-48 | | IFILMALSFL | faecalis | | AAAATAAATT |
| | (BACTE | | GLALFSASL | (Streptococcus | | TTCTAAGATTT |
| | RIOCIN | | QFLPIAHMA | faecalis) | | TTATTTTAATG |
| | AS-48) | | KEFGIPAAV | | | GCTTTGAGTTT |
| | | | AGTVLNVVE | | | TTTGGGGTTA |
| | | | AGGWVTTIV | | | GCCTTGTTTAG |
| | | | SILTAVGSG | | | TGCAAGTCTT |
| | | | GLSLLAAAG | | | CAGTTTTTGCC |
| | | | RESIKAYLK | | | CATTGCACAT |
| | | | KEIKKKGKR | | | ATGGCTAAAG |
| | | | AVIAW | | | AGTTCGGTAT |
| | | | | | | ACCAGCAGCA |
| | | | | | | GTTGCAGGAA |
| | | | | | | CTGTGCTTAAT |
| | | | | | | GTAGTTGAAG |
| | | | | | | CTGGTGGATG |
| | | | | | | GGTCACTACT |
| | | | | | | ATTGTATCAAT |
| | | | | | | TCTTACTGCTG |
| | | | | | | TAGGTAGCGG |
| | | | | | | AGGTCTTTCTT |
| | | | | | | TACTCGCTGC |
| | | | | | | AGCAGGAAGA |
| | | | | | | GAGTCAATTA |
| | | | | | | AAGCATACCT |
| | | | | | | TAAGAAAGAA |
| | | | | | | ATTAAGAAAA |
| | | | | | | AAGGAAAAAG |
| | | | | | | AGCAGTTATT |
| | | | | | | GCTTGGTAA |
| 98 | Enterocin | class IIc, | MQNVKELST | Enterococcus | 99 | ATGCAAAATG |
| | B | non | KEMKQIIGG | faecium | | TAAAAGAATT |
| | | subgrouped | ENDHRMPNE | (Streptococcus | | AAGTACGAAA |
| | | bacteriocins | LNRPNNLSK | faecium) | | GAGATGAAAC |
| | | (problematic) | GGAKCGAAI | | | AAATTATCGG |
| | | | AGGLFGIPK | | | TGGAGAAAAT |
| | | | GPLAWAAGL | | | GATCACAGAA |
| | | | ANVYSKCN | | | TGCCTAATGA |
| | | | | | | GTTAAATAGA |
| | | | | | | CCTAACAACT |
| | | | | | | TATCTAAAGG |
| | | | | | | TGGAGCAAAA |
| | | | | | | TGTGGTGCTG |
| | | | | | | CAATTGCTGG |
| | | | | | | GGGATTATTT |
| | | | | | | GGAATCCCAA |
| | | | | | | AAGGACCACT |
| | | | | | | AGCATGGGCT |
| | | | | | | GCTGGGTTAG |
| | | | | | | CAAATGTATA |
| | | | | | | CTCTAAATGC |
| | | | | | | AACTAA |
| 100 | Enterocin | Class IIa | MKKLTSKE | Enterococcus | 101 | TTGAAGAAAT |
| | CRL35 | | MAQVVGGK | mundtii | | TAACATCAAA |
| | (Mundticin | | YYGNGVSC | | | AGAAATGGCA |
| | KS) | | NKKGCSVD | | | CAAGTAGTAG |
| | | | WGKAIGIIGN | | | GTGGAAAATA |
| | | | NSAANLATG | | | CTACGGTAAT |
| | | | GAAGWKS | | | GGAGTCTCAT |
| | | | | | | GTAATAAAAA |
| | | | | | | AGGGTGCAGT |
| | | | | | | GTTGATTGGG |
| | | | | | | GAAAAGCTAT |
| | | | | | | TGGCATTATT |
| | | | | | | GGAAATAATT |
| | | | | | | CTGCTGCGAA |
| | | | | | | TTTAGCTACTG |
| | | | | | | GTGGAGCAGC |
| | | | | | | TGGTTGGAAA |
| | | | | | | AGTTAA |
| 102 | Enterocin | Unclassified | MLAKIKAMI | Enterococcus | 103 | ATGTTAGCAA |
| | EJ97 | | KKFPNPYTL | faecalis | | AAATTAAAGC |
| | | | AAKLTTYEI | (Streptococcus | | GATGATTAAG |
| | | | NWYKQQYG | faecalis) | | AAGTTTCCGA |
| | | | RYPWERPVA | | | ACCCTTATACT |
| | | | | | | TTAGCAGCTA |
| | | | | | | AGCTAACGAC |
| | | | | | | TTACGAAATT |
| | | | | | | AATTGGTATA |
| | | | | | | AACAACAATA |
| | | | | | | CGGTCGTTAT |
| | | | | | | CCTTGGGAGC |
| | | | | | | GCCCTGTAGC |
| | | | | | | ATAA |
| 104 | Enterocin | Class | MRKKLFSLA | Enterococcus | 105 | ATGAGAAAAA |
| | P | IIa, IIb | LIGIFGLVVT | faecium | | AATTATTTAGT |
| | | and IIc | NFGTKVDAA | (Streptococcus | | TTAGCTCTTAT |
| | | (problematic) | TRSYGNGVY | faecium) | | TGGAATATTT |
| | | | CNNSKCWV | | | GGGTTAGTTG |
| | | | NWGEAKENI | | | TGACAAATTTT |
| | | | AGIVISGWA | | | GGTACAAAAG |
| | | | SGLAGMGH | | | TTGATGCAGC |
| | | | | | | TACGCGTTCA |
| | | | | | | TATGGTAATG |
| | | | | | | GTGTTTATTGT |
| | | | | | | AATAATAGTA |
| | | | | | | AATGCTGGGT |
| | | | | | | TAACTGGGGA |
| | | | | | | GAAGCTAAAG |
| | | | | | | AGAATATTGC |
| | | | | | | AGGAATCGTT |
| | | | | | | ATTAGTGGCT |
| | | | | | | GGGCTTCTGG |
| | | | | | | TTTGGCAGGT |
| | | | | | | ATGGGACATT |
| | | | | | | AA |
| 106 | Enterocin | Class IIc | MNFLKNGIA | Enterococcus | 107 | ATGAATTTTCT |
| | Q | | KWMTGAEL | faecium | | TAAAAATGGT |
| | | | QAYKKKYG | (Streptococcus | | ATCGCAAAAT |
| | | | CLPWEKISC | faecium) | | GGATGACCGG |
| | | | | | | TGCTGAATTG |
| | | | | | | CAAGCGTATA |
| | | | | | | AAAAGAAATA |
| | | | | | | TGGATGCTTG |
| | | | | | | CCATGGGAAA |
| | | | | | | AAATTTCTTGT |
| | | | | | | TAA |
| 108 | Enterocin | Class IIa | MKKKLVKG | Enterococcus | 109 | ATGAAAAAGA |
| | SE-K4 | | LVICGMIGIG | faecalis | | AATTAGTTAA |
| | | | FTALGTNVE | (Streptococcus | | AGGCTTAGTT |
| | | | AATYYGNG | faecalis) | | ATTTGTGGCA |
| | | | VYCNKQKC | | | TGATTGGGAT |
| | | | WVDWSRAR | | | TGGTTTTACA |
| | | | SEIIDRGVKA | | | GCATTAGGAA |
| | | | YVNGFTKVL | | | CAAATGTAGA |
| | | | GGIGGR | | | AGCCGCCACG |
| | | | | | | TATTACGGAA |
| | | | | | | ATGGTGTCTA |
| | | | | | | TTGCAATAAG |
| | | | | | | CAAAAATGTT |
| | | | | | | GGGTAGATTG |
| | | | | | | GAGTAGAGCA |
| | | | | | | CGTTCTGAAA |
| | | | | | | TTATAGACAG |
| | | | | | | AGGCGTAAAA |
| | | | | | | GCATACGTCA |
| | | | | | | ATGGATTTAC |
| | | | | | | GAAAGTGTTA |
| | | | | | | GGTGGTATAG |
| | | | | | | GTGGAAGATA |
| | | | | | | A |
| 110 | Enterocin | class IIb | MKKEELVG | Enterococcus | ill | ATGAAAAAAG |
| | W alfa | | MAKEDFLNV | faecalis | | AAGAATTAGT |
| | | | ICENDNKLE | (Streptococcus | | AGGAATGGCT |
| | | | NSGAKCPW | faecalis) | | AAGGAAGACT |
| | | | WNLSCHLGN | | | TTTTAAATGTT |
| | | | DGKICTYSH | | | ATTTGTGAAA |
| | | | ECTAGCNA | | | ATGACAACAA |
| | | | | | | ACTAGAAAAT |
| | | | | | | AGTGGAGCAA |
| | | | | | | AATGTCCTTG |
| | | | | | | GTGGAATCTT |
| | | | | | | TCTTGTCATTT |
| | | | | | | AGGCAATGAT |
| | | | | | | GGTAAAATTT |
| | | | | | | GCACTTATTCA |
| | | | | | | CATGAATGTA |
| | | | | | | CCGCAGGTTG |
| | | | | | | TAATGCATAA |
| 112 | Enterocin | class IIb | MTELNKRLQ | Enterococcus | 113 | ATGACTGAAC |
| | W beta | | LKRDVSTEN | faecalis | | TTAACAAAAG |
| | | | SLKKISNTDE | (Streptococcus | | ATTACAATTA |
| | | | THGGVTTSIP | faecalis) | | AAAAGAGATG |
| | | | CTVMVSAA | | | TTTCAACAGA |
| | | | VCPTLVCSN | | | AAATAGTTTG |
| | | | KCGGRG | | | AAAAAAATTT |
| | | | | | | CTAATACTGA |
| | | | | | | TGAAACACAT |
| | | | | | | GGGGGAGTTA |
| | | | | | | CTACATCAATT |
| | | | | | | CCATGTACAG |
| | | | | | | TAATGGTTAG |
| | | | | | | TGCGGCAGTA |
| | | | | | | TGTCCTACCCT |
| | | | | | | TGTTTGCTCGA |
| | | | | | | ATAAATGTGG |
| | | | | | | CGGTAGAGGC |
| | | | | | | TAG |
| 114 | Enterocin | class IIb | MQNVKEVS | Enterococcus | 115 | ATGCAAAATG |
| | Xalpha | | VKEMKQIIG | faecium | | TAAAAGAAGT |
| | | | GSNDSLWY | (Streptococcus | | TTCTGTAAAA |
| | | | GVGQFMGK | faecium) | | GAGATGAAAC |
| | | | QANCITNHP | | | AAATTATCGG |
| | | | VKHMIIPGY | | | TGGTTCTAAT |
| | | | CLSKILG | | | GATAGTCTTT |
| | | | | | | GGTATGGTGT |
| | | | | | | AGGACAATTT |
| | | | | | | ATGGGTAAAC |
| | | | | | | AAGCAAACTG |
| | | | | | | TATAACAAAC |
| | | | | | | CATCCTGTTAA |
| | | | | | | ACACATGATA |
| | | | | | | ATTCCTGGAT |
| | | | | | | ATTGTTTATCG |
| | | | | | | AAAATTTTAG |
| | | | | | | GGTAA |
| 116 | Enterocin | class IIb | MKKYNELSK | Enterococcus | 117 | ATGAAAAAAT |
| | Xbeta | | KELLQIQGGI | faecium | | ATAATGAGTT |
| | | | APIIVAGLGY | (Streptococcus | | ATCTAAAAAA |
| | | | LVKDAWDH | faecium) | | GAACTTCTAC |
| | | | SDQIISGFKK | | | AGATTCAAGG |
| | | | GWNGGRRK | | | AGGAATAGCA |
| | | | | | | CCTATTATAGT |
| | | | | | | TGCTGGCCTT |
| | | | | | | GGCTATTTAG |
| | | | | | | TAAAAGATGC |
| | | | | | | ATGGGATCAC |
| | | | | | | TCAGATCAAA |
| | | | | | | TAATCTCAGG |
| | | | | | | ATTTAAAAAA |
| | | | | | | GGTTGGAATG |
| | | | | | | GTGGACGTAG |
| | | | | | | AAAATAA |
| 118 | Enterolysin | class III | MKNILLSILG | Enterococcus | 119 | ATGAAAAATA |
| | A | | VLSIWSLAF | faecalis | | TTTTACTTTCT |
| | | | SSYSVNAAS | (Streptococcus | | ATTCTAGGGG |
| | | | NEWSWPLG | faecalis) | | TATTATCTATC |
| | | | KPYAGRYEE | | | GTTGTTTCTTT |
| | | | GQQFGNTAF | | | GGCGTTTTCTT |
| | | | NRGGTYFHD | | | CTTATTCTGTC |
| | | | GFDFGSAIY | | | AACGCAGCTT |
| | | | GNGSVYAV | | | CTAATGAGTG |
| | | | HDGKILYAG | | | GTCGTGGCCA |
| | | | WDPVGGGS | | | CTGGGCAAAC |
| | | | LGAFIVLQA | | | CATATGCGGG |
| | | | GNTNVIYQE | | | AAGATATGAA |
| | | | FSRNVGDIK | | | GAAGGACAAC |
| | | | VSTGQTVKK | | | AATTCGGGAA |
| | | | GQLIGKFTSS | | | CACTGCATTTA |
| | | | HLHLGMTK | | | ACCGAGGAGG |
| | | | KEWRSAHSS | | | TACTTATTTCC |
| | | | WNKDDGTW | | | ATGATGGGTT |
| | | | FNPIPILQGG | | | TGACTTTGGTT |
| | | | STPTPPNPGP | | | CTGCTATTTAT |
| | | | KNFTTNVRY | | | GGAAATGGCA |
| | | | GLRVLGGSW | | | GTGTGTATGC |
| | | | LPEVTNFNN | | | TGTGCATGAT |
| | | | TNDGFAGYP | | | GGTAAAATTT |
| | | | NRQHDMLYI | | | TATATGCTGG |
| | | | KVDKGQMK | | | TTGGGATCCT |
| | | | YRVHTAQSG | | | GTAGGTGGAG |
| | | | WLPWVSKG | | | GCTCATTAGG |
| | | | DKSDTVNGA | | | TGCATTTATTG |
| | | | AGMPGQAID | | | TACTACAAGC |
| | | | GVQLNYITP | | | GGGAAACACA |
| | | | KGEKLSQAY | | | AATGTGATTT |
| | | | YRSQTTKRS | | | ATCAAGAATT |
| | | | GWLKVSAD | | | TAGCCGAAAT |
| | | | NGSIPGLDSY | | | GTTGGAGATA |
| | | | AGIFGEPLDR | | | TTAAAGTTAG |
| | | | LQIGISQSNP | | | CACTGGACAA |
| | | | F | | | ACTGTTAAAA |
| | | | | | | AAGGACAGCT |
| | | | | | | GATAGGAAAG |
| | | | | | | TTTACTTCTAG |
| | | | | | | TCATTTACATT |
| | | | | | | TAGGAATGAC |
| | | | | | | AAAAAAAGAA |
| | | | | | | TGGCGTTCTG |
| | | | | | | CTCATTCTTCT |
| | | | | | | TGGAATAAAG |
| | | | | | | ATGATGGCAC |
| | | | | | | TTGGTTTAACC |
| | | | | | | CAATTCCTATA |
| | | | | | | CTTCAAGGAG |
| | | | | | | GATCTACGCC |
| | | | | | | TACGCCTCCA |
| | | | | | | AATCCAGGAC |
| | | | | | | CAAAAAATTT |
| | | | | | | CACAACAAAT |
| | | | | | | GTTCGTTACG |
| | | | | | | GATTGCGGGT |
| | | | | | | CCTCGGAGGT |
| | | | | | | TCATGGTTAC |
| | | | | | | CAGAAGTAAC |
| | | | | | | CAACTTTAAC |
| | | | | | | AATACCAATG |
| | | | | | | ATGGTTTCGC |
| | | | | | | AGGTTACCCT |
| | | | | | | AATCGTCAAC |
| | | | | | | ATGATATGCT |
| | | | | | | TTATATAAAG |
| | | | | | | GTAGATAAAG |
| | | | | | | GGCAAATGAA |
| | | | | | | ATATCGTGTTC |
| | | | | | | ACACGGCTCA |
| | | | | | | AAGTGGATGG |
| | | | | | | TTGCCTTGGG |
| | | | | | | TAAGTAAAGG |
| | | | | | | GGATAAGAGC |
| | | | | | | GATACAGTAA |
| | | | | | | ATGGAGCGGC |
| | | | | | | AGGTATGCCT |
| | | | | | | GGACAAGCAA |
| | | | | | | TTGATGGTGT |
| | | | | | | TCAGCTAAAC |
| | | | | | | TATATAACTCC |
| | | | | | | TAAGGGAGAA |
| | | | | | | AAATTATCAC |
| | | | | | | AGGCTTACTA |
| | | | | | | TCGTTCACAA |
| | | | | | | ACTACGAAAC |
| | | | | | | GATCAGGCTG |
| | | | | | | GTTAAAAGTA |
| | | | | | | AGTGCAGATA |
| | | | | | | ATGGTTCTATT |
| | | | | | | CCTGGACTAG |
| | | | | | | ACAGTTATGC |
| | | | | | | AGGAATCTTT |
| | | | | | | GGAGAACCGT |
| | | | | | | TGGATCGCTT |
| | | | | | | GCAAATAGGT |
| | | | | | | ATTTCACAGTC |
| | | | | | | AAATCCATTTT |
| | | | | | | AA |
| 120 | Epicidin | Lantibiotic | MENKKDLFD | Staphylococcus | 121 | ATGGAAAACA |
| | 280 | | LEIKKDNME | epidermidis | | AAAAAGATTT |
| | | | NNNELEAQS | | | ATTTGATTTAG |
| | | | LGPAIKATR | | | AAATCAAAAA |
| | | | QVCPKATRF | | | AGATAATATG |
| | | | VTVSCKKSD | | | GAAAATAATA |
| | | | CQ | | | ATGAATTAGA |
| | | | | | | AGCTCAATCT |
| | | | | | | CTTGGTCCTGC |
| | | | | | | AATTAAGGCA |
| | | | | | | ACTAGACAGG |
| | | | | | | TATGTCCTAA |
| | | | | | | AGCAACACGT |
| | | | | | | TTTGTTACAGT |
| | | | | | | TTCTTGTAAAA |
| | | | | | | AAAGTGATTG |
| | | | | | | TCAATAG |
| 122 | Epidermicin | Unclassified | MAAFMKLIQ | Staphylococcus | 123 | ATGGCAGCAT |
| | NI01 | | FLATKGQKY | epidermidis | | TTATGAAGTT |
| | | | VSLAWKHK | | | AATTCAGTTCT |
| | | | GTILKWINA | | | TAGCAACTAA |
| | | | GQSFEWIYK | | | AGGTCAAAAG |
| | | | QIKKLWA | | | TATGTTTCACT |
| | | | | | | TGCATGGAAA |
| | | | | | | CATAAAGGTA |
| | | | | | | CTATTTTAAAA |
| | | | | | | TGGATTAACG |
| | | | | | | CCGGTCAAAG |
| | | | | | | TTTTGAATGG |
| | | | | | | ATTTATAAAC |
| | | | | | | AAATCAAAAA |
| | | | | | | ATTATGGGCA |
| | | | | | | TAA |
| 124 | Epidermin | Lantibiotic | MEAVKEKN | Staphylococcus | 125 | ATGGAAGCAG |
| | | | DLFNLDVKV | epidermidis | | TAAAAGAAAA |
| | | | NAKESNDSG | | | AAATGATCTTT |
| | | | AEPRIASKFI | | | TTAATCTTGAT |
| | | | CTPGCAKTG | | | GTTAAAGTTA |
| | | | SFNSYCC | | | ATGCAAAAGA |
| | | | | | | ATCTAACGAT |
| | | | | | | TCAGGAGCTG |
| | | | | | | AACCAAGAAT |
| | | | | | | TGCTAGTAAA |
| | | | | | | TTTATATGTAC |
| | | | | | | TCCTGGATGT |
| | | | | | | GCAAAAACAG |
| | | | | | | GTAGTTTTAA |
| | | | | | | CAGTTATTGTT |
| | | | | | | GTTAA |
| 126 | Epilancin | Lantibiotic | MNNSLFDLN | Staphylococcus | 127 | ATGAATAACT |
| | K7 | | LNKGVETQK | epidermidis | | CATTATTCGAT |
| | | | SDLSPQSAS | Staphylococcus | | TTAAACCTAA |
| | | | VLKTSIKVSK | | | ACAAAGGTGT |
| | | | KYCKGVTLT | | | AGAAACTCAA |
| | | | CGCNITGGK | | | AAGAGTGATT |
| | | | | | | TAAGTCCGCA |
| | | | | | | ATCTGCTAGT |
| | | | | | | GTCTTGAAGA |
| | | | | | | CTTCTATTAAA |
| | | | | | | GTATCTAAAA |
| | | | | | | AATATTGTAA |
| | | | | | | AGGTGTTACT |
| | | | | | | TTAACATGCG |
| | | | | | | GTTGCAATAT |
| | | | | | | TACTGGTGGT |
| | | | | | | AAATAA |
| 128 | Gallidermin | Lantibiotic | MEAVKEKN | Staphylococcus | 129 | ATGGAAGCAG |
| | | | ELFDLDVKV | gallinarum | | TAAAAGAGAA |
| | | | NAKESNDSG | | | AAATGAACTT |
| | | | AEPRIASKFL | | | TTTGATCTTGA |
| | | | CTPGCAKTG | | | CGTTAAAGTA |
| | | | SFNSYCC | | | AATGCAAAAG |
| | | | | | | AGTCTAATGA |
| | | | | | | TTCAGGCGCA |
| | | | | | | GAACCACGAA |
| | | | | | | TTGCTAGTAA |
| | | | | | | ATTTTTATGTA |
| | | | | | | CTCCTGGATG |
| | | | | | | TGCCAAAACA |
| | | | | | | GGTAGCTTCA |
| | | | | | | ATAGCTACTG |
| | | | | | | TTGTTAA |
| 130 | Garvicin | IId | MENNNYTV | Lactococcus | 131 | ATGGAAAACA |
| | A | | LSDEELQKID | garvieae | | ACAATTACAC |
| | | | GGIGGALGN | | | AGTACTTTCA |
| | | | ALNGLGTW | | | GATGAAGAAC |
| | | | ANMMNGGG | | | TACAAAAAAT |
| | | | FVNQWQVY | | | TGATGGTGGA |
| | | | ANKGKINQY | | | ATCGGCGGGG |
| | | | RPY | | | CTCTTGGTAAT |
| | | | | | | GCTCTCAACG |
| | | | | | | GATTAGGTAC |
| | | | | | | CTGGGCAAAC |
| | | | | | | ATGATGAACG |
| | | | | | | GTGGAGGATT |
| | | | | | | TGTTAATCAG |
| | | | | | | TGGCAAGTTT |
| | | | | | | ATGCTAATAA |
| | | | | | | AGGAAAAATA |
| | | | | | | AATCAATACC |
| | | | | | | GTCCGTATTA |
| | | | | | | A |
| 132 | Garvicin | Unclassified | MFDLVATG | Lactococcus | 133 | ATGTTTGATTT |
| | ML | | MAAGVAKTI | garvieae | | AGTCGCGACT |
| | | | VNAVSAGM | | | GGAATGGCTG |
| | | | DIATALSLFS | | | CAGGTGTAGC |
| | | | GAFTAAGGI | | | AAAAACTATT |
| | | | MALIKKYAQ | | | GTTAATGCCG |
| | | | KKLWKQLIA | | | TTAGTGCTGG |
| | | | A | | | TATGGATATT |
| | | | | | | GCCACTGCTTT |
| | | | | | | ATCATTGTTCT |
| | | | | | | CAGGAGCTTT |
| | | | | | | TACTGCAGCT |
| | | | | | | GGGGGAATTA |
| | | | | | | TGGCACTCAT |
| | | | | | | TAAAAAATAT |
| | | | | | | GCTCAAAAGA |
| | | | | | | AATTATGGAA |
| | | | | | | ACAGCTTATT |
| | | | | | | GCTGCATAA |
| 134 | Gassericin | Unclassified | MVTKYGRN | Lactobacillus | 135 | ATGGTTACTA |
| | A | | LGLNKVELF | gasseri | | AGTACGGACG |
| | | | AIWAVLVVA | | | TAATTTAGGTT |
| | | | LLLTTANIY | | | TGAACAAGGT |
| | | | WIADQFGIH | | | AGAGTTGTTT |
| | | | LATGTARKL | | | GCAATTTGGG |
| | | | LDAMASGAS | | | CGGTTTTAGT |
| | | | LGTAFAAIL | | | AGTTGCTCTTT |
| | | | GVTLPAWAL | | | TATTGACCAC |
| | | | AAAGALGAT | | | AGCGAACATT |
| | | | AA | | | TATTGGATTG |
| | | | | | | CTGATCAATTC |
| | | | | | | GGGATTCATT |
| | | | | | | TAGCGACTGG |
| | | | | | | AACAGCCCGT |
| | | | | | | AAGTTATTAG |
| | | | | | | ATGCAATGGC |
| | | | | | | TTCTGGTGCCT |
| | | | | | | CATTGGGAAC |
| | | | | | | TGCCTTTGCTG |
| | | | | | | CTATTTTGGGC |
| | | | | | | GTGACATTAC |
| | | | | | | CTGCATGGGC |
| | | | | | | TTTGGCAGCT |
| | | | | | | GCAGGAGCAT |
| | | | | | | TGGGAGCGAC |
| | | | | | | TGCAGCCTAG |
| 136 | Gassericin | Unclassified | MKNFNTLSF | Lactobacillus | 137 | ATGAAAAATT |
| | T | | ETLANIVGG | gasseri | | TTAATACATTA |
| | (gassericin | | RNNWAANIG | | | TCATTTGAAA |
| | K7 B) | | GVGGATVA | | | CATTGGCTAA |
| | | | GWALGNAV | | | CATAGTTGGT |
| | | | CGPACGFVG | | | GGGAGAAATA |
| | | | AHYVPIAWA | | | ATTGGGCTGC |
| | | | GVTAATGGF | | | TAATATAGGT |
| | | | GKIRK | | | GGAGTAGGTG |
| | | | | | | GAGCGACAGT |
| | | | | | | CGCTGGATGG |
| | | | | | | GCTCTTGGAA |
| | | | | | | ATGCAGTTTG |
| | | | | | | CGGTCCTGCTT |
| | | | | | | GTGGCTTTGTT |
| | | | | | | GGAGCACACT |
| | | | | | | ATGTTCCAAT |
| | | | | | | AGCATGGGCT |
| | | | | | | GGCGTAACGG |
| | | | | | | CAGCTACTGG |
| | | | | | | TGGATTCGGA |
| | | | | | | AAGATAAGAA |
| | | | | | | AGTAG |
| 138 | Glycocin | Unclassified | MSKLVKTLT | Lactobacillus | 139 | ATGAGTAAAT |
| | F | | ISEISKAQNN | plantarum | | TGGTTAAGAC |
| | | | GGKPAWCW | | | ACTTACTATA |
| | | | YTLAMCGA | | | AGTGAAATTT |
| | | | GYDSGTCDY | | | CTAAGGCTCA |
| | | | MYSHCFGIK | | | AAACAACGGT |
| | | | HHSSGSSSY | | | GGAAAACCTG |
| | | | HC | | | CATGGTGTTG |
| | | | | | | GTATACTTTA |
| | | | | | | GCAATGTGTG |
| | | | | | | GTGCTGGTTA |
| | | | | | | TGATTCGGGA |
| | | | | | | ACCTGTGATT |
| | | | | | | ATATGTATTC |
| | | | | | | GCATTGTTTTG |
| | | | | | | GTATAAAGCA |
| | | | | | | TCATAGTAGT |
| | | | | | | GGTAGTAGCA |
| | | | | | | GTTATCATTGT |
| | | | | | | TAG |
| 140 | Halocin | Unclassified | MSKDRDGR | Haloferax | 141 | ATGTCGAAAG |
| | H4 | | RTSRRGTLK | mediterranei | | ACAGAGATGG |
| | | | KIGGFSLGAL | (strain ATCC | | GAGAAGGACA |
| | | | SFGAVGRTQ | 33500/DSM | | AGTCGGCGAG |
| | | | AATGSSVTT | 1411/JCM | | GCACGTTAAA |
| | | | ADIAPPGPN | 8866/ | | GAAAATCGGC |
| | | | GDPKSVQID | NBRC 14739 | | GGTTTCAGTCT |
| | | | DKYTGAEM | /NCIMB | | CGGAGCGCTT |
| | | | YGEGDFRVG | 2177/R-4) | | AGTTTCGGGG |
| | | | LGTDLTMYP | (Halobacterium | | CAGTCGGACG |
| | | | PVYRESLGN | mediterranei) | | AACTCAAGCG |
| | | | GSGGWEFDF | | | GCGACCGGCT |
| | | | TVCGSTACR | | | CATCGGTTAC |
| | | | FVDSNGDVK | | | GACCGCTGAT |
| | | | EDDKAKEM | | | ATCGCACCTC |
| | | | WWQEINFND | | | CCGGACCGAA |
| | | | INQDLYSRN | | | CGGAGACCCG |
| | | | DSDWVGSTP | | | AAGAGTGTTC |
| | | | ADTQPEFDY | | | AGATAGATGA |
| | | | TEFALARDG | | | TAAATACACC |
| | | | VTLALTALN | | | GGAGCCGAGA |
| | | | PAMGSLALG | | | TGTACGGCGA |
| | | | ATYFLSDMV | | | GGGTGACTTC |
| | | | NWIASQHED | | | AGAGTCGGTC |
| | | | DSSLKRKWD | | | TCGGAACTGA |
| | | | YDGLSGPLY | | | CCTGACGATG |
| | | | ADSSTYLLA | | | TATCCGCCCG |
| | | | RDEMTSNSY | | | TGTACCGTGA |
| | | | ESFTIDNIAV | | | GAGTCTTGGA |
| | | | AFPEFPVRTK | | | AATGGAAGCG |
| | | | YYVTFTAPD | | | GGGGTTGGGA |
| | | | DPSTQSISTL | | | ATTCGACTTCA |
| | | | EEEGIYRVP | | | CCGTTTGTGG |
| | | | ATEVAAARP | | | GTCCACTGCC |
| | | | PGSRRSKSA | | | TGTCGATTTGT |
| | | | ADEMVYVA | | | GGACAGTAAC |
| | | | DPKKFIEVEP | | | GGTGACGTCA |
| | | | VKNPSIPDRI | | | AAGAGGACGA |
| | | | YEEIEQKKK | | | CAAGGCGAAA |
| | | | QRSRKQ | | | GAAATGTGGT |
| | | | | | | GGCAGGAAAT |
| | | | | | | TAACTTCAAC |
| | | | | | | GACATAAATC |
| | | | | | | AGGATTTATA |
| | | | | | | CAGTCGGAAC |
| | | | | | | GATTCCGACT |
| | | | | | | GGGTCGGGTC |
| | | | | | | GACCCCTGCC |
| | | | | | | GATACCCAAC |
| | | | | | | CGGAGTTCGA |
| | | | | | | TTACACCGAC |
| | | | | | | TTTGCGCTCGC |
| | | | | | | TCGGGACGGA |
| | | | | | | GTGACGCTCG |
| | | | | | | CTCTCACGGC |
| | | | | | | ACTCAACCCC |
| | | | | | | GCAATGGGGA |
| | | | | | | GTCTTGCACTC |
| | | | | | | GGTGCCACGT |
| | | | | | | ACTTCCTCAGC |
| | | | | | | GACATGGTGA |
| | | | | | | ACTGGATTGC |
| | | | | | | GAGCCAGCAC |
| | | | | | | GAAGACGACA |
| | | | | | | GTTCGCTCAA |
| | | | | | | GAGAAAATGG |
| | | | | | | GATTACGACG |
| | | | | | | GGCTAAGTGG |
| | | | | | | GCCGTTGTAC |
| | | | | | | GCCGATTCGT |
| | | | | | | CGACGTACCT |
| | | | | | | ACTGGCACGC |
| | | | | | | GACGAGATGA |
| | | | | | | CTTCGAACTC |
| | | | | | | GTACGAATCA |
| | | | | | | TTCACGATCG |
| | | | | | | ATAACATCGC |
| | | | | | | CGTTGCCTTCC |
| | | | | | | CAGAGTTCCC |
| | | | | | | CGTCCGGACC |
| | | | | | | AAGTACTACG |
| | | | | | | TCACATTCACT |
| | | | | | | GCGCCGGATG |
| | | | | | | ACCCGTCAAC |
| | | | | | | GCAGTCGATA |
| | | | | | | TCTACGCTCG |
| | | | | | | AAGAGGAGGG |
| | | | | | | AATCTACCGA |
| | | | | | | GTGCCCGCTA |
| | | | | | | CGGAAGTGGC |
| | | | | | | TGCGGCCAGA |
| | | | | | | CCACCGGGGT |
| | | | | | | CCCGACGTTC |
| | | | | | | CAAATCGGCA |
| | | | | | | GCCGACGAGA |
| | | | | | | TGGTGTACGT |
| | | | | | | TGCCGATCCG |
| | | | | | | AAGAAGTTCA |
| | | | | | | TAGAGGTCGA |
| | | | | | | GCCGGTGAAG |
| | | | | | | AACCCAAGTA |
| | | | | | | TCCCGGACCG |
| | | | | | | AATCTACGAG |
| | | | | | | GAGATAGAGC |
| | | | | | | AAAAAAAGAA |
| | | | | | | ACAACGGAGT |
| | | | | | | AGGAAACAGT |
| | | | | | | AG |
| 142 | Halocin- | Unclassified | MSDKDSINR | Haloarchaeon | 143 | ATGTCGGATA |
| | S8 | | RNVLRKIGGI | S8a | | AAGACAGCAT |
| | | | GVASAVGFS | | | TAACAGAAGA |
| | | | GLASGESLS | | | AATGTATTAA |
| | | | DDEKQDVID | | | GAAAAATTGG |
| | | | TIYKSQRVE | | | CGGTATCGGT |
| | | | QIKKKFGGV | | | GTGGCTTCAG |
| | | | NIEPKKVQS | | | CTGTCGGATTT |
| | | | VTTNQSGDL | | | TCTGGTTTGG |
| | | | VTAKLSVSD | | | CAAGCGGGGA |
| | | | GDLVYSSVK | | | AAGTCTTAGC |
| | | | DTTVIVQFD | | | GATGATGAGA |
| | | | RSASEIGES | | | AACAAGATGT |
| | | | WPKNTEAFI | | | TATTGACACA |
| | | | KSTSSGVDL | | | ATTTACAAAT |
| | | | LRTATDEEIK | | | CACAAAGAGT |
| | | | DVTEGVNTS | | | TGAACAGATA |
| | | | EIESADAVNI | | | AAGAAAAAGT |
| | | | FIDPESQTYY | | | TCGGAGGAGT |
| | | | MEKYDFNN | | | GAATATTGAG |
| | | | KVLEMFELA | | | CCGAAAAAGG |
| | | | TGGTSSGKIS | | | TTCAATCTGTA |
| | | | PTREDQNHE | | | ACGACCAATC |
| | | | YNVREHKVF | | | AGAGCGGAGA |
| | | | NSEKQNIQL | | | TCTTGTTACGG |
| | | | QSDCNINSN | | | CGAAGCTGTC |
| | | | TAADVILCF | | | GGTTAGTGAT |
| | | | NQVGSCALC | | | GGGGATTTGG |
| | | | SPTLVGGPV | | | TATATTCGAG |
| | | | PTVACLLVV | | | TGTCAAAGAT |
| | | | CFGTPNAVS | | | ACAACTGTAA |
| | | | AILEEVDNS | | | TAGTTCAGTTC |
| | | | CFNLIKDVIS | | | GATAGATCGG |
| | | | CWDEWTSF | | | CTTCTGAAATT |
| | | | W | | | GGTGAAAGTT |
| | | | | | | GGCCCAAGAA |
| | | | | | | TACTGAGGCA |
| | | | | | | TTCATCAAATC |
| | | | | | | GACGTCCTCT |
| | | | | | | GGGGTCGATC |
| | | | | | | TTCTACGTACA |
| | | | | | | GCAACTGATG |
| | | | | | | AAGAAATAAA |
| | | | | | | GGACGTTACT |
| | | | | | | GAGGGAGTCA |
| | | | | | | ACACATCTGA |
| | | | | | | AATTGAATCT |
| | | | | | | GCGGATGCTG |
| | | | | | | TTAACATATTT |
| | | | | | | ATTGATCCTG |
| | | | | | | AATCACAGAC |
| | | | | | | ATACTATATG |
| | | | | | | GAGAAATATG |
| | | | | | | ACTTTAATAAT |
| | | | | | | AAGGTACTTG |
| | | | | | | AGATGTTTGA |
| | | | | | | ATTAGCGACA |
| | | | | | | GGTGGGACAA |
| | | | | | | GTAGTGGTAA |
| | | | | | | AATCTCCCCC |
| | | | | | | ACACGTGAAG |
| | | | | | | ACCAGAATCA |
| | | | | | | CGAATATAAT |
| | | | | | | GTTAGGGAAC |
| | | | | | | ATAAAGTATT |
| | | | | | | TAACTCAGAA |
| | | | | | | AAACAGAATA |
| | | | | | | TACAACTTCA |
| | | | | | | GAGTGACTGT |
| | | | | | | AATATAAACA |
| | | | | | | GTAACACCGC |
| | | | | | | TGCTGATGTT |
| | | | | | | ATTCTATGCTT |
| | | | | | | CAACCAGGTT |
| | | | | | | GGTTCTTGTG |
| | | | | | | CACTCTGCTCC |
| | | | | | | CCGACTTTAG |
| | | | | | | TCGGAGGTCC |
| | | | | | | AGTCCCTACA |
| | | | | | | GTTGCATGTCT |
| | | | | | | CTTAGTCGTCT |
| | | | | | | GTTTCGGCAC |
| | | | | | | TCCAAATGCT |
| | | | | | | GTGTCCGCGA |
| | | | | | | TACTTGAAGA |
| | | | | | | AGTCGATAAT |
| | | | | | | TCTTGCTTTAA |
| | | | | | | CTTGATCAAG |
| | | | | | | GATGTAATTT |
| | | | | | | CGTGTTGGGA |
| | | | | | | TGAATGGACT |
| | | | | | | AGCTTCTGGT |
| | | | | | | GA |
| 144 | Helveticin | Unclassified | MKHLNETTN | Lactobacillus | 145 | ATGAAGCATT |
| | -J | | VRILSQFDM | helveticus | | TAAATGAAAC |
| | | | DTGYQAVV | (Lactobacillus | | AACTAATGTT |
| | | | QKGNVGSK | suntoryeus) | | AGAATTTTAA |
| | | | YVYGLQLRK | | | GTCAATTTGA |
| | | | GATTILRGY | | | TATGGATACT |
| | | | RGSKINNPIL | | | GGCTATCAAG |
| | | | ELSGQAGGH | | | CAGTAGTTCA |
| | | | TQTWEFAGD | | | AAAAGGCAAT |
| | | | RKDINGEER | | | GTAGGTTCAA |
| | | | AGQWFIGVK | | | AATATGTATA |
| | | | PSKIEGSKII | | | TGGATTACAA |
| | | | WAKQIARVD | | | CTTCGCAAAG |
| | | | LRNQMGPH | | | GTGCTACTAC |
| | | | YSNTDFPRL | | | TATCTTGCGTG |
| | | | SYLNRAGSN | | | GTTACCGTGG |
| | | | PFAGNKMTH | | | AAGTAAAATT |
| | | | AEAAVSPDY | | | AATAACCCTA |
| | | | TKFLIATVEN | | | TTCTTGAATTA |
| | | | NCIGHFTIYN | | | TCTGGTCAAG |
| | | | LDTINEKLD | | | CAGGTGGTCA |
| | | | EKGNSEDVN | | | CACACAGACA |
| | | | LETVKYEDS | | | TGGGAATTTG |
| | | | FIIDNLYGDD | | | CTGGTGATCG |
| | | | NNSIVNSIQG | | | TAAAGACATT |
| | | | YDLDNDGNI | | | AATGGTGAAG |
| | | | YISSQKAPDF | | | AAAGAGCAGG |
| | | | DGSYYAHH | | | TCAATGGTTT |
| | | | KQIVKIPYYA | | | ATAGGTGTTA |
| | | | RSKESEDQW | | | AACCATCGAA |
| | | | RAVNLSEFG | | | AATTGAAGGA |
| | | | GLDIPGKHS | | | AGCAAAATTA |
| | | | EVESIQIIGE | | | TTTGGGCAAA |
| | | | NHCYLTVAY | | | GCAAATTGCA |
| | | | HSKNKAGEN | | | AGAGTTGATC |
| | | | KTTLNEIYEL | | | TTAGAAATCA |
| | | | SWN | | | AATGGGACCT |
| | | | | | | CATTATTCAA |
| | | | | | | ATACTGACTTT |
| | | | | | | CCTCGATTATC |
| | | | | | | CTACTTGAATC |
| | | | | | | GCGCCGGTTC |
| | | | | | | TAATCCATTTG |
| | | | | | | CTGGTAATAA |
| | | | | | | GATGACGCAT |
| | | | | | | GCCGAAGCCG |
| | | | | | | CAGTATCACC |
| | | | | | | TGATTATACTA |
| | | | | | | AGTTTTTAATT |
| | | | | | | GCTACTGTTG |
| | | | | | | AAAATAACTG |
| | | | | | | TATTGGTCATT |
| | | | | | | TTACTATATAC |
| | | | | | | AATTTAGATA |
| | | | | | | CAATTAATGA |
| | | | | | | AAAACTTGAT |
| | | | | | | GAAAAGGGAA |
| | | | | | | ATAGTGAAGA |
| | | | | | | TGTTAATCTCG |
| | | | | | | AAACTGTTAA |
| | | | | | | ATACGAAGAT |
| | | | | | | AGTTTTATCAT |
| | | | | | | TGATAATTTAT |
| | | | | | | ATGGTGATGA |
| | | | | | | TAATAATTCTA |
| | | | | | | TTGTAAATTCA |
| | | | | | | ATTCAAGGGT |
| | | | | | | ATGATTTGGA |
| | | | | | | TAATGATGGA |
| | | | | | | AATATTTATAT |
| | | | | | | TTCCAGTCAA |
| | | | | | | AAAGCGCCAG |
| | | | | | | ATTTTGATGG |
| | | | | | | CTCTTATTATG |
| | | | | | | CACATCATAA |
| | | | | | | GCAGATTGTT |
| | | | | | | AAGATTCCAT |
| | | | | | | ATTATGCTCG |
| | | | | | | GTCTAAAGAA |
| | | | | | | AGCGAAGACC |
| | | | | | | AATGGAGAGC |
| | | | | | | TGTAAATTTA |
| | | | | | | AGCGAATTCG |
| | | | | | | GTGGCTTGGA |
| | | | | | | TATTCCAGGT |
| | | | | | | AAACATAGTG |
| | | | | | | AAGTTGAAAG |
| | | | | | | CATCCAAATT |
| | | | | | | ATTGGTGAGA |
| | | | | | | ATCATTGTTAC |
| | | | | | | TTAACTGTTGC |
| | | | | | | ATATCATTCTA |
| | | | | | | AAAATAAAGC |
| | | | | | | GGGTGAAAAT |
| | | | | | | AAAACTACTT |
| | | | | | | TGAATGAGAT |
| | | | | | | TTATGAATTAT |
| | | | | | | CTTGGAATTA |
| | | | | | | G |
| 146 | Hiracin | Class II | MKKKVLKH | Enterococcus | 147 | ATGAAAAAGA |
| | JM79 | sec- | CVILGILGTC | hirae | | AAGTATTAAA |
| | | depende | LAGIGTGIKV | | | ACATTGTGTT |
| | | nt | DAATYYGN | | | ATTCTAGGAA |
| | | | GLYCNKEKC | | | TATTAGGAAC |
| | | | WVDWNQAK | | | TTGTCTAGCTG |
| | | | GEIGKIIVNG | | | GCATCGGTAC |
| | | | WVNHGPWA | | | AGGAATAAAA |
| | | | PRR | | | GTTGATGCAG |
| | | | | | | CTACTTACTAT |
| | | | | | | GGAAATGGTC |
| | | | | | | TTTATTGTAAC |
| | | | | | | AAAGAAAAAT |
| | | | | | | GTTGGGTAGA |
| | | | | | | TTGGAATCAA |
| | | | | | | GCTAAAGGAG |
| | | | | | | AAATTGGAAA |
| | | | | | | AATTATTGTTA |
| | | | | | | ATGGTTGGGT |
| | | | | | | TAATCATGGT |
| | | | | | | CCATGGGCAC |
| | | | | | | CTAGAAGGTA |
| | | | | | | G |
| 148 | Lactacin- | class IIB | MKQFNYLSH | Lactobacillus | 149 | ATGAAACAAT |
| | F (lafA) | | KDLAVVVG | johnsonii | | TTAATTATTTA |
| | | | GRNNWQTN | (strain | | TCACATAAAG |
| | | | VGGAVGSA | CNCM I- | | ATTTAGCAGT |
| | | | MIGATVGGT | 12250/Lal/ | | CGTTGTTGGT |
| | | | ICGPACAVA | NCC 533) | | GGAAGAAATA |
| | | | GAHYLPILW | | | ATTGGCAAAC |
| | | | TAVTAATGG | | | AAATGTGGGA |
| | | | FGKIRK | | | GGAGCAGTGG |
| | | | | | | GATCAGCTAT |
| | | | | | | GATTGGGGCT |
| | | | | | | ACAGTTGGTG |
| | | | | | | GTACAATTTG |
| | | | | | | TGGACCTGCA |
| | | | | | | TGTGCTGTAG |
| | | | | | | CTGGTGCCCA |
| | | | | | | TTATCTTCCTA |
| | | | | | | TTTTATGGAC |
| | | | | | | AGCGGTTACA |
| | | | | | | GCTGCAACAG |
| | | | | | | GTGGTTTTGG |
| | | | | | | CAAGATAAGA |
| | | | | | | AAGTAG |
| 150 | Lactacin- | class IIB | MKLNDKELS | Lactobacillus | 151 | ATGAAATTAA |
| | F (lafX) | | KIVGGNRWG | johnsonii | | ATGACAAAGA |
| | | | DTVLSAASG | (strain | | ATTATCAAAG |
| | | | AGTGIKACK | CNCM I- | | ATTGTTGGTG |
| | | | SFGPWGMAI | 12250/Lal/ | | GAAATCGATG |
| | | | CGVGGAAIG | NCC 533) | | GGGAGATACT |
| | | | GYFGYTHN | | | GTTTTATCAGC |
| | | | | | | TGCTAGTGGC |
| | | | | | | GCAGGAACTG |
| | | | | | | GTATTAAAGC |
| | | | | | | ATGTAAAAGT |
| | | | | | | TTTGGCCCAT |
| | | | | | | GGGGAATGGC |
| | | | | | | AATTTGTGGT |
| | | | | | | GTAGGAGGTG |
| | | | | | | CAGCAATAGG |
| | | | | | | AGGTTATTTTG |
| | | | | | | GCTATACTCAT |
| | | | | | | AATTAA |
| 152 | Lacticin | Lantibiotic | MNKNEIETQ | Lactococcus | 153 | ATGAACAAAA |
| | 3147 A1 | | PVTWLEEVS | lactis subsp. | | ATGAAATTGA |
| | | | DQNFDEDVF | lactis | | AACACAACCA |
| | | | GACSTNTFS | (Streptococcus | | GTTACATGGT |
| | | | LSDYWGNN | lactis) | | TGGAAGAAGT |
| | | | GAWCTLTHE | | | ATCTGATCAA |
| | | | CMAWCK | | | AATTTTGATG |
| | | | | | | AAGATGTATT |
| | | | | | | TGGTGCGTGT |
| | | | | | | AGTACTAACA |
| | | | | | | CATTCTCGCTC |
| | | | | | | AGTGATTACT |
| | | | | | | GGGGAAATAA |
| | | | | | | CGGGGCTTGG |
| | | | | | | TGTACACTCA |
| | | | | | | CTCATGAATG |
| | | | | | | TATGGCTTGG |
| | | | | | | TGTAAATAA |
| 154 | Lacticin | Lantibiotic | MKEKNMKK | Lactococcus | 155 | ATGAAAGAAA |
| | 3147 A2 | | NDTIELQLG | lactis subsp. | | AAAATATGAA |
| | | | KYLEDDMIE | lactis | | AAAGAATGAC |
| | | | LAEGDESHG | (Streptococcus | | ACTATTGAATT |
| | | | GTTPATPAIS | lactis) | | ACAATTGGGA |
| | | | ILSAYISTNT | | | AAATACCTTG |
| | | | CPTTKCTRA | | | AAGATGATAT |
| | | | C | | | GATTGAATTA |
| | | | | | | GCTGAAGGGG |
| | | | | | | ATGAGTCTCA |
| | | | | | | TGGAGGAACA |
| | | | | | | ACACCAGCAA |
| | | | | | | CTCCTGCAATC |
| | | | | | | TCTATTCTCAG |
| | | | | | | TGCATATATTA |
| | | | | | | GTACCAATAC |
| | | | | | | TTGTCCAACA |
| | | | | | | ACAAAATGTA |
| | | | | | | CACGTGCTTG |
| | | | | | | TTAA |
| 156 | Lacticin | Lantibiotic | MKEQNSFNL | Lactococcus | 157 | ATGAAAGAAC |
| | 481 | | LQEVTESEL | lactis subsp. | | AAAACTCTTTT |
| | (Lactococ | | DLILGAKGG | lactis | | AATCTTCTTCA |
| | cin DR) | | SGVIHTISHE | (Streptococcus | | AGAAGTGACA |
| | | | CNMNSWQF | lactis) | | GAAAGTGAAT |
| | | | VFTCCS | | | TGGACCTTATT |
| | | | | | | TTAGGTGCAA |
| | | | | | | AAGGCGGCAG |
| | | | | | | TGGAGTTATT |
| | | | | | | CATACAATTTC |
| | | | | | | TCATGAATGT |
| | | | | | | AATATGAATA |
| | | | | | | GCTGGCAATT |
| | | | | | | TGTATTTACTT |
| | | | | | | GCTGCTCTTA |
| | | | | | | A |
| 158 | Lacticin Q | Unclassified | MAGFLKVV | Lactococcus | 159 | ATGGCAGGGT |
| | | | QLLAKYGSK | lactis | | TTTTAAAAGT |
| | | | AVQWAWAN | | | AGTTCAATTA |
| | | | KGKILDWLN | | | CTAGCTAAAT |
| | | | AGQAIDWV | | | ATGGTTCTAA |
| | | | VSKIKQILGI | | | AGCTGTACAA |
| | | | K | | | TGGGCTTGGG |
| | | | | | | CAAACAAGGG |
| | | | | | | TAAGATTTTA |
| | | | | | | GATTGGCTTA |
| | | | | | | ATGCAGGTCA |
| | | | | | | GGCTATTGAT |
| | | | | | | TGGGTAGTTT |
| | | | | | | CGAAAATTAA |
| | | | | | | GCAAATTTTA |
| | | | | | | GGTATTAAGT |
| | | | | | | AA |
| 160 | Lacticin Z | Unclassified | MAGFLKVV | Lactococcus | 161 | ATGGCAGGGT |
| | | | QILAKYGSK | lactis | | TTTTAAAAGT |
| | | | AVQWAWAN | | | AGTCCAAATT |
| | | | KGKILDWIN | | | TTGGCTAAGT |
| | | | AGQAIDWV | | | ATGGTTCTAA |
| | | | VEKIKQILGI | | | AGCCGTACAA |
| | | | K | | | TGGGCATGGG |
| | | | | | | CAAATAAAGG |
| | | | | | | AAAAATCTTA |
| | | | | | | GATTGGATTA |
| | | | | | | ATGCAGGTCA |
| | | | | | | AGCTATTGAC |
| | | | | | | TGGGTAGTTG |
| | | | | | | AAAAGATTAA |
| | | | | | | GCAAATTTTG |
| | | | | | | GGTATTAAAT |
| | | | | | | AA |
| 162 | Lactobin- | class IIB | MKQLNSEQL | Lactobacillus | 163 | ATGAAACAAT |
| | A | | QNIIGGNRW | amylovorus | | TGAATTCAGA |
| | (Amylovo | | TNAYSAALG | | | ACAATTACAA |
| | rin-L471) | | CAVPGVKYG | | | AATATTATCG |
| | | | KKLGGVWG | | | GTGGAAATAG |
| | | | AVIGGVGGA | | | ATGGACTAAT |
| | | | AVCGLAGY | | | GCATACAGCG |
| | | | VRKG | | | CAGCTTTGGG |
| | | | | | | ATGCGCTGTC |
| | | | | | | CCTGGAGTTA |
| | | | | | | AATATGGAAA |
| | | | | | | AAAACTTGGT |
| | | | | | | GGCGTATGGG |
| | | | | | | GTGCTGTAAT |
| | | | | | | TGGTGGCGTA |
| | | | | | | GGCGGTGCAG |
| | | | | | | CAGTCTGTGG |
| | | | | | | CTTGGCGGGT |
| | | | | | | TATGTTCGTA |
| | | | | | | AAGGCTAA |
| 164 | Lactocin- | Lantibiotic | MKTEKKVL | Lactobacillus | 165 | ATGAAAACAG |
| | S | | DELSLHASA | sakei L45 | | AAAAAAAGGT |
| | | | KMGARDVE | | | TTTAGATGAA |
| | | | SSMNADSTP | | | CTGAGCTTAC |
| | | | VLASVAVSM | | | ACGCTTCTGC |
| | | | ELLPTASVL | | | AAAAATGGGA |
| | | | YSDVAGCFK | | | GCACGTGATG |
| | | | YSAKHHC | | | TTGAATCCAG |
| | | | | | | CATGAATGCA |
| | | | | | | GACTCAACAC |
| | | | | | | CAGTTTTAGC |
| | | | | | | ATCAGTCGCT |
| | | | | | | GTATCCATGG |
| | | | | | | AATTATTGCC |
| | | | | | | AACTGCGTCT |
| | | | | | | GTTCTTTATTC |
| | | | | | | GGATGTTGCA |
| | | | | | | GGTTGCTTCA |
| | | | | | | AATATTCTGC |
| | | | | | | AAAACATCAT |
| | | | | | | TGTTAG |
| 166 | Lactococc | Unclassified | MKTKSLVLA | Lactococcus | 167 | ATGAAAACCA |
| | in 972 | | LSAVTLFSA | lactis subsp. | | AGTCTCTCGT |
| | | | GGIVAQAEG | lactis | | ATTGGCATTA |
| | | | TWQHGYGV | (Streptococcus | | TCTGCGGTTA |
| | | | SSAYSNYHH | lactis) | | CGTTATTCTCT |
| | | | GSKTHSATV | | | GCCGGAGGAA |
| | | | VNNNTGRQ | | | TTGTAGCTCA |
| | | | GKDTQRAG | | | AGCTGAAGGA |
| | | | VWAKATVG | | | ACATGGCAAC |
| | | | RNLTEKASF | | | ATGGATATGG |
| | | | YYNFW | | | TGTTAGTTCG |
| | | | | | | GCATATTCAA |
| | | | | | | ATTATCATCAT |
| | | | | | | GGTAGCAAAA |
| | | | | | | CTCATTCAGCC |
| | | | | | | ACAGTTGTAA |
| | | | | | | ATAATAATAC |
| | | | | | | TGGCCGACAA |
| | | | | | | GGTAAGGATA |
| | | | | | | CACAACGTGC |
| | | | | | | CGGTGTTTGG |
| | | | | | | GCAAAAGCTA |
| | | | | | | CTGTTGGACG |
| | | | | | | TAACTTAACT |
| | | | | | | GAAAAAGCTT |
| | | | | | | CATTTTATTAT |
| | | | | | | AACTTTTGGT |
| | | | | | | AA |
| 168 | Lactococcin- | Unclassified | MKNQLNFNI | Lactococcus | 169 | ATGAAAAATC |
| | A | | VSDEELSEA | lactis subsp. | | AATTAAATTTT |
| | | | NGGKLTFIQ | cremoris | | AATATTGTTTC |
| | | | STAAGDLYY | (Streptococcus | | AGATGAAGAA |
| | | | NTNTHKYV | cremoris) | | CTTTCAGAAG |
| | | | YQQTQNAFG | | | CTAACGGAGG |
| | | | AAANTIVNG | | | AAAATTAACA |
| | | | WMGGAAGG | | | TTTATTCAATC |
| | | | FGLHH | | | GACAGCGGCT |
| | | | | | | GGAGATTTAT |
| | | | | | | ATTACAATAC |
| | | | | | | TAATACACAC |
| | | | | | | AAATATGTTT |
| | | | | | | ACCAACAAAC |
| | | | | | | TCAAAACGCT |
| | | | | | | TTTGGGGCTG |
| | | | | | | CTGCTAATAC |
| | | | | | | CATTGTTAAT |
| | | | | | | GGATGGATGG |
| | | | | | | GTGGCGCTGC |
| | | | | | | TGGAGGTTTC |
| | | | | | | GGGTTGCACC |
| | | | | | | ATTGA |
| 170 | Lactococcin- | Unclassified | MKNQLNFNI | Lactococcus | 171 | ATGAAAAATC |
| | B | | VSDEELAEV | lactis subsp. | | AATTAAATTTT |
| | | | NGGSLQYV | cremoris | | AATATTGTTTC |
| | | | MSAGPYTW | (Streptococcus | | TGATGAAGAA |
| | | | YKDTRTGKT | cremoris) | | CTTGCAGAAG |
| | | | ICKQTIDTAS | | | TTAATGGAGG |
| | | | YTFGVMAE | | | AAGCTTGCAG |
| | | | GWGKTFH | | | TATGTTATGA |
| | | | | | | GTGCTGGACC |
| | | | | | | ATATACTTGG |
| | | | | | | TATAAAGATA |
| | | | | | | CTAGAACAGG |
| | | | | | | AAAAACAATA |
| | | | | | | TGTAAACAGA |
| | | | | | | CAATTGACAC |
| | | | | | | AGCAAGTTAT |
| | | | | | | ACATTTGGTG |
| | | | | | | TAATGGCAGA |
| | | | | | | AGGATGGGGA |
| | | | | | | AAAACATTCC |
| | | | | | | ACTAA |
| 172 | Lactocyclicin | Unclassified | MKLIDHLGA | Lactococcus | 173 | ATGAAATTAA |
| | Q | | PRWAVDTIL | sp. QU 12 | | TTGATCATTTA |
| | | | GAIAVGNLA | | | GGTGCTCCAA |
| | | | SWVLALVPG | | | GATGGGCCGT |
| | | | PGWAVKAG | | | TGATACTATTT |
| | | | LATAAAIVK | | | TAGGTGCAAT |
| | | | HQGKAAAA | | | CGCAGTTGGG |
| | | | AW | | | AACTTAGCAA |
| | | | | | | GTTGGGTTCT |
| | | | | | | AGCGCTTGTC |
| | | | | | | CCTGGTCCAG |
| | | | | | | GGTGGGCAGT |
| | | | | | | AAAAGCTGGT |
| | | | | | | TTAGCAACTG |
| | | | | | | CTGCTGCCAT |
| | | | | | | CGTTAAACAT |
| | | | | | | CAAGGTAAAG |
| | | | | | | CTGCCGCTGC |
| | | | | | | TGCTTGGTAA |
| 174 | Laterosporulin | Unclassified | MACQCPDAI | Brevibacillus | 175 | ATGGCTTGCC |
| | | | SGWTHTDY | sp. GI-9 | | AATGTCCAGA |
| | | | QCHGLENK | | | TGCGATCTCA |
| | | | MYRHVYAIC | | | GGTTGGACGC |
| | | | MNGTQVYC | | | ATACAGATTA |
| | | | RTEWGSSC | | | CCAGTGTCAC |
| | | | | | | GGTTTGGAGA |
| | | | | | | ATAAAATGTA |
| | | | | | | TAGACATGTT |
| | | | | | | TATGCAATTT |
| | | | | | | GCATGAACGG |
| | | | | | | TACTCAAGTA |
| | | | | | | TATTGCAGAA |
| | | | | | | CAGAGTGGGG |
| | | | | | | TAGCAGCTGC |
| | | | | | | TAG |
| 176 | Leucocin | class IId | MNKEYNSIS | Leuconostoc | 177 | ATGAATAAAG |
| | N | | NFKKITNKD | pseudo | | AATATAATAG |
| | | | LQNINGGFIG | mesenteroides | | CATTAGCAAT |
| | | | RAIGDFVYF | | | TTTAAAAAAA |
| | | | GAKGLRESG | | | TTACTAATAA |
| | | | KLLNYYYKH | | | AGACTTGCAA |
| | | | KH | | | AACATAAATG |
| | | | | | | GTGGATTTATT |
| | | | | | | GGTAGGGCAA |
| | | | | | | TAGGTGACTT |
| | | | | | | TGTGTACTTTG |
| | | | | | | GAGCGAAGGG |
| | | | | | | ACTAAGAGAA |
| | | | | | | TCTGGTAAAC |
| | | | | | | TACTTAATTAT |
| | | | | | | TACTATAAGC |
| | | | | | | ATAAGCATTG |
| | | | | | | A |
| 178 | Leucocin | class IId | MKNQLMSFE | Leuconostoc | 179 | ATGAAAAATC |
| | Q | | VISEKELSTV | pseudo- | | AGTTAATGTC |
| | | | QGGKGLGKL | mesenteroides | | TTTCGAAGTG |
| | | | IGIDWLLGQ | | | ATATCAGAAA |
| | | | AKDAVKQY | | | AAGAATTGTC |
| | | | KKDYKRWH | | | CACGGTACAA |
| | | | | | | GGTGGCAAAG |
| | | | | | | GCTTAGGTAA |
| | | | | | | ACTCATAGGA |
| | | | | | | ATTGATTGGC |
| | | | | | | TTTTGGGTCA |
| | | | | | | AGCTAAGGAC |
| | | | | | | GCTGTTAAAC |
| | | | | | | AGTACAAGAA |
| | | | | | | GGATTACAAA |
| | | | | | | CGTTGGCACT |
| | | | | | | AA |
| 180 | Leucocin- | class | MMNMKPTE | Leuconostoc | 181 | ATGATGAACA |
| | A | IIA/YG | SYEQLDNSA | gelidum | | TGAAACCTAC |
| | (Leucocin | NGV | LEQVVGGK | | | GGAAAGCTAT |
| | A-UAL | | YYGNGVHC | | | GAGCAATTGG |
| | 187) | | TKSGCSVNW | | | ATAATAGTGC |
| | | | GEAFSAGVH | | | TCTCGAACAA |
| | | | RLANGGNGF | | | GTCGTAGGAG |
| | | | W | | | GTAAGTATTA |
| | | | | | | TGGTAACGGA |
| | | | | | | GTTCATTGCA |
| | | | | | | CAAAAAGTGG |
| | | | | | | TTGTTCTGTAA |
| | | | | | | ACTGGGGAGA |
| | | | | | | AGCCTTTTCA |
| | | | | | | GCTGGAGTAC |
| | | | | | | ATCGTTTAGC |
| | | | | | | AAATGGTGGA |
| | | | | | | AATGGTTTCT |
| | | | | | | GGTAA |
| 182 | Leucocin- | class | MNNMKSAD | Leuconostoc | 183 | ATGAATAACA |
| | B | IIA/YG | NYQQLDNN | carnosum | | TGAAATCTGC |
| | (Leucocin | NGV | ALEQVVGGK | | | GGATAATTAT |
| | B-Ta11a) | | YYGNGVHC | | | CAGCAATTGG |
| | | | TKSGCSVNW | | | ATAATAATGC |
| | | | GEAFSAGVH | | | TCTCGAACAA |
| | | | RLANGGNGF | | | GTCGTAGGAG |
| | | | W | | | GTAAGTATTA |
| | | | | | | TGGTAACGGA |
| | | | | | | GTTCATTGCA |
| | | | | | | CAAAAAGTGG |
| | | | | | | TTGTTCTGTAA |
| | | | | | | ACTGGGGAGA |
| | | | | | | AGCCTTTTCA |
| | | | | | | GCTGGAGTAC |
| | | | | | | ATCGTTTAGC |
| | | | | | | AAATGGTGGA |
| | | | | | | AATGGTTTCT |
| | | | | | | GGTAA |
| 184 | Leucocyclicin | Unclassified | MFLVNQLGI | Leuconostoc | 185 | ATGTTCTTGGT |
| | Q | tied | SKSLANTILG | mesenteroides | | AAATCAGTTA |
| | | | AIAVGNLAS | | | GGGATTTCAA |
| | | | WLLALVPGP | | | AATCGTTAGC |
| | | | GWATKAAL | | | TAATACTATTC |
| | | | ATAETIVKH | | | TTGGTGCAAT |
| | | | EGKAAAIAW | | | TGCTGTTGGT |
| | | | | | | AATTTGGCCA |
| | | | | | | GTTGGTTATTA |
| | | | | | | GCTTTGGTTCC |
| | | | | | | TGGTCCGGGT |
| | | | | | | TGGGCAACAA |
| | | | | | | AAGCAGCACT |
| | | | | | | TGCGACAGCT |
| | | | | | | GAAACAATTG |
| | | | | | | TGAAGCATGA |
| | | | | | | AGGAAAAGCA |
| | | | | | | GCTGCTATTG |
| | | | | | | CGTGGTAA |
| 186 | Lichenicidin | Lantibiotic | MSKKEMILS | Bacillus | 187 | ATGTCAAAAA |
| | A1 | (two- | WKNPMYRT | licheniformis | | AGGAAATGAT |
| | | peptide) | ESSYHPAGNI | (strain DSM | | TCTTTCATGGA |
| | | | LKELQEEEQ | 13/ATCC | | AAAATCCTAT |
| | | | HSIAGGTITL | 14580) | | GTATCGCACT |
| | | | STCAILSKPL | | | GAATCTTCTTA |
| | | | GNNGYLCTV | | | TCATCCAGCA |
| | | | TKECMPSCN | | | GGGAACATCC |
| | | | | | | TTAAAGAACT |
| | | | | | | CCAGGAAGAG |
| | | | | | | GAACAGCACA |
| | | | | | | GCATCGCCGG |
| | | | | | | AGGCACAATC |
| | | | | | | ACGCTCAGCA |
| | | | | | | CTTGTGCCATC |
| | | | | | | TTGAGCAAGC |
| | | | | | | CGTTAGGAAA |
| | | | | | | TAACGGATAC |
| | | | | | | CTGTGTACAG |
| | | | | | | TGACAAAAGA |
| | | | | | | ATGCATGCCA |
| | | | | | | AGCTGTAACT |
| | | | | | | AA |
| 188 | Linocin | Unclassified | MNNLYRELA | Brevibacterium | 189 | GTGAATAACC |
| | M18 | | PIPGPAWAEI | linens | | TCTATCGCGA |
| | | | EEEARRTFK | | | GCTTGCCCCC |
| | | | RNIAGRRIV | | | ATCCCCGGCC |
| | | | DVAGPTGFE | | | CGGCCTGGGC |
| | | | TSAVTTGHI | | | GGAGATCGAG |
| | | | RDVQSETSG | | | GAGGAGGCTC |
| | | | LQVKQRIVQ | | | GACGGACATT |
| | | | EYIELRTPFT | | | CAAACGCAAT |
| | | | VTRQAIDDV | | | ATCGCCGGCC |
| | | | ARGSGDSD | | | GCCGGATCGT |
| | | | WQPVKDAA | | | CGATGTCGCA |
| | | | TTIAMAEDR | | | GGGCCCACGG |
| | | | AILHGLDAA | | | GCTTCGAGAC |
| | | | GIGGIVPGSS | | | CTCCGCGGTG |
| | | | NAAVAIPDA | | | ACCACTGGCC |
| | | | VEDFADAVA | | | ACATCCGAGA |
| | | | QALSVLRTV | | | CGTCCAGTCG |
| | | | GVDGPYSLL | | | GAGACGAGCG |
| | | | LSSAEYTKV | | | GACTGCAGGT |
| | | | SESTDHGYPI | | | TAAGCAGCGC |
| | | | REHLSRQLG | | | ATCGTGCAGG |
| | | | AGEIIWAPAL | | | AATACATCGA |
| | | | EGALLVSTR | | | GCTGCGGACC |
| | | | GGDYELHLG | | | CCATTCACCGT |
| | | | QDLSIGYYS | | | GACTCGGCAG |
| | | | HDSETVELY | | | GCCATCGATG |
| | | | LQETFGFLA | | | ACGTGGCCCG |
| | | | LTDESSVPLS | | | CGGGTCCGGT |
| | | | L | | | GACTCGGACT |
| | | | | | | GGCAGCCCGT |
| | | | | | | CAAGGATGCG |
| | | | | | | GCCACGACGA |
| | | | | | | TCGCGATGGC |
| | | | | | | TGAAGATCGG |
| | | | | | | GCCATTCTCCA |
| | | | | | | CGGGCTCGAT |
| | | | | | | GCGGCCGGGA |
| | | | | | | TCGGCGGAAT |
| | | | | | | CGTTCCCGGC |
| | | | | | | AGCTCGAATG |
| | | | | | | CCGCAGTGGC |
| | | | | | | CATCCCCGAC |
| | | | | | | GCCGTCGAGG |
| | | | | | | ACTTCGCGGA |
| | | | | | | CGCCGTCGCC |
| | | | | | | CAGGCGCTGA |
| | | | | | | GTGTGCTGCG |
| | | | | | | CACGGTGGGA |
| | | | | | | GTCGACGGGC |
| | | | | | | CCTACAGCCT |
| | | | | | | GTTGCTCTCCT |
| | | | | | | CCGCGGAGTA |
| | | | | | | CACCAAGGTC |
| | | | | | | TCCGAGTCCA |
| | | | | | | CCGACCACGG |
| | | | | | | CTACCCGATC |
| | | | | | | CGCGAGCACC |
| | | | | | | TCTCCCGGCA |
| | | | | | | GCTCGGCGCC |
| | | | | | | GGAGAGATCA |
| | | | | | | TCTGGGCGCC |
| | | | | | | CGCGCTCGAA |
| | | | | | | GGGGCGCTGC |
| | | | | | | TCGTCTCCAC |
| | | | | | | GCGCGGGGGT |
| | | | | | | GACTACGAGC |
| | | | | | | TCCACCTCGG |
| | | | | | | CCAGGACCTG |
| | | | | | | TCGATCGGTT |
| | | | | | | ACTACAGCCA |
| | | | | | | CGACAGCGAG |
| | | | | | | ACCGTCGAAC |
| | | | | | | TCTATCTGCA |
| | | | | | | GGAGACCTTC |
| | | | | | | GGATTCCTCG |
| | | | | | | CGCTGACCGA |
| | | | | | | CGAATCCAGT |
| | | | | | | GTGCCTTTGA |
| | | | | | | GCCTCTGA |
| 190 | Listeriocin | Class IIa | MKKAALKFII | Listeria | 191 | TTGAAGAAGG |
| | 743A | | VIAILGFSFSF | innocua | | CAGCGTTAAA |
| | | | FSIQSEAKSY | | | ATTTATTATTG |
| | | | GNGVQCNK | | | TTATTGCTATT |
| | | | KKCWVDWG | | | CTAGGTTTCA |
| | | | SAISTIGNNS | | | GTTTTTCTTTC |
| | | | AANWATGG | | | TTTAGCATAC |
| | | | AAGWKS | | | AATCTGAAGC |
| | | | | | | TAAATCTTATG |
| | | | | | | GAAATGGAGT |
| | | | | | | TCAGTGTAAT |
| | | | | | | AAGAAAAAAT |
| | | | | | | GTTGGGTAGA |
| | | | | | | TTGGGGTAGT |
| | | | | | | GCTATAAGTA |
| | | | | | | CTATTGGAAA |
| | | | | | | TAATTCTGCA |
| | | | | | | GCGAATTGGG |
| | | | | | | CTACAGGTGG |
| | | | | | | AGCAGCTGGT |
| | | | | | | TGGAAAAGCT |
| | | | | | | GA |
| 192 | Mersacidin | Lantibiotic, | MSQEAIIRS | Bacillus sp. | 193 | ATGAGTCAAG |
| | | type | WKDPFSREN | (strain HIL- | | AAGCTATCAT |
| | | B | STQNPAGNP | Y85/54728) | | TCGTTCATGG |
| | | | FSELKEAQM | | | AAAGATCCTT |
| | | | DKLVGAGD | | | TTTCCCGTGA |
| | | | MEAACTFTL | | | AAATTCTACA |
| | | | PGGGGVCTL | | | CAAAATCCAG |
| | | | TSECIC | | | CTGGTAACCC |
| | | | | | | ATTCAGTGAG |
| | | | | | | CTGAAAGAAG |
| | | | | | | CACAAATGGA |
| | | | | | | TAAGTTAGTA |
| | | | | | | GGTGCGGGAG |
| | | | | | | ACATGGAAGC |
| | | | | | | AGCATGTACT |
| | | | | | | TTTACATTGCC |
| | | | | | | TGGTGGCGGC |
| | | | | | | GGTGTTTGTA |
| | | | | | | CTCTAACTTCT |
| | | | | | | GAATGTATTT |
| | | | | | | GTTAA |
| 194 | Mesentericin | class | MTNMKSVE | Leuconostoc | 195 | ATGACGAATA |
| | Y105 | IIA/YG | AYQQLDNQ | mesenteroides | | TGAAGTCTGT |
| | | NGV | NLKKVVGG | | | GGAAGCATAT |
| | | | KYYGNGVH | | | CAGCAATTAG |
| | | | CTKSGCSVN | | | ATAACCAGAA |
| | | | WGEAASAGI | | | TCTCAAGAAA |
| | | | HRLANGGN | | | GTTGTTGGTG |
| | | | GFW | | | GAAAGTATTA |
| | | | | | | TGGGAATGGT |
| | | | | | | GTTCACTGTA |
| | | | | | | CAAAAAGTGG |
| | | | | | | ATGCTCTGTTA |
| | | | | | | ACTGGGGAGA |
| | | | | | | AGCTGCCTCA |
| | | | | | | GCTGGCATAC |
| | | | | | | ATCGTTTGGC |
| | | | | | | CAATGGTGGA |
| | | | | | | AATGGATTTT |
| | | | | | | GGTAA |
| 196 | Michiganin- | Lantibiotic | MNDILETET | Clavibacter | 197 | ATGAACGACA |
| | A | | PVMVSPRW | michiganensis | | TCCTCGAGAC |
| | | | DMLLDAGE | subsp. | | GGAGACCCCC |
| | | | DTSPSVQTQI | michiganensis | | GTCATGGTCA |
| | | | DAEFRRVVS | | | GCCCCCGGTG |
| | | | PYMSSSGWL | | | GGACATGCTG |
| | | | CTLTIECGTII | | | CTCGACGCGG |
| | | | CACR | | | GCGAGGACAC |
| | | | | | | CAGCCCGTCC |
| | | | | | | GTCCAGACCC |
| | | | | | | AGATCGACGC |
| | | | | | | GGAGTTCCGT |
| | | | | | | CGCGTCGTGA |
| | | | | | | GCCCGTACAT |
| | | | | | | GTCCAGCAGC |
| | | | | | | GGCTGGCTCT |
| | | | | | | GCACGCTCAC |
| | | | | | | CATCGAATGT |
| | | | | | | GGCACCATCA |
| | | | | | | TCTGCGCGTG |
| | | | | | | TCGCTGA |
| 198 | Microcin | Unclassified | MELKASEFG | Escherichia | 199 | ATGGAATTAA |
| | B17 | | VVLSVDALK | coli | | AAGCGAGTGA |
| | (MccB17) | | LSRQSPLGV | | | ATTTGGTGTA |
| | | | GIGGGGGGG | | | GTTTTGTCCGT |
| | | | GGGSCGGQ | | | TGATGCTCTTA |
| | | | GGGCGGCSN | | | AATTATCACG |
| | | | GCSGGNGGS | | | CCAGTCTCCAT |
| | | | GGSGSHI | | | TAGGTGTTGG |
| | | | | | | CATTGGTGGT |
| | | | | | | GGTGGCGGCG |
| | | | | | | GCGGCGGCGG |
| | | | | | | CGGTAGCTGC |
| | | | | | | GGTGGTCAAG |
| | | | | | | GTGGCGGTTG |
| | | | | | | TGGTGGTTGC |
| | | | | | | AGCAACGGTT |
| | | | | | | GTAGTGGTGG |
| | | | | | | AAACGGTGGC |
| | | | | | | AGCGGCGGAA |
| | | | | | | GTGGTTCACA |
| | | | | | | TATC |
| 200 | Microcin | Unclassified | MRTGNAN | Escherichia | 201 | ATGCGTACTG |
| | C7 | | | coli | | GTAATGCAAA |
| | | | | | | CTAA |
| 202 | Microcin | Unclassified | MREISQKDL | Klebsiella | 203 | ATGAGAGAAA |
| | E492 | | NLAFGAGET | pneumoniae | | TTAGTCAAAA |
| | | | DPNTQLLND | | | GGACTTAAAT |
| | | | LGNNMAWG | | | CTTGCTTTTGG |
| | | | AALGAPGGL | | | TGCAGGAGAG |
| | | | GSAALGAAG | | | ACCGATCCAA |
| | | | GALQTVGQ | | | ATACTCAACTT |
| | | | GLIDHGPVN | | | CTAAACGACC |
| | | | VPIPVLIGPS | | | TTGGAAATAA |
| | | | WNGSGSGY | | | TATGGCATGG |
| | | | NSATSSSGS | | | GGTGCTGCTC |
| | | | GS | | | TTGGCGCTCCT |
| | | | | | | GGCGGATTAG |
| | | | | | | GATCAGCAGC |
| | | | | | | TTTGGGGGCC |
| | | | | | | GCGGGAGGTG |
| | | | | | | CATTACAAAC |
| | | | | | | TGTAGGGCAA |
| | | | | | | GGATTAATTG |
| | | | | | | ACCATGGTCC |
| | | | | | | TGTAAATGTC |
| | | | | | | CCCATCCCTGT |
| | | | | | | ACTCATCGGG |
| | | | | | | CCAAGCTGGA |
| | | | | | | ATGGTAGCGG |
| | | | | | | TAGTGGTTAT |
| | | | | | | AACAGCGCAA |
| | | | | | | CATCCAGTTCC |
| | | | | | | GGTAGTGGTA |
| | | | | | | GTTAA |
| 204 | Microcin | Unclassified | MREITESQL | Escherichia | 205 | ATGCGAGAAA |
| | H47 | | RYISGAGGA | coli | | TAACAGAATC |
| | | | PATSANAAG | | | ACAGTTAAGA |
| | | | AAAIVGALA | | | TATATTTCCGG |
| | | | GIPGGPLGV | | | GGCGGGAGGT |
| | | | VVGAVSAGL | | | GCGCCAGCGA |
| | | | TTAIGSTVGS | | | CTTCAGCTAAT |
| | | | GSASSSAGG | | | GCCGCAGGTG |
| | | | GS | | | CTGCAGCTAT |
| | | | | | | TGTTGGAGCT |
| | | | | | | CTCGCCGGAA |
| | | | | | | TACCTGGTGG |
| | | | | | | TCCACTTGGG |
| | | | | | | GTTGTAGTTG |
| | | | | | | GAGCCGTATC |
| | | | | | | TGCCGGTTTG |
| | | | | | | ACAACAGCAA |
| | | | | | | TTGGCTCGAC |
| | | | | | | CGTGGGAAGT |
| | | | | | | GGTAGTGCCA |
| | | | | | | GTTCTTCTGCT |
| | | | | | | GGTGGCGGTA |
| | | | | | | GCTAA |
| 206 | Microcin | Unclassified | MIKHFHFNK | Escherichia | 207 | ATGATTAAGC |
| | J25 | | LSSGKKNNV | coli | | ATTTTCATTTT |
| | | | PSPAKGVIQI | | | AATAAACTGT |
| | | | KKSASQLTK | | | CTTCTGGTAA |
| | | | GGAGHVPEY | | | AAAAAATAAT |
| | | | FVGIGTPISF | | | GTTCCATCTCC |
| | | | YG | | | TGCAAAGGGG |
| | | | | | | GTTATACAAA |
| | | | | | | TAAAAAAATC |
| | | | | | | AGCATCGCAA |
| | | | | | | CTCACAAAAG |
| | | | | | | GTGGTGCAGG |
| | | | | | | ACATGTGCCT |
| | | | | | | GAGTATTTTGT |
| | | | | | | GGGGATTGGT |
| | | | | | | ACACCTATAT |
| | | | | | | CTTTCTATGGC |
| | | | | | | TGA |
| 208 | Microcin- | Unclassified | MYMRELDR | Escherichia | 209 | ATGTATATGA |
| | 24 | | EELNCVGGA | coli | | GAGAGTTAGA |
| | | | GDPLADPNS | | | TAGAGAGGAA |
| | | | QIVRQIMSN | | | TTAAATTGCG |
| | | | AAWGPPLVP | | | TTGGTGGGGC |
| | | | ERFRGMAVG | | | TGGAGATCCG |
| | | | AAGGVTQT | | | CTTGCAGATC |
| | | | VLQGAAAH | | | CTAATTCCCA |
| | | | MPVNVPIPK | | | AATTGTAAGA |
| | | | VPMGPSWN | | | CAGATAATGT |
| | | | GSKG | | | CTAATGCGGC |
| | | | | | | ATGGGGCCCG |
| | | | | | | CCTTTGGTGCC |
| | | | | | | AGAGCGGTTT |
| | | | | | | AGGGGAATGG |
| | | | | | | CTGTTGGAGC |
| | | | | | | CGCAGGTGGG |
| | | | | | | GTTACGCAGA |
| | | | | | | CAGTTCTTCAA |
| | | | | | | GGAGCAGCAG |
| | | | | | | CTCATATGCC |
| | | | | | | GGTTAATGTC |
| | | | | | | CCTATACCTA |
| | | | | | | AAGTTCCGAT |
| | | | | | | GGGACCCTCA |
| | | | | | | TGGAACGGAA |
| | | | | | | GTAAAGGATA |
| | | | | | | A |
| 210 | Mundticin | Unclassified | MSQVVGGK | Enterococcus | 211 | ATGTCACAGG |
| | KS | | YYGNGVSC | mundtii | | TAGTAGGTGG |
| | | | NKKGCSVD | | | AAAATACTAC |
| | | | WGKAIGIIGN | | | GGTAATGGAG |
| | | | NSAANLATG | | | TCTCATGTAAT |
| | | | GAAGWKS | | | AAAAAAGGGT |
| | | | | | | GCAGTGTTGA |
| | | | | | | TTGGGGAAAA |
| | | | | | | GCGATTGGCA |
| | | | | | | TTATTGGAAA |
| | | | | | | TAATTCTGCTG |
| | | | | | | CGAATTTAGC |
| | | | | | | TACTGGTGGA |
| | | | | | | GCAGCTGGTT |
| | | | | | | GGAAAAGTTA |
| | | | | | | A |
| 212 | Mundticin | class | MKKLTSKE | Enterococcus | 213 | TTGAAGAAAT |
| | L | IIA/YG | MAQVVGGK | mundtii | | TAACATCAAA |
| | | NGV | YYGNGLSCN | | | AGAAATGGCA |
| | | | KKGCSVDW | | | CAAGTAGTAG |
| | | | GKAIGIIGNN | | | GTGGGAAATA |
| | | | SAANLATGG | | | CTACGGTAAT |
| | | | AAGWKS | | | GGATTATCAT |
| | | | | | | GTAATAAAAA |
| | | | | | | AGGGTGCAGT |
| | | | | | | GTTGATTGGG |
| | | | | | | GAAAAGCTAT |
| | | | | | | TGGCATTATT |
| | | | | | | GGAAATAATT |
| | | | | | | CTGCTGCGAA |
| | | | | | | TTTAGCTACTG |
| | | | | | | GTGGAGCAGC |
| | | | | | | TGGTTGGAAA |
| | | | | | | AGTTAA |
| 214 | Mutacin | Lantibiotic | MSNTQLLEV | Streptococcus | 215 | ATGTCAAACA |
| | 1140 | | LGTETFDVQ | mutans | | CACAATTATT |
| | (Mutacin | | EDLFAFDTT | | | AGAAGTCCTT |
| | III) | | DTTIVASND | | | GGTACTGAAA |
| | | | DPDTRFKSW | | | CTTTTGATGTT |
| | | | SLCTPGCAR | | | CAAGAAGATC |
| | | | TGSFNSYCC | | | TCTTTGCTTTT |
| | | | | | | GATACAACAG |
| | | | | | | ATACTACTATT |
| | | | | | | GTGGCAAGCA |
| | | | | | | ACGACGATCC |
| | | | | | | AGATACTCGT |
| | | | | | | TTCAAAAGTT |
| | | | | | | GGAGCCTTTG |
| | | | | | | TACGCCTGGT |
| | | | | | | TGTGCAAGGA |
| | | | | | | CAGGTAGTTT |
| | | | | | | CAATAGTTAC |
| | | | | | | TGTTGCTGA |
| 216 | Mutacin-2 | Lantibiotic | MNKLNSNA | Streptococcus | 217 | ATGAACAAGT |
| | | | VVSLNEVSD | mutans | | TAAACAGTAA |
| | | | SELDTILGGN | | | CGCAGTAGTT |
| | | | RWWQGVVP | | | TCTTTGAATG |
| | | | TVSYECRMN | | | AAGTTTCAGA |
| | | | SWQHVFTCC | | | TTCTGAATTG |
| | | | | | | GATACTATTTT |
| | | | | | | GGGTGGTAAT |
| | | | | | | CGTTGGTGGC |
| | | | | | | AAGGTGTTGT |
| | | | | | | GCCAACGGTC |
| | | | | | | TCATATGAGT |
| | | | | | | GTCGCATGAA |
| | | | | | | TTCATGGCAA |
| | | | | | | CATGTTTTCAC |
| | | | | | | TTGCTGTTAA |
| 218 | Nisin A | Lantibiotic | MSTKDFNLD | Lactococcus | 219 | ATGAGTACAA |
| | | | LVSVSKKDS | lactis subsp. | | AAGATTTTAA |
| | | | GASPRITSISL | lactis | | CTTGGATTTG |
| | | | CTPGCKTGA | (Streptococcus | | GTATCTGTTTC |
| | | | LMGCNMKT | lactis) | | GAAGAAAGAT |
| | | | ATCHCSIHV | | | TCAGGTGCAT |
| | | | SK | | | CACCACGCAT |
| | | | | | | TACAAGTATTT |
| | | | | | | CGCTATGTAC |
| | | | | | | ACCCGGTTGT |
| | | | | | | AAAACAGGAG |
| | | | | | | CTCTGATGGG |
| | | | | | | TTGTAACATG |
| | | | | | | AAAACAGCAA |
| | | | | | | CTTGTCATTGT |
| | | | | | | AGTATTCACG |
| | | | | | | TAAGCAAATA |
| | | | | | | A |
| 220 | Nisin F | Lantibiotic | MSTKDFNLD | Lactococcus | 221 | ATGAGTACAA |
| | | | LVSVSKKDS | lactis | | AAGATTTCAA |
| | | | GASPRITSISL | | | CTTGGATTTG |
| | | | CTPGCKTGA | | | GTATCTGTTTC |
| | | | LMGCNMKT | | | GAAGAAAGAT |
| | | | ATCNCSVHV | | | TCAGGTGCAT |
| | | | SK | | | CACCACGCAT |
| | | | | | | TACAAGTATTT |
| | | | | | | CGCTATGTAC |
| | | | | | | ACCCGGTTGT |
| | | | | | | AAAACAGGAG |
| | | | | | | CTCTGATGGG |
| | | | | | | TTGTAACATG |
| | | | | | | AAAACAGCAA |
| | | | | | | CTTGTAATTGT |
| | | | | | | AGCGTTCACG |
| | | | | | | TAAGCAAA |
| 222 | Nisin Q | Lantibiotic | MSTKDFNLD | Lactococcus | 223 | ATGAGTACAA |
| | | | LVSVSKTDS | lactis | | AAGATTTCAA |
| | | | GASTRITSIS | | | CTTAGATTTG |
| | | | LCTPGCKTG | | | GTATCTGTTTC |
| | | | VLMGCNLKT | | | AAAAACAGAT |
| | | | ATCNCSVHV | | | TCTGGCGCTTC |
| | | | SK | | | AACACGTATT |
| | | | | | | ACCAGCATTT |
| | | | | | | CGCTTTGTAC |
| | | | | | | ACCAGGTTGT |
| | | | | | | AAAACAGGTG |
| | | | | | | TTCTGATGGG |
| | | | | | | ATGTAACCTG |
| | | | | | | AAAACAGCAA |
| | | | | | | CTTGTAATTGT |
| | | | | | | AGCGTTCACG |
| | | | | | | TAAGCAAATA |
| | | | | | | A |
| 224 | Nisin U | Lantibiotic | MNNEDFNL | Streptococcus | 225 | ATGAACAATG |
| | | | DLIKISKENN | uberis | | AAGATTTTAA |
| | | | SGASPRITSK | | | TTTGGATCTCA |
| | | | SLCTPGCKT | | | TCAAAATCTC |
| | | | GILMTCPLK | | | AAAGGAAAAC |
| | | | TATCGCHFG | | | AACTCAGGAG |
| | | | | | | CTTCACCTCGA |
| | | | | | | ATAACTAGTA |
| | | | | | | AATCATTATGT |
| | | | | | | ACTCCTGGAT |
| | | | | | | GTAAGACGGG |
| | | | | | | TATTTTGATGA |
| | | | | | | CTTGTCCACTA |
| | | | | | | AAAACTGCAA |
| | | | | | | CCTGTGGTTG |
| | | | | | | TCATTTTGGAT |
| | | | | | | AA |
| 226 | Nisin Z | Lantibiotic | MSTKDFNLD | Lactococcus | 227 | ATGAGTACAA |
| | | | LVSVSKKDS | lactis subsp. | | AAGATTTTAA |
| | | | GASPRITSISL | lactis | | CTTGGATTTG |
| | | | CTPGCKTGA | (Streptococcus | | GTATCTGTTTC |
| | | | LMGCNMKT | lactis) | | GAAGAAAGAT |
| | | | ATCNCSIHV | | | TCAGGTGCAT |
| | | | SK | | | CACCACGCAT |
| | | | | | | TACAAGTATTT |
| | | | | | | CGCTATGTAC |
| | | | | | | ACCCGGTTGT |
| | | | | | | AAAACAGGAG |
| | | | | | | CTCTGATGGG |
| | | | | | | TTGTAACATG |
| | | | | | | AAAACAGCAA |
| | | | | | | CTTGTAATTGT |
| | | | | | | AGTATTCACG |
| | | | | | | TAAGCAAATA |
| | | | | | | A |
| 228 | Nukacin | Lantibiotic | MENSKVMK | Staphylococcus | 229 | ATGGAAAATT |
| | ISK-1 | | DIEVANLLE | warneri | | CTAAAGTTAT |
| | | | EVQEDELNE | | | GAAGGACATT |
| | | | VLGAKKKSG | | | GAAGTAGCAA |
| | | | VIPTVSHDC | | | ATTTATTAGA |
| | | | HMNSFQFVF | | | AGAGGTTCAA |
| | | | TCCS | | | GAAGATGAAT |
| | | | | | | TGAATGAAGT |
| | | | | | | CTTAGGAGCT |
| | | | | | | AAGAAAAAGT |
| | | | | | | CAGGAGTAAT |
| | | | | | | CCCAACTGTG |
| | | | | | | TCACACGATT |
| | | | | | | GCCATATGAA |
| | | | | | | TTCTTTCCAAT |
| | | | | | | TTGTATTTACT |
| | | | | | | TGTTGTTCATA |
| | | | | | | A |
| 230 | Paenicidin | Lantibiotic | MAENLFDLD | Paenibacillus | 231 | ATGGCTGAAA |
| | A | | IQVNKSQGS | polymyxa | | ACTTATTTGAT |
| | | | VEPQVLSIV | (Bacillus | | CTGGACATTC |
| | | | ACSSGCGSG | polymyxa) | | AAGTAAACAA |
| | | | KTAASCVET | | | ATCTCAAGGT |
| | | | CGNRCFTNV | | | TCTGTAGAGC |
| | | | GSLC | | | CTCAGGTTCT |
| | | | | | | GAGCATTGTT |
| | | | | | | GCATGTTCTA |
| | | | | | | GCGGATGTGG |
| | | | | | | TAGCGGTAAA |
| | | | | | | ACAGCTGCCA |
| | | | | | | GTTGTGTTGA |
| | | | | | | AACTTGTGGC |
| | | | | | | AACCGGTGCT |
| | | | | | | TTACTAACGTT |
| | | | | | | GGTTCACTCT |
| | | | | | | GCTAA |
| 232 | Pediocin | class | MKKIEKLTE | Pediococcus | 233 | ATGAAAAAAA |
| | PA-1 | IIA/YG | KEMANIIGG | acidilactici | | TTGAAAAATT |
| | (Pediocin | NGV | KYYGNGVT | | | AACTGAAAAA |
| | ACH) | | CGKHSCSVD | | | GAAATGGCCA |
| | | | WGKATTCII | | | ATATCATTGG |
| | | | NNGAMAWA | | | TGGTAAATAC |
| | | | TGGHQGNH | | | TACGGTAATG |
| | | | KC | | | GGGTTACTTG |
| | | | | | | TGGCAAACAT |
| | | | | | | TCCTGCTCTGT |
| | | | | | | TGACTGGGGT |
| | | | | | | AAGGCTACCA |
| | | | | | | CTTGCATAATC |
| | | | | | | AATAATGGAG |
| | | | | | | CTATGGCATG |
| | | | | | | GGCTACTGGT |
| | | | | | | GGACATCAAG |
| | | | | | | GTAATCATAA |
| | | | | | | ATGCTAG |
| 234 | Penocin A | class | MTEIKVLND | Pediococcus | 235 | ATGACTGAAA |
| | | IIA/YG | KELKNVVGG | pentosaceus | | TTAAAGTACT |
| | | NGV | KYYGNGVH | (strain ATCC | | AAACGATAAG |
| | | | CGKKTCYVD | 25745/183- | | GAACTAAAAA |
| | | | WGQATASIG | iw) | | ATGTCGTAGG |
| | | | KIIVNGWTQ | | | AGGAAAGTAT |
| | | | HGPWAHR | | | TACGGTAACG |
| | | | | | | GAGTGCATTG |
| | | | | | | TGGTAAAAAG |
| | | | | | | ACTTGCTATGT |
| | | | | | | GGACTGGGGA |
| | | | | | | CAAGCTACAG |
| | | | | | | CTAGCATTGG |
| | | | | | | AAAAATTATA |
| | | | | | | GTGAACGGAT |
| | | | | | | GGACACAACA |
| | | | | | | CGGGCCTTGG |
| | | | | | | GCACATAGAT |
| | | | | | | AA |
| 236 | Pep5 | Lantibiotic | MKNNKNLF | Staphylococcus | 237 | ATGAAAAATA |
| | | | DLEIKKETSQ | epidermidis | | ACAAAAATTT |
| | | | NTDELEPQT | | | ATTTGATTTAG |
| | | | AGPAIRASV | | | AAATTAAAAA |
| | | | KQCQKTLKA | | | AGAAACAAGT |
| | | | TRLFTVSCK | | | CAAAACACTG |
| | | | GKNGCK | | | ATGAACTTGA |
| | | | | | | ACCTCAAACT |
| | | | | | | GCTGGACCAG |
| | | | | | | CGATTAGAGC |
| | | | | | | TTCTGTGAAA |
| | | | | | | CAATGTCAGA |
| | | | | | | AAACTTTGAA |
| | | | | | | AGCTACGCGT |
| | | | | | | TTATTTACAGT |
| | | | | | | GTCTTGCAAA |
| | | | | | | GGAAAAAACG |
| | | | | | | GATGTAAATA |
| | | | | | | G |
| 238 | Piscicolin | class | MKTVKELSV | Carnobacteriumum | 239 | ATGAAAACTG |
| | 126 | IIA/YG | KEMQLTTGG | maltaromaticum | | TTAAAGAACT |
| | | NGV | KYYGNGVS | (Carnobacterium | | TAGCGTTAAA |
| | | | CNKNGCTV | piscicola) | | GAAATGCAAC |
| | | | DWSKAIGIIG | | | TAACTACAGG |
| | | | NNAAANLTT | | | AGGTAAGTAT |
| | | | GGAAGWNK | | | TACGGAAATG |
| | | | G | | | GCGTTTCCTGT |
| | | | | | | AATAAAAATG |
| | | | | | | GTTGTACTGT |
| | | | | | | AGATTGGAGC |
| | | | | | | AAAGCTATTG |
| | | | | | | GGATTATAGG |
| | | | | | | AAACAATGCA |
| | | | | | | GCAGCAAATT |
| | | | | | | TGACTACAGG |
| | | | | | | TGGAGCCGCT |
| | | | | | | GGTTGGAACA |
| | | | | | | AAGGATAA |
| 240 | Plantaricin | Unclassified | MYKELTVDE | Lactobacillus | 241 | ATGTATAAAG |
| | 1.25 β | | LALIDGGKK | plantarum | | AATTAACAGT |
| | | | KKKKVACT | | | TGATGAATTA |
| | | | WGNAATAA | | | GCATTGATTG |
| | | | ASGAVXGIL | | | ATGGAGGAAA |
| | | | GGPTGALAG | | | AAAGAAGAAG |
| | | | AIWGVSQCA | | | AAAAAAGTAG |
| | | | SNNLHGMH | | | CTTGTACTTGG |
| | | | | | | GGAAATGCAG |
| | | | | | | CAACAGCCGC |
| | | | | | | TGCTTCTGGT |
| | | | | | | GCAGTTANGG |
| | | | | | | GTATTCTTGGT |
| | | | | | | GGGCCTACTG |
| | | | | | | GTGCACTGGC |
| | | | | | | TGGAGCTATC |
| | | | | | | TGGGGCGTTT |
| | | | | | | CACAATGCGC |
| | | | | | | GTCTAACAAC |
| | | | | | | TTACACGGCA |
| | | | | | | TGCACTAA |
| 242 | Plantaricin | class IIa | MMKKIEKLT | Lactobacillus | 243 | ATGATGAAAA |
| | 423 | | EKEMANIIG | plantarum | | AAATTGAAAA |
| | | | GKYYGNGV | | | ATTAACTGAA |
| | | | TCGKHSCSV | | | AAAGAAATGG |
| | | | NWGQAFSCS | | | CCAATATCATT |
| | | | VSHLANFGH | | | GGTGGTAAAT |
| | | | GKC | | | ACTATGGTAA |
| | | | | | | TGGGGTTACT |
| | | | | | | TGTGGTAAAC |
| | | | | | | ATTCCTGCTCT |
| | | | | | | GTTAACTGGG |
| | | | | | | GCCAAGCATT |
| | | | | | | TTCTTGTAGTG |
| | | | | | | TGTCACATTTA |
| | | | | | | GCTAACTTCG |
| | | | | | | GTCATGGAAA |
| | | | | | | GTGCTAA |
| 244 | Plantaricin | Unclassified | MSKLVKTLT | Lactobacillus | 245 | ATGAGTAAAC |
| | ASM1 | | VDEISKIQTN | plantarum | | TAGTTAAAAC |
| | | | GGKPAWCW | | | ATTAACTGTC |
| | | | YTLAMCGA | | | GATGAAATCT |
| | | | GYDSGTCDY | | | CTAAGATTCA |
| | | | MYSHCFGVK | | | AACCAATGGT |
| | | | HSSGGGGSY | | | GGAAAACCTG |
| | | | HC | | | CATGGTGTTG |
| | | | | | | GTACACATTG |
| | | | | | | GCAATGTGCG |
| | | | | | | GTGCTGGTTA |
| | | | | | | TGATTCAGGC |
| | | | | | | ACTTGTGATT |
| | | | | | | ATATGTATTCA |
| | | | | | | CACTGCTTTG |
| | | | | | | GTGTAAAACA |
| | | | | | | CTCTAGCGGT |
| | | | | | | GGTGGCGGTA |
| | | | | | | GCTACCATTG |
| | | | | | | TTAG |
| 246 | Plantaricin | Unclassified | MLQFEKLQY | Lactobacillus | 247 | ATGCTACAGT |
| | E | | SRLPQKKLA | plantarum | | TTGAGAAATT |
| | | | KISGGFNRG | | | ACAATATTCC |
| | | | GYNFGKSVR | | | AGGTTGCCGC |
| | | | HVVDAIGSV | | | AAAAAAAGCT |
| | | | AGIRGILKSI | | | TGCCAAAATA |
| | | | R | | | TCTGGTGGTTT |
| | | | | | | TAATCGGGGC |
| | | | | | | GGTTATAACT |
| | | | | | | TTGGTAAAAG |
| | | | | | | TGTTCGACAT |
| | | | | | | GTTGTTGATG |
| | | | | | | CAATTGGTTC |
| | | | | | | AGTTGCAGGC |
| | | | | | | ATTCGTGGTA |
| | | | | | | TTTTGAAAAG |
| | | | | | | TATTCGTTAA |
| 248 | Plantaricin | class IIb | MKKFLVLRD | Lactobacillus | 249 | ATGAAAAAAT |
| | F | | RELNAISGG | plantarum | | TTCTAGTTTTG |
| | | | VFHAYSARG | | | CGTGACCGTG |
| | | | VRNNYKSAV | | | AATTAAATGC |
| | | | GPADWVISA | | | TATTTCAGGT |
| | | | VRGFIHG | | | GGCGTTTTCC |
| | | | | | | ATGCCTATAG |
| | | | | | | CGCGCGTGGC |
| | | | | | | GTTCGGAATA |
| | | | | | | ATTATAAAAG |
| | | | | | | TGCTGTTGGG |
| | | | | | | CCTGCCGATT |
| | | | | | | GGGTCATTAG |
| | | | | | | CGCTGTCCGA |
| | | | | | | GGATTCATCC |
| | | | | | | ACGGATAG |
| 250 | Plantaricin | class IIb | MTVNKMIK | Lactobacillus | 251 | ATGACTGTGA |
| | J | | DLDVVDAFA | plantarum | | ACAAAATGAT |
| | | | PISNNKLNG | | | TAAGGATTTG |
| | | | VVGGGAWK | | | GATGTAGTAG |
| | | | NFWSSLRKG | | | ATGCATTTGC |
| | | | FYDGEAGRA | | | ACCTATTTCTA |
| | | | IRR | | | ATAATAAGTT |
| | | | | | | GAACGGGGTT |
| | | | | | | GTTGGGGGAG |
| | | | | | | GCGCTTGGAA |
| | | | | | | AAATTTCTGG |
| | | | | | | TCTAGTTTAA |
| | | | | | | GAAAAGGATT |
| | | | | | | TTATGATGGC |
| | | | | | | GAAGCTGGCA |
| | | | | | | GAGCAATCCG |
| | | | | | | TCGTTAA |
| 252 | Plantaricin | Unclassified | MKIKLTVLN | Lactobacillus | 253 | ATGAAAATTA |
| | K | | EFEELTADA | plantarum | | AATTAACTGTT |
| | | | EKNISGGRR | | | TTAAATGAAT |
| | | | SRKNGIGYAI | | | TTGAAGAATT |
| | | | GYAFGAVER | | | AACTGCTGAC |
| | | | AVLGGSRDY | | | GCTGAAAAGA |
| | | | NK | | | ATATTTCTGGT |
| | | | | | | GGCCGTCGGA |
| | | | | | | GTCGTAAAAA |
| | | | | | | TGGAATTGGA |
| | | | | | | TACGCTATTG |
| | | | | | | GTTATGCGTTT |
| | | | | | | GGCGCGGTTG |
| | | | | | | AACGGGCCGT |
| | | | | | | GCTTGGTGGT |
| | | | | | | TCAAGGGATT |
| | | | | | | ATAATAAGTG |
| | | | | | | A |
| 254 | Plantaricin | Unclassified | MDKFEKIST | Lactobacillus | 255 | ATGGATAAAT |
| | NC8 α | | SNLEKISGG | plantarum | | TTGAAAAAAT |
| | | | DLTTKLWSS | | | TAGTACATCT |
| | | | WGYYLGKK | | | AACCTAGAAA |
| | | | ARWNLKHP | | | AGATCTCTGG |
| | | | YVQF | | | CGGTGATTTA |
| | | | | | | ACAACCAAGT |
| | | | | | | TATGGAGCTC |
| | | | | | | TTGGGGATAT |
| | | | | | | TATCTTGGCA |
| | | | | | | AGAAAGCACG |
| | | | | | | TTGGAATTTA |
| | | | | | | AAGCACCCAT |
| | | | | | | ATGTTCAATTT |
| 256 | Plantaricin | Unclassified | MNNLNKFST | Lactobacillus | 257 | ATGAATAACT |
| | NC8 β | | LGKSSLSQIE | plantarum | | TGAATAAATT |
| | | | GGSVPTSVY | | | TTCTACTCTAG |
| | | | TLGIKILWSA | | | GCAAGAGTAG |
| | | | YKHRKTIEK | | | CTTGTCTCAAA |
| | | | SFNKGFYH | | | TTGAGGGCGG |
| | | | | | | ATCAGTCCCA |
| | | | | | | ACTTCAGTAT |
| | | | | | | ATACGCTTGG |
| | | | | | | AATTAAAATT |
| | | | | | | CTATGGTCTG |
| | | | | | | CGTATAAGCA |
| | | | | | | TCGCAAAACG |
| | | | | | | ATTGAAAAAA |
| | | | | | | GTTTTAATAA |
| | | | | | | AGGCTTTTATC |
| | | | | | | ATTAA |
| 258 | Plantaricin | Unclassified | MNNALSFEQ | Lactobacillus | 259 | ATGAATAACG |
| | S α | | QFTDFSTLSD | plantarum | | CATTAAGTTTT |
| | | | SELESVEGG | | | GAACAACAAT |
| | | | RNKLAYNM | | | TTACAGACTTC |
| | | | GHYAGKATI | | | AGCACCTTAT |
| | | | FGLAAWALL | | | CGGACTCTGA |
| | | | A | | | ATTAGAATCC |
| | | | | | | GTTGAGGGTG |
| | | | | | | GCCGAAATAA |
| | | | | | | GCTTGCATAT |
| | | | | | | AATATGGGGC |
| | | | | | | ATTACGCTGG |
| | | | | | | TAAGGCAACC |
| | | | | | | ATTTTTGGACT |
| | | | | | | TGCAGCATGG |
| | | | | | | GCACTCCTTG |
| | | | | | | CATGA |
| 260 | Plantaricin | Unclassified | MDKIIKFQGI | Lactobacillus | 261 | ATGGATAAGA |
| | S β | | SDDQLNAVI | plantarum | | TTATTAAGTTT |
| | | | GGKKKKQS | | | CAAGGGATTT |
| | | | WYAAAGDAI | | | CTGATGATCA |
| | | | VSFGEGFLN | | | ATTAAATGCT |
| | | | AW | | | GTTATCGGTG |
| | | | | | | GGAAAAAGAA |
| | | | | | | AAAACAATCT |
| | | | | | | TGGTACGCAG |
| | | | | | | CAGCTGGTGA |
| | | | | | | TGCAATCGTT |
| | | | | | | AGTTTTGGTG |
| | | | | | | AAGGATTTTT |
| | | | | | | AAATGCTTGG |
| | | | | | | TAA |
| 262 | Plantaricin | Lantibiotic | MKISKIEAQ | Lactobacillus | 263 | ATGAAAATTT |
| | W α | (two- | ARKDFFKKI | plantarum | | CTAAGATTGA |
| | | peptide) | DTNSNLLNV | | | AGCTCAGGCT |
| | | | NGAKCKWW | | | CGTAAAGATT |
| | | | NISCDLGNN | | | TTTTTAAAAA |
| | | | GHVCTLSHE | | | AATCGATACT |
| | | | CQVSCN | | | AACTCGAACT |
| | | | | | | TATTAAATGT |
| | | | | | | AAATGGTGCC |
| | | | | | | AAATGCAAGT |
| | | | | | | GGTGGAATAT |
| | | | | | | TTCGTGTGATT |
| | | | | | | TAGGAAATAA |
| | | | | | | TGGCCATGTTT |
| | | | | | | GTACCTTGTC |
| | | | | | | ACATGAATGC |
| | | | | | | CAAGTATCTT |
| | | | | | | GTAACTAA |
| 264 | Plantaricin | Lantibiotic | MTKTSRRKN | Lactobacillus | 265 | ATGACTAAAA |
| | W β | (two- | AIANYLEPV | plantarum | | CTAGTCGTCG |
| | | peptide) | DEKSINESFG | | | TAAGAATGCT |
| | | | AGDPEARSG | | | ATTGCTAATTA |
| | | | IPCTIGAAVA | | | TTTAGAACCA |
| | | | ASIAVCPTTK | | | GTCGACGAAA |
| | | | CSKRCGKRK | | | AAAGTATTAA |
| | | | K | | | TGAATCTTTTG |
| | | | | | | GGGCTGGGGA |
| | | | | | | TCCGGAAGCA |
| | | | | | | AGATCCGGAA |
| | | | | | | TTCCATGTACA |
| | | | | | | ATCGGCGCAG |
| | | | | | | CTGTCGCAGC |
| | | | | | | ATCAATTGCA |
| | | | | | | GTTTGTCCAA |
| | | | | | | CTACTAAGTG |
| | | | | | | TAGTAAACGT |
| | | | | | | TGTGGCAAGC |
| | | | | | | GTAAGAAATA |
| | | | | | | A |
| 266 | Plantaricin- | Unclassified | MKIQIKGMK | Lactobacillus | 267 | ATGAAAATTC |
| | A | | QLSNKEMQK | plantarum | | AAATTAAAGG |
| | | | IVGGKSSAY | (strain ATCC | | TATGAAGCAA |
| | | | SLQMGATAI | BAA-793/ | | CTTAGTAATA |
| | | | KQVKKLFKK | NCIMB | | AGGAAATGCA |
| | | | WGW | 8826/ | | AAAAATAGTA |
| | | | | WCFS1) | | GGTGGAAAGA |
| | | | | | | GTAGTGCGTA |
| | | | | | | TTCTTTGCAGA |
| | | | | | | TGGGGGCAAC |
| | | | | | | TGCAATTAAA |
| | | | | | | CAGGTAAAGA |
| | | | | | | AACTGTTTAA |
| | | | | | | AAAATGGGGA |
| | | | | | | TGGTAA |
| 268 | Propionicin | Unclassified | MNKTHKMA | Propioni | 269 | ATGAACAAAA |
| | SM1 | | TLVIAAILAA | bacterium | | CACACAAAAT |
| | | | GMTAPTAYA | jensenii | | GGCGACGCTG |
| | | | DSPGNTRITA | | | GTAATTGCCG |
| | | | SEQSVLTQIL | | | CGATCTTGGC |
| | | | GHKPTQTEY | | | CGCCGGAATG |
| | | | NRYVETYGS | | | ACCGCACCAA |
| | | | VPTEADINA | | | CTGCCTATGC |
| | | | YIEASESEGS | | | AGATTCTCCT |
| | | | SSQTAAHDD | | | GGAAACACCA |
| | | | STSPGTSTEI | | | GAATTACAGC |
| | | | YTQAAPARF | | | CAGCGAGCAA |
| | | | SMFFLSGTW | | | AGCGTCCTTA |
| | | | ITRSGVVSLS | | | CCCAGATACT |
| | | | LKPRKGGIG | | | CGGCCACAAA |
| | | | NEGDERTW | | | CCTACACAAA |
| | | | KTVYDKFHN | | | CTGAATATAA |
| | | | AGQWTRYK | | | CCGATACGTT |
| | | | NNGVDASM | | | GAGACTTACG |
| | | | KKQYMCHF | | | GAAGCGTACC |
| | | | KYGMVKTP | | | GACCGAAGCA |
| | | | WNLEPHKK | | | GACATCAACG |
| | | | AADVSPVKC | | | CATATATAGA |
| | | | N | | | AGCGTCTGAA |
| | | | | | | TCTGAGGGAT |
| | | | | | | CATCAAGTCA |
| | | | | | | AACGGCTGCT |
| | | | | | | CACGATGACT |
| | | | | | | CGACATCACC |
| | | | | | | CGGCACGAGT |
| | | | | | | ACCGAAATCT |
| | | | | | | ACACGCAGGC |
| | | | | | | AGCCCCTGCC |
| | | | | | | AGGTTCTCAA |
| | | | | | | TGTTTTTCCTG |
| | | | | | | TCCGGAACTT |
| | | | | | | GGATCACTAG |
| | | | | | | GAGTGGTGTA |
| | | | | | | GTATCGCTCTC |
| | | | | | | CTTGAAGCCA |
| | | | | | | AGGAAGGGTG |
| | | | | | | GTATTGGCAA |
| | | | | | | CGAGGGGGAC |
| | | | | | | GAGCGTACCT |
| | | | | | | GGAAGACTGT |
| | | | | | | ATACGACAAA |
| | | | | | | TTCCATAACG |
| | | | | | | CTGGGCAATG |
| | | | | | | GACACGATAC |
| | | | | | | AAGAACAACG |
| | | | | | | GCGTAGACGC |
| | | | | | | CAGCATGAAA |
| | | | | | | AAGCAGTACA |
| | | | | | | TGTGCCACTTC |
| | | | | | | AAGTACGGGA |
| | | | | | | TGGTGAAGAC |
| | | | | | | GCCATGGAAT |
| | | | | | | CTGGAGCCCC |
| | | | | | | ACAAGAAGGC |
| | | | | | | TGCAGACGTC |
| | | | | | | AGTCCAGTCA |
| | | | | | | AGTGCAACTA |
| | | | | | | G |
| 270 | Propionicin | Unclassified | MKKTLLRSG | Propionibacterium | 271 | ATGAAGAAGA |
| | T1 | | TIALATAAAF | thoenii | | CCCTCCTGCG |
| | | | GASLAAAPS | | | AAGTGGAACG |
| | | | AMAVPGGC | | | ATCGCACTGG |
| | | | TYTRSNRDV | | | CGACCGCGGC |
| | | | IGTCKTGSG | | | TGCATTTGGC |
| | | | QFRIRLDCN | | | GCATCATTGG |
| | | | NAPDKTSVW | | | CAGCCGCCCC |
| | | | AKPKVMVS | | | ATCTGCCATG |
| | | | VHCLVGQPR | | | GCCGTTCCTG |
| | | | SISFETK | | | GTGGTTGCAC |
| | | | | | | GTACACAAGA |
| | | | | | | AGCAATCGCG |
| | | | | | | ACGTCATCGG |
| | | | | | | TACCTGCAAG |
| | | | | | | ACTGGAAGCG |
| | | | | | | GCCAGTTCCG |
| | | | | | | AATCCGACTT |
| | | | | | | GACTGCAACA |
| | | | | | | ACGCTCCAGA |
| | | | | | | CAAAACTTCA |
| | | | | | | GTCTGGGCCA |
| | | | | | | AGCCCAAGGT |
| | | | | | | AATGGTGTCG |
| | | | | | | GTTCACTGTCT |
| | | | | | | TGTTGGTCAA |
| | | | | | | CCGAGGTCCA |
| | | | | | | TCTCGTTCGA |
| | | | | | | GACCAAGTGA |
| 272 | Propionicin- | Unclassified | MNTKAVNL | Propioni | 273 | ATGAATACCA |
| | F | | KSENTTKLV | bacterium | | AAGCTGTAAA |
| | | | SYLTENQLD | freudenreichii | | TCTGAAGTCA |
| | | | EFIRRIRIDG | subsp. | | GAAAACACGA |
| | | | ALVEEVSQN | freudenreichii | | CTAAGTTGGT |
| | | | AKQALDNTG | | | GAGCTACCTT |
| | | | LNGWINTDC | | | ACGGAAAATC |
| | | | DEGLLSDFIS | | | AATTGGATGA |
| | | | KIASARWIPL | | | GTTTATTAGA |
| | | | AESIRPAVTD | | | AGGATTCGCA |
| | | | RDKYRVSC | | | TTGATGGCGC |
| | | | WFYQGMNI | | | TCTTGTGGAA |
| | | | AIYANIGGV | | | GAGGTCAGTC |
| | | | ANIIGYTEAA | | | AAAATGCTAA |
| | | | VATLLGAVV | | | GCAGGCCTTA |
| | | | AVAPVVPGT | | | GATAATACTG |
| | | | PTPPKDKSS | | | GGCTCAATGG |
| | | | QYKEVPLAV | | | CTGGATAAAT |
| | | | RLSETYHEE | | | ACTGATTGCG |
| | | | GVRGLFDEL | | | ATGAAGGCCT |
| | | | NYSESRMIS | | | TCTCTCTGATT |
| | | | TLRRASTDG | | | TCATTTCAAA |
| | | | VLINSWNDG | | | GATAGCAAGT |
| | | | QDTILLKKY | | | GCTAGATGGA |
| | | | NFQDLQLTV | | | TTCCATTAGCT |
| | | | RSRIVGNQTI | | | GAGTCAATTC |
| | | | IEECKITDGR | | | GACCTGCGGT |
| | | | KTLSDETV | | | GACTGACAGG |
| | | | | | | GATAAGTATC |
| | | | | | | GAGTAAGTTG |
| | | | | | | CTGGTTCTACC |
| | | | | | | AGGGGATGAA |
| | | | | | | TATAGCAATTT |
| | | | | | | ACGCAAATAT |
| | | | | | | CGGTGGCGTG |
| | | | | | | GCCAATATTA |
| | | | | | | TCGGCTATAC |
| | | | | | | GGAGGCCGCA |
| | | | | | | GTCGCAACAC |
| | | | | | | TCCTTGGTGC |
| | | | | | | AGTTGTGGCG |
| | | | | | | GTAGCTCCTG |
| | | | | | | TGGTCCCTGG |
| | | | | | | AACTCCAACC |
| | | | | | | CCTCCAAAGG |
| | | | | | | ACAAGAGTTC |
| | | | | | | GCAATATAAG |
| | | | | | | GAGGTTCCCC |
| | | | | | | TTGCCGTTCGT |
| | | | | | | CTTTCCGAAA |
| | | | | | | CATACCACGA |
| | | | | | | AGAGGGAGTA |
| | | | | | | CGAGGTCTAT |
| | | | | | | TCGACGAGCT |
| | | | | | | GAACTACTCC |
| | | | | | | GAGAGCCGTA |
| | | | | | | TGATCTCTACT |
| | | | | | | CTAAGGCGAG |
| | | | | | | CATCAACCGA |
| | | | | | | TGGAGTCCTA |
| | | | | | | ATTAATTCTTG |
| | | | | | | GAACGATGGG |
| | | | | | | CAGGATACAA |
| | | | | | | TTCTGCTTAAG |
| | | | | | | AAGTACAATT |
| | | | | | | TCCAAGACTT |
| | | | | | | GCAACTGACT |
| | | | | | | GTCAGGAGCC |
| | | | | | | GCATTGTTGG |
| | | | | | | GAATCAAACA |
| | | | | | | ATAATTGAAG |
| | | | | | | AATGCAAAAT |
| | | | | | | CACTGATGGT |
| | | | | | | AGAAAAACTC |
| | | | | | | TTTCAGACGA |
| | | | | | | GACTGTGTAG |
| 274 | Pyocin S1 | Unclassified | MARPIADLIH | Pseudomonas | 275 | ATGGCACGAC |
| | | | FNSTTVTAS | aeruginosa | | CCATTGCTGA |
| | | | GDVYYGPG | | | CCTTATCCACT |
| | | | GGTGIGPIAR | | | TCAACTCTAC |
| | | | PIEHGLDSST | | | AACTGTCACG |
| | | | ENGWQEFES | | | GCAAGCGGAG |
| | | | YADVGVDP | | | ACGTTTATTAC |
| | | | RRYVPLQVK | | | GGCCCTGGGG |
| | | | EKRREIELQF | | | GAGGTACCGG |
| | | | RDAEKKLEA | | | CATTGGCCCC |
| | | | SVQAELDKA | | | ATTGCCAGAC |
| | | | DAALGPAKN | | | CTATAGAGCA |
| | | | LAPLDVINRS | | | CGGCTTGGAT |
| | | | LTIVGNALQ | | | TCGTCCACTG |
| | | | QKNQKLLLN | | | AAAATGGCTG |
| | | | QKKITSLGA | | | GCAAGAGTTT |
| | | | KNFLTRTAE | | | GAAAGTTATG |
| | | | EIGEQAVRE | | | CTGATGTGGG |
| | | | GNINGPEAY | | | CGTTGACCCC |
| | | | MRFLDREME | | | AGACGCTATG |
| | | | GLTAAYNVK | | | TTCCTCTTCAG |
| | | | LFTEAISSLQI | | | GTTAAAGAAA |
| | | | RMNTLTAAK | | | AACGCAGGGA |
| | | | ASIEAAAAN | | | GATCGAGCTT |
| | | | KAREQAAAE | | | CAGTTCCGAG |
| | | | AKRKAEEQA | | | ATGCCGAGAA |
| | | | RQQAAIRAA | | | AAAACTTGAG |
| | | | NTYAMPAN | | | GCGTCGGTAC |
| | | | GSVVATAAG | | | AAGCCGAGCT |
| | | | RGLIQVAQG | | | GGATAAGGCT |
| | | | AASLAQAIS | | | GATGCCGCTC |
| | | | DAIAVLGRV | | | TTGGTCCGGC |
| | | | LASAPSVMA | | | AAAGAATCTT |
| | | | VGFASLTYS | | | GCACCATTGG |
| | | | SRTAEQWQ | | | ACGTCATCAA |
| | | | DQTPDSVRY | | | CCGCAGTCTG |
| | | | ALGMDAAK | | | ACCATCGTTG |
| | | | LGLPPSVNL | | | GAAACGCCCT |
| | | | NAVAKASGT | | | CCAGCAAAAG |
| | | | VDLPMRLTN | | | AATCAAAAAC |
| | | | EARGNTTTL | | | TACTGCTGAA |
| | | | SVVSTDGVS | | | TCAGAAGAAG |
| | | | VPKAVPVRM | | | ATTACCAGCC |
| | | | AAYNATTGL | | | TGGGTGCAAA |
| | | | YEVTVPSTT | | | GAATTTCCTTA |
| | | | AEAPPLILTW | | | CCCGTACGGC |
| | | | TPASPPGNQ | | | GGAAGAGATC |
| | | | NPSSTTPVVP | | | GGTGAACAAG |
| | | | KPVPVYEGA | | | CGGTGCGAGA |
| | | | TLTPVKATP | | | AGGCAATATT |
| | | | ETYPGVITLP | | | AACGGGCCTG |
| | | | EDLIIGFPAD | | | AAGCCTATAT |
| | | | SGIKPIYVMF | | | GCGCTTCCTC |
| | | | RDPRDVPGA | | | GACAGGGAAA |
| | | | ATGKGQPVS | | | TGGAAGGTCT |
| | | | GNWLGAAS | | | CACGGCAGCT |
| | | | QGEGAPIPSQ | | | TATAACGTAA |
| | | | IADKLRGKT | | | AACTCTTCACC |
| | | | FKNWRDFRE | | | GAAGCGATCA |
| | | | QFWIAVAND | | | GTAGTCTCCA |
| | | | PELSKQFNP | | | GATCCGCATG |
| | | | GSLAVMRD | | | AATACGTTGA |
| | | | GGAPYVRES | | | CCGCCGCCAA |
| | | | EQAGGRIKIE | | | AGCAAGTATT |
| | | | IHHKVRVAD | | | GAGGCGGCCG |
| | | | GGGVYNMG | | | CAGCAAACAA |
| | | | NLVAVTPKR | | | GGCGCGTGAA |
| | | | HIEIHKGGK | | | CAAGCAGCGG |
| | | | | | | CTGAGGCCAA |
| | | | | | | ACGCAAAGCC |
| | | | | | | GAAGAGCAGG |
| | | | | | | CCCGCCAGCA |
| | | | | | | AGCGGCGATA |
| | | | | | | AGAGCTGCCA |
| | | | | | | ATACCTATGC |
| | | | | | | CATGCCGGCC |
| | | | | | | AATGGCAGCG |
| | | | | | | TTGTCGCCAC |
| | | | | | | CGCCGCAGGC |
| | | | | | | CGGGGTCTGA |
| | | | | | | TCCAGGTCGC |
| | | | | | | ACAAGGCGCC |
| | | | | | | GCATCCCTTG |
| | | | | | | CTCAAGCGAT |
| | | | | | | CTCCGATGCG |
| | | | | | | ATTGCCGTCCT |
| | | | | | | GGGCCGGGTC |
| | | | | | | CTGGCTTCAG |
| | | | | | | CACCCTCGGT |
| | | | | | | GATGGCCGTG |
| | | | | | | GGCTTTGCCA |
| | | | | | | GTCTGACCTA |
| | | | | | | CTCCTCCCGG |
| | | | | | | ACTGCCGAGC |
| | | | | | | AATGGCAGGA |
| | | | | | | CCAAACGCCC |
| | | | | | | GATAGCGTTC |
| | | | | | | GTTACGCCCT |
| | | | | | | GGGCATGGAT |
| | | | | | | GCCGCTAAAT |
| | | | | | | TGGGGCTTCC |
| | | | | | | CCCAAGCGTA |
| | | | | | | AACCTGAACG |
| | | | | | | CGGTTGCAAA |
| | | | | | | AGCCAGCGGT |
| | | | | | | ACCGTCGATC |
| | | | | | | TGCCGATGCG |
| | | | | | | CCTGACCAAC |
| | | | | | | GAGGCACGAG |
| | | | | | | GCAACACGAC |
| | | | | | | GACCCTTTCG |
| | | | | | | GTGGTCAGCA |
| | | | | | | CCGATGGTGT |
| | | | | | | GAGCGTTCCG |
| | | | | | | AAAGCCGTTC |
| | | | | | | CGGTCCGGAT |
| | | | | | | GGCGGCCTAC |
| | | | | | | AATGCCACGA |
| | | | | | | CAGGCCTGTA |
| | | | | | | CGAGGTTACG |
| | | | | | | GTTCCCTCTAC |
| | | | | | | GACCGCAGAA |
| | | | | | | GCGCCGCCAC |
| | | | | | | TGATCCTGAC |
| | | | | | | CTGGACGCCG |
| | | | | | | GCGAGTCCTC |
| | | | | | | CAGGAAACCA |
| | | | | | | GAACCCTTCG |
| | | | | | | AGTACCACTC |
| | | | | | | CGGTCGTACC |
| | | | | | | GAAGCCGGTG |
| | | | | | | CCGGTATATG |
| | | | | | | AGGGAGCGAC |
| | | | | | | CCTTACACCG |
| | | | | | | GTGAAGGCTA |
| | | | | | | CCCCGGAAAC |
| | | | | | | CTATCCTGGG |
| | | | | | | GTGATTACAC |
| | | | | | | TACCGGAAGA |
| | | | | | | CCTGATCATC |
| | | | | | | GGCTTCCCGG |
| | | | | | | CCGACTCGGG |
| | | | | | | GATCAAGCCG |
| | | | | | | ATCTATGTGA |
| | | | | | | TGTTCAGGGA |
| | | | | | | TCCGCGGGAT |
| | | | | | | GTACCTGGTG |
| | | | | | | CTGCGACTGG |
| | | | | | | CAAGGGACAG |
| | | | | | | CCCGTCAGCG |
| | | | | | | GTAATTGGCT |
| | | | | | | CGGCGCCGCC |
| | | | | | | TCTCAAGGTG |
| | | | | | | AGGGGGCTCC |
| | | | | | | AATTCCAAGC |
| | | | | | | CAGATTGCGG |
| | | | | | | ATAAACTACG |
| | | | | | | TGGTAAGACA |
| | | | | | | TTCAAAAACT |
| | | | | | | GGCGGGACTT |
| | | | | | | TCGGGAACAA |
| | | | | | | TTCTGGATAG |
| | | | | | | CTGTGGCTAA |
| | | | | | | TGATCCTGAG |
| | | | | | | TTAAGTAAAC |
| | | | | | | AGTTTAATCCT |
| | | | | | | GGTAGTTTAG |
| | | | | | | CTGTAATGAG |
| | | | | | | AGATGGAGGG |
| | | | | | | GCTCCTTATGT |
| | | | | | | CAGAGAGTCA |
| | | | | | | GAACAGGCTG |
| | | | | | | GCGGGAGAAT |
| | | | | | | AAAGATCGAA |
| | | | | | | ATCCACCACA |
| | | | | | | AGGTTCGAGT |
| | | | | | | AGCAGATGGA |
| | | | | | | GGCGGCGTTT |
| | | | | | | ACAATATGGG |
| | | | | | | GAACCTTGTT |
| | | | | | | GCAGTAACGC |
| | | | | | | CAAAACGTCA |
| | | | | | | TATAGAAATC |
| | | | | | | CACAAGGGAG |
| | | | | | | GGAAGTGA |
| 276 | Pyocin S2 | colicin/ | MAVNDYEP | Pseudomonas | 277 | ATGGCTGTCA |
| | | pyosin | GSMVITHVQ | aeruginosa | | ATGATTACGA |
| | | nuclease | GGGRDIIQYI | (strain ATCC | | ACCTGGTTCG |
| | | family | PARSSYGTPP | 15692/ | | ATGGTTATTA |
| | | | FVPPGPSPYV | PAO1/1C/ | | CACATGTGCA |
| | | | GTGMQEYR | PRS 101/ | | GGGTGGTGGG |
| | | | KLRSTLDKS | LMG 12228) | | CGTGACATAA |
| | | | HSELKKNLK | | | TCCAGTATATT |
| | | | NETLKEVDE | | | CCTGCTCGAT |
| | | | LKSEAGLPG | | | CAAGCTACGG |
| | | | KAVSANDIR | | | TACTCCACCAT |
| | | | DEKSIVDAL | | | TTGTCCCACCA |
| | | | MDAKAKSL | | | GGACCAAGTC |
| | | | KAIEDRPAN | | | CGTATGTCGG |
| | | | LYTASDFPQ | | | TACTGGAATG |
| | | | KSESMYQSQ | | | CAGGAGTACA |
| | | | LLASRKFYG | | | GGAAGCTAAG |
| | | | EFLDRHMSE | | | AAGTACGCTT |
| | | | LAKAYSADI | | | GATAAGTCCC |
| | | | YKAQIAILK | | | ATTCAGAACT |
| | | | QTSQELENK | | | CAAGAAAAAC |
| | | | ARSLEAEAQ | | | CTGAAAAATG |
| | | | RAAAEVEAD | | | AAACCCTGAA |
| | | | YKARKANV | | | GGAGGTTGAT |
| | | | EKKVQSELD | | | GAACTCAAGA |
| | | | QAGNALPQL | | | GTGAAGCGGG |
| | | | TNPTPEQWL | | | GTTGCCAGGT |
| | | | ERATQLVTQ | | | AAAGCGGTCA |
| | | | AIANKKKLQ | | | GTGCCAATGA |
| | | | TANNALIAK | | | CATCCGCGAT |
| | | | APNALEKQK | | | GAAAAGAGTA |
| | | | ATYNADLLV | | | TCGTTGATGC |
| | | | DEIASLQARL | | | ACTCATGGAT |
| | | | DKLNAETAR | | | GCCAAAGCAA |
| | | | RKEIARQAAI | | | AATCGCTAAA |
| | | | RAANTYAM | | | GGCCATTGAG |
| | | | PANGSVVAT | | | GATCGCCCGG |
| | | | AAGRGLIQV | | | CCAATCTTTAT |
| | | | AQGAASLAQ | | | ACGGCTTCAG |
| | | | AISDAIAVLG | | | ACTTTCCTCAG |
| | | | RVLASAPSV | | | AAGTCAGAGT |
| | | | MAVGFASLT | | | CGATGTACCA |
| | | | YSSRTAEQW | | | GAGTCAGTTG |
| | | | QDQTPDSVR | | | CTGGCCAGCC |
| | | | YALGMDAA | | | GAAAATTCTA |
| | | | KLGLPPSVN | | | TGGAGAGTTC |
| | | | LNAVAKASG | | | CTGGATCGCC |
| | | | TVDLPMRLT | | | ATATGAGTGA |
| | | | NEARGNTTT | | | GCTGGCCAAA |
| | | | LSVVSTDGV | | | GCGTACAGCG |
| | | | SVPKAVPVR | | | CCGATATCTAT |
| | | | MAAYNATT | | | AAGGCGCAAA |
| | | | GLYEVTVPS | | | TCGCTATCTTG |
| | | | TTAEAPPLIL | | | AAACAAACGT |
| | | | TWTPASPPG | | | CTCAAGAGCT |
| | | | NQNPSSTTP | | | GGAGAATAAA |
| | | | VVPKPVPVY | | | GCCCGGTCAT |
| | | | EGATLTPVK | | | TGGAAGCAGA |
| | | | ATPETYPGVI | | | AGCCCAGCGA |
| | | | TLPEDLIIGFP | | | GCCGCTGCTG |
| | | | ADSGIKPIYV | | | AGGTGGAGGC |
| | | | MFRDPRDVP | | | GGACTACAAG |
| | | | GAATGKGQP | | | GCCAGGAAGG |
| | | | VSGNWLGA | | | CAAATGTCGA |
| | | | ASQGEGAPIP | | | GAAAAAAGTG |
| | | | SQIADKLRG | | | CAGTCCGAGC |
| | | | KTFKNWRDF | | | TTGACCAGGC |
| | | | REQFWIAVA | | | TGGGAATGCT |
| | | | NDPELSKQF | | | TTGCCTCAACT |
| | | | NPGSLAVMR | | | GACCAATCCA |
| | | | DGGAPYVRE | | | ACGCCAGAGC |
| | | | SEQAGGRIKI | | | AGTGGCTTGA |
| | | | EIHHKVRIA | | | ACGCGCTACT |
| | | | DGGGVYNM | | | CAACTGGTTA |
| | | | GNLVAVTPK | | | CGCAGGCGAT |
| | | | RHIEIHKGG | | | CGCCAATAAG |
| | | | K | | | AAGAAATTGC |
| | | | | | | AGACTGCAAA |
| | | | | | | CAATGCCTTG |
| | | | | | | ATTGCCAAGG |
| | | | | | | CACCCAATGC |
| | | | | | | ACTGGAGAAA |
| | | | | | | CAAAAGGCAA |
| | | | | | | CCTACAACGC |
| | | | | | | CGATCTCCTA |
| | | | | | | GTGGATGAAA |
| | | | | | | TCGCCAGCCT |
| | | | | | | GCAAGCACGG |
| | | | | | | CTGGACAAGC |
| | | | | | | TGAACGCCGA |
| | | | | | | AACGGCAAGG |
| | | | | | | CGCAAGGAAA |
| | | | | | | TCGCTCGTCA |
| | | | | | | AGCGGCGATC |
| | | | | | | AGGGCTGCCA |
| | | | | | | ATACTTATGCC |
| | | | | | | ATGCCAGCCA |
| | | | | | | ATGGCAGCGT |
| | | | | | | TGTCGCCACC |
| | | | | | | GCCGCAGGCC |
| | | | | | | GGGGTCTGAT |
| | | | | | | CCAGGTCGCA |
| | | | | | | CAAGGCGCCG |
| | | | | | | CATCCCTTGCT |
| | | | | | | CAAGCGATCT |
| | | | | | | CCGATGCGAT |
| | | | | | | TGCCGTCCTG |
| | | | | | | GGCCGGGTCC |
| | | | | | | TGGCTTCAGC |
| | | | | | | ACCCTCGGTG |
| | | | | | | ATGGCCGTGG |
| | | | | | | GCTTTGCCAG |
| | | | | | | TCTGACCTACT |
| | | | | | | CCTCCCGGAC |
| | | | | | | TGCCGAGCAA |
| | | | | | | TGGCAGGACC |
| | | | | | | AAACGCCCGA |
| | | | | | | TAGCGTTCGTT |
| | | | | | | ACGCCCTGGG |
| | | | | | | CATGGATGCC |
| | | | | | | GCTAAATTGG |
| | | | | | | GGCTTCCCCC |
| | | | | | | AAGCGTAAAC |
| | | | | | | CTGAACGCGG |
| | | | | | | TTGCAAAAGC |
| | | | | | | CAGCGGTACC |
| | | | | | | GTCGATCTGC |
| | | | | | | CGATGCGCCT |
| | | | | | | GACCAACGAG |
| | | | | | | GCACGAGGCA |
| | | | | | | ACACGACGAC |
| | | | | | | CCTTTCGGTG |
| | | | | | | GTCAGCACCG |
| | | | | | | ATGGTGTGAG |
| | | | | | | CGTTCCGAAA |
| | | | | | | GCCGTTCCGG |
| | | | | | | TCCGGATGGC |
| | | | | | | GGCCTACAAT |
| | | | | | | GCCACGACAG |
| | | | | | | GCCTGTACGA |
| | | | | | | GGTTACGGTT |
| | | | | | | CCCTCTACGA |
| | | | | | | CCGCAGAAGC |
| | | | | | | GCCGCCACTG |
| | | | | | | ATCCTGACCT |
| | | | | | | GGACGCCGGC |
| | | | | | | GAGTCCTCCA |
| | | | | | | GGAAACCAGA |
| | | | | | | ACCCTTCGAG |
| | | | | | | TACCACTCCG |
| | | | | | | GTCGTACCGA |
| | | | | | | AGCCGGTGCC |
| | | | | | | GGTATATGAG |
| | | | | | | GGAGCGACCC |
| | | | | | | TTACACCGGT |
| | | | | | | GAAGGCTACC |
| | | | | | | CCGGAAACCT |
| | | | | | | ATCCTGGGGT |
| | | | | | | GATTACACTA |
| | | | | | | CCGGAAGACC |
| | | | | | | TGATCATCGG |
| | | | | | | CTTCCCGGCC |
| | | | | | | GACTCGGGGA |
| | | | | | | TCAAGCCGAT |
| | | | | | | CTATGTGATG |
| | | | | | | TTCAGGGATC |
| | | | | | | CGCGGGATGT |
| | | | | | | ACCTGGTGCT |
| | | | | | | GCGACTGGCA |
| | | | | | | AGGGACAGCC |
| | | | | | | CGTCAGCGGT |
| | | | | | | AATTGGCTCG |
| | | | | | | GCGCCGCCTC |
| | | | | | | TCAAGGTGAG |
| | | | | | | GGGGCTCCAA |
| | | | | | | TTCCAAGCCA |
| | | | | | | GATTGCGGAT |
| | | | | | | AAACTACGTG |
| | | | | | | GTAAGACATT |
| | | | | | | CAAAAACTGG |
| | | | | | | CGGGACTTTC |
| | | | | | | GGGAACAATT |
| | | | | | | CTGGATAGCT |
| | | | | | | GTGGCTAATG |
| | | | | | | ATCCTGAGTT |
| | | | | | | AAGTAAACAG |
| | | | | | | TTTAATCCTGG |
| | | | | | | TAGTTTAGCT |
| | | | | | | GTAATGAGAG |
| | | | | | | ATGGAGGGGC |
| | | | | | | TCCTTATGTCA |
| | | | | | | GAGAGTCAGA |
| | | | | | | ACAGGCTGGC |
| | | | | | | GGGAGAATAA |
| | | | | | | AGATCGAAAT |
| | | | | | | CCACCACAAG |
| | | | | | | GTTCGAATAG |
| | | | | | | CAGATGGAGG |
| | | | | | | CGGCGTTTAC |
| | | | | | | AATATGGGGA |
| | | | | | | ACCTTGTTGC |
| | | | | | | AGTAACGCCA |
| | | | | | | AAACGTCATA |
| | | | | | | TAGAAATCCA |
| | | | | | | CAAGGGAGGG |
| | | | | | | AAGTGA |
| 278 | Ruminococcin- | Lantibiotic | MRNDVLTLT | Ruminococcus | 279 | ATGAGAAATG |
| | A | | NPMEEKELE | gnavus | | ACGTATTAAC |
| | | | QILGGGNGV | | | ATTAACAAAC |
| | | | LKTISHECN | | | CCAATGGAAG |
| | | | MNTWQFLFT | | | AGAACGAACT |
| | | | CC | | | GGAGCAGATC |
| | | | | | | TTAGGTGGTG |
| | | | | | | GCAATGGTGT |
| | | | | | | GTTAAAAACG |
| | | | | | | ATTAGCCACG |
| | | | | | | AATGCAATAT |
| | | | | | | GAACACATGG |
| | | | | | | CAGTTCCTGTT |
| | | | | | | TACTTGTTGCT |
| | | | | | | AA |
| 280 | Sakacin G | Class IIa | MKNAKSLTI | Lactobacillus | 281 | ATGAAAAACG |
| | | | QEMKSITGG | sakei | | CAAAAAGCCT |
| | | | KYYGNGVS | | | AACAATTCAA |
| | | | CNSHGCSVN | | | GAAATGAAAT |
| | | | WGQAWTCG | | | CTATTACAGG |
| | | | VNHLANGG | | | TGGTAAATAC |
| | | | HGVC | | | TATGGTAATG |
| | | | | | | GCGTTAGCTG |
| | | | | | | TAACTCTCAC |
| | | | | | | GGCTGTTCAG |
| | | | | | | TAAATTGGGG |
| | | | | | | GCAAGCATGG |
| | | | | | | ACTTGTGGAG |
| | | | | | | TAAACCATCT |
| | | | | | | AGCTAATGGC |
| | | | | | | GGTCATGGAG |
| | | | | | | TTTGTTAA |
| 282 | Sakacin-A | class | MNNVKELS | Lactobacillus | 283 | ATGAATAATG |
| | | IIA/YG | MTELQTITG | sakei | | TAAAAGAATT |
| | | NGV | GARSYGNG | | | AAGTATGACA |
| | | | VYCNNKKC | | | GAATTACAAA |
| | | | WVNRGEAT | | | CAATTACCGG |
| | | | QSIIGGMISG | | | CGGTGCTAGA |
| | | | WASGLAGM | | | TCATATGGCA |
| | | | | | | ACGGTGTTTA |
| | | | | | | CTGTAATAAT |
| | | | | | | AAAAAATGTT |
| | | | | | | GGGTAAATCG |
| | | | | | | GGGTGAAGCA |
| | | | | | | ACGCAAAGTA |
| | | | | | | TTATTGGTGG |
| | | | | | | TATGATTAGC |
| | | | | | | GGCTGGGCTA |
| | | | | | | GTGGTTTAGC |
| | | | | | | TGGAATGTAA |
| 284 | Sakacin-P | class | MEKFIELSLK | Lactobacillus | 285 | ATGGAAAAGT |
| | (Sakacin | IIA/YG | EVTAITGGK | sakei | | TTATTGAATTA |
| | 674) | NGV | YYGNGVHC | | | TCTTTAAAAG |
| | | | GKHSCTVD | | | AAGTAACAGC |
| | | | WGTAIGNIG | | | AATTACAGGT |
| | | | NNAAANWA | | | GGAAAATATT |
| | | | TGGNAGWN | | | ATGGTAACGG |
| | | | K | | | TGTACACTGT |
| | | | | | | GGAAAACATT |
| | | | | | | CATGTACCGT |
| | | | | | | AGACTGGGGA |
| | | | | | | ACAGCTATTG |
| | | | | | | GAAATATCGG |
| | | | | | | AAATAATGCA |
| | | | | | | GCTGCAAACT |
| | | | | | | GGGCCACAGG |
| | | | | | | CGGAAACGCT |
| | | | | | | GGCTGGAATA |
| | | | | | | AATAA |
| 286 | Salivaricin | lantibioti | MKSTNNQSI | Streptococcus | 287 | ATGAAATCAA |
| | 9 | c | AEIAAVNSL | salivarius | | CAAATAATCA |
| | | | QEVSMEELD | | | AAGTATCGCA |
| | | | QIIGAGNGV | | | GAAATTGCAG |
| | | | VLTLTHECN | | | CAGTAAACTC |
| | | | LATWTKKLK | | | ACTACAAGAA |
| | | | CC | | | GTAAGTATGG |
| | | | | | | AGGAACTAGA |
| | | | | | | CCAAATTATT |
| | | | | | | GGTGCCGGAA |
| | | | | | | ACGGAGTGGT |
| | | | | | | TCTTACTCTTA |
| | | | | | | CTCATGAATG |
| | | | | | | TAACCTAGCA |
| | | | | | | ACTTGGACAA |
| | | | | | | AAAAACTAAA |
| | | | | | | ATGTTGCTAA |
| 288 | Salivaricin | Lantibiotic | MSFMKNSK | Streptococcus | 289 | ATGAGTTTTAT |
| | A | | DILTNAIEEV | pyogenes | | GAAAAATTCA |
| | | | SEKELMEVA | serotype | | AAGGATATTT |
| | | | GGKKGSGW | M28 (strain | | TGACTAATGC |
| | | | FATITDDCPN | MGAS6180) | | TATCGAAGAA |
| | | | SVFVCC | | | GTTTCTGAAA |
| | | | | | | AAGAACTTAT |
| | | | | | | GGAAGTAGCT |
| | | | | | | GGTGGTAAAA |
| | | | | | | AAGGTTCCGG |
| | | | | | | TTGGTTTGCA |
| | | | | | | ACTATTACTG |
| | | | | | | ATGACTGTCC |
| | | | | | | GAACTCAGTA |
| | | | | | | TTCGTTTGTTG |
| | | | | | | TTAA |
| 290 | Salivaricin | Lantibiotic | MKNSKDVL | Streptococcus | 291 | ATGAAAAACT |
| | A3 | | NNAIEEVSE | salivarius | | CAAAAGATGT |
| | | | KELMEVAG | | | TTTGAACAAT |
| | | | GKKGPGWIA | | | GCTATCGAAG |
| | | | TITDDCPNSI | | | AGGTTTCTGA |
| | | | FVCC | | | AAAAGAACTT |
| | | | | | | ATGGAAGTAG |
| | | | | | | CTGGTGGTAA |
| | | | | | | AAAAGGTCCA |
| | | | | | | GGTTGGATTG |
| | | | | | | CAACTATTACT |
| | | | | | | GATGACTGTC |
| | | | | | | CAAACTCAAT |
| | | | | | | ATTCGTTTGTT |
| | | | | | | GTTAA |
| 292 | Salivaricin | Lantibiotic | MKNSKDILN | Streptococcus | 293 | ATGAAAAACT |
| | -A sa | | NAIEEVSEKE | salivarius | | CAAAAGATAT |
| | | | LMEVAGGK | | | TTTGAACAAT |
| | | | RGSGWIATIT | | | GCTATCGAAG |
| | | | DDCPNSVFV | | | AAGTTTCTGA |
| | | | CC | | | AAAAGAACTT |
| | | | | | | ATGGAAGTAG |
| | | | | | | CTGGTGGTAA |
| | | | | | | AAGAGGTTCA |
| | | | | | | GGTTGGATTG |
| | | | | | | CAACTATTACT |
| | | | | | | GATGACTGTC |
| | | | | | | CAAACTCAGT |
| | | | | | | ATTCGTTTGTT |
| | | | | | | GTTAA |
| 294 | Staphylococcin | Lantibiotic | MKSSFLEKDI | Staphylococcus | 295 | ATGAAAAGTT |
| | C55 | (two- | EEQVTWFEE | aureus | | CTTTTTTAGAA |
| | alpha | peptide) | VSEQEFDDD | | | AAAGATATAG |
| | | | IFGACSTNTF | | | AAGAACAAGT |
| | | | SLSDYWGNK | | | GACATGGTTC |
| | | | GNWCTATH | | | GAGGAAGTTT |
| | | | ECMSWCK | | | CAGAACAAGA |
| | | | | | | ATTTGACGAT |
| | | | | | | GATATTTTTGG |
| | | | | | | AGCTTGTAGT |
| | | | | | | ACAAACACTT |
| | | | | | | TTTCTTTGAGT |
| | | | | | | GACTATTGGG |
| | | | | | | GTAATAAAGG |
| | | | | | | AAATTGGTGT |
| | | | | | | ACTGCTACTC |
| | | | | | | ACGAATGTAT |
| | | | | | | GTCTTGGTGT |
| | | | | | | AAATAA |
| 296 | Staphylococcin | Lantibiotic | MKNELGKFL | Staphylococcus | 297 | ATGAAAAATG |
| | C55 | (two- | EENELELGK | aureus | | AATTAGGTAA |
| | beta | peptide) | FSESDMLEIT | | | GTTTTTAGAA |
| | | | DDEVYAAG | | | GAAAACGAAT |
| | | | TPLALLGGA | | | TAGAGTTAGG |
| | | | ATGVIGYISN | | | TAAATTTTCAG |
| | | | QTCPTTACT | | | AATCAGACAT |
| | | | RAC | | | GCTAGAAATT |
| | | | | | | ACTGATGATG |
| | | | | | | AAGTATATGC |
| | | | | | | AGCTGGAACA |
| | | | | | | CCTTTAGCCTT |
| | | | | | | ATTGGGTGGA |
| | | | | | | GCTGCCACCG |
| | | | | | | GGGTGATAGG |
| | | | | | | TTATATTTCTA |
| | | | | | | ACCAAACATG |
| | | | | | | TCCAACAACT |
| | | | | | | GCTTGTACAC |
| | | | | | | GCGCTTGCTA |
| | | | | | | G |
| 298 | Streptin | lantibioti | MNNTIKDFD | Streptococcus | 299 | ATGAATAACA |
| | | c | LDLKTNKKD | pyogenes | | CAATTAAAGA |
| | | | TATPYVGSR | | | CTTTGATCTCG |
| | | | YLCTPGSCW | | | ATTTGAAAAC |
| | | | KLVCFTTTV | | | AAATAAAAAA |
| | | | K | | | GACACTGCTA |
| | | | | | | CACCTTATGTT |
| | | | | | | GGTAGCCGTT |
| | | | | | | ACCTATGTAC |
| | | | | | | CCCTGGTTCTT |
| | | | | | | GTTGGAAATT |
| | | | | | | AGTTTGCTTTA |
| | | | | | | CAACAACTGT |
| | | | | | | TAAATAA |
| 300 | Streptococcin | Lantibiotic | MEKNNEVIN | Streptococcus | 301 | ATGGAAAAAA |
| | A- | | SIQEVSLEEL | pyogenes | | ATAATGAAGT |
| | FF22 | | DQIIGAGKN | | | AATCAACTCT |
| | | | GVFKTISHEC | | | ATTCAAGAAG |
| | | | HLNTWAFLA | | | TTAGTCTTGA |
| | | | TCCS | | | AGAACTCGAT |
| | | | | | | CAAATTATCG |
| | | | | | | GTGCTGGAAA |
| | | | | | | AAATGGTGTG |
| | | | | | | TTTAAAACAA |
| | | | | | | TTTCTCATGAG |
| | | | | | | TGTCATTTGA |
| | | | | | | ATACATGGGC |
| | | | | | | ATTCCTTGCTA |
| | | | | | | CTTGTTGTTCA |
| | | | | | | TAA |
| 302 | Streptococcin | Lantibiotic | MTKEHEIINS | Streptococcus | 303 | ATGGAAAAAA |
| | A- | | IQEVSLEELD | pyogenes | | ATAATGAAGT |
| | M49 | | QIIGAGKNG | serotype | | AATCAACTCT |
| | | | VFKTISHECH | M49 | | ATTCAAGAAG |
| | | | LNTWAFLAT | | | TTAGTCTTGA |
| | | | CCS | | | AGAACTCGAT |
| | | | | | | CAAATTATCG |
| | | | | | | GTGCTGGAAA |
| | | | | | | AAATGGTGTG |
| | | | | | | TTTAAAACAA |
| | | | | | | TTTCTCATGAG |
| | | | | | | TGTCATTTGA |
| | | | | | | ATACATGGGC |
| | | | | | | ATTCCTTGCTA |
| | | | | | | CTTGTTGCTCA |
| | | | | | | TAA |
| 304 | Sublancin | Lantibiotic | MEKLFKEVK | Bacillus | 305 | ATGGAAAAGC |
| | 168 | | LEELENQKG | subtilis | | TATTTAAAGA |
| | | | SGLGKAQCA | (strain 168) | | AGTTAAACTA |
| | | | ALWLQCASG | | | GAGGAACTCG |
| | | | GTIGCGGGA | | | AAAACCAAAA |
| | | | VACQNYRQF | | | AGGTAGTGGA |
| | | | CR | | | TTAGGAAAAG |
| | | | | | | CTCAGTGTGC |
| | | | | | | TGCGTTGTGG |
| | | | | | | CTACAATGTG |
| | | | | | | CTAGTGGCGG |
| | | | | | | TACAATTGGT |
| | | | | | | TGTGGTGGCG |
| | | | | | | GAGCTGTTGC |
| | | | | | | TTGTCAAAAC |
| | | | | | | TATCGTCAATT |
| | | | | | | CTGCAGATAA |
| 306 | Subtilin | Lantibiotic | MSKFDDFDL | Bacillus | 307 | ATGTCAAAGT |
| | | | DVVKVSKQ | subtilis | | TCGATGATTTC |
| | | | DSKITPQWK | | | GATTTGGATG |
| | | | SESLCTPGC | | | TTGTGAAAGT |
| | | | VTGALQTCF | | | CTCTAAACAA |
| | | | LQTLTCNCK | | | GACTCAAAAA |
| | | | ISK | | | TCACTCCGCA |
| | | | | | | ATGGAAAAGT |
| | | | | | | GAATCACTTT |
| | | | | | | GTACACCAGG |
| | | | | | | ATGTGTAACT |
| | | | | | | GGTGCATTGC |
| | | | | | | AAACTTGCTTC |
| | | | | | | CTTCAAACAC |
| | | | | | | TAACTTGTAA |
| | | | | | | CTGCAAAATC |
| | | | | | | TCTAAATAA |
| 308 | Subtilosin | Unclassified | MKLPVQQV | Bacillus | 309 | TTGAAATTGC |
| | | | YSVYGGKDL | subtilis | | CGGTGCAACA |
| | | | PKGHSHSTM | (strain 168) | | GGTCTATTCG |
| | | | PFLSKLQFLT | | | GTCTATGGGG |
| | | | KIYLLDIHTQ | | | GTAAGGATCT |
| | | | PFFI | | | CCCAAAAGGG |
| | | | | | | CATAGTCATTC |
| | | | | | | TACTATGCCCT |
| | | | | | | TTTTAAGTAA |
| | | | | | | ATTACAATTTT |
| | | | | | | TAACTAAAAT |
| | | | | | | CTACCTCTTGG |
| | | | | | | ATATACATAC |
| | | | | | | ACAACCGTTTT |
| | | | | | | TCATTTGA |
| 310 | Subtilosin | Unclassified | MKKAVIVEN | Bacillus | 311 | ATGAAAAAAG |
| | -A | | KGCATCSIG | subtilis | | CTGTCATTGTA |
| | | | AACLVDGPI | (strain 168) | | GAAAACAAAG |
| | | | PDFEIAGAT | | | GTTGTGCAAC |
| | | | GLFGLWG | | | ATGCTCGATC |
| | | | | | | GGAGCCGCTT |
| | | | | | | GTCTAGTGGA |
| | | | | | | CGGTCCTATC |
| | | | | | | CCTGATTTTGA |
| | | | | | | AATTGCCGGT |
| | | | | | | GCAACAGGTC |
| | | | | | | TATTCGGTCTA |
| | | | | | | TGGGGGTAA |
| 312 | Thermophilin | Lantibiotic | MMNATENQI | Streptococcus | 313 | ATGATGAATG |
| | 1277 | | FVETVSDQE | thermophilus | | CTACTGAAAA |
| | | | LEMLIGGAD | | | CCAAATTTTTG |
| | | | RGWIKTLTK | | | TTGAGACTGT |
| | | | DCPNVISSIC | | | GAGTGACCAA |
| | | | AGTIITACKN | | | GAATTAGAAA |
| | | | CA | | | TGTTAATTGGT |
| | | | | | | GGTGCAGATC |
| | | | | | | GTGGATGGAT |
| | | | | | | TAAGACTTTA |
| | | | | | | ACAAAAGATT |
| | | | | | | GTCCAAATGT |
| | | | | | | AATTTCTTCAA |
| | | | | | | TTTGTGCAGG |
| | | | | | | TACAATTATTA |
| | | | | | | CAGCCTGTAA |
| | | | | | | AAATTGTGCT |
| | | | | | | TAA |
| 314 | Thermophilin | Unclassified | MKQYNGFE | Streptococcus | 315 | ATGAAGCAGT |
| | 13 | | VLHELDLAN | thermophilus | | ATAATGGTTTT |
| | | | VTGGQINWG | | | GAGGTTCTAC |
| | | | SVVGHCIGG | | | ATGAACTTGA |
| | | | AIIGGAFSGG | | | CTTAGCAAAT |
| | | | AAAGVGCL | | | GTAACTGGCG |
| | | | VGSGKAIIN | | | GTCAAATTAA |
| | | | GL | | | TTGGGGATCA |
| | | | | | | GTTGTAGGAC |
| | | | | | | ACTGTATAGG |
| | | | | | | TGGAGCTATT |
| | | | | | | ATCGGAGGTG |
| | | | | | | CATTTTCAGG |
| | | | | | | AGGTGCAGCG |
| | | | | | | GCTGGAGTAG |
| | | | | | | GATGCCTTGTT |
| | | | | | | GGGAGCGGAA |
| | | | | | | AGGCAATCAT |
| | | | | | | AAATGGATTA |
| | | | | | | TAA |
| 316 | Thermophilin | Unclassified | MNTITICKFD | Streptococcus | 317 | ATGAATACAA |
| | A | | VLDAELLST | thermophilus | | TAACTATTTGT |
| | | | VEGGYSGKD | | | AAATTTGATG |
| | | | CLKDMGGY | | | TTTTAGATGCT |
| | | | ALAGAGSGA | | | GAACTTCTTTC |
| | | | LWGAPAGG | | | GACAGTTGAG |
| | | | VGALPGAFV | | | GGTGGATACT |
| | | | GAHVGAIAG | | | CTGGTAAGGA |
| | | | GFACMGGMI | | | TTGTTTAAAA |
| | | | GNKFN | | | GACATGGGAG |
| | | | | | | GATATGCATT |
| | | | | | | GGCAGGAGCT |
| | | | | | | GGAAGTGGAG |
| | | | | | | CTCTGTGGGG |
| | | | | | | AGCTCCAGCA |
| | | | | | | GGAGGTGTTG |
| | | | | | | GAGCACTTCC |
| | | | | | | AGGTGCATTT |
| | | | | | | GTCGGAGCTC |
| | | | | | | ATGTTGGGGC |
| | | | | | | AATTGCAGGA |
| | | | | | | GGCTTTGCAT |
| | | | | | | GTATGGGTGG |
| | | | | | | AATGATTGGT |
| | | | | | | AATAAGTTTA |
| | | | | | | ACTAA |
| 318 | Thiocillin | Unclassified | MSEIKKALN | Bacillus | 319 | ATGAGTGAAA |
| | (Micrococcin | | TLEIEDFDAI | cereus (strain | | TTAAAAAAGC |
| | P1) | | EMVDVDAM | ATCC 14579 | | ATTAAATACG |
| | (Micrococcin | | PENEALEIM | /DSM31) | | CTTGAAATTG |
| | P2) | | GASCTTCVC | | | AAGATTTTGA |
| | (Thiocillin | | TCSCCTT | | | TGCAATTGAA |
| | I) | | | | | ATGGTTGATG |
| | (Thiocillin | | | | | TTGATGCTAT |
| | II) | | | | | GCCAGAAAAC |
| | (Thiocillin | | | | | GAAGCGCTTG |
| | III) | | | | | AAATTATGGG |
| | (Thiocillin | | | | | AGCGTCATGT |
| | IV) | | | | | ACGACATGCG |
| | (Antibiotic | | | | | TATGTACATG |
| | YM- | | | | | CAGTTGTTGT |
| | 266183) | | | | | ACAACTTGA |
| | (Antibiotic | | | | | |
| | YM- | | | | | |
| | 266184) | | | | | |
| 320 | Thuricin | two- | MEVMNNALI | Bacillus | 321 | ATGGAAGTTA |
| | CD alpha | peptide | TKVDEEIGG | cereus | | TGAACAATGC |
| | | lantibiotic | NAACVIGCI | 95/8201 | | TTTAATTACAA |
| | | | GSCVISEGIG | | | AAGTAGATGA |
| | | | SLVGTAFTL | | | GGAGATTGGA |
| | | | G | | | GGAAACGCTG |
| | | | | | | CTTGTGTAATT |
| | | | | | | GGTTGTATTG |
| | | | | | | GCAGTTGCGT |
| | | | | | | AATTAGTGAA |
| | | | | | | GGAATTGGTT |
| | | | | | | CACTTGTAGG |
| | | | | | | AACAGCATTT |
| | | | | | | ACTTTAGGTT |
| | | | | | | AA |
| 322 | Thuricin | two- | MEVLNKQN | Bacillus | 323 | ATGGAAGTTT |
| | CD beta | peptide | VNIIPESEEV | cereus | | TAAACAAACA |
| | | lantibiotic | GGWVACVG | 95/8201 | | AAATGTAAAT |
| | | | ACGTVCLAS | | | ATTATTCCAG |
| | | | GGVGTEFAA | | | AATCTGAAGA |
| | | | ASYFL | | | AGTAGGTGGA |
| | | | | | | TGGGTAGCAT |
| | | | | | | GTGTTGGAGC |
| | | | | | | ATGTGGTACA |
| | | | | | | GTATGTCTTGC |
| | | | | | | TAGTGGTGGT |
| | | | | | | GTTGGAACAG |
| | | | | | | AGTTTGCAGC |
| | | | | | | TGCATCTTATT |
| | | | | | | TCCTATAA |
| 324 | Thuricin- | class IId | METPVVQPR | Bacillus | 325 | ATGGAAACAC |
| | 17 | | DWTCWSCL | thuringiensis | | CAGTAGTACA |
| | | | VCAACSVEL | | | ACCAAGGGAT |
| | | | LNLVTAATG | | | TGGACTTGTT |
| | | | ASTAS | | | GGAGTTGCTT |
| | | | | | | AGTATGTGCA |
| | | | | | | GCATGTTCTGT |
| | | | | | | GGAATTATTA |
| | | | | | | AATTTAGTTAC |
| | | | | | | TGCGGCAACA |
| | | | | | | GGGGCTAGTA |
| | | | | | | CTGCAAGCTA |
| | | | | | | A |
| 326 | Trifolitoxin | Unclassified | MDNKVAKN | Rhizobium | 327 | ATGGATAACA |
| | | tied | VEVKKGSIK | leguminosarum | | AGGTTGCGAA |
| | | | ATFKAAVLK | bv. trifolii | | GAATGTCGAA |
| | | | SKTKVDIGG | | | GTGAAGAAGG |
| | | | SRQGCVA | | | GCTCCATCAA |
| | | | | | | GGCGACCTTC |
| | | | | | | AAGGCTGCTG |
| | | | | | | TTCTGAAGTC |
| | | | | | | GAAGACGAAG |
| | | | | | | GTCGACATCG |
| | | | | | | GAGGTAGCCG |
| | | | | | | TCAGGGCTGC |
| | | | | | | GTCGCTTAA |
| 328 | Ubericin | Class IIa | MNTIEKFENI | Streptococcus | 329 | ATGAATACAA |
| | A | | KLFSLKKIIG | uberis | | TTGAAAAATT |
| | | | GKTVNYGN | | | TGAAAATATT |
| | | | GLYCNQKKC | | | AAACTTTTTTC |
| | | | WVNWSETA | | | ACTAAAGAAA |
| | | | TTIVNNSIM | | | ATTATCGGTG |
| | | | NGLTGGNA | | | GCAAAACTGT |
| | | | GWHSGGRA | | | AAATTATGGT |
| | | | | | | AATGGCCTTT |
| | | | | | | ATTGTAACCA |
| | | | | | | AAAAAAATGC |
| | | | | | | TGGGTAAACT |
| | | | | | | GGTCAGAAAC |
| | | | | | | TGCTACAACA |
| | | | | | | ATAGTAAATA |
| | | | | | | ATTCCATCATG |
| | | | | | | AACGGGCTCA |
| | | | | | | CAGGTGGTAA |
| | | | | | | TGCGGGTTGG |
| | | | | | | CACTCAGGCG |
| | | | | | | GGAGAGCATA |
| | | | | | | A |
| 330 | Uberolysin | Unclassified | MDILLELAG | Streptococcus | 331 | ATGGACATTT |
| | | | YTGIASGTA | uberis | | TATTAGAACT |
| | | | KKVVDAIDK | | | CGCAGGATAT |
| | | | GAAAFVIISII | | | ACTGGGATAG |
| | | | STVISAGAL | | | CCTCAGGTAC |
| | | | GAVSASADF | | | TGCAAAAAAA |
| | | | IILTVKNYIS | | | GTTGTTGATG |
| | | | RNLKAQAVI | | | CCATTGATAA |
| | | | W | | | AGGAGCTGCA |
| | | | | | | GCCTTTGTTAT |
| | | | | | | TATTTCAATTA |
| | | | | | | TCTCAACAGT |
| | | | | | | AATTAGTGCG |
| | | | | | | GGAGCATTGG |
| | | | | | | GAGCAGTTTC |
| | | | | | | AGCCTCAGCT |
| | | | | | | GATTTTATTAT |
| | | | | | | TTTAACTGTAA |
| | | | | | | AAAATTACAT |
| | | | | | | TAGTAGAAAT |
| | | | | | | TTAAAAGCAC |
| | | | | | | AAGCTGTCAT |
| | | | | | | TTGGTAA |
| 332 | UviB | Unclassified | MDSELFKLM | Clostridium | 333 | ATGGATAGTG |
| | | | ATQGAFAILF | perfringens | | AATTATTTAA |
| | | | SYLLFYVLK | | | GTTAATGGCA |
| | | | ENSKREDKY | | | ACACAAGGAG |
| | | | QNIIEELTEL | | | CCTTTGCAATA |
| | | | LPKIKEDVE | | | TTATTTTCGTA |
| | | | DIKEKLNK | | | TTTATTGTTTT |
| | | | | | | ATGTTTTAAA |
| | | | | | | AGAGAATAGT |
| | | | | | | AAAAGAGAAG |
| | | | | | | ATAAGTATCA |
| | | | | | | AAATATAATA |
| | | | | | | GAGGAGCTTA |
| | | | | | | CAGAATTATT |
| | | | | | | GCCAAAAATA |
| | | | | | | AAAGAAGATG |
| | | | | | | TAGAAGATAT |
| | | | | | | AAAAGAAAAA |
| | | | | | | CTTAATAAAT |
| | | | | | | AG |
| 334 | Variacin | Lantibiotic, | MTNAFQALD | Micrococcus | 335 | ATGACGAACG |
| | | Type | EVTDAELDA | varians | | CATTTCAGGC |
| | | A | ILGGGSGVIP | | | ACTGGACGAA |
| | | | TISHECHMN | | | GTCACGGACG |
| | | | SFQFVFTCCS | | | CCGAGCTCGA |
| | | | | | | CGCCATCCTT |
| | | | | | | GGCGGGGGCA |
| | | | | | | GTGGTGTTAT |
| | | | | | | TCCCACGATC |
| | | | | | | AGCCACGAGT |
| | | | | | | GCCACATGAA |
| | | | | | | CTCCTTCCAGT |
| | | | | | | TCGTGTTCACC |
| | | | | | | TGCTGCTCCTG |
| | | | | | | A |
| 336 | Zoocin A | Unclassified | MKRIFFAFLS | Streptococcus | 337 | ATGAAACGTA |
| | | | LCLFIFGTQT | equi subsp. | | TATTTTTTGCT |
| | | | VSAATYTRP | zooepidemicus | | TTCTTAAGTTT |
| | | | LDTGNITTGF | | | ATGCTTATTTA |
| | | | NGYPGHVG | | | TATTCGGAAC |
| | | | VDYAVPVGT | | | ACAAACGGTA |
| | | | PVRAVANGT | | | TCTGCAGCTA |
| | | | VKFAGNGA | | | CTTATACTCG |
| | | | NHPWMLWM | | | GCCATTAGAT |
| | | | AGNCVLIQH | | | ACGGGAAATA |
| | | | ADGMHTGY | | | TCACTACAGG |
| | | | AHLSKISVST | | | GTTTAACGGA |
| | | | DSTVKQGQII | | | TACCCTGGTC |
| | | | GYTGATGQ | | | ATGTTGGAGT |
| | | | VTGPHLHFE | | | CGATTATGCA |
| | | | MLPANPNW | | | GTACCCGTTG |
| | | | QNGFSGRID | | | GAACTCCGGT |
| | | | PTGYIANAP | | | TAGAGCAGTT |
| | | | VFNGTTPTE | | | GCAAATGGTA |
| | | | pttpttnlki | | | CAGTCAAATT |
| | | | YKVDDLQKI | | | TGCAGGTAAT |
| | | | NGIWQVRN | | | GGGGCTAATC |
| | | | NILVPTDFT | | | ACCCATGGAT |
| | | | WVDNGIAA | | | GCTTTGGATG |
| | | | DDVIEVTSN | | | GCTGGAAACT |
| | | | GTRTSDQVL | | | GTGTTCTAATT |
| | | | QKGGYFVIN | | | CAACATGCTG |
| | | | PNNVKSVGT | | | ACGGGATGCA |
| | | | PMKGSGGLS | | | TACTGGATAT |
| | | | WAQVNFTT | | | GCACACTTAT |
| | | | GGNVWLNT | | | CAAAAATTTC |
| | | | TSKDNLLYG | | | AGTTAGCACA |
| | | | K | | | GATAGTACAG |
| | | | | | | TTAAACAAGG |
| | | | | | | ACAAATCATA |
| | | | | | | GGTTATACTG |
| | | | | | | GTGCCACCGG |
| | | | | | | CCAAGTTACC |
| | | | | | | GGTCCACATT |
| | | | | | | TGCATTTTGA |
| | | | | | | AATGTTGCCA |
| | | | | | | GCAAATCCTA |
| | | | | | | ACTGGCAAAA |
| | | | | | | TGGTTTTTCTG |
| | | | | | | GAAGAATAGA |
| | | | | | | TCCAACCGGA |
| | | | | | | TACATCGCTA |
| | | | | | | ATGCCCCTGT |
| | | | | | | ATTTAATGGA |
| | | | | | | ACAACACCTA |
| | | | | | | CAGAACCTAC |
| | | | | | | TACTCCTACA |
| | | | | | | ACAAATTTAA |
| | | | | | | AAATCTATAA |
| | | | | | | AGTTGATGAT |
| | | | | | | TTACAAAAAA |
| | | | | | | TTAATGGTATT |
| | | | | | | TGGCAAGTAA |
| | | | | | | GAAATAACAT |
| | | | | | | ACTTGTACCA |
| | | | | | | ACTGATTTCAC |
| | | | | | | ATGGGTTGAT |
| | | | | | | AATGGAATTG |
| | | | | | | CAGCAGATGA |
| | | | | | | TGTAATTGAA |
| | | | | | | GTAACTAGCA |
| | | | | | | ATGGAACAAG |
| | | | | | | AACCTCTGAC |
| | | | | | | CAAGTTCTTCA |
| | | | | | | AAAAGGTGGT |
| | | | | | | TATTTTGTCAT |
| | | | | | | CAATCCTAAT |
| | | | | | | AATGTTAAAA |
| | | | | | | GTGTTGGAAC |
| | | | | | | TCCGATGAAA |
| | | | | | | GGTAGTGGTG |
| | | | | | | GTCTATCTTGG |
| | | | | | | GCTCAAGTAA |
| | | | | | | ACTTTACAAC |
| | | | | | | AGGTGGAAAT |
| | | | | | | GTCTGGTTAA |
| | | | | | | ATACTACTAG |
| | | | | | | CAAAGACAAC |
| | | | | | | TTACTTTACGG |
| | | | | | | AAAATAA |
| 338 | Fulvocin- | Unclassified | ANCSCSTAS | Myxococcus | 339 | GCGAACTGCA |
| | C | | DYCPILTFCT | fulvus | | GCTGCAGCAC |
| | | | TGTACSYTP | | | CGCGAGCGAT |
| | | | TGCGTGWV | | | TATTGCCCGA |
| | | | YCACNGNFY | | | TTCTGACCTTT |
| | | | | | | TGCACCACCG |
| | | | | | | GCACCGCGTG |
| | | | | | | CAGCTATACC |
| | | | | | | CCGACCGGCT |
| | | | | | | GCGGCACCGG |
| | | | | | | CTGGGTGTAT |
| | | | | | | TGCGCGTGCA |
| | | | | | | ACGGCAACTT |
| | | | | | | TTAT |
| 340 | Duramycin- | Lantibiotic | CANSCSYGP | Streptomyces | 341 | TGCGCGAACA |
| | C | | LTWSCDGNT | griseoluteus | | GCTGCAGCTA |
| | | | K | | | TGGCCCGCTG |
| | | | | | | ACCTGGAGCT |
| | | | | | | GCGATGGCAA |
| | | | | | | CACCAAA |
| 342 | Duramycin | Lantibiotic | CKQSCSFGPF | Strepto- | 343 | TGCAAACAGA |
| | (duramycin-B) | B | TFVCDGNTK | verticiilium | | GCTGCAGCTT |
| | (Leucopeptin) | | | griseo | | TGGCCCGTTT |
| | | | | verticillatum | | ACCTTTGTGTG |
| | | | | | | CGATGGCAAC |
| | | | | | | ACCAAA |
| 344 | Carnocin | lantibioti | GSEIQPR | Carnobacterium | 345 | GGCAGCGAAA |
| | UI49 | c | | sp. (strain | | TTCAGCCGCG |
| | | | | UI49) | | C |
| 346 | Lactococc | Unclassified | GTWDDIGQG | Lactococcus | 347 | GGCACCTGGG |
| | in-G α | tied | IGRVAYWVG | lactis subsp. | | ATGATATTGG |
| | | | KAMGNMSD | lactis | | CCAGGGCATT |
| | | | VNQASRINR | (Streptococcus | | GGCCGCGTGG |
| | | | KKKH | lactis) | | CGTATTGGGT |
| | | | | | | GGGCAAAGCG |
| | | | | | | ATGGGCAACA |
| | | | | | | TGAGCGATGT |
| | | | | | | GAACCAGGCG |
| | | | | | | AGCCGCATTA |
| | | | | | | ACCGCAAAAA |
| | | | | | | AAAACAT |
| 348 | Lactococc | Unclassified | KKWGWLAW | Lactococcus | 349 | AAAAAATGGG |
| | in-G β | | VDPAYEFIK | lactis subsp. | | GCTGGCTGGC |
| | | | GFGKGAIKE | lactis | | GTGGGTGGAT |
| | | | GNKDKWKN | (Streptococcus | | CCGGCGTATG |
| | | | 1 | lactis) | | AATTTATTAA |
| | | | | | | AGGCTTTGGC |
| | | | | | | AAAGGCGCGA |
| | | | | | | TTAAAGAAGG |
| | | | | | | CAACAAAGAT |
| | | | | | | AAATGGAAAA |
| | | | | | | ACATT |
| 350 | Ancovenin | Lantibiotic | CVQSCSFGP | Streptomyces | 351 | TGCGTGCAGA |
| | | | LTWSCDGNT | sp. (strain | | GCTGCAGCTT |
| | | | K | A647P-2) | | TGGCCCGCTG |
| | | | | | | ACCTGGAGCT |
| | | | | | | GCGATGGCAA |
| | | | | | | CACCAAA |
| 352 | Actagardine | Lantibiotic | SSGWVCTLT | Actinoplanes | 353 | AGCAGCGGCT |
| | (Gardimycin) | | IECGTVICAC | liguriae | | GGGTGTGCAC |
| | | | | | | CCTGACCATT |
| | | | | | | GAATGCGGCA |
| | | | | | | CCGTGATTTG |
| | | | | | | CGCGTGC |
| 354 | Curvaticin | Unclassified | YTAKQCLQA | Lactobacillus | 355 | TATACCGCGA |
| | FS47 | | IGSCGIAGTG | curvatus | | AACAGTGCCT |
| | | | AGAAGGPA | | | GCAGGCGATT |
| | | | GAFVGAXV | | | GGCAGCTGCG |
| | | | VXI | | | GCATTGCGGG |
| | | | | | | CACCGGCGCG |
| | | | | | | GGCGCGGCGG |
| | | | | | | GCGGCCCGGC |
| | | | | | | GGGCGCGTTT |
| | | | | | | GTGGGCGCGN |
| | | | | | | NNGTGGTGNN |
| | | | | | | NATT [IN |
| | | | | | | WHICH NNN = |
| | | | | | | ANY AMINO- |
| | | | | | | ACID CODING |
| | | | | | | TRIPLET] |
| 356 | Bavaricin- | class | TKYYGNGV | Lactobacillus | 357 | ACCAAATATT |
| | MN | IIA/YG | YCNSKKCW | sakei | | ATGGCAACGG |
| | | NGV | VDWGQAAG | | | CGTGTATTGC |
| | | | GIGQTVVXG | | | AACAGCAAAA |
| | | | WLGGAIPGK | | | AATGCTGGGT |
| | | | | | | GGATTGGGGC |
| | | | | | | CAGGCGGCGG |
| | | | | | | GCGGCATTGG |
| | | | | | | CCAGACCGTG |
| | | | | | | GTGNNNGGCT |
| | | | | | | GGCTGGGCGG |
| | | | | | | CGCGATTCCG |
| | | | | | | GGCAAA[IN |
| | | | | | | WHICH NNN = |
| | | | | | | ANY AMINO- |
| | | | | | | ACID CODING |
| | | | | | | TRIPLET] |
| 358 | Mutacin | Lantibiotic | FKSWSFCTP | Streptococcus | 359 | TTTAAAAGCT |
| | B-Ny266 | | GCAKTGSFN | mutans | | GGAGCTTTTG |
| | | | SYCC | | | CACCCCGGGC |
| | | | | | | TGCGCGAAAA |
| | | | | | | CCGGCAGCTT |
| | | | | | | TAACAGCTAT |
| | | | | | | TGCTGCTTTAA |
| | | | | | | AAGCTGGAGC |
| | | | | | | TTTTGCACCCC |
| | | | | | | GGGCTGCGCG |
| | | | | | | AAAACCGGCA |
| | | | | | | GCTTTAACAG |
| | | | | | | CTATTGCTGC |
| 360 | Mundticin | class | KYYGNGVS | Enterococcus | 361 | AAATATTATG |
| | | IIA/YG | CNKKGCSVD | mundtii | | GCAACGGCGT |
| | | NGV | WGKAIGIIGN | | | GAGCTGCAAC |
| | | | NSAANLATG | | | AAAAAAGGCT |
| | | | GAAGWSK | | | GCAGCGTGGA |
| | | | | | | TTGGGGCAAA |
| | | | | | | GCGATTGGCA |
| | | | | | | TTATTGGCAA |
| | | | | | | CAACAGCGCG |
| | | | | | | GCGAACCTGG |
| | | | | | | CGACCGGCGG |
| | | | | | | CGCGGCGGGC |
| | | | | | | TGGAGCAAA |
| 362 | Bavaricin- | class | KYYGNGVH | Lactobacillus | 363 | AAATATTATG |
| | A | IIA/YG | XGKHSXTVD | sakei | | GCAACGGCGT |
| | | NGV | WGTAIGNIG | | | GCATNNNGGC |
| | | | NNAAANXA | | | AAACATAGCN |
| | | | TGXNAGG | | | NNACCGTGGA |
| | | | | | | TTGGGGCACC |
| | | | | | | GCGATTGGCA |
| | | | | | | ACATTGGCAA |
| | | | | | | CAACGCGGCG |
| | | | | | | GCGAACNNNG |
| | | | | | | CGACCGGCNN |
| | | | | | | NAACGCGGGC |
| | | | | | | GGC [IN |
| | | | | | | WHICH NNN = |
| | | | | | | ANY AMINO- |
| | | | | | | ACID CODING |
| | | | | | | TRIPLET] |
| 364 | Lactocin- | class IIb | GMSGYIQGI | Lactobacillus | 365 | GGCATGAGCG |
| | 705 | | PDFLKGYLH | paracasei | | GCTATATTCA |
| | | | GISAANKHK | | | GGGCATTCCG |
| | | | KGRL | | | GATTTTCTGA |
| | | | | | | AAGGCTATCT |
| | | | | | | GCATGGCATT |
| | | | | | | AGCGCGGCGA |
| | | | | | | ACAAACATAA |
| | | | | | | AAAAGGCCGC |
| | | | | | | CTG |
| 366 | Leucocin- | Unclassified | KGKGFWSW | Leuconostoc | 367 | AAAGGCAAAG |
| | B | | ASKATSWLT | mesenteroides | | GCTTTTGGAG |
| | | | GPQQPGSPL | | | CTGGGCGAGC |
| | | | LKKHR | | | AAAGCGACCA |
| | | | | | | GCTGGCTGAC |
| | | | | | | CGGCCCGCAG |
| | | | | | | CAGCCGGGCA |
| | | | | | | GCCCGCTGCT |
| | | | | | | GAAAAAACAT |
| | | | | | | CGC |
| 368 | Leucocin | class | KNYGNGVH | Leuconostoc | 369 | AAAAACTATG |
| | C | IIA/YG | CTKKGCSVD | mesenteroides | | GCAACGGCGT |
| | | NGV | WGYAWTNI | | | GCATTGCACC |
| | | | ANNSVMNG | | | AAAAAAGGCT |
| | | | LTGGNAGW | | | GCAGCGTGGA |
| | | | HN | | | TTGGGGCTAT |
| | | | | | | GCGTGGACCA |
| | | | | | | ACATTGCGAA |
| | | | | | | CAACAGCGTG |
| | | | | | | ATGAACGGCC |
| | | | | | | TGACCGGCGG |
| | | | | | | CAACGCGGGC |
| | | | | | | TGGCATAAC |
| 370 | LCI | Unclassified | AIKLVQSPN | Bacillus | 371 | GCGATTAAAC |
| | | | GNFAASFVL | subtilis | | TGGTGCAGAG |
| | | | DGTKWIFKS | | | CCCGAACGGC |
| | | | KYYDSSKGY | | | AACTTTGCGG |
| | | | WVGIYEVW | | | CGAGCTTTGT |
| | | | DRK | | | GCTGGATGGC |
| | | | | | | ACCAAATGGA |
| | | | | | | TTTTTAAAAGC |
| | | | | | | AAATATTATG |
| | | | | | | ATAGCAGCAA |
| | | | | | | AGGCTATTGG |
| | | | | | | GTGGGCATTT |
| | | | | | | ATGAAGTGTG |
| | | | | | | GGATCGCAAA |
| 372 | Lichenin | Unclassified | ISLEICXIFHD | Bacillus | 373 | ATTAGCCTGG |
| | | | N | licheniformis | | AAATTTGCNN |
| | | | | | | NATTTTTCATG |
| | | | | | | ATAAC [IN |
| | | | | | | WHICH NNN = |
| | | | | | | ANY AMINO- |
| | | | | | | ACID CODING |
| | | | | | | TRIPLET] |
| 374 | Lactococcin | class | TSYGNGVHC | Lactococcus | 375 | ACCAGCTATG |
| | MMFII | IIA/YG | NKSKCWIDV | lactis subsp. | | GCAACGGCGT |
| | | NGV | SELETYKAG | lactis | | GCATTGCAAC |
| | | | TVSNPKDIL | (Streptococcus | | AAAAGCAAAT |
| | | | W | lactis) | | GCTGGATTGA |
| | | | | | | TGTGAGCGAA |
| | | | | | | CTGGAAACCT |
| | | | | | | ATAAAGCGGG |
| | | | | | | CACCGTGAGC |
| | | | | | | AACCCGAAAG |
| | | | | | | ATATTCTGTG |
| | | | | | | G |
| 376 | Serracin-P | Phage- | DYHHGVRV | Serratia | 377 | GATTATCATC |
| | | Tail- | L | plymuthica | | ATGGCGTGCG |
| | | Like | | | | CGTGCTG |
| 378 | Halocin- | Unclassified | DIDITGCSAC | Halobacterium | 379 | GATATTGATA |
| | C8 | | KYAAG | sp. (strain | | TTACCGGCTG |
| | | | | AS7092) | | CAGCGCGTGC |
| | | | | | | AAATATGCGG |
| | | | | | | CGGGC |
| 380 | Subpeptin | Unclassified | XXKEIXHIFH | Bacillus | 381 | NNNNNNAAAG |
| | JM4-B | | DN | subtilis | | AAATTNNNCA |
| | | | | | | TATTTTTCATG |
| | | | | | | ATAAC [IN |
| | | | | | | WHICH NNN = |
| | | | | | | ANY AMINO- |
| | | | | | | ACID CODING |
| | | | | | | TRIPLET] |
| 382 | Curvalicin | Unclassified | TPVVNPPFL | Lactobacillus | 383 | ACCCCGGTGG |
| | -28a | | QQT | curvatus | | TGAACCCGCC |
| | | | | | | GTTTCTGCAG |
| | | | | | | CAGACC |
| 384 | Curvalicin | Unclassified | VAPFPEQFL | Lactobacillus | 385 | GTGGCGCCGT |
| | -28b | | X | curvatus | | TTCCGGAACA |
| | | | | | | GTTTCTGNNN |
| | | | | | | [IN WHICH |
| | | | | | | NNN = ANY |
| | | | | | | AMINO-ACID |
| | | | | | | CODING |
| | | | | | | TRIPLET] |
| 386 | Curvalicin | Unclassified | NIPQLTPTP | Lactobacillus | 387 | AACATTCCGC |
| | -28c | | | curvatus | | AGCTGACCCC |
| | | | | | | GACCCCG |
| 388 | Thuricin- | Unclassified | DWTXWSXL | Bacillus | 389 | GATTGGACCN |
| | S | | VXAACSVEL | thuringiensis | | NNTGGAGCNN |
| | | | L | subsp. | | NCTGGTGNNN |
| | | | | entomocidus | | GCGGCGTGCA |
| | | | | | | GCGTGGAACT |
| | | | | | | GCTG [IN |
| | | | | | | WHICH NNN = |
| | | | | | | ANY AMINO- |
| | | | | | | ACID CODING |
| | | | | | | TRIPLET] |
| 390 | Curvaticin | Unclassified | AYPGNGVH | Lactobacillus | 391 | GCGTATCCGG |
| | L442 | | CGKYSCTVD | curvatus | | GCAACGGCGT |
| | | | KQTAIGNIG | | | GCATTGCGGC |
| | | | NNAA | | | AAATATAGCT |
| | | | | | | GCACCGTGGA |
| | | | | | | TAAACAGACC |
| | | | | | | GCGATTGGCA |
| | | | | | | ACATTGGCAA |
| | | | | | | CAACGCGGCG |
| 392 | Divergicin | class | TKYYGNGV | Carnobacterium | 393 | ACCAAATATT |
| | M35 | IIa/YGN | YCNSKKCW | divergens | | ATGGCAACGG |
| | | GV | VDWGTAQG | (Lactobacillus | | CGTGTATTGC |
| | | | CIDVVIGQL | divergens) | | AACAGCAAAA |
| | | | GGGIPGKGK | | | AATGCTGGGT |
| | | | C | | | GGATTGGGGC |
| | | | | | | ACCGCGCAGG |
| | | | | | | GCTGCATTGA |
| | | | | | | TGTGGTGATT |
| | | | | | | GGCCAGCTGG |
| | | | | | | GCGGCGGCAT |
| | | | | | | TCCGGGCAAA |
| | | | | | | GGCAAATGC |
| 394 | Enterocin | class IIb | NRWYCNSA | Enterococcus | 395 | AACCGCTGGT |
| | E-760 | | AGGVGGAA | sp. | | ATTGCAACAG |
| | | | VCGLAGYV | | | CGCGGCGGGC |
| | | | GEAKENIAG | | | GGCGTGGGCG |
| | | | EVRKGWGM | | | GCGCGGCGGT |
| | | | AGGFTHNKA | | | GTGCGGCCTG |
| | | | CKSFPGSGW | | | GCGGGCTATG |
| | | | ASG | | | TGGGCGAAGC |
| | | | | | | GAAAGAAAAC |
| | | | | | | ATTGCGGGCG |
| | | | | | | AAGTGCGCAA |
| | | | | | | AGGCTGGGGC |
| | | | | | | ATGGCGGGCG |
| | | | | | | GCTTTACCCAT |
| | | | | | | AACAAAGCGT |
| | | | | | | GCAAAAGCTT |
| | | | | | | TCCGGGCAGC |
| | | | | | | GGCTGGGCGA |
| | | | | | | GCGGC |
| 396 | Bacteriocin | Unclassified | TTKNYGNG | Enterococcus | 397 | ACCACCAAAA |
| | E50-52 | | VCNSVNWC | faecium | | ACTATGGCAA |
| | | | QCGNVWAS | (Streptococcus | | CGGCGTGTGC |
| | | | CNLATGCAA | faecium) | | AACAGCGTGA |
| | | | WLCKLA | | | ACTGGTGCCA |
| | | | | | | GTGCGGCAAC |
| | | | | | | GTGTGGGCGA |
| | | | | | | GCTGCAACCT |
| | | | | | | GGCGACCGGC |
| | | | | | | TGCGCGGCGT |
| | | | | | | GGCTGTGCAA |
| | | | | | | ACTGGCG |
| 398 | Paenibacillin | Unclassified | ASIIKTTIKVS | Paenibacillus | 399 | GCGAGCATTA |
| | | | KAVCKTLTC | polymyxa | | TTAAAACCAC |
| | | | ICTGSCSNCK | (Bacillus | | CATTAAAGTG |
| | | | | polymyxa) | | AGCAAAGCGG |
| | | | | | | TGTGCAAAAC |
| | | | | | | CCTGACCTGC |
| | | | | | | ATTTGCACCG |
| | | | | | | GCAGCTGCAG |
| | | | | | | CAACTGCAAA |
| 400 | Epilancin | Unclassified | SASIVKTTIK | Staphylococcus | 401 | AGCGCGAGCA |
| | 15x | | ASKKLCRGF | epidermidis | | TTGTGAAAAC |
| | | | TLTCGCHFT | | | CACCATTAAA |
| | | | GKK | | | GCGAGCAAAA |
| | | | | | | AACTGTGCCG |
| | | | | | | CGGCTTTACC |
| | | | | | | CTGACCTGCG |
| | | | | | | GCTGCCATTTT |
| | | | | | | ACCGGCAAAA |
| | | | | | | AA |
| 402 | Enterocin | class IIa | KYYGNGVS | Enterococcus | 403 | AAATATTATG |
| | -HF | | CNKKGCSVD | faecium | | GCAACGGCGT |
| | | | WGKAIGIIGN | (Streptococcus | | GAGCTGCAAC |
| | | | NAAANLTTG | faecium) | | AAAAAAGGCT |
| | | | GKAAWAC | | | GCAGCGTGGA |
| | | | | | | TTGGGGCAAA |
| | | | | | | GCGATTGGCA |
| | | | | | | TTATTGGCAA |
| | | | | | | CAACGCGGCG |
| | | | | | | GCGAACCTGA |
| | | | | | | CCACCGGCGG |
| | | | | | | CAAAGCGGCG |
| | | | | | | TGGGCGTGC |
| 404 | Bacillocin | Class IIa | ATYYGNGL | Paenibacillus | 405 | GCGACCTATT |
| | 602 | | YCNKQKHY | polymyxa | | ATGGCAACGG |
| | | | TWVDWNKA | (Bacillus | | CCTGTATTGC |
| | | | SREIGKITVN | polymyxa) | | AACAAACAGA |
| | | | GWVQH | | | AACATTATAC |
| | | | | | | CTGGGTGGAT |
| | | | | | | TGGAACAAAG |
| | | | | | | CGAGCCGCGA |
| | | | | | | AATTGGCAAA |
| | | | | | | ATTACCGTGA |
| | | | | | | ACGGCTGGGT |
| | | | | | | GCAGCAT |
| 406 | Bacillocin | Class IIa | VNYGNGVS | Bacillus | 407 | GTGAACTATG |
| | 1580 | | CSKTKCSVN | circulans | | GCAACGGCGT |
| | | | WGIITHQAF | | | GAGCTGCAGC |
| | | | RVTSGVASG | | | AAAACCAAAT |
| | | | | | | GCAGCGTGAA |
| | | | | | | CTGGGGCATT |
| | | | | | | ATTACCCATC |
| | | | | | | AGGCGTTTCG |
| | | | | | | CGTGACCAGC |
| | | | | | | GGCGTGGCGA |
| | | | | | | GCGGC |
| 408 | Bacillocin | Unclassified | FVYGNGVTS | Paenibacillus | 409 | TTTGTGTATG |
| | B37 | | ILVQAQFLV | polymyxa | | GCAACGGCGT |
| | | | NGQRRFFYT | (Bacillus | | GACCAGCATT |
| | | | PDK | polymyxa) | | CTGGTGCAGG |
| | | | | | | CGCAGTTTCT |
| | | | | | | GGTGAACGGC |
| | | | | | | CAGCGCCGCT |
| | | | | | | TTTTTTATACC |
| | | | | | | CCGGATAAA |
| 410 | Rhamnosin | Unclassified | AVPAVRKTN | Lactobacillus | 411 | GCGGTGCCGG |
| | A | | ETLD | rhamnosus | | CGGTGCGCAA |
| | | | | | | AACCAACGAA |
| | | | | | | ACCCTGGAT |
| 412 | Lichenicidin | Lantibiotic | MKNSAARE | Bacillus | 413 | ATGAAAAACA |
| | A2 | (two- | AFKGANHPA | licheniformis | | GCGCGGCGCG |
| | | peptide) | GMVSEEELK | (strain DSM | | CGAAGCGTTT |
| | | | ALVGGNDV | 13/ATCC | | AAAGGCGCGA |
| | | | NPETTPATTS | 14580) | | ACCATCCGGC |
| | | | SWTCITAGV | | | GGGCATGGTG |
| | | | TVSASLCPTT | | | AGCGAAGAAG |
| | | | KCTSRC | | | AACTGAAAGC |
| | | | | | | GCTGGTGGGC |
| | | | | | | GGCAACGATG |
| | | | | | | TGAACCCGGA |
| | | | | | | AACCACCCCG |
| | | | | | | GCGACCACCA |
| | | | | | | GCAGCTGGAC |
| | | | | | | CTGCATTACC |
| | | | | | | GCGGGCGTGA |
| | | | | | | CCGTGAGCGC |
| | | | | | | GAGCCTGTGC |
| | | | | | | CCGACCACCA |
| | | | | | | AATGCACCAG |
| | | | | | | CCGCTGC |
| 414 | Plantaricin | Class IIa | KYYGNGLSC | Lactobacillus | 415 | AAATATTATG |
| | C19 | | SKKGCTVN | plantarum | | GCAACGGCCT |
| | | | WGQAFSCG | | | GAGCTGCAGC |
| | | | VNRVATAG | | | AAAAAAGGCT |
| | | | HGK | | | GCACCGTGAA |
| | | | | | | CTGGGGCCAG |
| | | | | | | GCGTTTAGCT |
| | | | | | | GCGGCGTGAA |
| | | | | | | CCGCGTGGCG |
| | | | | | | ACCGCGGGCC |
| | | | | | | ATGGCAAA |
| 416 | Acidocin | class IIb | GNPKVAHCA | Lactobacillus | 417 | GGCAACCCGA |
| | J1132 β | | SQIGRSTAW | acidophilus | | AAGTGGCGCA |
| | | | GAVSGA | | | TTGCGCGAGC |
| | | | | | | CAGATTGGCC |
| | | | | | | GCAGCACCGC |
| | | | | | | GTGGGGCGCG |
| | | | | | | GTGAGCGGCG |
| | | | | | | CG |
| 418 | factor | Unclassified | WLPPAGLLG | Enterococcus | 419 | TGGCTGCCGC |
| | with anti- | | RCGRWFRP | faecalis | | CGGCGGGCCT |
| | Candida | | WLLWLQSG | | | GCTGGGCCGC |
| | activity | | AQYKWLGN | | | TGCGGCCGCT |
| | | | LFGLGPK | | | GGTTTCGCCC |
| | | | | | | GTGGCTGCTG |
| | | | | | | TGGCTGCAGA |
| | | | | | | GCGGCGCGCA |
| | | | | | | GTATAAATGG |
| | | | | | | CTGGGCAACC |
| | | | | | | TGTTTGGCCT |
| | | | | | | GGGCCCGAAA |
| 420 | Ava_1098 | Unclassified | NLDQWLTE | Anabaena | 421 | TAATTTAGATC |
| | (putative | | QVHEFQDM | variabilis | | AGTGGTTAAC |
| | heterocyst | | YLEPQAISN | ATCC 29413 | | AGAACAAGTT |
| | differentiation | | QDITFKLSDL | | | CATGAGTTTC |
| | protein) | | DFIHN | | | AAGATATGTA |
| | | | | | | CTTGGAACCA |
| | | | | | | CAAGCAATAT |
| | | | | | | CCAATCAAGA |
| | | | | | | CATTACCTTCA |
| | | | | | | AACTATCTGA |
| | | | | | | CCTAGATTTTA |
| | | | | | | TTCATAATTGA |
| 422 | alr2818 | Unclassified | NLDQWLTE | Nostoc sp | 423 | AATTTAGATC |
| | (putative | | QVHEFQDM | 7120 | | AATGGTTAAC |
| | heterocyst | | YLEPQAISN | | | AGAACAAGTT |
| | differentiation | | QDITFKLSDL | | | CATGAGTTTC |
| | protein) | | DFIHN | | | AAGATATGTA |
| | | | | | | CTTGGAACCA |
| | | | | | | CAAGCAATAT |
| | | | | | | CCAATCAAGA |
| | | | | | | CATTACCTTCA |
| | | | | | | AACTGTCAGA |
| | | | | | | CCTAGATTTTA |
| | | | | | | TTCATAATTGA |
| 424 | Aazo_072 | Unclassified | HREKKSA | Nostoc | 425 | CACAGAGAGA |
| | 4 | | | azollae 0708 | | AAAAATCAGC |
| | (putative | | | | | ATAG |
| | heterocyst | | | | | |
| | differentiation | | | | | |
| | protein) | | | | | |
| 426 | AM1_401 | Unclassified | TSNNWLAK | Acaryochloris | 427 | ACAAGCAATA |
| | 0 | | NYLSMWNK | marina | | ACTGGCTAGC |
| | (putative | | KSSNPNL | MBIC11017 | | CAAAAACTAT |
| | heterocyst | | | | | CTTTCTATGTG |
| | differentiation | | | | | GAATAAAAAG |
| | protein) | | | | | AGCAGTAATC |
| | | | | | | CAAACCTTTA |
| | | | | | | G |
| 428 | PCC8801 | Unclassified | FRYFWW | Cyanothece | 429 | TTTAGATATTT |
| | _3266 | | | PCC 8801 | | TTGGTGGTAA |
| | (putative | | | | | |
| | heterocyst | | | | | |
| | differentiation | | | | | |
| | protein) | | | | | |
| 430 | Cyan8802 | Unclassified | FRYFWW | Cyanothece | 431 | TTTAGATATTT |
| | _2855 | | | PCC 8802 | | TTGGTGGTAA |
| | (putative | | | | | |
| | heterocyst | | | | | |
| | differentiation | | | | | |
| | protein) | | | | | |
| 432 | PCC7424 | Unclassified | CGEKWRIFS | Cyanothece | 433 | TGTGGAGAAA |
| | 3517 | | | PCC 7424 | | AATGGAGAAT |
| | | | | | | TTTTAGC |
| 434 | cce_2677 | Unclassified | FRLQLWQF | Cyanothece | 435 | TTTCGCTTACA |
| | (putative | | | ATCC 51142 | | ACTGTGGCAA |
| | HetP | | | | | TTT |
| | protein) | | | | | |
| 436 | CY0110_ | Unclassified | LGCNQSSIW | Cyanothece | 437 | CTAGGATGTA |
| | 11572 | | SIFFWNH | CCY0110 | | ACCAGAGCAG |
| | (putative | | | | | TATCTGGTCA |
| | heterocyst | | | | | ATTTTTTTCTG |
| | differentiation | | | | | GAATCATTAA |
| | protein) | | | | | |
| 438 | MC7420_ | Unclassified | YNLQGLPAI | Microcoleus | 439 | TATAACCTAC |
| | 4637 | | ESEDCIPDSV | chthonoplastes | | AGGGGTTGCC |
| | | | APSDDWFSG | PCC 7420 | | AGCAATTGAG |
| | | | VSSLFNRLT | | | TCAGAAGACT |
| | | | GLG | | | GTATCCCAGA |
| | | | | | | TTCTGTAGCG |
| | | | | | | CCTTCGGATG |
| | | | | | | ATTGGTTTTCA |
| | | | | | | GGCGTATCGT |
| | | | | | | CTCTGTTTAAC |
| | | | | | | CGCTTGACTG |
| | | | | | | GGTTGGGTTA |
| | | | | | | G |
| 440 | asr1611 | Unclassified | WMAIRRILR | Nostoc sp | 441 | TGGATGGCGA |
| | (putative | | CHPFHPGGY | 7120 | | TTCGCCGCATT |
| | DUF37 | | DPVPELGEH | | | TTGCGTTGTCA |
| | family | | CCHHDSGNK | | | TCCATTCCACC |
| | protein) | | G | | | CAGGGGGTTA |
| | | | | | | TGATCCTGTA |
| | | | | | | CCAGAGTTGG |
| | | | | | | GTGAGCATTG |
| | | | | | | TTGTCATCATG |
| | | | | | | ATAGCGGGAA |
| | | | | | | TAAGGGGTGA |
| 442 | Ava_4222 | Unclassified | WMGIRRILR | Anabaena | 443 | TGGATGGGGA |
| | (putative | | CHPFHPGGY | variabilis | | TTCGCCGCATT |
| | DUF37 | | DPVPEVGEH | ATCC 29413 | | TTGCGTTGTCA |
| | family | | CCHHDSGK | | | TCCATTCCACC |
| | protein) | | | | | CAGGCGGTTA |
| | | | | | | TGATCCTGTA |
| | | | | | | CCAGAGGTGG |
| | | | | | | GTGAGCATTG |
| | | | | | | TTGTCATCATG |
| | | | | | | ATAGCGGGAA |
| | | | | | | GTAG |
| 444 | N9414_0 | Unclassified | WMATRRILR | Nodularia | 445 | TGGATGGCGA |
| | 7129 | | CHPFHPGGY | spumigena | | CTCGGCGGAT |
| | (putative | | DPVPEVKHN | CCY9414 | | TTTGCGTTGTC |
| | DUF37 | | CCDQHLSDS | | | ATCCCTTCCAT |
| | family | | GKQTTEDHH | | | CCTGGTGGAT |
| | protein) | | KGS | | | ATGATCCAGT |
| | | | | | | TCCAGAGGTA |
| | | | | | | AAACACAATT |
| | | | | | | GCTGCGATCA |
| | | | | | | GCATCTGTCC |
| | | | | | | GATTCTGGGA |
| | | | | | | AACAGACCAC |
| | | | | | | AGAAGACCAT |
| | | | | | | CACAAAGGCT |
| | | | | | | CGTAG |
| 446 | Aazo_008 | Unclassified | WMATLRILC | Nostoc | 447 | TGGATGGCAA |
| | 3 | | HPFHPGGYD | azollae 0708 | | CTTTGCGGATT |
| | (putative | | PVPGLAEKS | | | TTACGCTGTC |
| | DUF37 | | CCDHHD | | | ATCCTTTCCAT |
| | family | | | | | CCTGGTGGTT |
| | protein) | | | | | ATGATCCTGT |
| | | | | | | ACCAGGACTA |
| | | | | | | GCGGAAAAAT |
| | | | | | | CCTGTTGTGA |
| | | | | | | CCATCATGATT |
| | | | | | | GA |
| 448 | S7335_34 | Unclassified | WLTAKRFCR | Synechococcus | 449 | TGGCTAACAG |
| | 09 | | CHPLHPGGY | PCC 7335 | | CCAAGCGCTT |
| | (putative | | DPVPEKKSV | | | TTGTCGCTGTC |
| | DUF37 | | L | | | ATCCGCTTCAT |
| | family | | | | | CCTGGCGGGT |
| | protein) | | | | | ATGATCCGGT |
| | | | | | | ACCGGAGAAG |
| | | | | | | AAATCGGTAC |
| | | | | | | TCTAA |
| 450 | P9303_21 | Unclassified | WLTLRRLSR | Prochlorococcus | 451 | TGGCTCACCC |
| | 151 | | CHPFTPCGC | marinus | | TGCGGCGCCT |
| | (putative | | DPVPD | MIT 9303 | | GTCTCGTTGCC |
| | DUF37 | | | | | ATCCTTTTACC |
| | family | | | | | CCCTGTGGTT |
| | protein) | | | | | GCGACCCGGT |
| | | | | | | GCCTGATTAA |

As used herein “bacteriocin polynucleotide” refers to a polynucleotide encoding a bacteriocin. In some embodiments, the host cell comprises at least one bacteriocin.

### Bacteriocin Immunity Modulators

Exemplary bacteriocin immunity modulators are shown in Table 2. While the immunity modulators in Table 2 are naturally-occuring, the skilled artisan will appreciate that variants of the immunity modulators of Table 2, naturally-occuring immunity modulators other than the immunity modulators of Table 2, or synthetic immunity modulators can be used according to some embodiments herein.

In some embodiments, a particular immunity modulator or particular combination of immunity modulators confers immunity to a particular bacteriocin, particular class or category of bacteriocins, or particular combination of bacteriocins. Exemplary bacteriocins to which immunity modulators can confer immunity are identified in Table 2. While Table 2 identifies an “organism of origin” for exemplary immunity modulators, these immunity modulators can readily be expressed in other naturally-occurring, genetically modified, or synthetic microorganisms to provide a desired bacteriocin immunity activity in accordance with some embodiments herein. As such, as used herein “immunity modulator” refers not only to structures expressly provided herein, but also to structure that have substantially the same effect as the “immunity modulator” structures described herein, including fully synthetic immunity modulators, and immunity modulators that provide immunity to bacteriocins that are functionally equivalent to the bacteriocins disclosed herein.

Exemplary polynucleotide sequences encoding the polypeptides of Table 2 are indicated in Table 2. The skilled artisan will readily understand that the genetic code is degenerate, and moreover, codon usage can vary based on the particular organism in which the gene product is being expressed, and as such, a particular polypeptide can be encoded by more than one polynucleotide. In some embodiments, a polynucleotide encoding a bacteriocin immunity modulator is selected based on the codon usage of the organism expressing the bacteriocin immunity modulator. In some embodiments, a polynucleotide encoding a bacteriocin immunity modulator is codon optimized based on the particular organism expressing the bacteriocin immunity modulator. A vast range of functional immunity modulators can incorporate features of immunity modulators disclosed herein, thus providing for a vast degree of identity to the immunity modulators in Table 2. In some embodiments, an immunity modulator has at least about 50% identity, for example, at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the polypeptides of Table 2.

TABLE 2
Exemplary bacteriocin immunity modulators

| Poly- | | | | Poly- | |
|---|---|---|---|---|---|
| peptide | | | | nucleotide | |
| SEQ | | Polypeptide | Organism | SEQ | Polynucleotide |
| ID NO: | Name | Sequence | of origin | ID NO: | Sequence |
| 452 | Microcin | MSYKKLY | Escherichia | 453 | ATGAGTTATAAAAAAC |
| | H47 | QLTAIFSLP | coli | | TGTACCAATTGACGGCT |
| | immunity | LTILLVSLS | | | ATATTTAGTTTACCTCT |
| | modulator | SLRIVGEG | | | TACTATCTTATTGGTTT |
| | Mehl | NSYVDVFL | | | CACTTTCATCCCTTCGG |
| | | SFIIFLGFIE | | | ATTGTTGGCGAAGGGA |
| | | LIHGIRKIL | | | ATTCTTATGTTGACGTT |
| | | VWSGWKN | | | TTTCTAAGCTTTATAAT |
| | | GS | | | ATTTCTTGGTTTTATTG |
| | | | | | AGCTGATTCATGGGATT |
| | | | | | CGAAAGATTTTGGTCTG |
| | | | | | GTCAGGCTGGAAAAAC |
| | | | | | GGAAGTTAA |
| 454 | Colicin-E3 | MGLKLDLT | Escherichia | 455 | ATGGGACTTAAATTGG |
| | immunity | WFDKSTED | coli | | ATTTAACTTGGTTTGAT |
| | modulator | FKGEEYSK | | | AAAAGTACAGAAGATT |
| | (Colicin-E3 | DFGDDGSV | | | TTAAGGGTGAGGAGTA |
| | chain B) | MESLGVPF | | | TTCAAAAGATTTTGGAG |
| | (ImmE3) | KDNVNNG | | | ATGACGGTTCAGTTATG |
| | (Microcin- | CFDVIAEW | | | GAAAGTCTAGGTGTGC |
| | E3 | VPLLQPYF | | | CTTTTAAGGATAATGTT |
| | immunity | NHQIDISD | | | AATAACGGTTGCTTTGA |
| | modulator) | NEYFVSFD | | | TGTTATAGCTGAATGG |
| | | YRDGDW | | | GTACCTTTGCTACAACC |
| | | | | | ATACTTTAATCATCAAA |
| | | | | | TTGATATTTCCGATAAT |
| | | | | | GAGTATTTTGTTTCGTT |
| | | | | | TGATTATCGTGATGGTG |
| | | | | | ATTGGTGA |
| 456 | Colicin-E1 | MSLRYYIK | Escherichia | 457 | ATGAGCTTAAGATACTA |
| | immunity | NILFGLYC | coli | | CATAAAAAATATTTTAT |
| | modulator | TLIYIYLIT | | | TTGGCCTGTACTGCACA |
| | (ImmE1) | KNSEGYYF | | | CTTATATATATATACCT |
| | (Microcin- | LVSDKML | | | TATAACAAAAAACAGC |
| | E1 | YAIVISTIL | | | GAAGGGTATTATTTCCT |
| | immunity | CPYSKYAI | | | TGTGTCAGATAAGATG |
| | modulator) | EYIAFNFIK | | | CTATATGCAATAGTGAT |
| | | KDFFERRK | | | AAGCACTATTCTATGTC |
| | | NLNNAPVA | | | CATATTCAAAATATGCT |
| | | KLNLFMLY | | | ATTGAATACATAGCTTT |
| | | NLLCLVLA | | | TAACTTCATAAAGAAA |
| | | IPFGLLGLF | | | GATTTTTTCGAAAGAAG |
| | | ISIKNN | | | AAAAAACCTAAATAAC |
| | | | | | GCCCCCGTAGCAAAATT |
| | | | | | AAACCTATTTATGCTAT |
| | | | | | ATAATCTACTTTGTTTG |
| | | | | | GTCCTAGCAATCCCATT |
| | | | | | TGGATTGCTAGGACTTT |
| | | | | | TTATATCAATAAAGAAT |
| | | | | | AATTAA |
| 458 | Cloacin | MGLKLHIH | Escherichia | 459 | ATGGGGCTTAAATTAC |
| | immunity | WFDKKTEE | coli | | ATATTCATTGGTTTGAT |
| | modulator | FKGGEYSK | | | AAGAAAACCGAAGAGT |
| | | DFGDDGSV | | | TTAAAGGCGGTGAATA |
| | | IESLGMPL | | | CTCAAAAGACTTCGGT |
| | | KDNINNG | | | GATGATGGTTCTGTCAT |
| | | WFDVEKP | | | TGAAAGTCTGGGGATG |
| | | WVSILQPH | | | CCTTTAAAGGATAATAT |
| | | FKNVIDISK | | | TAATAATGGTTGGTTTG |
| | | FDYFVSFV | | | ATGTTGAAAAACCATG |
| | | YRDGNW | | | GGTTTCGATATTACAGC |
| | | | | | CACACTTTAAAAATGTA |
| | | | | | ATCGATATTAGTAAATT |
| | | | | | TGATTACTTTGTATCCT |
| | | | | | TTGTTTACCGGGATGGT |
| | | | | | AACTGGTAA |
| 460 | Colicin-E2 | MELKHSIS | Escherichia | 461 | ATGGAACTGAAACATA |
| | immunity | DYTEAEFL | coli | | GTATTAGTGATTATACC |
| | modulator | EFVKKICR | | | GAGGCTGAATTTCTGG |
| | (ImmE2) | AEGATEED | | | AGTTTGTAAAAAAAAT |
| | (Microcin- | DNKLVREF | | | ATGTAGAGCTGAAGGT |
| | E2 | ERLTEHPD | | | GCTACTGAAGAGGATG |
| | immunity | GSDLIYYP | | | ACAATAAATTAGTGAG |
| | modulator) | RDDREDSP | | | AGAGTTTGAGCGATTA |
| | | EGIVKEIKE | | | ACTGAGCACCCAGATG |
| | | WRAANGK | | | GTTCAGATCTGATTTAT |
| | | SGFKQG | | | TATCCTCGCGATGACAG |
| | | | | | GGAAGATAGTCCTGAA |
| | | | | | GGGATTGTCAAGGAAA |
| | | | | | TTAAAGAATGGCGAGC |
| | | | | | TGCTAACGGTAAGTCA |
| | | | | | GGATTTAAACAGGGCT |
| | | | | | GA |
| 462 | Colicin-A | MMNEHSID | Citrobacter | 463 | ATGATGAATGAACACT |
| | immunity | TDNRKAN | freundii | | CAATAGATACGGACAA |
| | modulator | NALYLFIII | | | CAGAAAGGCCAATAAC |
| | (Microcin- | GLIPLLCIF | | | GCATTGTATTTATTTAT |
| | A immunity | VVYYKTPD | | | AATAATCGGATTAATAC |
| | modulator) | ALLLRKIA | | | CATTATTGTGCATTTTT |
| | | TSTENLPSI | | | GTTGTTTACTACAAAAC |
| | | TSSYNPLM | | | GCCAGACGCTTTACTTT |
| | | TKVMDIYC | | | TACGTAAAATTGCTACA |
| | | KTAPFLALI | | | AGCACTGAGAATCTCCC |
| | | LYILTFKIR | | | GTCAATAACATCCTCCT |
| | | KLINNTDR | | | ACAACCCATTAATGACA |
| | | NTVLRSCL | | | AAGGTTATGGATATTTA |
| | | LSPLVYAA | | | TTGTAAAACAGCGCCTT |
| | | IVYLFCFR | | | TCCTTGCCTTAATACTA |
| | | NFELTTAG | | | TACATCCTAACCTTTAA |
| | | RPVRLMAT | | | AATCAGAAAATTAATC |
| | | NDATLLLF | | | AACAACACCGACAGGA |
| | | YIGLYSIIFF | | | ACACTGTACTTAGATCT |
| | | TTYITLFTP | | | TGTTTATTAAGTCCATT |
| | | VTAFKLLK | | | GGTCTATGCAGCAATTG |
| | | KRQ | | | TTTATCTATTCTGCTTC |
| | | | | | CGAAATTTTGAGTTAAC |
| | | | | | AACAGCCGGAAGGCCT |
| | | | | | GTCAGATTAATGGCCA |
| | | | | | CCAATGACGCAACACT |
| | | | | | ATTGTTATTTTATATTG |
| | | | | | GTCTGTACTCAATAATT |
| | | | | | TTCTTTACAACCTATAT |
| | | | | | CACGCTATTCACACCAG |
| | | | | | TCACTGCATTTAAATTA |
| | | | | | TTAAAAAAAAGGCAGT |
| | | | | | AA |
| 464 | Colicin-Ia | MNRKYYF | Escherichia | 465 | ATGAACAGAAAATATT |
| | immunity | NNMWWG | coli | | ATTTTAATAATATGTGG |
| | modulator | WVTGGYM | | | TGGGGATGGGTGACGG |
| | | LYMSWDY | | | GGGGATATATGCTGTA |
| | | EFKYRLLF | | | TATGTCATGGGATTATG |
| | | WCISLCGM | | | AGTTTAAATACAGATTA |
| | | VLYPVAK | | | CTGTTCTGGTGTATTTC |
| | | WYIEDTAL | | | TCTCTGCGGAATGGTTT |
| | | KFTRPDFW | | | TGTATCCGGTTGCAAAA |
| | | NSGFFADT | | | TGGTATATTGAAGATAC |
| | | PGKMGLLA | | | AGCTCTAAAATTTACCC |
| | | VYTGTVFI | | | GGCCTGATTTCTGGAAC |
| | | LSLPLSMIY | | | AGCGGTTTTTTTGCTGA |
| | | ILSVIIKRLS | | | TACACCTGGAAAAATG |
| | | VR | | | GGGTTGCTTGCGGTTTA |
| | | | | | TACGGGTACTGTTTTCA |
| | | | | | TATTATCTCTTCCGTTA |
| | | | | | AGTATGATATATATTCT |
| | | | | | TTCTGTTATTATAAAAA |
| | | | | | GGCTGTCTGTAAGATA |
| | | | | | G |
| 466 | Colicin-Ib | MKLDISVK | Escherichia | 467 | ATGAAACTGGATATATC |
| | immunity | YLLKSLIPI | coli | | TGTAAAGTATTTACTGA |
| | modulator | LIILTVFYL | | | AAAGCCTGATACCAAT |
| | | GWKDNQE | | | CCTCATTATTCTTACAG |
| | | NARMFYAF | | | TTTTTTATCTGGGATGG |
| | | IGCIISAITF | | | AAAGATAACCAGGAAA |
| | | PFSMRIIQK | | | ATGCAAGAATGTTTTAT |
| | | MVIRFTGK | | | GCGTTCATCGGATGCAT |
| | | EFWQKDFF | | | TATCAGTGCCATTACTT |
| | | TNPVGGSL | | | TTCCTTTTTCAATGAGG |
| | | TAIFELFCF | | | ATAATACAGAAAATGG |
| | | VISVPVVAI | | | TAATAAGGTTTACAGG |
| | | YLIFILCKA | | | GAAAGAATTCTGGCAA |
| | | LSGK | | | AAAGACTTCTTTACAAA |
| | | | | | TCCAGTTGGCGGAAGC |
| | | | | | TTAACTGCAATATTTGA |
| | | | | | ATTATTCTGTTTCGTTA |
| | | | | | TATCAGTTCCTGTGGTT |
| | | | | | GCCATTTACTTAATTTT |
| | | | | | TATACTCTGCAAAGCCC |
| | | | | | TTTCAGGAAAATGA |
| 468 | Colicin-N | MHNTLLEK | Escherichia | 469 | ATGCACAATACACTCCT |
| | immunity | IIAYLSLPG | coli | | CGAAAAAATCATCGCA |
| | modulator | FHSLNNPP | | | TACCTATCCCTACCAGG |
| | (Microcin- | LSEAFNLY | | | ATTTCATTCATTAAACA |
| | N immunity | VHTAPLAA | | | ACCCGCCCCTAAGCGA |
| | modulator) | TSLFIFTHK | | | AGCATTCAATCTCTATG |
| | | ELELKPKS | | | TTCATACAGCCCCTTTA |
| | | SPLRALKIL | | | GCTGCAACCAGCTTATT |
| | | TPFTILYIS | | | CATATTCACACACAAAG |
| | | MIYCFLLT | | | AATTAGAGTTAAAACC |
| | | DTELTLSS | | | AAAGTCGTCACCTCTGC |
| | | KTFVLIVK | | | GGGCACTAAAGATATT |
| | | KRSVFVFF | | | AACTCCTTTCACTATTC |
| | | LYNTIYWD | | | TTTATATATCCATGATA |
| | | IYIHIFVLL | | | TACTGTTTCTTGCTAAC |
| | | VPYRNI | | | TGACACAGAACTAACC |
| | | | | | TTGTCATCAAAAACATT |
| | | | | | TGTATTAATAGTCAAAA |
| | | | | | AACGATCTGTTTTTGTC |
| | | | | | TTTTTTCTATATAACAC |
| | | | | | TATATATTGGGATATAT |
| | | | | | ATATTCACATATTTGTA |
| | | | | | CTTTTGGTTCCTTATAG |
| | | | | | GAACATATAA |
| 470 | Colicin-E8 | MELKNSIS | Escherichia | 471 | ATGGAACTGAAAAACA |
| | immunity | DYTETEFK | coli | | GCATTAGTGATTACACT |
| | modulator | KIIEDIINCE | | | GAAACTGAATTCAAAA |
| | (ImmE8) | GDEKKQD | | | AAATTATTGAAGACATC |
| | (Microcin- | DNLEHFIS | | | ATCAATTGTGAAGGTG |
| | E8 | VTEHPSGS | | | ATGAAAAAAAACAGGA |
| | immunity | DLIYYPEG | | | TGATAACCTCGAGCATT |
| | modulator) | NNDGSPEA | | | TTATAAGTGTTACTGAG |
| | | VIKEIKEW | | | CATCCTAGTGGTTCTGA |
| | | RAANGKSG | | | TCTGATTTATTACCCAG |
| | | FKQG | | | AAGGTAATAATGATGG |
| | | | | | TAGCCCTGAAGCTGTTA |
| | | | | | TTAAAGAGATTAAAGA |
| | | | | | ATGGCGAGCTGCTAAC |
| | | | | | GGTAAGTCAGGATTTA |
| | | | | | AACAGGGCTGA |
| 472 | Lactococcin- | MKKKQIEF | Lactococcus | 473 | ATGAAAAAAAAACAAA |
| | A | ENELRSML | lactis | | TAGAATTTGAAAACGA |
| | immunity | ATALEKDI | subsp. | | GCTAAGAAGTATGTTG |
| | modulator | SQEERNAL | lactis | | GCTACCGCCCTTGAAAA |
| | | NIAEKALD | (Streptococcus | | AGACATTAGTCAAGAG |
| | | NSEYLPKII | lactis) | | GAAAGAAATGCTCTGA |
| | | LNLRKALT | | | ATATTGCAGAAAAGGC |
| | | PLAINRTL | | | GCTTGACAATTCTGAAT |
| | | NHDLSELY | | | ATTTACCAAAAATTATT |
| | | KFITSSKAS | | | TTAAACCTCAGAAAAG |
| | | NKNLGGG | | | CCCTAACTCCATTAGCT |
| | | LIMSWGRL | | | ATAAATCGAACACTTAA |
| | | F | | | CCATGATTTATCTGAAC |
| | | | | | TGTATAAATTCATTACA |
| | | | | | AGTTCCAAAGCATCAA |
| | | | | | ACAAAAATTTAGGTGG |
| | | | | | TGGTTTAATTATGTCGT |
| | | | | | GGGGACGACTATTCTA |
| | | | | | A |
| 474 | Lactococcin- | MKKKQIEF | Lactococcus | 475 | ATGAAAAAAAAACAAA |
| | A | ENELRSML | lactis | | TAGAATTTGAAAACGA |
| | immunity | ATALEKDI | subsp. | | GCTAAGAAGTATGTTG |
| | modulator | SQEERNAL | cremoris | | GCTACCGCCCTTGAAAA |
| | | NIAEKALD | (Streptococcus | | AGACATTAGTCAAGAG |
| | | NSEYLPKII | cremoris) | | GAAAGAAATGCTCTGA |
| | | LNLRKALT | | | ATATTGCAGAAAAGGC |
| | | PLAINRTL | | | GCTTGACAATTCTGAAT |
| | | NHDLSELY | | | ATTTACCAAAAATTATT |
| | | KFITSSKAS | | | TTAAACCTCAGAAAAG |
| | | NKNLGGG | | | CCCTAACTCCATTAGCT |
| | | LIMSWGRL | | | ATAAATCGAACACTTAA |
| | | F | | | CCATGATTTATCTGAAC |
| | | | | | TGTATAAATTCATTACA |
| | | | | | AGTTCCAAAGCATCAA |
| | | | | | ACAAAAATTTAGGTGG |
| | | | | | TGGTTTAATTATGTCGT |
| | | | | | GGGGACGACTATTCTA |
| | | | | | A |
| 476 | Colicin-D | MNKMAMI | Escherichia | 477 | ATGATCGATTTGGCGA |
| | immunity | DLAKLFLA | coli | | AATTATTTTTAGCTTCG |
| | modulator | SKITAIEFS | | | AAAATTACAGTGATTG |
| | (Microcin- | ERICVERR | | | AGTTTTCAGAGCGAATT |
| | D immunity | RLYGVKDL | | | TGTGTTGAACGGAGAA |
| | modulator) | SPNILNCG | | | GATTGTATGGTGTTAAG |
| | | EELFMAAE | | | GATTTGTCTCCGAATAT |
| | | RFEPDADR | | | ATTAAATTGTGGGGAA |
| | | ANYEIDDN | | | GAGTTGTCTATGGCTGC |
| | | GLKVEVRS | | | TGAGCGATTTGAGCCT |
| | | ILEKFKL | | | GATGCAGATAGGGCTA |
| | | | | | ATTATGAAATTGATGAT |
| | | | | | AATGGACTTAAGGTCG |
| | | | | | AGGTCCGATCTATCTTG |
| | | | | | GAAAAACTTAAATCAT |
| | | | | | AA |
| 478 | Colicin-E5 | MKLSPKAA | Escherichia | 479 | ATGAAGTTATCACCAA |
| | immunity | IEVCNEAA | coli | | AAGCTGCAATAGAAGT |
| | modulator | KKGLWILG | | | TTGTAATGAAGCAGCG |
| | (ImmE5) | IDGGHWLN | | | AAAAAAGGCTTATGGA |
| | (Microcin- | PGFRIDSSA | | | TTTTGGGCATTGATGGT |
| | E5 | SWTYDMP | | | GGGCATTGGCTGAATC |
| | immunity | EEYKSKIPE | | | CTGGATTCAGGATAGA |
| | modulator) | NNRLAIENI | | | TAGTTCAGCATCATGGA |
| | | KDDIENGY | | | CATATGATATGCCGGA |
| | | TAFIITLKM | | | GAATACAAATCAAAAA |
| | | | | | TCCCTGAAAATAATAG |
| | | | | | ATTGGCTATTGAAAATA |
| | | | | | TTAAAGATGATATTGA |
| | | | | | GAATGGATACACTGCTT |
| | | | | | TCATTATCACGTTAA |
| 480 | Colicin-E6 | MGLKLHIN | Escherichia | 481 | ATGGGGCTTAAATTAC |
| | immunity | WFDKRTEE | coli | | ATATTAATTGGTTTGAT |
| | modulator | FKGGEYSK | | | AAGACGACCGAGGAAT |
| | (ImmE6) | DFGDDGSV | | | TTAAAGGTGGTGAGTA |
| | (Microcin- | IERLGMPF | | | TTCAAAAGATTTTGGAG |
| | E6 | KDNINNG | | | ATGATGGCTCGGTCATT |
| | immunity | WFDVIAEW | | | GAACGTCTTGGAATGC |
| | modulator) | VPLLQPYF | | | CTTTAAAAGATAATATC |
| | | NHQIDISD | | | AATAATGGTTGGTTTGA |
| | | NEYFVSFD | | | TGTTATAGCTGAATGG |
| | | YRDGDW | | | GTACCTTTGCTACAACC |
| | | | | | ATACTTTAATCATCAAA |
| | | | | | TTGATATTTCCGATAAT |
| | | | | | GAGTATTTTGTTTCGTT |
| | | | | | TGATTATCGTGATGGTG |
| | | | | | ATTGGTGA |
| 482 | Colicin-E8 | MELKKSIG | Escherichia | 483 | GTGGAGCTAAAGAAAA |
| | immunity | DYTETEFK | coli | | GTATTGGTGATTACACT |
| | modulator | KIIENIINCE | | | GAAACCGAATTCAAAA |
| | in ColE6 | GDEKKQD | | | AAATTATTGAAAACATC |
| | (E8Imm[E | DNLEHFIS | | | ATCAATTGTGAAGGTG |
| | 6]) | VTEHPSGS | | | ATGAAAAAAAACAGGA |
| | | DLIYYPEG | | | TGATAACCTCGAGCATT |
| | | NNDGSPEA | | | TTATAAGTGTTACTGAG |
| | | VIKEIKEW | | | CATCCTAGTGGTTCTGA |
| | | RAANGKSG | | | TCTGATTTATTACCCAG |
| | | FKQG | | | AAGGTAATAATGATGG |
| | | | | | TAGCCCTGAAGCTGTTA |
| | | | | | TTAAAGAGATTAAAGA |
| | | | | | ATGGCGAGCTGCTAAC |
| | | | | | GGTAAGTCAGGATTTA |
| | | | | | AACAGGGCTGA |
| 484 | Colicin-E9 | MELKHSIS | Escherichia | 485 | ATGGAACTGAAGCATA |
| | immunity | DYTEAEFL | coli | | GCATTAGTGATTATACA |
| | modulator | QLVTTICN | | | GAAGCTGAATTTTTACA |
| | (ImmE9) | ADTSSEEE | | | ACTTGTAACAACAATTT |
| | (Microcin- | LVKLVTHF | | | GTAATGCGAACACTTCC |
| | E9 | EEMTEHPS | | | AGTGAAGAAGAACTGG |
| | immunity | GSDLIYYP | | | TTAAATTGGTTACACAC |
| | modulator) | KEGDDDSP | | | TTTGAGGAAATGACTG |
| | | SGIVNTVK | | | AGCACCCTAGTGGTAG |
| | | QWRAANG | | | TGATTTAATATATTACC |
| | | KSGFKQG | | | CAAAAGAAGGTGATGA |
| | | | | | TGACTCACCTTCAGGTA |
| | | | | | TTGTAAACACAGTAAA |
| | | | | | ACAATGGCGAGCCGCT |
| | | | | | AACGGTAAGTCAGGAT |
| | | | | | TTAAACAGGGCTAA |
| 486 | Colicin-M | MLTLYGYI | Escherichia | 487 | ATGAAAGTAATTAGCA |
| | immunity | RNVFLYR | coli | | TGAAATTTATTTTTATT |
| | modulator | MNDRSCG | | | TTAACGATTATTGCTCT |
| | (Microcin- | DFMKVISM | | | TGCTGCTGTTTTTTTCT |
| | M | KFIFILTIIA | | | GGTCTGAAGATAAAGG |
| | immunity | LAAVFFWS | | | TCCGGCATGCTATCAGG |
| | modulator) | EDKGPACY | | | TCAGCGATGAACAGGC |
| | | QVSDEQAR | | | CAGAACGTTTGTAAAA |
| | | TFVKNDYL | | | AATGATTACCTGCAAA |
| | | QRMKRWD | | | GAATGAAACGCTGGGA |
| | | NDVQLLGT | | | CAACGATGTACAACTTC |
| | | EIPKITWEK | | | TTGGTACAGAAATCCC |
| | | IERSLTDVE | | | GAAAATTACATGGGAA |
| | | DEKTLLVP | | | AAGATTGAGAGAAGTT |
| | | FKAEGPDG | | | TAACAGATGTTGAAGA |
| | | KRMYYGM | | | TGAAAAAACACTTCTTG |
| | | YHCEEGY | | | TCCCATTTAAAGCTGAA |
| | | VEYAND | | | GGCCCGGACGGTAAGA |
| | | | | | GAATGTATTATGGCATG |
| | | | | | TACCATTGTGAGGAGG |
| | | | | | GATATGTTGAATATGCG |
| | | | | | AATGACTAA |
| 488 | Colicin-B | MTSNKDK | Escherichia | 489 | ATGACCAGCAATAAAG |
| | immunity | NKKANEIL | coli | | ATAAGAACAAGAAAGC |
| | modulator | YAFSIIGIIP | | | AAACGAAATATTATAT |
| | (Microcin- | LMAILILRI | | | GCATTTTCCATAATCGG |
| | B immunity | NDPYSQVL | | | GATTATTCCATTAATGG |
| | modulator) | YYLYNKV | | | CTATATTAATACTTCGA |
| | | AFLPSITSL | | | ATAAATGATCCATATTC |
| | | HDPVMTTL | | | TCAAGTGCTGTACTACT |
| | | MSNYNKT | | | TATATAATAAGGTGGC |
| | | APVMGILV | | | ATTTCTCCCTTCTATTA |
| | | FLCTYKTR | | | CATCATTGCATGATCCC |
| | | E1IKPVTRK | | | GTCATGACAACACTTAT |
| | | LVVQSCFW | | | GTCAAACTACAACAAG |
| | | GPVFYAILI | | | ACAGCGCCAGTTATGG |
| | | YITLFYNLE | | | GTATTCTCGTTTTTCTT |
| | | LTTAGGFF | | | TGCACATATAAGACAA |
| | | KLLSHNVI | | | GAGAAATCATAAAGCC |
| | | TLFILYCSI | | | AGTAACAAGAAAACTT |
| | | YFTVLTMT | | | GTTGTGCAATCCTGTTT |
| | | YAILLMPL | | | CTGGGGGCCCGTTTTTT |
| | | LVIKYFKG | | | ATGCCATTCTGATTTAT |
| | | RQ | | | ATCACACTGTTCTATAA |
| | | | | | TCTGGAACTAACAACA |
| | | | | | GCAGGTGGTTTTTTTAA |
| | | | | | ATTATTATCTCATAATG |
| | | | | | TCATCACTCTGTTTATT |
| | | | | | TTATATTGCTCCATTTA |
| | | | | | CTTTACTGTTTTAACCA |
| | | | | | TGACATATGCGATTTTA |
| | | | | | CTGATGCCATTACTTGT |
| | | | | | CATTAAATATTTTAAAG |
| | | | | | GGAGGCAGTAA |
| 490 | Colicin-V | MDRKRTK | Escherichia | 491 | ATGGATAGAAAAAGAA |
| | immunity | LELLFAFII | coli | | CAAAATTAGAGTTGTTA |
| | modulator | NATAIYIAL | | | TTTGCATTTATAATAAA |
| | (Microcin- | AIYDCVFR | | | TGCCACCGCAATATATA |
| | V immunity | GKDFLSMH | | | TTGCATTAGCTATATAT |
| | modulator) | TFCFSALM | | | GATTGTGTTTTTAGAGG |
| | | SAICYFVG | | | AAAGGACTTTTTATCCA |
| | | DNYYSISD | | | TGCATACATTTTGCTTC |
| | | KIKRRSYE | | | TCTGCATTAATGTCTGC |
| | | NSDSK | | | AATATGTTACTTTGTTG |
| | | | | | GTGATAATTATTATTCA |
| | | | | | ATATCCGATAAGATAA |
| | | | | | AAAGGAGATCATATGA |
| | | | | | GAACTCTGACTCTAAAT |
| | | | | | GA |
| 492 | Colicin- | MSLRYYIK | Shigella | 493 | ATGAGTTTAAGATACTA |
| | E1* | NILFGLYC | sonnei | | CATAAAAAATATTTTGT |
| | immunity | ALIYIYLIT | | | TTGGCCTATACTGCGCA |
| | modulator | KNNEGYYF | | | CTTATATATATATACCT |
| | (ImmE1) | LASDKMLY | | | TATAACAAAAAACAAC |
| | (Microcin- | AIVISTILCP | | | GAAGGGTATTATTTCCT |
| | E1* | YSKYAIEHI | | | AGCGTCAGATAAGATG |
| | immunity | FFKFIKKDF | | | CTATACGCAATAGTGAT |
| | modulator) | FRKRKNLN | | | AAGCACTATTCTATGCC |
| | | KCPRGKIK | | | CATATTCAAAATATGCT |
| | | PYLCVYNL | | | ATTGAACACATATTTTT |
| | | LCLVLAIPF | | | TAAGTTCATAAAGAAA |
| | | GLLGLVYI | | | GATTTTTTCAGAAAAAG |
| | | NKE | | | AAAAAACCTAAATAAA |
| | | | | | TGCCCCCGTGGCAAAA |
| | | | | | TTAAACCGTATTTATGC |
| | | | | | GTATACAATCTACTTTG |
| | | | | | TTTGGTCCTAGCAATCC |
| | | | | | CATTTGGATTGCTAGGA |
| | | | | | CTTGTTTATATCAATAA |
| | | | | | AGAATAA |
| 494 | Colicin-E1 | MSLRYYIK | Escherichia | 495 | ATGAGCTTAAGATACTA |
| | immunity | NILFGLYC | coli | | CATAAAAAATATTTTAT |
| | modulator | TLIYIYLIT | | | TTGGCCTGTACTGCACA |
| | (ImmE1) | KNSEEYYF | | | CTTATATATATATACCT |
| | (Microcin- | LVTDKML | | | TATAACAAAAAACAGC |
| | E1 | YAIVISTIL | | | GAAGAGTATTATTTCCT |
| | immunity | CPYSKYAI | | | TGTGACAGATAAGATG |
| | modulator) | EHIAFNFIK | | | CTATATGCAATAGTGAT |
| | | KHFFERRK | | | AAGCACTATTCTATGTC |
| | | NLNNAPVA | | | CATATTCAAAATATGCT |
| | | KLNLFMLY | | | ATTGAACACATAGCTTT |
| | | NLLCLVLA | | | TAACTTCATAAAGAAAC |
| | | IPFGLLGLF | | | ATTTTTTCGAAAGAAGA |
| | | ISIKNN | | | AAAAACCTAAATAACG |
| | | | | | CCCCCGTAGCAAAATTA |
| | | | | | AACCTATTTATGCTATA |
| | | | | | TAATCTACTTTGTTTGG |
| | | | | | TCCTAGCAATCCCATTT |
| | | | | | GGATTGCTAGGACTTTT |
| | | | | | TATATCAATAAAGAATA |
| | | | | | ATTAA |
| 496 | Probable | MRKNNILL | Leuconostoc | 497 | TTGAGAAAAAATAACA |
| | leucocin-A | DDAKIYTN | gelidum | | TTTTATTGGACGATGCT |
| | immunity | KLYLLLID | | | AAAATATACACGAACA |
| | modulator | RKDDAGY | | | AACTCTATTTGCTATTA |
| | | GDICDVLF | | | ATCGATAGAAAAGATG |
| | | QVSKKLDS | | | ACGCTGGGTATGGAGA |
| | | TKNVEALI | | | TATTTGTGATGTTTTGT |
| | | NRLVNYIRI | | | TTCAGGTATCCAAAAA |
| | | TASTNRIKF | | | ATTAGATAGCACAAAA |
| | | SKDEEAVII | | | AATGTAGAAGCATTGA |
| | | ELGVIGQK | | | TTAACCGATTGGTCAAT |
| | | AGLNGQY | | | TATATACGAATTACCGC |
| | | MADFSDKS | | | TTCAACAAACAGAATTA |
| | | QFYSIFER | | | AGTTTTCAAAAGATGA |
| | | | | | AGAGGCTGTAATTATA |
| | | | | | GAACTTGGTGTAATTG |
| | | | | | GTCAGAAGGCTGGATT |
| | | | | | AAACGGCCAATACATG |
| | | | | | GCTGATTTTTCTGACAA |
| | | | | | ATCTCAGTTTTATAGTA |
| | | | | | TCTTTGAAAGATAA |
| 498 | Lactococcin- | MKKKVDT | Lactococcus | 499 | ATGAAAAAAAAAGTTG |
| | B | EKQITSWA | lactis | | ATACAGAAAAACAAAT |
| | immunity | SDLASKNE | subsp. | | TACTTCTTGGGCATCTG |
| | modulator | TKVQEKLI | cremoris | | ACTTAGCTTCCAAAAAT |
| | | LSSYIQDIE | (Streptococcus | | GAAACAAAGGTTCAAG |
| | | NHVYFPKA | cremoris) | | AAAAATTAATACTGTCT |
| | | MISLEKKL | | | TCTTATATTCAGGACAT |
| | | RDQNNICA | | | CGAAAACCATGTTTACT |
| | | LSKEVNQF | | | TTCCAAAAGCAATGATT |
| | | YFKVVEVN | | | TCTTTAGAAAAAAAATT |
| | | QRKSWMV | | | ACGAGACCAAAATAAT |
| | | GLIV | | | ATTTGCGCTTTATCAAA |
| | | | | | AGAAGTCAATCAGTTTT |
| | | | | | ATTTTAAAGTTGTTGAA |
| | | | | | GTAAATCAAAGAAAAT |
| | | | | | CCTGGATGGTAGGTTTG |
| | | | | | ATAGTTTAA |
| 500 | Pediocin | MNKTKSE | Pediococcus | 501 | ATGAATAAGACTAAGT |
| | PA-1 | HIKQQALD | acidilactici | | CGGAACATATTAAACA |
| | immunity | LFTRLQFLL | | | ACAAGCTTTGGACTTAT |
| | modulator | QKHDTIEP | | | TTACTAGGCTACAGTTT |
| | (Pediocin | YQYVLDIL | | | TTACTACAGAAGCACG |
| | ACH | ETGISKTK | | | ATACTATCGAACCTTAC |
| | immunity | HNQQTPER | | | CAGTACGTTTTAGATAT |
| | modulator) | QARVVYN | | | TCTGGAGACTGGTATCA |
| | | KIASQALV | | | GTAAAACTAAACATAA |
| | | DKLHFTAE | | | CCAGCAAACGCCTGAA |
| | | ENKVLAAI | | | CGACAAGCTCGTGTAG |
| | | NELAHSQK | | | TCTACAACAAGATTGCC |
| | | GWGEFNM | | | AGCCAAGCGTTAGTAG |
| | | LDTTNTWP | | | ATAAGTTACATTTTACT |
| | | SQ | | | GCCGAAGAAAACAAAG |
| | | | | | TTCTAGCAGCCATCAAT |
| | | | | | GAATTGGCGCATTCTCA |
| | | | | | AAAAGGGTGGGGCGAG |
| | | | | | TTTAACATGCTAGATAC |
| | | | | | TACCAATACGTGGCCTA |
| | | | | | GCCAATAG |
| 502 | Putative | MIKDEKIN | Carnobacterium | 503 | ATGATAAAAGATGAAA |
| | carno- | KIYALVKS | maltaromaticum | | AAATAAATAAAATCTAT |
| | bacteriocin- | ALDNTDV | (Carnobacterium | | GCTTTAGTTAAGAGCGC |
| | BM1 | KNDKKLSL | piscicola) | | ACTTGATAATACGGAT |
| | immunity | LLMRIQET | | | GTTAAGAATGATAAAA |
| | modulator | SINGELFY | | | AACTTTCTTTACTTCTT |
| | | DYKKELQP | | | ATGAGAATACAAGAAA |
| | | AISMYSIQ | | | CATCAATTAATGGAGA |
| | | HNFRVPDD | | | ACTATTTTACGATTATA |
| | | LVKLLALV | | | AAAAAGAATTACAGCC |
| | | QTPKAWS | | | AGCTATTAGTATGTACT |
| | | GF | | | CTATTCAACATAACTTT |
| | | | | | CGGGTTCCTGACGATCT |
| | | | | | AGTAAAACTGTTAGCAT |
| | | | | | TAGTTCAAACACCTAAA |
| | | | | | GCTTGGTCAGGGTTTTA |
| | | | | | A |
| 504 | Putative | MDIKSQTL | Carnobacterium | 505 | ATGGATATAAAGTCTCA |
| | carno- | YLNLSEAY | maltaromaticum | | AACATTATATTTGAATC |
| | bacteriocin- | KDPEVKAN | (Carnobacterium | | TAAGCGAGGCATATAA |
| | B2 | EFLSKLVV | piscicola) | | AGACCCTGAAGTAAAA |
| | immunity | QCAGKLTA | | | GCTAATGAATTCTTATC |
| | modulator | SNSENSYIE | | | AAAATTAGTTGTACAAT |
| | (Carnocin- | VISLLSRGI | | | GTGCTGGGAAATTAAC |
| | CP52 | SSYYLSHK | | | AGCTTCAAACAGTGAG |
| | immunity | RIIPSSMLTI | | | AACAGTTATATTGAAGT |
| | modulator) | YTQIQKDI | | | AATATCATTGCTATCTA |
| | | KNGNIDTE | | | GGGGTATTTCTAGTTAT |
| | | KLRKYEIA | | | TATTTATCCCATAAACG |
| | | KGLMSVPY | | | TATAATTCCTTCAAGTA |
| | | IYF | | | TGTTAACTATATATACT |
| | | | | | CAAATACAAAAGGATA |
| | | | | | TAAAAAACGGGAATAT |
| | | | | | TGACACCGAAAAATTA |
| | | | | | AGGAAATATGAGATAG |
| | | | | | CAAAAGGATTAATGTC |
| | | | | | CGTTCCTTATATATATT |
| | | | | | TCTAA |
| 506 | Nisin | MRRYLILI | Lactococcus | 507 | ATGAGAAGATATTTAAT |
| | immunity | VALIGITGL | lactis | | ACTTATTGTGGCCTTAA |
| | modulator | SGCYQTSH | subsp. | | TAGGGATAACAGGTTT |
| | | KKVRFDEG | lactis | | ATCAGGGTGTTATCAA |
| | | SYTNFIYD | (Streptococcus | | ACAAGTCATAAAAAGG |
| | | NKSYFVTD | lactis) | | TGAGGTTTGACGAAGG |
| | | KEIPQENV | | | AAGTTATACTAATTTTA |
| | | NNSKVKFY | | | TTTATGATAATAAATCG |
| | | KLLIVDMK | | | TATTTCGTAACTGATAA |
| | | SEKLLSSSN | | | GGAGATTCCTCAGGAG |
| | | KNSVTLVL | | | AACGTTAACAATTCCAA |
| | | NNIYEASD | | | AGTAAAATTTTATAAGC |
| | | KSLCMGIN | | | TGTTGATTGTTGACATG |
| | | DRYYKILP | | | AAAAGTGAGAAACTTT |
| | | ESDKGAVK | | | TATCAAGTAGCAACAA |
| | | ALRLQNFD | | | AAATAGTGTGACTTTGG |
| | | VTSDISDD | | | TCTTAAATAATATTTAT |
| | | NFVIDKND | | | GAGGCTTCTGACAAGT |
| | | SRKIDYMG | | | CGCTATGTATGGGTATT |
| | | NIYSISDTT | | | AACGACAGATACTATA |
| | | VSDEELGE | | | AGATACTTCCAGAAAG |
| | | YQDVLAE | | | TGATAAGGGGGCGGTC |
| | | VRVFDSVS | | | AAAGCTTTGAGATTACA |
| | | GKSIPRSE | | | AAACTTTGATGTGACAA |
| | | WGRIDKD | | | GCGATATTTCTGATGAT |
| | | GSNSKQSR | | | AATTTTGTTATTGATAA |
| | | TEWDYGEI | | | AAATGATTCACGAAAA |
| | | HSIRGKSLT | | | ATTGACTATATGGGAA |
| | | EAFAVEIN | | | ATATTTACAGTATATCG |
| | | DDFKLATK | | | GACACCACCGTATCTGA |
| | | VGN | | | TGAAGAATTGGGAGAA |
| | | | | | TATCAGGATGTTTTAGC |
| | | | | | TGAAGTACGTGTGTTTG |
| | | | | | ATTCAGTTAGTGGCAA |
| | | | | | AAGTATCCCGAGGTCT |
| | | | | | GAATGGGGGAGAATTG |
| | | | | | ATAAGGATGGTTCAAA |
| | | | | | TTCCAAACAGAGTAGG |
| | | | | | ACGGAATGGGATTATG |
| | | | | | GCGAAATCCATTCTATT |
| | | | | | AGAGGAAAATCTCTTA |
| | | | | | CTGAAGCATTTGCCGTT |
| | | | | | GAGATAAATGATGATT |
| | | | | | TTAAGCTTGCAACGAA |
| | | | | | GGTAGGAAACTAG |
| 508 | Trifolitoxin | MNDEICLT | Rhizobium | 509 | ATGAATGATGAGATTT |
| | immunity | GGGRTTVT | leguminosarum | | GCCTGACAGGTGGCGG |
| | modulator | RRGGVVY | bv. | | ACGAACGACTGTCACG |
| | | REGGPWSS | trifolii | | CGGCGCGGCGGAGTCG |
| | | TVISLLRHL | | | TGTATCGCGAAGGCGG |
| | | EASGFAEA | | | CCCGTGGTCATCAACCG |
| | | PSVVGTGF | | | TCATTTCGCTCCTGCGG |
| | | DERGRETL | | | CATCTGGAAGCCTCTGG |
| | | SFIEGEFVH | | | CTTCGCTGAAGCTCCTT |
| | | PGPWSEEA | | | CCGTTGTCGGCACCGGT |
| | | FPQFGMML | | | TTCGATGAGCGCGGCC |
| | | RRLHDATA | | | GGGAGACATTATCGTTT |
| | | SFKPPENS | | | ATCGAGGGTGAGTTTG |
| | | MWRDWFG | | | TTCACCCAGGCCCTTGG |
| | | RNLGEGQH | | | TCGGAGGAGGCTTTTCC |
| | | VIGHCDTG | | | GCAATTTGGAATGATGT |
| | | PWNIVCRS | | | TGCGGCGACTGCACGA |
| | | GLPVGLID | | | TGCCACCGCCTCGTTCA |
| | | WEVAGPV | | | AACCTCCCGAAAACTC |
| | | RADIELAQ | | | GATGTGGCGCGATTGG |
| | | ACWLNAQ | | | TTCGGGCGTAACCTCG |
| | | LYDDDIAE | | | GTGAGGGTCAACACGT |
| | | RVGLGSVT | | | AATAGGACACTGCGAC |
| | | MRAHQVR | | | ACAGGCCCATGGAACA |
| | | LLLDGYGL | | | TTGTTTGCCGGTCAGGA |
| | | SRKQRGGF | | | TTGCCTGTCGGGTTGAT |
| | | VDKLITFA | | | AGATTGGGAGGTGGCT |
| | | VHDAAEQ | | | GGGCCTGTCAGGGCGG |
| | | AKEAAVTP | | | ATATCGAATTGGCCCA |
| | | ESNDAEPL | | | GGCTTGTTGGCTGAATG |
| | | WAIAWRT | | | CCCAGCTCTACGATGAC |
| | | RSASWML | | | GACATTGCGGAGAGGG |
| | | HHRQTLEA | | | TCGGATTAGGCTCTGTG |
| | | ALA | | | ACCATGAGAGCGCATC |
| | | | | | AAGTTCGCCTGCTGCTT |
| | | | | | GACGGCTATGGTCTGTC |
| | | | | | TCGGAAGCAACGCGGC |
| | | | | | GGCTTCGTCGACAAGCT |
| | | | | | AATCACGTTCGCAGTTC |
| | | | | | ACGATGCGGCCGAGCA |
| | | | | | GGCGAAAGAGGCGGCT |
| | | | | | GTCACGCCAGAGTCGA |
| | | | | | ACGATGCGGAACCGCT |
| | | | | | ATGGGCAATTGCCTGG |
| | | | | | CGCACTAGAAGTGCCT |
| | | | | | CCTGGATGCTCCATCAT |
| | | | | | CGGCAAACACTGGAAG |
| | | | | | CAGCGCTGGCATAG |
| 510 | Anti- | MNNIIPIMS | Bacillus | 511 | ATGAATAACATAATCCC |
| | listerial | LLFKQLYS | subtilis | | TATCATGTCTTTGCTGT |
| | bacteriocin | RQGKKDAI | (strain 168) | | TCAAACAGCTTTACAGC |
| | subtilosin | RIAAGLVIL | | | CGGCAAGGGAAAAAGG |
| | bio- | AVFEIGLIR | | | ACGCCATCCGCATTGCC |
| | synthesis | QAGIDESV | | | GCAGGCCTTGTCATTCT |
| | protein | LRKTYIILA | | | GGCCGTGTTTGAAATC |
| | AlbD | LLLMNTY | | | GGGCTGATCCGCCAGG |
| | | MVFLSVTS | | | CCGGCATTGATGAATC |
| | | QWKESYM | | | GGTGTTGCGCAAAACG |
| | | KLSCLLPIS | | | TATATCATACTCGCGCT |
| | | SRSFWLAQ | | | TCTTTTGATGAACACAT |
| | | SVVLFVDT | | | ATATGGTGTTTCTTTCC |
| | | CLRRTLFFF | | | GTGACATCACAATGGA |
| | | ILPLFLFGN | | | AGGAATCTTATATGAA |
| | | GTLSGAQT | | | GCTGAGCTGCCTGCTGC |
| | | LFWLGRFS | | | CGATTTCTTCACGGAGC |
| | | FFTVYSIIF | | | TTTTGGCTCGCCCAGAG |
| | | GVVLSNHF | | | TGTCGTTTTGTTTGTCG |
| | | VKKKNLM | | | ATACCTGTTTGAGAAG |
| | | FLLHAAIFA | | | AACTTTATTCTTTTTTA |
| | | CVCISAAL | | | TTTTACCGCTGTTCTTA |
| | | MPAATIPL | | | TTTGGAAACGGAACGC |
| | | CAVHILWA | | | TGTCAGGGGCGCAAAC |
| | | VVIDFPVFL | | | ATTGTTTTGGCTCGGCA |
| | | QAPPQQGK | | | GGTTTTCGTTTTTTACC |
| | | MHSFMRRS | | | GTTTACTCCATTATTTT |
| | | EFSFYKRE | | | CGGAGTTGTGCTAAGC |
| | | WNRFISSK | | | AACCACTTCGTCAAAAA |
| | | AMLLNYA | | | GAAGAACTTGATGTTTC |
| | | VMAVFSGF | | | TGCTGCATGCGGCGAT |
| | | FSFQMMNT | | | ATTCGCCTGTGTATGTA |
| | | GIFNQQVI | | | TCAGCGCCGCTTTGATG |
| | | YIVISALLL | | | CCGGCCGCCACGATTCC |
| | | ICSPIALLY | | | GCTTTGCGCGGTTCATA |
| | | SIEKNDRM | | | TCCTGTGGGCGGTGGT |
| | | LLITLPIKR | | | CATTGACTTTCCTGTCT |
| | | KTMFWAK | | | TTCTGCAGGCGCCTCCG |
| | | YRFYSGLL | | | CAGCAGGGCAAGATGC |
| | | AGGFLLVV | | | ATTCATTTATGCGGCGA |
| | | MIVGFISGR | | | TCTGAATTTTCGTTTTA |
| | | SISVLTFLQ | | | CAAAAGAGAATGGAAC |
| | | CIELLLAG | | | CGATTTATCTCTTCTAA |
| | | AYIRLTAD | | | AGCGATGCTGTTAAATT |
| | | EKRPSFSW | | | ACGCGGTAATGGCGGT |
| | | QTEQQLWS | | | ATTCAGCGGCTTCTTTT |
| | | GFSKYRSY | | | CGTTCCAGATGATGAA |
| | | LFCLPLFLA | | | CACCGGCATCTTCAATC |
| | | ILAGTAVS | | | AGCAAGTGATTTATATC |
| | | LAVIPIAGL | | | GTGATTTCCGCGCTTTT |
| | | VIVYYLQK | | | GCTCATCTGCTCGCCGA |
| | | QDGGFFDT | | | TCGCCCTTTTGTATTCG |
| | | SKRERLGS | | | ATTGAAAAAAATGACC |
| | | | | | GGATGCTGCTCATCACG |
| | | | | | CTTCCGATCAAGCGAA |
| | | | | | AAACGATGTTTTGGGC |
| | | | | | GAAATATCGCTTTTATT |
| | | | | | CAGGCCTATTGGCAGG |
| | | | | | CGGATTTCTCCTTGTCG |
| | | | | | TGATGATTGTGGGTTTC |
| | | | | | A |
| 512 | Putative | MSILDIHD | Bacillus | 513 | GCATTTTGGATATACAC |
| | ABC | VSVWYER | subtilis | | GATGTATCCGTTTGGTA |
| | transporter | DNVILEQV | (strain 168) | | TGAACGGGACAACGTC |
| | ATP- | DLHLEKGA | | | ATCTTAGAGCACGTGG |
| | binding | VYGLLGV | | | ACTTACACTTAGAAAAA |
| | protein | NGAGKTTL | | | GGCGCCGTTTACGGATT |
| | AlbC | INTLTGVN | | | GCTTGGGGTAAACGGT |
| | (Anti- | RNFSGRFT | | | GCCGGCAAAACAACAC |
| | listerial | LCGIEAEA | | | TGATCAATACGCTGACA |
| | bacteriocin | GMPQKTSD | | | GGAGTGAACCGCAATT |
| | subtilosin | QLKTHRYF | | | ACAGCGGGGGCTTTAC |
| | biosynthesis | AADYPLLF | | | GCTGTGCGGCATTGAA |
| | protein | TEITAKDY | | | GCTGAGGCCGGCATGC |
| | AlbC) | VSFVHSLY | | | CGCAGAAAACATCAGA |
| | | QKDFSEQQ | | | TCAACTGAAGATTCACC |
| | | FASLAEAF | | | GTTACTTCGCCGCTGAT |
| | | HFSKYINR | | | TATCCGCTGCTGTTTAC |
| | | RISELSLGN | | | AGAAATTACGGCGAAG |
| | | RQKVVLM | | | GACTATGTGTCTTTCGT |
| | | TGLLLRAP | | | CCATTCGCTTTATCAAA |
| | | LFILDEPLV | | | AGGATTTTTCAGAGCG |
| | | GLDVESIE | | | ACAGTTTGCCAGTTTGG |
| | | VFYQKMR | | | CTGAGGCCTTTCATTTT |
| | | EYCEAGGT | | | TCAAAATACATCAACA |
| | | ILFSSHLLD | | | GGAGAATCTCGGAGCT |
| | | VVQRFCDY | | | GTCCTTGGGGAACAGG |
| | | AAILHNKQ | | | CAAAAGGTTGTGTTGAT |
| | | IQKVIPIGE | | | GACAGGATTATTGCTGC |
| | | ETDLRREF | | | GGGCTCCCCTGTTTATT |
| | | FEVIGHE | | | TTGGATGAGCCGCTCGT |
| | | | | | CGGTTTGGATGTGGAA |
| | | | | | TCAATAGAGGTCTTTTA |
| | | | | | TCAGAAAATGCGGGAG |
| | | | | | TACTGTGAGGAAGGCG |
| | | | | | GAACCATTTTGTTTTCT |
| | | | | | TCCCATCTGCTCGATGT |
| | | | | | CGTGCAGAGATTTTGTG |
| | | | | | ATTTTGCGGCCATTCTG |
| | | | | | CACAACAAACAGATCC |
| | | | | | AAAAGGTCATTCCGATT |
| | | | | | GGGGAGGAGACCGATC |
| | | | | | TGCGGCGGGAATTTTTT |
| | | | | | GAGGTTATCGGCCATG |
| | | | | | AATAA |
| 514 | Anti- | MSPAQRRI | Bacillus | 515 | TTGTCACCAGCACAAA |
| | listerial | LLYILSFIF | subtilis | | GAAGAATTTTACTGTAT |
| | bacteriocin | VIGAVVYF | (strain 168) | | ATCCTTTCATTTATCTT |
| | subtilosin | VKSDYLFT | | | TGTCATCGGCGCAGTC |
| | bio- | LIFIAIAILF | | | GTCTATTTTGTCAAAAG |
| | synthesis | GMRARKA | | | CGATTATCTGTTTACGC |
| | protein | DSR | | | TGATTTTCATTGCCATT |
| | AlbB | | | | GCCATTCTGTTCGGGAT |
| | | | | | GCGCGCGCGGAAGGCT |
| | | | | | GACTCGCGATGA |
| 516 | Colicin-E7 | MELKNSIS | Escherichia | 517 | ATGGAACTGAAAAATA |
| | immunity | DYTEAEFV | coli | | GTATTAGTGATTACACA |
| | modulator | QLLKEIEK | | | GAGGCTGAGTTTGTTCA |
| | (ImmE7) | ENVAATD | | | ACTTCTTAAGGAAATTG |
| | (Microcin- | DVLDVLLE | | | AAAAAGAGAATGTTGC |
| | E7 | HFVKITEH | | | TGCAACTGATGATGTGT |
| | immunity | PDGTDLIY | | | TAGATGTGTTACTCGAA |
| | modulator) | YPSDNRDD | | | CACTTTGTAAAAATTAC |
| | | SPEGIVKEI | | | TGAGCATCCAGATGGA |
| | | KEWRAAN | | | ACGGATCTGATTTATTA |
| | | GKPGFKQG | | | TCCTAGTGATAATAGA |
| | | | | | GACGATAGCCCCGAAG |
| | | | | | GGATTGTCAAGGAAAT |
| | | | | | TAAAGAATGGCGAGCT |
| | | | | | GCTAACGGTAAGCCAG |
| | | | | | GATTTAAACAGGGCTG |
| | | | | | A |
| 518 | Pyocin-S1 | MKSKISEY | Pseudomonas | 519 | ATGAAGTCCAAGATTTC |
| | immunity | TEKEFLEF | aeruginosa | | CGAATATACGGAAAAA |
| | modulator | VEDIYTNN | | | GAGTTTCTTGAGTTTGT |
| | | KKKFPTEE | | | TGAAGACATATACACA |
| | | SHIQAVLE | | | AACAATAAGAAAAAGT |
| | | FKKLTEHP | | | TCCCTACCGAGGAGTCT |
| | | SGSDLLYY | | | CATATTCAAGCCGTGCT |
| | | PNENREDS | | | TGAATTTAAAAAACTAA |
| | | PAGVVKEV | | | CGGAACACCCAAGCGG |
| | | KEWRASK | | | CTCAGACCTTCTTTACT |
| | | GLPGFKAG | | | ACCCCAACGAAAATAG |
| | | | | | AGAAGATAGCCCAGCT |
| | | | | | GGAGTTGTAAAGGAAG |
| | | | | | TTAAAGAATGGCGTGC |
| | | | | | TTCCAAGGGGCTTCCTG |
| | | | | | GCTTTAAGGCCGGTTA |
| | | | | | G |
| 520 | Pyocin-S2 | MKSKISEY | Pseudomonas | 521 | ATGAAGTCCAAGATTTC |
| | immunity | TEKEFLEF | aeruginosa | | CGAATATACGGAAAAA |
| | modulator | VKDIYTNN | (strain | | GAGTTTCTTGAGTTTGT |
| | | KKKFPTEE | ATCC | | TAAAGACATATACACA |
| | | SHIQAVLE | 15692/ | | AACAATAAGAAAAAGT |
| | | FKKLTEHP | PAOl/1C/ | | TCCCTACCGAGGAGTCT |
| | | SGSDLLYY | PRS 101/ | | CATATTCAAGCCGTGCT |
| | | PNENREDS | LMG | | TGAATTTAAAAAACTAA |
| | | PAGVVKEV | 12228) | | CGGAACACCCAAGCGG |
| | | KEWRASK | | | CTCAGACCTTCTTTACT |
| | | GLPGFKAG | | | ACCCCAACGAAAATAG |
| | | | | | AGAAGATAGCCCAGCT |
| | | | | | GGAGTTGTAAAGGAAG |
| | | | | | TTAAAGAATGGCGTGC |
| | | | | | TTCCAAGGGGCTTCCTG |
| | | | | | GCTTTAAGGCCGGTTA |
| | | | | | G |
| 522 | Hiracin- | MDFTKEEK | Enterococcus | 523 | ATGGATTTTACTAAAGA |
| | JM79 | LLNAISKV | hirae | | AGAAAAACTTTTAAAT |
| | immunity | YNEATIDD | | | GCAATTAGTAAAGTAT |
| | factor | YPDLKEKL | | | ACAATGAAGCAACTAT |
| | | FLYSKEISE | | | AGATGACTATCCTGACT |
| | | GKSVGEVS | | | TAAAAGAAAAGCTCTTT |
| | | MKLSSFLG | | | CTTTATTCTAAAGAAAT |
| | | RYILKHKF | | | CAGTGAGGGAAAAAGT |
| | | GLPKSLIEL | | | GTTGGTGAAGTTAGTAT |
| | | QEIVSKES | | | GAAATTAAGTAGTTTTC |
| | | QVYRGWA | | | TTGGAAGATATATTTTA |
| | | SIGIWS | | | AAACATAAATTTGGATT |
| | | | | | ACCTAAATCTTTAATAG |
| | | | | | AATTACAAGAAATTGTT |
| | | | | | AGTAAGGAATCTCAAG |
| | | | | | TATATAGAGGATGGGC |
| | | | | | TTCTATTGGTATTTGGA |
| | | | | | GTTAA |
| 524 | Probable | MKKKYRY | Leuconostoc | 525 | TTGAAAAAAAAGTATC |
| | mesenter- | LEDSKNYT | mesenteroides | | GGTATTTAGAAGATAG |
| | icin-Y105 | STLYSLLV | | | CAAAAATTACACTAGTA |
| | immunity | DNVDKPG | | | CACTCTATTCTCTGTTA |
| | modulator | YSDICDVL | | | GTTGATAATGTTGACAA |
| | | LQVSKKLD | | | ACCTGGATACTCAGATA |
| | | NTQSVEAL | | | TTTGCGATGTTTTGCTT |
| | | INRLVNYIR | | | CAAGTTTCTAAGAAGTT |
| | | ITASTYKIIF | | | GGATAATACTCAAAGT |
| | | SKKEEELII | | | GTTGAAGCGCTAATTA |
| | | KLGVIGQK | | | ATCGATTGGTTAATTAT |
| | | AGLNGQY | | | ATTCGTATTACTGCTTC |
| | | MADFSDKS | | | AACATACAAAATTATTT |
| | | QFYSVFDQ | | | TTTCAAAAAAAGAAGA |
| | | | | | GGAATTGATTATAAAA |
| | | | | | CTTGGTGTTATTGGACA |
| | | | | | AAAAGCTGGACTTAAT |
| | | | | | GGTCAGTATATGGCTG |
| | | | | | ATTTTTCAGACAAGTCT |
| | | | | | CAGTTTTACAGCGTTTT |
| | | | | | CGATCAGTAA |
| 526 | Microcin- | MSFLNFAF | Escherichia | 527 | ATGAGTTTTCTTAATTT |
| | 24 | SPVFFSIMA | coli | | TGCATTTTCTCCTGTAT |
| | immunity | CYFIVWRN | | | TCTTCTCCATTATGGCG |
| | modulator | KRNEFVCN | | | TGTTATTTCATTGTATG |
| | | RLLSIIIISFL | | | GAGAAATAAACGAAAC |
| | | ICFIYPWLN | | | GAATTTGTCTGCAATAG |
| | | YKIEVKYY | | | ATTGCTATCAATTATAA |
| | | IFEQFYLFC | | | TAATATCTTTTTTGATA |
| | | FLSSLVAV | | | TGCTTCATATATCCATG |
| | | VINLIVYFI | | | GCTAAATTACAAAATC |
| | | LYRRCI | | | GAAGTTAAATATTATAT |
| | | | | | ATTTGAACAGTTTTATC |
| | | | | | TTTTTTGTTTTTTATCGT |
| | | | | | CACTCGTGGCTGTTGTA |
| | | | | | ATAAACCTAATTGTATA |
| | | | | | CTTTATATTATACAGGA |
| | | | | | GATGTATATGA |
| 528 | Colicin-K | MHLKYYL | Escherichia | 529 | ATGCATTTAAAATACTA |
| | immunity | HNLPESLIP | coli | | CCTACATAATTTACCTG |
| | modulator | WILILIFND | | | AATCACTTATACCATGG |
| | | NDNTPLLFI | | | ATTCTTATTTTAATATT |
| | | FISSIHVLL | | | TAACGACAATGATAAC |
| | | YPYSKLTIS | | | ACTCCTTTGTTATTTAT |
| | | RYIKENTK | | | ATTTATATCATCAATAC |
| | | LKKEPWYL | | | ATGTATTGCTATATCCA |
| | | CKLSALFY | | | TACTCTAAATTAACCAT |
| | | LLMAIPVG | | | ATCTAGATATATCAAAG |
| | | LPSFIYYTL | | | AAAATACAAAGTTAAA |
| | | KRN | | | AAAAGAACCCTGGTAC |
| | | | | | TTATGCAAGTTATCTGC |
| | | | | | ATTGTTTTATTTATTAA |
| | | | | | TGGCAATCCCAGTAGG |
| | | | | | ATTGCCAAGTTTCATAT |
| | | | | | ATTACACTCTAAAGAG |
| | | | | | AAATTAA |
| 530 | Microcin | MMIQSHPL | Escherichia | 531 | ATGATGATACAATCTCA |
| | C7 self- | LAAPLAVG | coli | | TCCACTACTGGCCGCTC |
| | immunity | DTIGFFSSS | | | CCCTGGCAGTAGGAGA |
| | modulator | APATVTAK | | | TACAATTGGTTTCTTTT |
| | MccF | NRFFRGVE | | | CATCATCTGCTCCGGCA |
| | | FLQRKGFK | | | ACAGTTACTGCAAAAA |
| | | LVSGKLTG | | | ATCGTTTTTTTCGGGGA |
| | | KTDFYRSG | | | GTTGAGTTTCTTCAGAG |
| | | TIKERAQE | | | AAAGGGATTTAAGCTG |
| | | FNELVYNP | | | GTATCAGGGAAGCTTA |
| | | DITCIMSTI | | | CCGGTAAAACAGATTTT |
| | | GGDNSNSL | | | TATCGTTCAGGTACTAT |
| | | LPFLDYDA | | | TAAAGAAAGAGCTCAA |
| | | IIANPKIIIG | | | GAATTTAATGAGTTAGT |
| | | YSDTTALL | | | CTACAATCCTGATATTA |
| | | AGIYAKTG | | | CCTGTATAATGTCAACG |
| | | LITFYGPAL | | | ATCGGTGGAGATAACA |
| | | IPSFGEHPP | | | GTAATTCACTACTACCG |
| | | LVDITYESF | | | TTTCTGGACTATGATGC |
| | | IKILTRKQS | | | TATCATTGCAAACCCCA |
| | | GIYTYTLP | | | AAATTATCATAGGTTAC |
| | | EKWSDESI | | | TCAGATACAACTGCTTT |
| | | NWNENKIL | | | ATTAGCAGGAATATAT |
| | | RPKKLYKN | | | GCAAAAACAGGGTTAA |
| | | NCAFYGSG | | | TAACATTCTATGGACCA |
| | | KVEGRVIG | | | GCTCTTATTCCTTCGTT |
| | | GNLNTLTG | | | TGGTGAACATCCACCTC |
| | | IWGSEWM | | | TTGTGGATATAACATAT |
| | | PEILNGDIL | | | GAATCATTTATTAAAAT |
| | | FIEDSRKSI | | | ACTAACAAGAAAACAA |
| | | ATIERLFS | | | TCAGGAATATATACCTA |
| | | MLKLNRVF | | | CACATTACCTGAAAAGT |
| | | DKVSAIILG | | | GGAGTGATGAGAGCAT |
| | | KHELFDCA | | | AAACTGGAATGAAAAC |
| | | GSKRRPYE | | | AAGATATTAAGGCCTA |
| | | VLTEVLDG | | | AGAAGCTATATAAAAA |
| | | KQIPVLDG | | | CAACTGTGCCTTTTATG |
| | | FDCSHTHP | | | GTTCCGGAAAAGTTGA |
| | | MLTLPLGV | | | GGGGCGTGTAATTGGA |
| | | KLAIDFDN | | | GGAAATCTAAATACTTT |
| | | KNISITEQY | | | GACAGGTATATGGGGG |
| | | LSTEK | | | AGTGAATGGATGCCTG |
| | | | | | AAATTCTTAATGGAGAT |
| | | | | | ATATTGTTTATTGAGGA |
| | | | | | CAGTCGGAAAAGCATT |
| | | | | | GCAACAATTGAACGAT |
| | | | | | TATTCTCTATGCTAAAG |
| | | | | | CTTAATCGCGTGTTTGA |
| | | | | | TAAAGTTAGTGCAATA |
| | | | | | ATACTCGGGAAACATG |
| | | | | | AGCTTTTTGATTGTGCA |
| | | | | | GGAAGTAAACGCAGAC |
| | | | | | CATATGAAGTATTAACA |
| | | | | | GAGGTATTAGATGGGA |
| | | | | | AACAGATTCCTGTACTG |
| | | | | | GATGGATTTGATTGTTC |
| | | | | | ACATACACATCCAATGC |
| | | | | | TAACTCTTCCACTTGGT |
| | | | | | GTAAAATTAGCTATTGA |
| | | | | | CTTTGACAACAAAAATA |
| | | | | | TAT |
| 532 | Sakacin-A | MKADYKKI | Lactobacillus | 533 | GGCAGATTATAAAAAA |
| | immunity | NSILTYTST | sakei | | ATAAATTCAATACTAAC |
| | factor | ALKNPKIIK | | | TTACACATCTACTGCTT |
| | | DKDLVVLL | | | TAAAAAACCCTAAAATT |
| | | TIIQEEAKQ | | | ATAAAAGATAAAGATT |
| | | NRIFYDYK | | | TAGTAGTCCTTCTAACT |
| | | RKFRPAVT | | | ATTATTCAAGAAGAAG |
| | | RFTIDNNFE | | | CCAAACAAAATAGAAT |
| | | IPDCLVKL | | | CTTTTATGATTATAAAA |
| | | LSAVETPK | | | GAAAATTTCGTCCAGC |
| | | AWSGFS | | | GGTTACTCGCTTTACAA |
| | | | | | TTGATAATAATTTTGAG |
| | | | | | ATTCCTGATTGTTTGGT |
| | | | | | TAAACTACTGTCAGCTG |
| | | | | | TTGAAACACCTAAGGC |
| | | | | | GTGGTCTGGATTTAGTT |
| | | | | | AG |
| 534 | Colicin-E5 | MKLSPKAA | Escherichia | 535 | TGAAGTTATCACCAAA |
| | immunity | IEVCNEAA | coli | | AGCTGCAATAGAAGTT |
| | modulator | KKGLWILG | | | TGTAATGAAGCAGCGA |
| | in ColE9 | IDGGHWLN | | | AAAAAGGCTTATGGAT |
| | (E5Imm[E | PGFRIDSSA | | | TTTGGGCATTGATGGTG |
| | 9]) | SWTYDMP | | | GGCATTGGCTGAATCCT |
| | | EEYKSKTP | | | GGATTCAGGATAGATA |
| | | ENNRLAIE | | | GTTCAGCATCATGGAC |
| | | NIKDDIEN | | | ATATGATATGCCGGAG |
| | | GYTAFIITL | | | GAATACAAATCAAAAA |
| | | KM | | | CCCCTGAAAATAATAG |
| | | | | | ATTGGCTATTGAAAATA |
| | | | | | TTAAAGATGATATTGA |
| | | | | | GAATGGATACACTGCTT |
| | | | | | TCATTATCACGTTAAAG |
| | | | | | ATGTAA |
| 536 | Anti- | MNNIFPIM | Bacillus | 537 | TTGGGGAGGAGACCGA |
| | listerial | SLLFKQLY | subtilis | | TCTGCGGCGGGAATTTT |
| | bacteriocin | SRQGKKDA | | | TTGAGGTTATCGGCCAT |
| | subtilosin | IRIAAGLVI | | | GAATAACATATTCCCCA |
| | bio- | LAVFEIGLI | | | TCATGTCGTTGCTGTTC |
| | synthesis | RQAGIDES | | | AAACAGCTGTACAGCC |
| | protein | VLGKTYIIL | | | GGCAAGGGAAAAAGGA |
| | AlbD | ALLLMNTY | | | CGCTATCCGCATTGCTG |
| | | MVFLSVTS | | | CAGGGCTTGTGATTCTC |
| | | QWKESYM | | | GCCGTGTTTGAAATCG |
| | | KLSCLLPIS | | | GGCTGATCCGACAAGC |
| | | SRSFWLAQ | | | CGGCATTGACGAATCG |
| | | SVVLFVDT | | | GTGTTGGGAAAAACGT |
| | | CLRRTLFFF | | | ATATCATATTGGCGCTT |
| | | ILPLFLFGN | | | CTCTTAATGAACACGTA |
| | | GTLSGAQT | | | TATGGTGTTTCTTTCCG |
| | | LFWLGRFS | | | TGACATCACAATGGAA |
| | | FFTVYSILF | | | GGAATCTTATATGAAG |
| | | GVMLSNHF | | | CTGAGCTGTCTGCTGCC |
| | | VKKKNSM | | | GATTTCATCACGGAGCT |
| | | FLLHAAVF | | | TTTGGCTCGCCCAGAGT |
| | | AFVCLSAA | | | GTCGTTCTGTTTGTCGA |
| | | FMPAVTIP | | | TACCTGTTTGAGAAGA |
| | | LCAVHML | | | ACGTTATTCTTTTTTAT |
| | | WAVIIDFP | | | TTTACCGCTGTTCTTAT |
| | | VFLQAPPH | | | TTGGAAACGGAACGCT |
| | | QSKMHFF | | | GTCAGGGGCGCAAACA |
| | | MRRSEFSF | | | TTGTTTTGGCTTGGCAG |
| | | YKREWNR | | | ATTTTCGTTTTTTACCG |
| | | FISSKAMLL | | | TTTACTCGATTCTATTC |
| | | NYVVMAA | | | GGAGTTATGCTAAGCA |
| | | FSGFFSFQ | | | ACCATTTCGTCAAAAAG |
| | | MMNTGIFN | | | AAGAACTCGATGTTTCT |
| | | QQVIYIVIS | | | GCTGCATGCGGCGGTA |
| | | ALLLICSPI | | | TTCGCCTTTGTATGCCT |
| | | ALLYSIEK | | | CAGTGCCGCTTTTATGC |
| | | NDRMLLIT | | | CGGCCGTCACGATCCC |
| | | LPIKRRTM | | | GCTATGCGCGGTTCACA |
| | | FWAKYRF | | | TGCTATGGGCGGTGAT |
| | | YSGLLAGG | | | CATTGACTTTCCGGTCT |
| | | FLLVAIIVG | | | TTCTGCAGGCGCCTCCG |
| | | FISGRPISA | | | CATCAGAGCAAGATGC |
| | | LTFVQCME | | | ATTTTTTTATGCGGCGA |
| | | LLLAGAFIR | | | TCTGAATTTTCGTTTTA |
| | | LTADEKRP | | | CAAAAGAGAATGGAAC |
| | | SFGWQTEQ | | | CGATTTATTTCTTCTAA |
| | | QLWSGFSK | | | AGCGATGCTGTTAAATT |
| | | YRSYLFCL | | | ACGTGGTGATGGCGGC |
| | | PLFLATLA | | | GTTCAGCGGATTCTTTT |
| | | GTAVSLAV | | | CGTTCCAGATGATGAA |
| | | IPIAALIIVY | | | CACTGGCATCTTCAATC |
| | | YLQKQDG | | | AGCAAGTGATTTATATT |
| | | GFFDTSKR | | | GTGATTTCCGCTCTATT |
| | | ERIGS | | | GCTGATTTGCTCGCCGA |
| | | | | | TCGCCCTTTTGTACTCT |
| | | | | | ATTGAAAAAAACGATC |
| | | | | | GCATGCTGCTCATCACG |
| | | | | | CTTCCAATTAAAAGAA |
| | | | | | GAACGATGTTTTGGGC |
| | | | | | GAAATATCGCTTTTATT |
| | | | | | CAG |
| 538 | Microcin- | MERKQKN | Escherichia | 539 | ATGGAAAGAAAACAGA |
| | J25 export | SLFNYIYSL | coli | | AAAACTCATTATTTAAT |
| | ATP- | MDVRGKF | | | TATATTTATTCATTAAT |
| | binding/per | LFFSMLFIT | | | GGATGTAAGAGGTAAA |
| | mease | SLSSIIISISP | | | TTTTTATTCTTTTCCAT |
| | protein | LILAKITDL | | | GTTATTCATTACATCAT |
| | McjD | LSGSLSNFS | | | TATCATCGATAATCATA |
| | (Microcin- | YEYLVLLA | | | TCTATTTCACCATTGAT |
| | J25 | CLYMFCVI | | | TCTTGCAAAGATTACAG |
| | immunity | SNKASVFL | | | ATTTACTGTCTGGCTCA |
| | modulator) | FMILQSSLR | | | TTGTCAAATTTTAGTTA |
| | (Microcin- | INMQKKM | | | TGAATATCTGGTTTTAC |
| | J25 | SLKYLREL | | | TTGCCTGTTTATACATG |
| | secretion | YNENITNL | | | TTTTGCGTTATATCTAA |
| | ATP- | SKNNAGYT | | | TAAAGCAAGTGTTTTTT |
| | binding | TQSLNQAS | | | TATTTATGATACTGCAA |
| | protein | NDIYILVR | | | AGTAGTCTACGTATTAA |
| | McjD) | NVSQNILS | | | CATGCAGAAAAAAATG |
| | | PVIQLISTI | | | TCGCTAAAGTATTTGAG |
| | | VVVLSTKD | | | AGAATTGTATAACGAA |
| | | WFSAGVFF | | | AATATAACTAACTTGAG |
| | | LYILVFVIF | | | TAAAAATAATGCTGGA |
| | | NTRLTGSL | | | TATACAACGCAAAGTCT |
| | | ASLRKHSM | | | TAACCAGGCTTCAAATG |
| | | DITLNSYSL | | | ACATTTATATTCTTGTG |
| | | LSDTVDN | | | AGAAATGTTTCCCAGA |
| | | MIAAKKNN | | | ATATCCTGTCACCTGTT |
| | | ALRLISERY | | | ATACAACTTATTTCCAC |
| | | EDALTQEN | | | TATTGTTGTTGTTTTAT |
| | | NAQKKYW | | | CTACGAAGGACTGGTTT |
| | | LLSSKVLL | | | TCTGCCGGTGTGTTTTT |
| | | LNSLLAVIL | | | TCTCTATATTCTGGTAT |
| | | FGSVFIYNI | | | TTGTAATTTTTAATACC |
| | | LGVLNGV | | | AGACTGACTGGCAGTTT |
| | | VSIGHFIMI | | | AGCGTCTCTCAGAAAA |
| | | TSYIILLST | | | CACAGCATGGATATCA |
| | | PVENIGAL | | | CTCTTAACTCTTATAGT |
| | | LSEIRQSM | | | CTGTTATCTGATACTGT |
| | | SSLAGFIQR | | | TGATAACATGATAGCA |
| | | HAENKATS | | | GCTAAAAAGAATAATG |
| | | PSIPFLNME | | | CATTAAGACTTATTTCT |
| | | RKLNLSIRE | | | GAACGTTATGAAGATG |
| | | LSFSYSDD | | | CTCTCACTCAGGAAAAC |
| | | KKILNSVS | | | AATGCTCAGAAAAAAT |
| | | LDLFTGKM | | | ACTGGTTACTCAGTTCT |
| | | YSLTGPSG | | | AAAGTTCTTTTATTGAA |
| | | SGKSTLVK | | | CTCTTTACTTGCTGTAA |
| | | IISGYYKN | | | TATTATTTGGTTCTGTA |
| | | YFGDIYLN | | | TTCATATATAATATTTT |
| | | DISLRNISD | | | AGGTGTGCTGAATGGT |
| | | EDLNDAIY | | | GTAGTTAGTATCGGCCA |
| | | YLTQDDYI | | | CTTCATTATGATTACAT |
| | | FMDTLRFN | | | CATATATCATTCTTCTT |
| | | LRLANYDA | | | TCAACGCCAGTGGAAA |
| | | SENEIFKVL | | | ATATAGGGGCATTGCT |
| | | KLANLSVV | | | AAGTGAGATCAGGCAG |
| | | NNEPVSLD | | | TCAATGTCTAGCCTGGC |
| | | THLINRGN | | | AGGTTTTATTCAACGTC |
| | | NYSGGQK | | | ATGCCGAGAATAAAGC |
| | | QRISLARLF | | | CACATCTCCTTCAA |
| | | LRKPAIIIID | | | |
| | | EATSALDY | | | |
| | | INESEILSSI | | | |
| | | RTHFPDALI | | | |
| | | INISHRINL | | | |
| | | LECSDCVY | | | |
| | | VLNEGNIV | | | |
| | | ASGHFRDL | | | |
| | | MVSNEYIS | | | |
| | | GLASVTE | | | |
| 540 | Microcin | MTLLSFGF | Klebsiella | 541 | ATGACATTACTTTCATT |
| | E492 | SPVFFSVM | pneumoniae | | TGGATTTTCTCCTGTTT |
| | immunity | AFCIISRSK | | | TCTTTTCAGTCATGGCG |
| | modulator | FYPQRTRN | | | TTCTGTATCATTTCACG |
| | | KVIVLILLT | | | TAGTAAATTCTATCCGC |
| | | FFICFLYPL | | | AGAGAACGCGAAACAA |
| | | TKVYLVGS | | | AGTTATTGTTCTGATTT |
| | | YGIFDKFY | | | TACTAACTTTTTTTATT |
| | | LFCFISTLI | | | TGTTTTTTATATCCATT |
| | | AIAINVVIL | | | AACAAAAGTGTATCTG |
| | | TINGAKNE | | | GTGGGAAGTTACGGTA |
| | | RN | | | TATTTGACAAATTCTAC |
| | | | | | CTCTTTTGCTTTATTTC |
| | | | | | TACGTTAATTGCAATAG |
| | | | | | CAATTAACGTAGTGATA |
| | | | | | CTTACAATAAATGGAG |
| | | | | | CTAAGAATGAGAGAAA |
| | | | | | TTAG |

### Poison-Antidote Systems

It can be desirable to contain a particular microbial cell within a desired environment, for example by killing or arresting the growth of the microbial cell if it is no longer in the desired environment. Poison-antidote systems, which are distinct from bacteriocins, can be useful for accomplishing such containment, or for other selective growth of microbial cells. Exemplary poison antidote systems are described in U.S. Pat. Nos. 5,910,438, 6,180,407, 7,176,029, and 7,183,097, each of which is hereby incorporated by reference in its entirety. In some embodiments, a poison-antidote system comprises a cytotoxic (poison) polypeptide, and a corresponding antitoxin (antidote) polypeptide in a single cell. As used herein, a “poison polynucleotide” refers to a polynucleotide encoding a poison polypeptide, and an “antidote polynucleotide” refers to a polynucleotide encoding an antidote polypeptide.

In some embodiments, the poison polypeptide is expressed constitutively, while the antidote polypeptide is only expressed under desired conditions. In some embodiments, the poison polypeptide is only expressed under undesired conditions, while the antidote polypeptide is only expressed under desired conditions. For example, in some embodiments, a poison/antidote system is configured so that the microbial cell survives under desired environmental conditions, but dies under undesired environmental conditions. For example, in some embodiments, a poison antidote system is configured so that the microbial cell is killed if it escapes from the environment in which it is being used in an industrial process. In other embodiments, a poison antidote system is configured so that the microbial cell survives when a vector (e.g. a plasmid) encoding an antidote polypeptide is present, but dies when the vector is absent. In some embodiments, the poison polypeptide is encoded by a poison polynucleotide in the host genome, while the antidote polypeptide is encoded by an antidote polynucleotide on a vector (such as a plasmid or extrachromosomal array or episome or minichromosome), and as such is only expressed when the vector is present in the host cell. In some embodiments, the poison polypeptide is encoded by a poison polynucleotide on a first vector, while the antidote polypeptide is encoded by an antidote polynucleotide on a second vector, and as such is only expressed when the second vector is present. In some embodiments, the presence of the antidote polynucleotide (and thus the presence of the antidote polypeptide) depends on the presence or absence of a recombination event, for example the integration of a polynucleotide sequence encoding the antidote polynucleotide into the host genome. It should be appreciated that in some embodiments in which expression of the antidote polypeptide depends on the presence or absence of a vector or recombination event, the poison and antidote polypeptide can each be expressed constitutively. Optionally, in some embodiments in which expression of the antidote polypeptide depends on the presence or absence of a vector or a recombination event, expression of the poison polypeptide and/or antidote polypeptide is conditional, for example so that the poison is only expressed in conditions in which the microbial cell is not desired, and/or the antidote polypeptide is only expressed in conditions in which the microbial cell is desired.

Exemplary microbial toxin polypeptide/antitoxin polypeptide pairs (also referred to as “poison/antidote” pairs) that can used in poison antidote systems in conjunction with some embodiments herein include, but are not limited to RelE/RelB, CcdB/CcdA, Kis/Kid, SoK/HoK, PasB (or PasC)/PasA, PemK/PemI, Doc/Phd, MazE/MazF and ParE/ParD. Without being limited by any particular theory, many poison polypeptides, for example RelE, are highly conserved across Gram-positive and Gram-negative bacteria and Archae, and as such, can have cytotoxic activity in a broad range of naturally occurring, genetically modified, and fully synthetic microbial cells. Further, without being limited by any particular theory, it is contemplated that an antidote polypeptide can generally inhibit the activity of its poison polypeptide partner in a variety of host environments, and as such, poison/antidote pairs such as those described herein can readily be used in a broad range of naturally occurring, genetically modified, and fully synthetic microbial cells.

It is noted that a poison-antidote system is distinct from a bacteriocin system at least in that a poison-antidote system provides an endogenous system by which a microbial cell can kill or arrest itself, while a bacteriocin system provides an exogenous system by which a microbial cell can kill or arrest other cells. It is further noted, however, that, while a poison-antidote system cannot be used to kill or arrest cells other than the individual cell in which the poison is produced, in some embodiments, a poison-antidote system may be used along with a bacteriocin system as described herein. For example, in some embodiments a bacteriocin system as described herein may be used to kill or arrest the growth of cells other than the bacteriocin producing cell in a culture while the poison-antidote system may be used to kill or arrest the growth of the bacteriocin producing cell should it escape from its desired environment. A poison-antidote system may also be used to select for bacteriocin producing cells which have been genetically engineered to express a molecule useful in an industrial process (an “industrially useful molecule”). For example, in some embodiments, expression of an antidote can be tied to expression of an industrially useful molecule or bacteriocin by placing polynucleotides encoding the bacteriocin and the industrially useful molecule, or polynucleotides encoding the bacteriocin and antidote under the control of a single promoter. Accordingly, in some embodiments, a microbial cell encoding a bacteriocin or bacteriocin immunity modulator further comprises a poison antidote system. In some embodiments, the bacteriocin system is useful for regulating growth of the microbial cell or other microbial cells within a particular environment, while the poison-antidote system is useful for containing the microbial cell within a particular environment.

### Promoters

Promoters are well known in the art. A promoter can be used to drive the transcription of one or more genes. In some embodiments, a promoter drives expression of polynucleotide encoding a desired gene product as described herein. In some embodiments, a promoter drives expression of a bacteriocin polynucleotide as described herein. In some embodiments, a promoter drives expression of an immunity modulator polynucleotide as described herein. In some embodiments, a promoter drives expression of a bacteriocin nucleotide and an immunity modulator polynucleotide. In some embodiments, a promoter drives expression of polynucleotide encoding at least one of a bacteriocin, immunity modulator, industrially useful molecule, poison molecule, or antidote molecule. Some promoters can drive transcription at all times (“constitutive promoters”). Some promoters can drive transcription under only select circumstances (“conditional promoters”), for example depending on the presence or absence of an environmental condition, chemical compound, gene product, stage of the cell cycle, or the like.

The skilled artisan will appreciate that depending on the desired expression activity, an appropriate promoter can be selected, and placed in cis with a sequence to be expressed. Exemplary promoters with exemplary activities are provided in Table 3.1-3.11 herein. The skilled artisan will appreciate that some promoters are compatible with particular transcriptional machinery (e.g. RNA polymerases, general transcription factors, and the like). As such, while compatible “species” are identified for some promoters described herein, it is contemplated that according to some embodiments herein, these promoters can readily function in microorganisms other than the identified species, for example in species with compatible endogenous transcriptional machinery, genetically modified species comprising compatible transcriptional machinery, or fully synthetic microbial organisms comprising compatible transcriptional machinery.

The promoters of Tables 3.1-3.11 herein are publicly available from the Biobricks foundation. Per the Biobricks foundation, use of these promoters in accordance with BioBrick™ Public Agreement (BPA) is encouraged.

It should be appreciated that any of the “coding” polynucleotides described herein (for example a bacteriocin polynucleotide, immunity polynucleotide, poison polynucleotide, antidote polynucleotide, or product polynucleotide) is generally amenable to being expressed under the control of a desired promoter. In some embodiments, a single “coding” polynucleotide is under the control of a single promoter. In some embodiments, two or more “coding” polynucleotides are under the control of a single promoter, for example two, three, four, five, six, seven, eight, nine, or ten polynucleotides. As such, in some embodiments, a “cocktail” of different bacteriocins can be produced by a single microbial organism. In some embodiments, a bacteriocin polynucleotide is under the control of a promoter. In some embodiments, an immunity modulator is under the control of a promoter. In some embodiments, a polynucleotide encoding a desired gene product is under the control of a promoter. In some embodiments, the bacteriocin polynucleotide and the polynucleotide encoding a desired gene product are under the control of the same promoter. In some embodiments, a bacteriocin polynucleotide and the polynucleotide encoding a desired gene product are under the control of different promoters. In some embodiments, the immunity modulator polynucleotide and the polynucleotide encoding a desired gene product are under the control of the same promoter. In some embodiments, the bacteriocin polynucleotide and the immunity modulator polynucelotide are under the control of different promoters.

Generally, translation initiation for a particular transcript is regulated by particular sequences at or 5′ of the 5′ end of the coding sequence of a transcript. For example, a coding sequence can begin with a start codon configured to pair with an initiator tRNA. While naturally-occurring translation systems typically use Met (AUG) as a start codon, it will be readily appreciated that an initiator tRNA can be engineered to bind to any desired triplet or triplets, and accordingly, triplets other than AUG can also function as start codons in certain embodiments. Additionally, sequences near the start codon can facilitate ribosomal assembly, for example a Kozak sequence ((gcc)gccRccAUGG, SEQ ID NO: 542, in which R represents “A” or “G”) or Internal Ribosome Entry Site (IRES) in typical eukaryotic translational systems, or a Shine-Delgarno sequence (GGAGGU, SEQ ID NO: 543) in typical prokaryotic translation systems. As such in some embodiments, a transcript comprising a “coding” polynucleotide sequence, for example a bacteriocin polynucleotide or immunity modulator polynucleotide, or polynucleotide encoding a desired industrial product, comprises an appropriate start codon and translational initiation sequence. In some embodiments, for example if two or more “coding” polynucleotide sequences are positioned in cis on a transcript, each polynucleotide sequence comprises an appropriate start codon and translational initiation sequence(s). In some embodiments, for example if two or more “coding” polynucleotide sequences are positioned in cis on a transcript, the two sequences are under control of a single translation initiation sequence, and either provide a single polypeptide that can function with both encoded polypeptides in cis, or provide a means for separating two polypeptides encoded in cis, for example a 2A sequence or the like. In some embodiments, a translational intiator tRNA is regulatable, so as to regulate initiation of translation of a bacteriocin, immunity modulator, poison molecule, antidote molecule, or industrially useful molecule.

TABLE 3.1
Exemplary Metal-Sensitive Promoters

| SEQ | | | |
|---|---|---|---|
| ID | | | |
| NO: | Name | Description | Sequence |
| 544 | BBa_I721001 | Lead | gaaaaccttgtcaat |
| | | Promoter | gaagagcgatctatg |
| 545 | BBa_I731004 | FecA | ttctcgttcgactca |
| | | promoter | tagctgaacacaaca |
| 546 | BBa_I760005 | Cu-sensitive | atgacaaaattgtca |
| | | promoter | t |
| 547 | BBa_I765000 | Fe promoter | accaatgctgggaac |
| | | | ggccagggcacctaa |
| 548 | BBa_I765007 | Fe and UV | ctgaaagcgcatacc |
| | | promoters | gctatggagggggtt |
| 549 | BBa_J3902 | PrFe (PI + PII | tagatatgcctgaaa |
| | | rus operon) | gcgcataccgctatg |

TABLE 3.2
Exemplary Cell Signaling-Responsive Promoters

| SEQ | | | |
|---|---|---|---|
| ID | | | |
| NO: | Name | Description | Sequence |
| 550 | BBa_I1051 | Lux cassette right promoter | tgttatagtcgaatacctctggcggtgata |
| 551 | BBa_I14015 | P(Las) TetO | ttttggtacactccctatcagtgatagaga |
| 552 | BBa_I14016 | P(Las) CIO | ctttttggtacactacctctggcggtgata |
| 553 | BBa_I14017 | P(Rhl) | tacgcaagaaaatggtttgttatagtcgaa |
| 554 | BBa_I739105 | Double Promoter (LuxR/HSL, | cgtgcgtgttgataacaccgtgcgtgttga |
| | | positive/cI, negative) | |
| 555 | BBa_I746104 | P2 promoter in agr operon | agattgtactaaatcgtataatgacagtga |
| | | from S. aureus | |
| 556 | BBa_I751501 | plux-cI hybrid promoter | gtgttgatgcttttatcaccgccagtggta |
| 557 | BBa_I751502 | plux-lac hybrid promoter | agtgtgtggaattgtgagcggataacaatt |
| 558 | BBa_I761011 | CinR, CinL and glucose | acatcttaaaagttttagtatcatattcgt |
| | | controlled promotor | |
| 559 | BBa_J06403 | RhIR promoter repressible by | tacgcaagaaaatggtttgttatagtcgaa |
| | | CI | |
| 560 | BBa_J102001 | Reverse Lux Promoter | tcttgcgtaaacctgtacgatcctacaggt |
| 561 | BBa_J64000 | rhlI promoter | atcctcctttagtcttccccctcatgtgtg |
| 562 | BBa_J64010 | lasI promoter | taaaattatgaaatttgcataaattcttca |
| 563 | BBa_J64067 | LuxR + 3OC6HSL independent | gtgttgactattttacctctggcggtgata |
| | | R0065 | |
| 564 | BBa_J64712 | LasR/LasI Inducible & | gaaatctggcagtttttggtacacgaaagc |
| | | RHLR/RHLI repressible | |
| | | Promoter | |
| 565 | BBa_K091107 | pLux/cI Hybrid Promoter | acaccgtgcgtgttgatatagtcgaataaa |
| 566 | BBa_K091117 | pLas promoter | aaaattatgaaatttgtataaattcttcag |
| 567 | BBa_K091143 | pLas/cI Hybrid Promoter | ggttctttttggtacctctggcggtgataa |
| 568 | BBa_K091146 | pLas/Lux Hybrid Promoter | tgtaggatcgtacaggtataaattcttcag |
| 569 | BBa_K091156 | pLux | caagaaaatggtttgttatagtcgaataaa |
| 570 | BBa_K091157 | pLux/Las Hybrid Promoter | ctatctcatttgctagtatagtcgaataaa |
| 571 | BBa_K145150 | Hybrid promoter: HSL-LuxR | tagtttataatttaagtgttctttaatttc |
| | | activated, P22 C2 repressed | |
| 572 | BBa_K266000 | PAI + LasR -> LuxI (AI) | caccttcgggtgggcctttctgcgtttata |
| 573 | BBa_K266005 | PAI + LasR -> LasI & AI + | aataactctgatagtgctagtgtagatctc |
| | | LuxR --\|LasI | |
| 574 | BBa_K266006 | PAI + LasR -> LasI + GFP & | caccttcgggtgggcctttctgcgtttata |
| | | AI + LuxR --\| LasI + GFP | |
| 575 | BBa_K266007 | Complex QS -> LuxI & LasI | caccttcgggtgggcctttctgcgtttata |
| | | circuit | |
| 576 | BBa_K658006 | position 3 mutated promoter | caagaaaatggtttgttatagtcgaataaa |
| | | lux pR-3 (luxR & HSL | |
| | | regulated) | |
| 577 | BBa_K658007 | position 5 mutated promoter | caagaaaatggtttgttatagtcgaataaa |
| | | lux pR-5 (luxR & HSL | |
| | | regulated) | |
| 578 | BBa_K658008 | position 3&5 mutated | caagaaaatggtttgttatagtcgaataaa |
| | | promoter lux pR-3/5 (luxR & | |
| | | HSL regulated) | |
| 579 | BBa_R0061 | Promoter (HSL-mediated luxR | ttgacacctgtaggatcgtacaggtataat |
| | | repressor) | |
| 580 | BBa_R0062 | Promoter (luxR & HSL | caagaaaatggtttgttatagtcgaataaa |
| | | regulated -- lux pR) | |
| 581 | BBa_R0063 | Promoter (luxR & HSL | cacgcaaaacttgcgacaaacaataggtaa |
| | | regulated -- lux pL) | |
| 582 | BBa_R0071 | Promoter (RhlR & C4-HSL | gttagctttcgaattggctaaaaagtgttc |
| | | regulated) | |
| 583 | BBa_R0078 | Promoter (cinR and HSL | ccattctgctttccacgaacttgaaaacgc |
| | | regulated) | |
| 584 | BBa_R0079 | Promoter (LasR & PAI | ggccgcgggttctttttggtacacgaaagc |
| | | regulated) | |
| 585 | BBa_R1062 | Promoter, Standard (luxR and | aagaaaatggtttgttgatactcgaataaa |
| | | HSL regulated -- lux pR) | |

TABLE 3.3
Exemplary Constitutive E. coli σ⁷⁰ Promoters

| SEQ | | | |
|---|---|---|---|
| ID | | | |
| NO: | Name | Description | Sequence |
| 586 | BBa_I14018 | P(Bla) | gtttatacataggcgagtactctgttatgg |
| 587 | BBa_I14033 | P(Cat) | agaggttccaactttcaccataatgaaaca |
| 588 | BBa_I14034 | P(Kat) | taaacaactaacggacaattctacctaaca |
| 589 | BBa_I732021 | Template for Building Primer | acatcaagccaaattaaacaggattaacac |
| | | Family Member | |
| 590 | BBa_I742126 | Reverse lambda cI-regulated | gaggtaaaatagtcaacacgcacggtgtta |
| | | promoter | |
| 591 | BBa_J01006 | Key Promoter absorbs 3 | caggccggaataactccctataatgcgcca |
| 592 | BBa_J23100 | constitutive promoter family | ggctagctcagtcctaggtacagtgctagc |
| | | member | |
| 593 | BBa_J23101 | constitutive promoter family | agctagctcagtcctaggtattatgctagc |
| | | member | |
| 594 | BBa_J23102 | constitutive promoter family | agctagctcagtcctaggtactgtgctagc |
| | | member | |
| 595 | BBa_J23103 | constitutive promoter family | agctagctcagtcctagggattatgctagc |
| | | member | |
| 596 | BBa_J23104 | constitutive promoter family | agctagctcagtcctaggtattgtgctagc |
| | | member | |
| 597 | BBa_J23105 | constitutive promoter family | ggctagctcagtcctaggtactatgctagc |
| | | member | |
| 598 | BBa_J23106 | constitutive promoter family | ggctagctcagtcctaggtatagtgctagc |
| | | member | |
| 599 | BBa_J23107 | constitutive promoter family | ggctagctcagccctaggtattatgctagc |
| | | member | |
| 600 | BBa_J23108 | constitutive promoter family | agctagctcagtcctaggtataatgctagc |
| | | member | |
| 601 | BBa_J23109 | constitutive promoter family | agctagctcagtcctagggactgtgctagc |
| | | member | |
| 602 | BBa_J23110 | constitutive promoter family | ggctagctcagtcctaggtacaatgctagc |
| | | member | |
| 603 | BBa_J23111 | constitutive promoter family | ggctagctcagtcctaggtatagtgctagc |
| | | member | |
| 604 | BBa_J23112 | constitutive promoter family | agctagctcagtcctagggattatgctagc |
| | | member | |
| 605 | BBa_J23113 | constitutive promoter family | ggctagctcagtcctagggattatgctagc |
| | | member | |
| 606 | BBa_J23114 | constitutive promoter family | ggctagctcagtcctaggtacaatgctagc |
| | | member | |
| 607 | BBa_J23115 | constitutive promoter family | agctagctcagcccttggtacaatgctagc |
| | | member | |
| 608 | BBa_J23116 | constitutive promoter family | agctagctcagtcctagggactatgctagc |
| | | member | |
| 609 | BBa_J23117 | constitutive promoter family | agctagctcagtcctagggattgtgctagc |
| | | member | |
| 610 | BBa_J23118 | constitutive promoter family | ggctagctcagtcctaggtattgtgctagc |
| | | member | |
| 611 | BBa_J23119 | constitutive promoter family | agctagctcagtcctaggtataatgctagc |
| | | member | |
| 612 | BBa_J23150 | 1 bp mutant from J23107 | ggctagctcagtcctaggtattatgctagc |
| 613 | BBa_J23151 | 1 bp mutant from J23114 | ggctagctcagtcctaggtacaatgctagc |
| 614 | BBa_J44002 | pBAD reverse | aaagtgtgacgccgtgcaaataatcaatgt |
| 615 | BBa_J48104 | NikR promoter, a protein of | gacgaatacttaaaatcgtcatacttattt |
| | | the ribbon helix-helix family of | |
| | | trancription factors that repress | |
| | | expre | |
| 616 | BBa_J54200 | lacq_Promoter | aaacctttcgcggtatggcatgatagcgcc |
| 617 | BBa_J56015 | lacIQ-promoter sequence | tgatagcgcccggaagagagtcaattcagg |
| 618 | BBa_J64951 | E. Coli CreABCD phosphate | ttatttaccgtgacgaactaattgctcgtg |
| | | sensing operon promoter | |
| 619 | BBa_K088007 | GlnRS promoter | catacgccgttatacgttgtttacgctttg |
| 620 | BBa_K119000 | Constitutive weak promoter of | ttatgcttccggctcgtatgttgtgtggac |
| | | lacZ | |
| 621 | BBa_K119001 | Mutated LacZ promoter | ttatgcttccggctcgtatggtgtgtggac |
| 622 | BBa_K137029 | constitutive promoter with | atatatatatatatataatggaagcgtttt |
| | | (TA) 10 between −10 and −35 | |
| | | elements | |
| 623 | BBa_K137030 | constitutive promoter with | atatatatatatatataatggaagcgtttt |
| | | (TA)9 between −10 and −35 | |
| | | elements | |
| 624 | BBa_K137031 | constitutive promoter with | ccccgaaagcttaagaatataattgtaagc |
| | | (C)10 between −10 and −35 | |
| | | elements | |
| 625 | BBa_K137032 | constitutive promoter with | ccccgaaagcttaagaatataattgtaagc |
| | | (C)12 between −10 and −35 | |
| | | elements | |
| 626 | BBa_K137085 | optimized (TA) repeat | tgacaatatatatatatatataatgctagc |
| | | constitutive promoter with 13 | |
| | | bp between −10 and −35 | |
| | | elements | |
| 627 | BBa_K137086 | optimized (TA) repeat | acaatatatatatatatatataatgctagc |
| | | constitutive promoter with 15 | |
| | | bp between −10 and −35 | |
| | | elements | |
| 628 | BBa_K137087 | optimized (TA) repeat | aatatatatatatatatatataatgctagc |
| | | constitutive promoter with 17 | |
| | | bp between −10 and −35 | |
| | | elements | |
| 629 | BBa_K137088 | optimized (TA) repeat | tatatatatatatatatatataatgctagc |
| | | constitutive promoter with 19 | |
| | | bp between −10 and −35 | |
| | | elements | |
| 630 | BBa_K137089 | optimized (TA) repeat | tatatatatatatatatatataatgctagc |
| | | constitutive promoter with 21 | |
| | | bp between −10 and −35 | |
| | | elements | |
| 631 | BBa_K137090 | optimized (A) repeat | aaaaaaaaaaaaaaaaaatataatgctagc |
| | | constitutive promoter with 17 | |
| | | bp between −10 and −35 | |
| | | elements | |
| 632 | BBa_K137091 | optimized (A) repeat | aaaaaaaaaaaaaaaaaatataatgctagc |
| | | constitutive promoter with 18 | |
| | | bp between −10 and −35 | |
| | | elements | |
| 633 | BBa_K256002 | J23101:GFP | caccttcgggtgggcctttctgcgtttata |
| 634 | BBa_K256018 | J23119:IFP | caccttcgggtgggcctttctgcgtttata |
| 635 | BBa_K256020 | J23119:HO1 | caccttcgggtgggcctttctgcgtttata |
| 636 | BBa_K256033 | Infrared signal reporter | caccttcgggtgggcctttctgcgtttata |
| | | (J23119:IFP:J23119:HO1) | |
| 637 | BBa_K292000 | Double terminator + | ggctagctcagtcctaggtacagtgctagc |
| | | constitutive promoter | |
| 638 | BBa_K292001 | Double terminator + | tgctagctactagagattaaagaggagaaa |
| | | Constitutive promoter + Strong | |
| | | RBS | |
| 639 | BBa_K418000 | IPTG inducible Lac promoter | ttgtgagcggataacaagatactgagcaca |
| | | cassette | |
| 640 | BBa_K418002 | IPTG inducible Lac promoter | ttgtgagcggataacaagatactgagcaca |
| | | cassette | |
| 641 | BBa_K418003 | IPTG inducible Lac promoter | ttgtgagcggataacaagatactgagcaca |
| | | cassette | |
| 642 | BBa_M13101 | M13K07 gene I promoter | cctgtttttatgttattctctctgtaaagg |
| 643 | BBa_M13102 | M13K07 gene II promoter | aaatatttgcttatacaatcttcctgtttt |
| 644 | BBa_M13103 | M13K07 gene III promoter | gctgataaaccgatacaattaaaggctcct |
| 645 | BBa_M13104 | M13K07 gene IV promoter | ctcttctcagcgtcttaatctaagctatcg |
| 646 | BBa_M13105 | M13K07 gene V promoter | atgagccagttcttaaaatcgcataaggta |
| 647 | BBa_M13106 | M13K07 gene VI promoter | ctattgattgtgacaaaataaacttattcc |
| 648 | BBa_M13108 | M13K07 gene VIII promoter | gtttcgcgcttggtataatcgctgggggtc |
| 649 | BBa_M13110 | M13110 | ctttgcttctgactataatagtcagggtaa |
| 650 | BBa_M31519 | Modified promoter sequence of | aaaccgatacaattaaaggctcctgctagc |
| | | g3. | |
| 651 | BBa_R1074 | Constitutive Promoter I | caccacactgatagtgctagtgtagatcac |
| 652 | BBa_R1075 | Constitutive Promoter II | gccggaataactccctataatgcgccacca |
| 653 | BBa_S03331 | --Specify Parts List-- | ttgacaagcttttcctcagctccgtaaact |

TABLE 3.4
Exemplary Constitutive E. coli σ^{s} Promoters

| SEQ | | | |
|---|---|---|---|
| ID | | | |
| NO: | Name | Description | Sequence |
| 654 | BBa_ | Full-length | ggtttcaaaattgtg |
| | J45992 | stationary phase | atctatatttaacaa |
| | | osmY promoter | |
| 655 | BBa_ | Minimal stationary | ggtttcaaaattgtg |
| | I45993 | phase osmY | atctatatttaacaa |
| | | promoter | |

TABLE 3.5
Exemplary Constitutive E. coli σ³² Promoters

| SEQ | | | |
|---|---|---|---|
| ID | | | |
| NO: | Name | Description | Sequence |
| 656 | BBa_ | htpG Heat | tctattccaataaag |
| | J45504 | Shock Promoter | aaatcttcctgcgtg |

TABLE 3.6
Exemplary Constitutive B. subtilis σ^{A} Promoters

| SEQ | | | |
|---|---|---|---|
| ID | | | |
| NO: | Name | Description | Sequence |
| 657 | BBa_ | Promoter veg | aaaaatgggctcgtg |
| | K143012 | a constitutive | ttgtacaataaatgt |
| | | promoter for | |
| | | B. subtilis | |
| 658 | BBa_ | Promoter 43 a | aaaaaaagcgcgcga |
| | K143013 | constitutive | ttatgtaaaatataa |
| | | promoter for | |
| | | B. subtilis | |
| 659 | BBa_ | Strong | aattgcagtaggcat |
| | K780003 | constitutive | gacaaaatggactca |
| | | promoter | |
| | | for Bacillus | |
| | | subtilis | |
| 660 | BBa_ | PliaG | caagcttttccttta |
| | K823000 | | taatagaatgaatga |
| 661 | BBa_ | PlepA | tctaagctagtgtat |
| | K823002 | | tttgcgtttaatagt |
| 662 | BBa_ | Pveg | aatgggctcgtgttg |
| | K823003 | | tacaataaatgtagt |

TABLE 3.7
Exemplary Constitutive B. subtilis σ^{B} Promoters

| SEQ | | | |
|---|---|---|---|
| ID | | | |
| NO: | Name | Description | Sequence |
| 663 | BBa_ | Promoter etc | atccttatcgttatg |
| | K143010 | for B. subtilis | ggtattgtttgtaat |
| 664 | BBa_ | Promoter gsiB | taaaagaattgtgag |
| | K143011 | for B. subtilis | cgggaatacaacaac |
| 665 | BBa_ | Promoter 43 a | aaaaaaagcgcgcga |
| | K143013 | constitutive | ttatgtaaaatataa |
| | | promoter for | |
| | | B. subtilis | |

TABLE 3.8
Exemplary Constitutive Promoters
from miscellaneous prokaryotes

| SEQ | | | |
|---|---|---|---|
| ID | | | |
| NO: | Name | Description | Sequence |
| 666 | a_K112706 | Pspv2 from | tacaaaataattccc |
| | | Salmonella | ctgcaaacattatca |
| 667 | BBa_K112707 | Pspv from | tacaaaataattccc |
| | | Salmonella | ctgcaaacattatcg |

TABLE 3.9
Exemplary Constitutive Promoters
from bacteriophage T7

| SEQ | | | |
|---|---|---|---|
| ID | | | |
| NO: | Name | Description | Sequence |
| 668 | BBa_ | T7 promoter | agggaatacaagcta |
| | I712074 | (strong | cttgttctttttgca |
| | | promoter | |
| | | from T7 | |
| | | bacteriophage) | |
| 669 | BBa_ | T7 Promoter | taatacgactcacta |
| | I719005 | | tagggaga |
| 670 | BBa_ | T7 Promoter | gaatttaatacgact |
| | J34814 | | cactatagggaga |
| 671 | BBa_ | T7 consensus | taatacgactcacta |
| | J64997 | −10 | tagg |
| | | and rest | |
| 672 | BBa_ | overlapping | gagtcgtattaatac |
| | K113010 | T7 promoter | gactcactatagggg |
| 673 | BBa_ | more | agtgagtcgtactac |
| | K113011 | overlapping | gactcactatagggg |
| | | T7 | |
| | | promoter | |
| 674 | BBa_ | weaken | gagtcgtattaatac |
| | K113012 | overlapping | gactctctatagggg |
| | | T7 | |
| | | promoter | |
| 675 | BBa_R0085 | T7 Consensus | taatacgactcacta |
| | | Promoter | tagggaga |
| | | Sequence | |
| 676 | BBa_R0180 | T7 RNAP | ttatacgactcacta |
| | | promoter | tagggaga |
| 677 | BBa_R0181 | T7 RNAP | gaatacgactcacta |
| | | promoter | tagggaga |
| 678 | BBa_R0182 | T7 RNAP | taatacgtctcacta |
| | | promoter | tagggaga |
| 679 | BBa_R0183 | T7 RNAP | tcatacgactcacta |
| | | promoter | tagggaga |
| 680 | BBa_Z0251 | T7 strong | taatacgactcacta |
| | | promoter | tagggagaccacaac |
| 681 | BBa_Z0252 | T7 weak | taattgaactcacta |
| | | binding and | aagggagaccacagc |
| | | processivity | |
| 682 | BBa_Z0253 | T7 weak | cgaagtaatacgact |
| | | binding | cactattagggaaga |
| | | promoter | |

TABLE 3.10
Exemplary Constitutive Promoters from yeast

| SEQ | | | |
|---|---|---|---|
| ID | | | |
| NO: | Name | Description | Sequence |
| 683 | BBa_I766555 | pCyc | acaaacacaaataca |
| | | (Medium) | cacactaaattaata |
| | | Promoter | |
| 684 | BBa_I766556 | pAdh | ccaagcatacaatca |
| | | (Strong) | actatctcatataca |
| | | Promoter | |
| 685 | BBa_I766557 | pSte5 | gatacaggatacagc |
| | | (Weak) | ggaaacaacttttaa |
| | | Promoter | |
| 686 | BBa_J63005 | yeast | tttcaagctatacca |
| | | ADH1 | agcatacaatcaact |
| | | promoter | |
| 687 | BBa_K105027 | cyc100 | cctttgcagcataaa |
| | | minimal | ttactatacttctat |
| | | promoter | |
| 688 | BBa_K105028 | cyc70 | cctttgcagcataaa |
| | | minimal | ttactatacttctat |
| | | promoter | |
| 689 | BBa_K105029 | cyc43 | cctttgcagcataaa |
| | | minimal | ttactatacttctat |
| | | promoter | |
| 690 | BBa_K105030 | cyc28 | cctttgcagcataaa |
| | | minimal | ttactatacttctat |
| | | promoter | |
| 691 | BBa_K105031 | eye | cctttgcagcataaa |
| | | 16 | ttactatacttctat |
| | | minimal | |
| | | promoter | |
| 692 | BBa_K122000 | pPGK1 | ttatctactttttac |
| | | | aacaaatataaaaca |
| 693 | BBa_K124000 | pCYC | acaaacacaaataca |
| | | Yeast | cacactaaattaata |
| | | Promoter | |
| 694 | BBa_K124002 | Yeast | gtttcgaataaacac |
| | | GPD | acataaacaaacaaa |
| | | (TDH3) | |
| | | Promoter | |
| 695 | BBa_K319005 | yeast | ccaagcatacaatca |
| | | mid- | actatctcatataca |
| | | length | |
| | | ADH1 | |
| | | promoter | |
| 696 | BBa_M31201 | Yeast | accatcaaaggaagc |
| | | CLB1 | tttaatcttctcata |
| | | promoter | |
| | | region, | |
| | | G2/M | |
| | | cell | |
| | | cycle | |
| | | specific | |

TABLE 3.11
Exemplary Constitutive Promoters
from miscellaneous eukaryotes

| SEQ | | | |
|---|---|---|---|
| ID | | | |
| NO: | Name | Description | Sequence |
| 697 | BBa_ | CMV promoter | agaacccactgctta |
| | I712004 | | ctggcttatcgaaat |
| 698 | BBa_ | Ubc Promoter | ggccgtttttggctt |
| | K076017 | | ttttgttagacgaag |

The above-referenced promoters are provided by way of non-limiting example only. The skilled artisan will readily recognize that many variants of the above-referenced promoters, and many other promoters (including promoters isolated from naturally existing organisms, variations thereof, and fully synthetic promoters) can readily be used in accordance with some embodiments herein.

### Regulation of Gene Activity

Gene activity can be regulated to either increase or decrease activity of the gene product. In some embodiments, the gene product for which activity is regulated comprises a bacteriocin, immunity modulator, industrially useful molecule, poison molecule, or antidote molecule. In some embodiments, two or more of such gene products are regulated under a single gene regulation system. In some embodiments, gene activity is regulated at the level of gene expression. In some embodiments, gene activity is regulated at the transcriptional level, for example by activating or repressing a promoter. In some embodiments, gene activity is regulated at the post-transcriptional level, for example through regulation of RNA stability. In some embodiments, gene activity is regulated at the translational level, for example through regulation of initiation of translation. In some embodiments, gene activity is regulated at the post-translational level, for example through regulation of polypeptide stability, post-translational modifications to the polypeptide, or binding of an inhibitor to the polypeptide.

In some embodiments, gene activity is increased. In some embodiments, activity of at least one of a bacteriocin, immunity modulator, industrially useful molecule, poison molecule, or antidote molecule is increased. Conceptually, gene activity can be increased by directly activating gene activity, or by decreasing the activity of an inhibitor of gene activity. In some embodiments, gene activity is activated by at least one of: inducing promoter activity, inhibiting a transcriptional repressor, increasing RNA stability, inhibiting a post-transcriptional inhibitor (for example, inhibiting a ribozyme or antisense oligonucleotide), inducing translation (for example, via a regulatable tRNA), making a desired post-translational modification, or inhibiting a post-translational inhibitor (for example a protease directed to a polypeptide encoded by the gene). In some embodiments, a compound present in a desired environment induces a promoter. For example, the presence of iron in culture medium can induce transcription by an iron-sensitive promoter as described herein. In some embodiments, a compound present in a desired culture medium inhibits a transcriptional repressor. For example, the presence of tetracycline in an environment can inhibit the tet repressor, and thus allow activity from the tetO promoter. In some embodiments, a compound found only outside of a desired culture medium induces transcription.

In some embodiments, gene activity is decreased. Conceptually, gene activity can be decreased by directly inhibiting gene activity, or by decreasing the activity of an activator of gene activity. In some embodiments, gene activity is reduced, but some level of activity remains. In some embodiments, gene activity is fully inhibited. In some embodiments, gene activity is decreased by at least one of inhibiting promoter activity, activating a transcriptional repressor, decreasing RNA stability, activating a post-transcriptional inhibitor (for example, expressing a ribozyme or antisense oligonucleotide), inhibiting translation (for example, via a regulatable tRNA), failing to make a required post-translational modification, inactivating a polypeptide (for example by binding an inhibitor or via a polypeptide-specific protease), or failing to properly localize a polypeptide (e.g. failing to secrete a bacteriocin). In some embodiments, gene activity is decreased by removing a gene from a desired location, for example by excising a gene using a FLP-FRT or cre-lox cassette, or through loss or degradation of a plasmid. In some embodiments, a gene product (e.g. a polypeptide) or a product produced by a gene product (e.g. the product of an enzymatic reaction) inhibits further gene activity (e.g. a negative feedback loop).

### Genetic Modification of Microbial Organisms

Techniques of genetically modifying microorganisms are well known in the art. In some embodiments, a microorganism is genetically modified to comprise nucleic acid sequence regulating the expression of, and encoding, at least one of bacteriocins, immunity modulators, industrially useful molecules, poison molecules, or antidote molecules. Polynucleotides can be delivered to microorganisms, and can be stably integrated into the chromosomes of these microorganisms, or can exist free of the genome, for example in a plasmid, extrachromosomal array, episome, minichromosome, or the like.

Exemplary vectors for genetic modification of microbial cells include, but are not limited to, plasmids, viruses (including bacteriophage), and transposable elements. Additionally, it will be appreciated that entire microbial genomes comprising desired sequences can be synthesized and assembled in a cell (see, e.g. Gibson et al. (2010), Science 329: 52-56). As such, in some embodiments, a microbial genome (or portion thereof) is synthesized with desired features such as bacteriocin polynucleotide(s), and introduced into a microbial cell.

It can be useful to flexibly genetically modify a microbial cell, for example to engineer or reengineer a microbial cell to have a desired type and/or spectrum of bacteriocin or immunity modulator activity. In some embodiments, a cassette for inserting one or more desired bacteriocin and/or immunity modulator polynucleotides into a polynucleotide sequence is provided. Exemplary cassettes include, but are not limited to, a Cre/lox cassette or FLP/FRT cassette. In some embodiments, the cassette is positioned on a plasmid, so that a plasmid with the desired bacteriocin and/or immunity modulator combination can readily be introduced to the microbial cell. In some embodiments, the cassette is positioned in the genome of the microbial cell, so that a cassette with the desired bacteriocin and/or immunity modulator combination can be introduced to the desired location.

In some embodiments, plasmid conjugation can be used to introduce a desired plasmid from a “donor” microbial cell to a recipient microbial cell. Goñi-Moreno, et al. (2013) Multicellular Computing Using Conjugation for Wiring. PLoS ONE 8(6): e65986, hereby incorporated by reference in its entirety. In some embodiments, plasmid conjugation can genetically modify a recipient microbial cell by introducing a conjugation plasmid from a donor microbial cell to a recipient microbial cell. Without being limited by any particular theory, conjugation plasmids that comprise the same or functionally same set of replication genes typically cannot coexist in the same microbial cell. As such, in some embodiments, plasmid conjugation “reprograms” a recipient microbial cell by introducing a new conjugation plasmid to supplant another conjugation plasmid that was present in the recipient cell. In some embodiments, plasmid conjugation is used to engineer (or reengineer) a microbial cell with a particular combination of one or more bacteriocins and/or immunity modulators. According to some embodiments, a variety of conjugation plasmids comprising different combinations of bacteriocins and/or immunity modulators is provided. The plasmids can comprise additional genetic elements as described herein, for example promoters, translational initiation sites, and the like. In some embodiments the variety of conjugation plasmids is provided in a collection of donor cells, so that a donor cell comprising the desired plasmid can be selected for plasmid conjugation. In some embodiments, a particular combination of bacteriocins and/or immunity modulators is selected, and an appropriate donor cell is conjugated with a microbial cell of interest to introduce a conjugation plasmid comprising that combination into a recipient cell. In some embodiments, the recipient cell is a “newly engineered” cell, for example to be introduced into or for initiating a culture. In some embodiments, the recipient cell is a “reengineered cell,” for example to introduce a new bacteriocin (and optionally immunity modulator) activity to an existing culture that has encountered a new type of invader cell, and/or to remove a bacteriocin activity that is no longer desired in the culture.

### Culture Media

Microbial culture environments can comprise a wide variety of culture media, for example feedstocks. The selection of a particular culture medium can depend upon the desired application. Conditions of a culture medium include not only chemical composition, but also temperature, amounts of light, pH, CO₂ levels, and the like.

In some embodiments, a genetically engineered microorganism as described herein is added to a culture medium that comprises other microorganisms and at least one feedstock. In some embodiments, the culture medium comprises a compound that induces the activity or expression of a bacteriocin and/or immunity modulator. In some embodiments, the culture medium comprises a compound that represses the activity or expression of a bacteriocin and/or immunity modulator. In some embodiments, a compound that induces the activity of the bacteriocin is present outside of the feedstock, but not in the feedstock. In some embodiments, a compound that represses the activity of the immunity modulator is present outside the feedstock, but not in the feedstock.

The term “feedstock” is used herein in a broad sense to encompass material that can be consumed, fermented, purified, modified, or otherwise processed by microbial organisms, for example in the context of industrial processes. As such, “feedstock” is not limited to food or food products. As used herein a “feedstock” is a category of culture medium. Accordingly, as used herein “culture medium” includes, but it is not limited to feedstock. As such, whenever a “culture medium” is referred to herein, feedstocks are also expressly contemplated.

### Genetically Engineered Microbial Cells

In some embodiments, genetically modified microbial cells are provided. Genetically modified microbial cells can be configured for a wide variety of purposes. In some embodiments, microbial cells comprise genetic modifications to regulate the expression of at least one of bacteriocins, immunity modulators, industrially useful molecules, poison molecules, or antidote molecules. In some embodiments, microbial cells comprise genetic modifications to regulate the expression of bacteriocins. In some embodiments, microbial cells comprise genetic modifications to regulate the expression of immunity modulators.

In some embodiments, the genetically modified microbial cells are modified to produce a product. In some embodiments, the product is a gene product, for example a polypeptide or RNA. As such, polynucleotide “coding” sequence as referred to herein can refer to sequence encoding either a polypeptide or an RNA. In some embodiments, microbial cells can be configured to produce one or more gene products that contribute to synthesis of a desired product, for example a carbohydrate, biofuel, lipid, small molecule, or metal. In some embodiments, the product is synthesized via the activity of one or more gene products of the microbial cell. Optionally, synthesis of the product can also involve the activity of one or more gene products of one or more other microbial cells. In some embodiments, microbial cells can be configured to decontaminate or decompose one or more substances in a culture media, for example a feedstock. The decontamination can be mediated wholly, or partially by one or more gene products of the microbial cells. In some embodiments, microbial cells can be configured to scavenge for a material, for example a metal such as iron or a rare earth metal.

### Controlling the Growth of Microbial Cells

In some embodiments, genetically modified microbial cells are modified to regulate the growth of other microbial cells. In some embodiments, the microbial cells regulate the growth of other microbial cells of the same species or strain, for example their own clones. In some embodiments, the microbial cells regulated the growth of microbial cells of a different species or strain, for example invaders. In some embodiments, a microbial cell secretes a bacteriocin to regulate other microbial cells. The regulation of each of the other microbial cells can depend on its expression (or lack thereof) of an immunity modulator having protective effects against the particular the secreted bacteriocin.

As used herein “desired cell” and the like refer to a microbial cell with at least one characteristic for which survival, growth, and/or proliferation of the microbial cell is desired, or at least an absence of negative control of the cell's growth is desired. In some embodiments, a desired cell is in an appropriate environment, for example its industrially-applicable feedstock. In some embodiments, a desired cell is a cell that is positively selected for, for example a cell that has undergone a particular recombination even, or is expressing high levels of a useful gene product. In some embodiments, a desired cell is a cell configured to neutralize contaminating cells, for example pathogenic cells. In some embodiments a desired cell is positively selected for by its expression of an immunity modulator corresponding to at least one bacteriocin that can be present in the environment. Without being bound by any particular theory, it is contemplated that a microbial cell capable of neutralizing other microbial cells which lack a similar neutralizing function will have a competitive advantage. As such, in some embodiments, a desired cell is selected for through its ability to neutralize other cells. In some embodiments a desired cell is positively selected for by expressing both a bacteriocin and a corresponding immunity modulator.

As used herein “undesired cell” and the like refer to a microbial cell with at least one characteristic making survival, growth, or proliferation undesirable. In some embodiments, the undesired cell is an invading microbial cell, for example a contaminating cell that has entered a culture environment. In some embodiments, an undesired cell has escaped from an appropriate culture medium, for example its industrially-applicable feedstock. In some embodiments, an undesired cell has lost a particular plasmid, or has failed to undergo a particular recombination event. In some embodiments, an undesired cell has failed to produce, or produces low levels of desired gene product. In some embodiments, an undesired cell is selected against. In some embodiments, an undesired cell is selected against through by reducing the cell's expression or activity of an immunity modulator that protects against a bacteriocin in the environment. In some embodiments, an undesired cell is selected against through by reducing the cell's expression or activity of an immunity modulator that protects against a bacteriocin secreted by the cell and clones thereof. In some embodiments, an undesired cell is selected against by reducing the cell's expression of a bacteriocin, thereby putting the cell at a competitive disadvantage against other microbial cells.

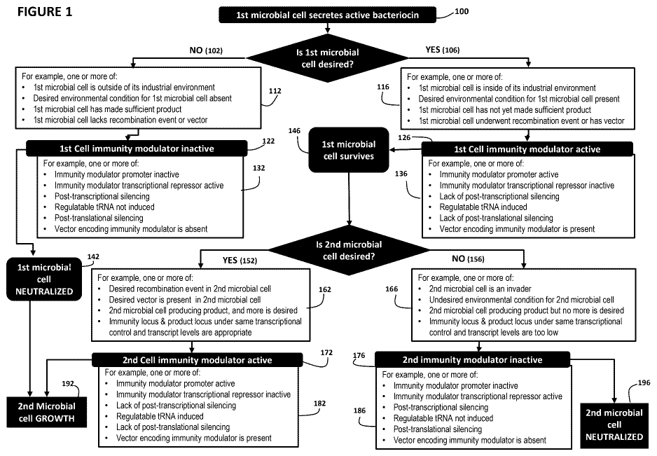

FIG. 1 is a flow diagram depicting options for configuring a microbial cell to control the growth of a second microbial cell according to some embodiments herein. In some embodiments, a first microbial cell is provided. In some embodiments, the first microbial cell secretes an active bacteriocin 100. In some embodiments, the first microbial cell is not desired 102. For example, in some embodiments, one or more of the first microbial cell being outside its industrial environment, a desired environmental conditional for the first microbial cell being absent, the first microbial cell having made sufficient product, or the first microbial cell lacking a recombination event or vector can make the first microbial cell undesirable in a particular environment at a particular time 112. As such, when the first microbial cell is not desired, its immunity modulator (corresponding to the bacteriocin) can be inactive 122. For example, one or more of an immunity modulator promoter can be inactive, an immunity modulator transcriptional repressor can be active, post-transcriptional silencing (e.g. by a ribozyme or antisense) can occur, a regulatable tRNA can not be induced, post-transcriptional silencing can occur (e.g. by a site-specific protease, or a silencing post-translational modification), or a vector encoding an immunity modulator can be absent 132. In some embodiments, when the first cell does not have an active immunity modulator, the first cell is neutralized by the bacteriocin 142 produced by other cells in the culture. In some embodiments, a second microbial cell proceeds with growth 192 as a result of the first cell being neutralized.

In some embodiments, the first microbial cell is desired 106. For example, one or more of the first microbial cell being inside of its industrial environment, a desired environmental condition for the first microbial cell being present, the first microbial cell having not yet made sufficent product yet, or the first microbial cell having undergone a recombination event or comprising a particular vector can make the microbial cell desirable in a particular environment at a particular time 116. As such, when the first microbial cell is desired, it can produce an active immunity modulator 126. For example, in some embodiments, the first microbial cell can be configured to have one or more of a constitutive promoter for the immunity modulator polynucleotide, an activated (but not necessarily constitutive) promoter for the immunity modulator polynucleotide, an inactive repressor of immunity modulator transcription, a regulatable tRNA that is induced to facilitate production of the immunity modulator, an absence of post-translational and post-transcriptional silencing of the immunity modulator, or a vector encoding the immunity modulator can be present 136. As such, the first microbial cell can survive 146 in the presence of bacteriocin secreted by the first microbial cell. As a result of the bacteriocin secreted by the first microbial cell, a second microbial cell can grow 192 or be neutralized 196, depending on whether the second microbial cell has 172 or does not have 176 immunity modulator activity.

In some embodiments, the second microbial cell is desired 152. For example, one or more of a desired recombination event having occurred in the second microbial cell, a desired vector present in the second microbial cell, the second microbial cell producing a product of which more is desired (e.g. a positive feedback loop), or the immunity locus and the desired product being under the same transcriptional control when appropriate levels of desired product are being transcribed can a make the second microbial cell desirable 162. When the second microbial cell is desired, it can provide immunity modulator activity to protect against the particular bacteriocin (or bacterocins) produced by the first microbial cell 172. For example, in some embodiments, the second microbial cell can be configured such that an immunity modulator promoter is active (for example, a constitutive promoter), an immunity modulator transcriptional repressor is inactive, there is a lack of post-transcriptional silencing, a regulatable tRNA being induced to facilitate the expression of the immunity modulator, a lack of post-translational silencing (e.g. by a site-specific protease) of the immunity modulator, or a vector encoding an immunity modulator can be present 182. As such, in some embodiments, when immunity modulator activity is provided, the second microbial cell can survive 192.

In some embodiments, a second microbial cell is not desired 156. For example, one or more of the second microbial cell being an invader (e.g. a contaminating cell), an undesired environmental condition for the second microbial cell (e.g. the presence of an undesired compound or condition, or the absence of a desired compound or condition), the second microbial cell having produced product, but no more product being desired (e.g. a negative feedback loop), or an immunity modulator locus and desired product locus being under the same transcriptional control and transcript levels being undesirably low (e.g. indicating an inability to produce a desired product) can make the second microbial cell undesirable 166. As such, in some embodiments, there can be no immunity modulator activity or an insufficient amount of an immunity modulator to protect against the action of the bacteriocin in the second microbial cell 176. For example, one or more of an immunity modulator promoter can be inactive, an immunity modulator transcriptional repressor can be active, post-transcriptional silencing of the immunity modulator (e.g. by a ribozyme or antisense oligonucleotide) can occur, a regulatable tRNA can not be induced (so that expression of the immunity modulator is not facilitated), post-transcriptional silencing of the immunity modulator can occur (e.g. by a site-specific protease, or a silencing post-translational modification), or a vector encoding an immunity modulator can be absent 186. In some embodiments, the first microbial cell provides secreted bacteriocin activity 100. As such, in some embodiments, the second microbial cell can be killed by the bacteriocin 196.

One skilled in the art will appreciate that, for this and other functions, structures, and processes, disclosed herein, the functions, structures and steps may be implemented or performed in differing order or sequence. Furthermore, the outlined functions and structures are only provided as examples, and some of these functions and structures may be optional, combined into fewer functions and structures, or expanded into additional functions and structures without detracting from the essence of the disclosed embodiments.

For a large variety of genetically modified microbial cells, it can be useful to control the growth of other microbial cells in the culture. In some embodiments, a microbial cell controls the growth of other microbial cells in the culture. Exemplary functions and configurations by which a first microbial cell can control the growth of one or more other microbial cells according to some embodiments herein are described in Table 4.

TABLE 4
Exemplary uses of bacteriocin systems in genetically modified microbial cells
according to some embodiments herein

| | Exemplary configurations (according to some |
|---|---|
| Exemplary Function | embodiments) |
| Biological containment: | Immunity modulator activity only in the desired culture |
| | medium, but not outside and bacteriocin activity at least |
| | outside of the desired culture medium; escape of the |
| | bacteriocin producing cell outside the desired culture |
| | environment results in cytotoxicity or growth inhibition of |
| | the bacteriocin producing cell |
| Genetic guard | Bacteriocin constitutively produced; genetic guard |
| | microbial organism does not produce gene products for |
| | modulating industrial process of interest; immunity |
| | modulator constitutively produced (e.g under control of |
| | constitutive promoter) and/or genetic guard microbial |
| | organism is insensitive to the bacteriocin (e.g. a S. |
| | cerevisiae genetic guard producing bacteriocins that |
| | target E. coli) |
| Selection of recombinants: | Desired recombination event causes an immunity |
| | modulator to be restored in a bacteriocin-expressing host. |
| | Alternatively the immunity modulator can be restored |
| | only after the desired recombination event. |
| Vector stability: | Immunity modulator (or at least one gene essential for |
| | immunity is encoded on a plasmid, and a corresponding |
| | bacteriocin locus is encoded on chromosome); clones that |
| | lose the desired plasmid lack immunity and are |
| | neutralized by the bacteriocin |
| Minimization of genetic drift | Immunity modulator activity dependent on production of |
| | industrial product (e.g. immunity modulator expression |
| | controlled by an operon, in which a repressor is active in |
| | the absence of industrial product, and inactive in the |
| | presence of industrial product); if a mutation causes the |
| | microbial organism’s production of industrial product to |
| | fall below a desired level or cease, the microbial |
| | organism ceases to produce immunity modulator, and is |
| | neutralized by the bacteriocin. |
| Selection for microbes presenting | Immunity modulator is co-expressed with the gene of |
| a high yield of expression | interest; microbial organisms producing high levels of |
| expression (and/or expressing | gene product of interest can be selected by increasing |
| clones) | bacteriocin concentration; microbial organisms producing |
| | low levels of gene product of interest (e.g. having a low |
| | “industrial fitness”) are neutralized |
| Destruction during fermentation | Desired microbial cells constitutively express at least one |
| of contaminating microbes. | type of bacteriocin; secreted bacteriocins neutralize |
| | invading microbial cells |
| | Desired microbial cells express at least one type of |
| | bacteriocin when in the desired environment (e.g. |
| | bacteriocin is under the control of an inducible promoter |
| | that is activated by an intermediate of the fermentation |
| | process); secreted bacteriocins neutralize contaminating |
| | cells |
| Control of the ratio of a | Immunity modulator activity is repressed by accumulated |
| microbial flora. | product made by a microbial cell; bacteriocins secreted by |
| | the microbial cell (or other cells) neutralize the microbial |
| | cell |

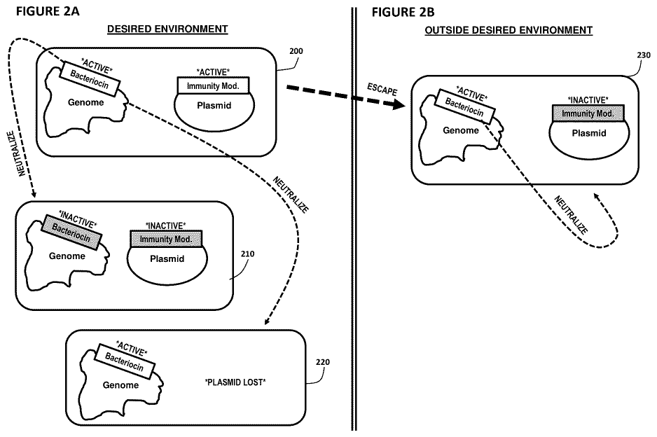

FIG. 2 is a schematic diagram depicting a genetically engineered microbial cell controlling the growth of at least one other microbial cell according to some embodiments herein. A first microbial cell 200 can comprise a bacteriocin polynucleotide and a corresponding immunity modulator polynucleotide. The bacteriocin polynucleotide can optionally be integrated into the cell's genome, while the immunity modulator polynucleotide can optionally be integrated into a plasmid present in the cell. In some embodiments an undesired clone of the cell 210 (a “non-expressing clone”) can lack immunity modulator activity, and optionally can lack bacteriocin activity. The bacteriocin activity of the first microbial cell 200 can neutralize the non-expressing clone 210. In some embodiments, an undesired clone of the cell 220 can lose a plasmid comprising the immunity modulator polynucleotide. The bacteriocin activity of the first microbial cell 200 can neutralize the undesired clone 220. In some embodiments, the microbial cell 230 can escape from the desired environment, causing the clone to lack immunity modulator activity. Bacteriocin activity from the escaped cell 230 and/or clones of the escaped cell can neutralize the escaped cell 230. In some embodiments, the escaped cell 230 further comprises a poison-antidote system to facilitate killing of the escaped cell upon its escape.

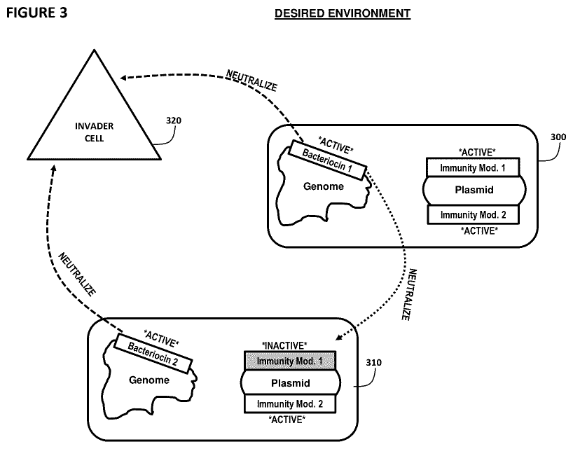

FIG. 3 is a schematic diagram of a first genetically engineered microbial cell 300 controlling the growth of a second genetically engineered microbial cell 310 and an invader cell 320 in a desired environment according to some embodiments herein. The first genetically engineered microbial cell 300 can comprise a first bacteriocin polynucleotide. The second genetically engineered microbial cell 310 can comprise a second bacteriocin polynucleotide. Each of the first and second genetically engineered microbial cells (300 and 310) can comprise a first immunity modulator polynucleotide encoding resistance to the first bacteriocin, and a second immunity modulator polynucleotide encoding resistance to the second bacteriocin. If the second genetically engineered microbial cell 310 becomes undesired, it can lose first immunity modulator activity via any of the mechanisms discussed herein, and thus be controlled by the first bacteriocin activity from the first genetically engineered microbial cell 300. If an invader cell 320 enters the desired environment, the first bacteriocin from the first genetically engineered microbial cell 300 and the second bacteriocin from the second genetically engineered microbial cell 310 can neutralize the invader cell.

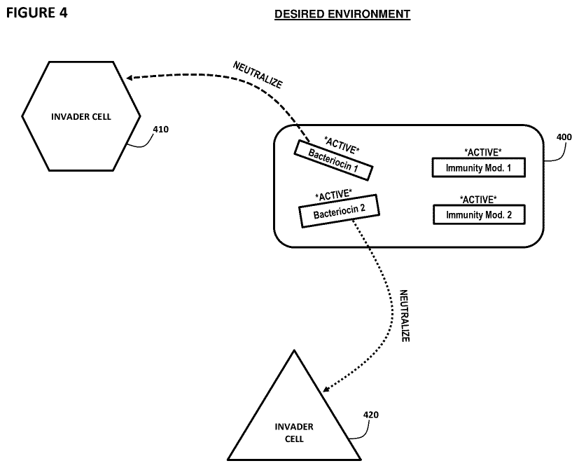

FIG. 4 is a schematic diagram of a first genetically engineered microbial cell 400 controlling the growth of a first invader cell 410 and a second invader cell 420 in a desired environment according to some embodiments herein. The first genetically engineered cell 400 can comprise at least a first bacteriocin polynucleotide encoding a first bacteriocin, and at least a second bacteriocin polynucleotide encoding a second bacteriocin. The first genetically engineered cell 400 can produce the first bacteriocin to neutralize a first invader cell 410. The first genetically engineered cell 410 can produce the second bacteriocin to neutralize a second invader cell 420. In some embodiments, the first invader cell is of a different strain or species from the second invader cell. In some embodiments, the first invader cell responds to a different spectrum of bacteriocin activity than the second invader cell. In some embodiments, the first invader cell typically occupies a different ecological niche than the second invader cell.

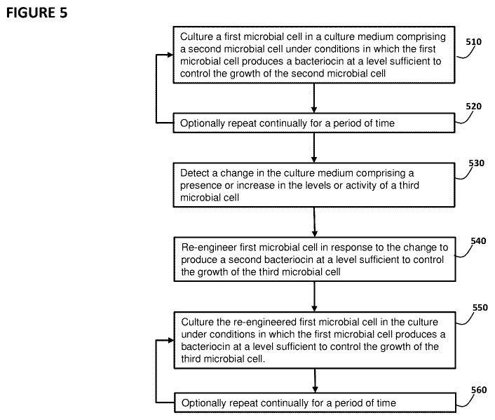

FIG. 5 is a flow diagram illustrating methods of controlling the growth of at least a second microbial cell in culture according to some embodiments herein. The method can comprise culturing a first microbial cell in a culture medium comprising a second microbial cell under conditions in which the first microbial cell produces a bacteriocin at a level sufficient to control the growth of the second microbial cell 510. The culturing of the first microbial cell can optionally be continually maintained for a period of time 520. In some embodiments, the culturing of the first microbial cell is maintained continually for at least 3 days, for example at least 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, 250, 300, 350, 400, 450, or 500 days, including ranges between any two of the listed values. A change in the culture medium comprising a presence or increase in the levels or activity of a third microbial cell can be detected 530. The first microbial cell can be re-engineered in response to the change to produce a second bacteriocin at a level sufficient to control the growth of the third microbial cell 540. The re-engineered first microbial cell can be cultured in the culture under conditions in which the first microbial cell produces a bacteriocin at a level sufficient to control the growth of the third microbial cell 550. The culture of the re-engineered microbial cell can be repeated continually for a period of time 560. In some embodiments, the culturing of the re-engineered microbial cell is maintained continually for at least 3 days, for example at least 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, 250, 300, 350, 400, 450, or 500 days, including ranges between any two of the listed values.

In some embodiments, a first microbial cell can control the growth of a second microbial cell. In some embodiments, a first microbial cell can control the growth of a second microbial cell of the same strain as the first microbial cell. Each cell of the strain can comprise a bacteriocin polynucleotide and an immunity modulator polynucleotide, such that the immunity modulator, if expressed, protects against the bacteriocin. As such, if a clone of the strain loses expression of the immunity modulator, it will be neutralized by bacteriocin activity from the same strain. In some embodiments, the immunity modulator polynucleotide is in cis to the bacteriocin polynucleotide. As such, even if the bacteriocin polynucleotide and immunity modulator polynucleotide are both eliminated (e.g. if a plasmid is lost or a FLP-FRT cassette is excised), bacteriocin activity from other cells can still neutralize the cell. In some embodiments, the immunity modulator polynucleotide is in trans to the bacteriocin polynucleotide. The immunity modulator activity can be lost when the microbial cell is undesired (for example, if a plasmid is lost, or if a particular environmental condition induces a loss of immunity modulator activity). Accordingly, bacteriocin activity from both the microbial cell and also other cells of the strain can induce the neutralizing of the microbial cell.

In some embodiments, a ratio of two or more microbial species or strains is controlled. An exemplary control of ratios is illustrated in FIG. 3 (see cells 300 and 310). In some embodiments, a first microbial strain or species loses an immunity modulator activity via any of the mechanisms discussed herein when it is less desired than a bacteriocin-producing second strain or species, increasing the ratio of second strain or species to the first strain or species. In some embodiments in which the ratio of a first and second strain or species is controlled, a bacteriostatic bacteriocin or bacteriocins are selected (as opposed to bacteriocitic bacteriocins) so that the control of growth can be readily reversible, and/or to minimize the risk of eliminating either of the strains or species. In some embodiments, a first microbial strain or species produces a first bacteriocin under the control of a promoter that is activated in the presence of a compound or substance of interest, for example an intermediate or a product such as an industrially useful molecule. As such, levels of the bacteriocin increase as the levels of the compound of interest increase. In some embodiments, a second microbial strain or species produces (or catalyzes the production of) the compound or substance of interest, but does not have immunity modulator activity for the bacteriocin. As levels of the compound or substance of interest increase, levels of the bacteriocin increase, thus neutralizing the second strain (which lacks an appropriate immunity modulator or which has an insufficient amount of an appropriate immunity modulator to protect against the action of the bacteriocin). As such, relative levels of the first strain compared to the second strain increase. In some embodiments, a first microbial strain produces a first product and first bacteriocin activity, and a second microbial strain produces a second product and second bacteriocin activity. In some embodiments, the first product and the second product are intermediates in the same biosynthetic pathway. The first microbial strain can provide a first and second immunity modulator activity, in which the second immunity modulator activity can protect against the second bacteriocin and is negatively regulated by accumulation of the first product (e.g. expression of the second immunity modulator is repressed by the presence of the first product), and the first immunity modulator activity can protect against the first bacteriocin. The second microbial strain can also provide a first and second immunity modulator activity, except that the first immunity modulator activity is negatively regulated by accumulation of the second product (e.g. expression of the first immunity modulator is repressed by the presence of the second product). As such, when a relatively high amount of the first product has accumulated, the second immunity modulator in the first microbial strain is inactivated, and the microbial cells of the first strain are neutralized by the second bacteriocin, thus increasing the ratio of the second strain to the first strain, and increasing the relative amount of second product to first product. When a relatively high amount of the second product has accumulated, the first immunity modulator in the second microbial strain is inactivated, and the microbial cells of the second strain are neutralized by the first bacteriocin, the increasing the ratio of the first strain to the second strain and increasing the relative amount of first product to second product. As such, the ratio of the first stain to the second strain can be adjusted, depending on relative levels of product. In some embodiments, an equilibrium of ratios of the first strain to the second strain is maintained. In some embodiments, an equilibrium of ratios of the first product to the second product is maintained. In some embodiments, the first microbial strain's second immunity modulator responds to a first environmental condition or compound, and the ratio between the first and second microbial strain is otherwise controlled as above. In some embodiments, the second microbial strain's first immunity modulator responds to a second environmental condition or compound, and the ratio between the first and second microbial strain is otherwise controlled as above.

In some embodiments, it is desired that a microbial cell be contained within a particular environment, for example so that the first microbial cell can only survive in a particular culture medium such as industrial feedstock. In some embodiments, a microbial cell comprises a bacteriocin polynucleotide and an immunity modulator polynucleotide, such that the immunity modulator corresponds to the bacteriocin. In some embodiments, when the microbial cell is in a desired environment, the microbial cell produces an active bacteriocin and corresponding immunity modulator, but when the microbial cell escapes the desired environment, the microbial cell produces the active bacteriocin but no active immunity modulator. As a result, the microbial cell can grow in the desired environment, but is neutralized by its own bacteriocin when it escapes. For example, in some embodiments, the bacteriocin encoded by the bacteriocin polynucleotide is constitutively expressed, while the immunity modulator is expressed only when the microbial cell is in a desired environment. For example, in some embodiments, the bacteriocin encoded by the bacteriocin polynucleotide is constitutively expressed, while the immunity modulator is expressed only when the microbial cell is in an environment. For example, in some embodiments, a transcriptional activator of the immunity modulator is only present in the desired environment. For example, in some embodiments, the bacteriocin encoded by the bacteriocin polynucleotide and the immunity modulator is constitutively expressed, but if the microbial cell escapes, the immunity modulator is deleted (for example via the FLP-FRT system). Without being limited to any particular theory, if a genetic system for neutralizing an escaped microbial cell is not used within the culture itself, there may be little or no selective pressure to maintain the system within the culture, so that mutations can accumulate which reduce or eliminate the functioning of that genetic system. As such, if the microbial cell escapes from the culture, there is a possibility that the genetic system will no longer function. In contrast, it is appreciated herein that if a bacteriocin/immunity modulator system is useful both within a culture (for example, to control the growth of other genetically engineered cells in the culture, and/or to neutralize invading microbial cells), and also outside of a culture (for example, to neutralize a microbial cell that has escaped from culture), the use within the culture can provide selective pressure for the bacteriocin system to continue to function. Such selective pressure in accordance with some embodiments herein can minimize genetic drift. Such selective pressure in accordance with some embodiments herein can help to ensure that if the microbial cell escapes from the desired culture environment, the bacteriocin/immunity modulator system will be functioning to appropriately neutralize the escaped cell. As such, in some embodiments a single genetically engineered circuit, for example a bacteriocin/immunity modulator system is useful both to neutralize other microbial cells within a desired culture environment, and further to neutralize a microbial cell and/or its clones upon escape from a desired culture environment. It is contemplated in accordance with some embodiments herein, any or all of the configuration of bacteriocins disclosed herein can be tuned so that upon escape from the desired culture environment, the escaping microbial organism will be neutralized by its own bacteriocins (and/or bacteriocins of its direct or indirect progeny, and/or bacteriocins of another escaped cell and/or its direct or indirect progeny).

In some embodiments, a microbial cell can control growth in two or more ways. In some embodiments, a microbial cell can perform two or more of the functions described in Table 4. In some embodiments, the microbial cell uses the same bacteriocin/immunity modulator pair for two or more different functions. In some embodiments, the microbial cell uses a first bacteriocin/immunity modulator pair for a first function, and a second bacteriocin/immunity modulator pair for a second function. For example, in some embodiments, a microbial cell can express a bacteriocin which limits the growth of “non-expressing” clones that have lost immunity modulator activity in a desired environment, and can also provide containment within the desired environment by failing to express its own immunity modulator (while still expressing bacteriocin) if the microbial cell is outside of a desired environment. A schematic illustration of such two forms of growth regulation is illustrated in FIG. 2. For example, in some embodiments, a first microbial cell can express a bacteriocin which limits the growth of a second microbial cell, and can also neutralize the invading cell. A schematic illustration of such two forms of growth regulation is illustrated in FIG. 3. In some embodiments, two or more forms of growth control are provided using the same bacteriocin-immunity modulator pair. In some embodiments, each form of growth control is provided using a different bacteriocin immunity modulator pair. For example, a first immunity locus can be present on a plasmid that also includes a polynucleotide encoding a desired product. A clone that loses the plasmid will be neutralized by a corresponding first bacteriocin. A second immunity modulator polynucleotide (corresponding to a second immunity modulator) can be integrated into the genome of the microbial cell and can be silenced when the microbial cell escapes from its desired environment (for example, the second immunity modulator polypeptide can be in a FLP-FRT cassette that is excised upon escape). As such, upon escape, the microbial cell can be neutralized by the second bacteriocin.

It is noted that some embodiments described herein are compatible with poison-antidote systems. As such, in some embodiments a microbial cell, in addition to a bacteriocin and immunity modulator further comprises a poison-antidote system configured to kill or arrest the cell when it is not in a desired environment.

It can be useful to control the growth of two or more different types of microbial cells. For example, an environment can comprise, or can potentially comprise, two or more different types of undesired microbial organisms. As different microbial organisms can have different susceptibility to bacteriocins (for example, by possessing different profiles of immunity modulators), a combination of two or more bacteriocins (e.g. a “cocktail” of bacteriocins) can be useful for controlling the growth of two or more microbial organisms. In some embodiments, a single microbial cell produces two or more different bacteriocins for example, at least 2, 3, 4, 5, 6, 7, 8, 9, 10, 11, 12, 13, 14, 15, 16, 17, 18, 19, 20, 25, 30, 35, 40, 45, or 50 different bacteriocins, including ranges between any two of the listed values. In some embodiments, a mixture of two or more different bacteriocin-producing microbial cells are provided, for example, 2, 3, 4, 5, 6, 7, 8, 9, 10, 11, 12, 13, 14, 15, 16, 17, 18, 19, 20, 25, 30, 35, 40, 45, or 50 different bacteriocin-producing microbial cells, including ranges between any two of the listed values. Optionally, one or more of the bacteriocin-producing microbial cells can produce two or more different bacteriocins.

It can be useful for a single microbial cell to regulate the growth of two or more different types of microbial cells. For example, it can be possible for a first type of invading cell to possess immunity to a first type of bacteriocin but not a second type of bacteriocin. As such, in some embodiments, a microbial cell comprises two or more bacteriocin polynucleotides, each of which encodes a different bacteriocin (see, e.g. FIG. 4). In some embodiments, the microbial cell comprises polynucleotides encoding at least three different bacteriocins, for example at least three, four five, six, seven, eight, nine, ten, eleven, twelve, thirteen, fourteen, fifteen, sixteen, seventeen, eighteen, nineteen, twenty, or more different bacteriocins, including ranges between any two of the listed values. In some embodiments, two or more bacteriocin polynucleotides are under control of a single promoter. In some embodiments, each bacteriocin polynucleotide under the control of a single promoter comprises its own translational initiation site. In some embodiments, each bacteriocin polynucleotide is under the control of a different promoter. In some embodiments, two different bacteriocins are under the control of two different but structurally or functionally identical promoters.

It can be useful for a microbial cell to control the growth of other microbial cells in its industrial environment, so as to help ensure the consistent production of an industrial product, regardless of the geographical location of the culture environment. Without being limited by any particular theory, certain industrial products manufactured via microbial culture may have certain characteristics that result from local microbial flora associated with a certain region (for example, Camembert cheese can have particular characteristics that result from local microbial flora in Camembert, France, or sourdough bread can have particular characteristics that result from local microbial flora in San Francisco, CA). As such, it can be desirable to control the microbial flora in a particular feedstock, so that a consistent industrial product can be produced in a variety of geographical locations. In some embodiments, a microbial cell is engineered to produce bacteriocins to neutralize invading microbial cells found in a variety of geographical locations, which can ensure more consistent industrial product characteristics for product produced in a variety of locations. For example, a microbial cell designed to be used in a particular industrial process and to be grown in a first geographic location may be engineered to express one or more bacteriocins effective against one or more invading organisms commonly encountered in the first geographic location. A microbial cell designed to be used in the same industrial process and to be grown in a second geographic location may be engineered to express one or more bacteriocins effective against one or more invading organisms commonly encountered in the second geographic location. Alternatively, a microbial cell designed to be used in a particular industrial process and to be grown in two different geographical locations may be engineered to express on or more bacteriocins effective against one or more invading organisms commonly encountered in each of the two geographical locations.

Frequently in industrial biotechnology, the goal is to work in continuous process, and it is contemplated that the longer the process continues, the higher the probability of contamination. Accordingly, the capacity to fight against contaminants can be useful for a continuous industrial process. Synthetic microorganisms designed in laboratories are frequently used in industrial processes. As such, it can be useful for these lab-engineered “champions” to fight against undesired invading microbial strains (for example wild-type strains from the environment and/or cross-contaminants from another industrial process) and also control their potential genetic drift and escape in the environment. In accordance with some embodiments herein, invading microbial strains can be fought, genetic drift can be minimized, and escape can be minimized by inducing suicidal bacteriocins based genetic circuits.

It can be useful for a microbial culture to remain stable for a continuous period of time, for example to ensure consistent industrial product characteristics over a continuous period of time. In some embodiments, a culture is stably maintained, at least in part, by bacteriocin-mediated neutralization of invading microbial cells. In some embodiments, a culture is stably maintained, at least in part, by bacteriocin-mediated control of ratios of two or more types of genetically engineered microbial cell in the culture. In some embodiments, a culture is stably maintained, at least in part, by reengineering a microbial cell already present in the culture. In some embodiments, the microbial cell is reengineered to add at least one additional bacteriocin activity (for example by adding a new bacteriocin, or expanding the expression of a bacteriocin already present) to neutralize a new type of invading microbial organism. In some embodiments, the microbial cell is reengineered to remove at least one bacteriocin activity that is no longer needed. Exemplary methods of maintaining a stable culture according to some embodiments herein are illustrated in FIG. 5. In some embodiments, a stable culture is maintained for at least about 3 days, for example about 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, 250, 300, 350, 400, 450, or 500 days, including ranges between any two of the listed values.

### Method for Detection of Ratios of Microbial Organisms

According to some embodiments herein, the ratios of two or more microbial strains or species can be controlled, depending on relative quantities of product, and/or compounds in the environment. Accordingly, in some embodiments, the ratios of the two or more microbial strains or species can be indicative of relative quantities of the product and/or compounds in the environment. In some embodiments, relative quantities of microbes of a first strain or species and second strain or species as described herein are detected, thereby indicating relative ratios or quantities of a first product or compound to a second product or compound. Relative quantities of each microbial strain or species can be detected in a variety of ways. In some embodiments, each strain or species comprises a unique oligonucleotide or polypeptide “bar code” sequence to facilitate specific detection. In some embodiments, each strain or species comprises a different bacteriocin (and thus a different bacteriocin polynucleotide), which can serve as a bar code. In some embodiments, at least one of quantitative PCR, oligonucleotide array analysis, flow cytometry, immunocytochemistry, in situ hybridization, ELISA, immunoblotting, oligonucleotide spot blotting, or the like is performed to determine relative quantities of the two different microbial strains or species.

### Method for Determining Modulation of Growth of Microbial Organisms in Industrial Medium

In some embodiments, growth of microbial organisms in industrial medium is modulated. Before adding a particular genetically engineered microbial cell or combination of genetically engineered cells to an existing industrial culture of microbial cells, it can be useful to determine the effects, if any, of the bacteriocins on the growth of the microbial cells in the existing industrial culture. In some embodiments, the effect of a particular bacteriocin or combination of bacteriocins produced by genetically engineered cells on microbial organisms is assessed. A medium or other composition comprising one or more bacteriocins produced by genetically engineered microbial cells as described herein can be provided. In some embodiments, the medium comprises a supernatant comprising one or more bacteriocins. In some embodiments, the composition comprises one or more enriched or purified bacteriocins. In some embodiments, the supernatant or composition is thermally stable, for example to facilitate elimination of any microbes therein through high-temperature incubation, while retaining the function of any bacteriocins therein. In some embodiments, the medium or composition comprises a lyophilized material comprising bacteriocins. In some embodiments, the medium or composition comprises a substrate bound to bacteriocins, for example a gel, a matrix, or beads. The medium or compositions comprising bacteriocins can be added to the existing culture. In some embodiments, the medium or composition is added to a culture in an industrial culture environment. In some embodiments, the medium or composition is contacted with a sample of a culture from an industrial culture environment. The growth or absence of growth of microbial organisms in the industrial culture can be assessed for example to determine whether the one or more bacteriocins are effective against a new invading organism which has appeared in the culture or to determine the effects of the one or more bacteriocins on the existing organisms in the culture.

Before a genetically engineered microbial cell is produced, it can be useful to simulate the effects of one or more bacteriocins on a particular culture environment. In some embodiments, a particular bacteriocin or combination of bacteriocins with desired activity in a known culture environment is identified, and a microbial cell is constructed to produce the desired bacteriocin combination of bacteriocins. In some embodiments, a candidate bacteriocin or combination of bacteriocins is contacted with a portion of an industrial culture of interest, and effects of the bacteriocin or bacteriocins on microbial organisms in the culture are identified. In some embodiments, a variety of bacteriocins is provided. In some embodiments, the variety of bacteriocins is provided in a kit. In some embodiments, the bacteriocins were produced by microbial cells. In some embodiments, the bacteriocins are in supernatant from one or more microbial cells as described herein. In some embodiments, the bacteriocins were chemically synthesized. One or more candidate bacteriocins or mixtures of bacteriocins can be prepared, and can be contacted with a portion of the industrial culture environment. In some embodiments, one or more bacteriocins are added to the supernatant of a bacteriocin-producing genetically engineered cell that is already present in culture, for example to ascertain the effects of engineering the cell to produce at least one additional bacteriocin. In some embodiments, a sample from the industrial culture environment is contacted with each candidate bacteriocin or mixture of bacteriocins. In some embodiments, each candidate bacteriocin or mixture of bacteriocins is added to the culture environment. In some embodiments, effects of each candidate bacteriocin or mixture of bacteriocins are observed, for example as effects on the growth of at least one desired microbial cell in the culture, and/or the growth of at least one undesired microbial cell in the culture.

Upon identification of a desired combination of bacteriocins, a microbial cell can be constructed to produce the desired combination of bacteriocins. In some embodiments, an existing microbial cell, for example a microbial cell that is producing a desired product or intermediate in industrial culture is reengineered to produce the desired combination of bacteriocins. In some embodiments, the microbial cell is reengineered via plasmid conjugation. In some embodiments, a new cell is engineered to produce the desired combination of bacteriocins and added to the industrial culture.

### Genetic Guard Microbial Organisms and Systems

It can be useful for a bacteriocin-producing microbial organism to protect other microbial organisms from undesired microbial organisms. Accordingly, in some embodiments, a “genetic guard microbial organism” is provided (which, as a shorthand, may also be referred to herein as a “genetic guard”). As used herein, a “genetic guard” refers to a microbial organism or collection of microbial organisms that produces one or more bacteriocins so as to protect a “protected” microbial organism that is immune to neutralizing effects of the bacteriocins, but does not itself produce the bacteriocins. The “protected” microbial organism can perform a desired industrial process (for example, fermentation), while, as used herein, the “genetic guard” itself does not perform the desired industrial process. The genetic guard microbial organsism can express and secrete one or more bacteriocins. Optionally, the genetic guard microbial organsism can constititvely express and secrete one or more of the bacteriocins. The genetic guard microbial organism can be non-susceptible to the bacteriocins produced by the genetic guard, for example by producing immunity modulator(s) to the bacteriocin(s) secreted by the genetic guard, and/or by being a type of microbial organism that is not susceptible to the to the bacteriocin(s) produced by the genetic guard (e.g. if the genetic guard comprises a yeast and secretes bacteriocins that specifically neutralize particular bacteria such as lactic acid bacteria). In some embodiments, the protected microbial organism produces immunity modulator(s) to the bacteriocin(s) produced by the genetic guard. In some embodiments, the protected microbial organism is not susceptible to the bacteriocins produced by the genetic guard (e.g. if the protected microbial organism comprises a yeast, and the genetic guard microbial organism produces bacteriocins that specifically neutralize particular bacteria). In some embodiments, the protected microbial organism is not genetically modified (“non-GMO”). In some embodiments, the protected microbial organism is non-GMO, but is from a strain selected to have desired properties, for example via selective pressure, and/or classical mutagenesis. It is contemplated that even if the protected microbial organism has desirable industrial properties, the protected microbial organism may be insufficient at fighting-off one or more undesired microbial organisms, for example invading local flora. Accordingly, in some embodiments herein, a genetic guard protects a protected microbial organism from undesired microbial organisms. By way of example, non-GMO microbial organisms can be useful in a number of processes, for example food production, or purification such as water purification. In some embodiments, non-GMO “protected” microbial organisms are selected based on their ability to destroy one or more contaminants (for example, known water contaminants), and a genetic guard is provided to protect the protected microbial organisms from known or potential invading undesired microbial organisms. In some embodiments, systems comprising a genetic guard as described herein are provided.

It can be useful to maintain a culture medium that does not contain genetically modified organisms, for example to perform particular industrial processes, and/or to comply with certain production standards or specifications. It is contemplated that in accordance with some embodiments herein, genetic guards can be separated from the “protected” microbial organism by a membrane that is permeable to bacteriocins, but not to the genetic guard microbial organisms. As such, bacteriocins produced by the genetic guard can enter a culture medium occupied by the protected microbial organisms, thus protecting the protected organisms from one or more undesired microbial organisms while the genetic guard remains separated from the microbial organism.

It is contemplated herein that a particular culture medium can be invaded by and/or subject to a variety of undesired microbial organisms, which may susceptible to different bacteriocins or combinations of bacteriocins. Accordingly, in some embodiments, the genetic guard microbial organism produces two or more different bacteriocins, for example, 2, 3, 4, 5, 6, 7, 8, 9, 10, 11, 12, 13, 14, 15, 16, 17, 18, 19, 20, 2, 30, 35, 40, 45, 50, 55, 60, 65, 70, 75, 80, 85, 90, 95, or 100 different bacteriocins, including ranges between any two of the listed values, for example 2 to 100, 2 to 50, 2 to 20, 2 to 10, 5 to 100, 5 to 50, 5 to 20, 5 to 10, 10 to 100, 10 to 50, 10 to 20, 20 to 100, 20 to 50, or 50 to 100 different bacteriocins. By way of example, in some embodiments, the genetic guard comprises a single E. coli strains, which produces 20 different bacteriocins. In some embodiments, the genetic guard produces a cocktail of bacteriocins. In some embodiments, the genetic guard comprises a mixture of two or more different bacteriocin-producing microbial organisms, for example, 2, 3, 4, 5, 6, 7, 8, 9, 10, 11, 12, 13, 14, 15, 16, 17, 18, 19, 20, 2, 30, 35, 40, 45, or 50 different bacteriocin-producing microbial organisms, so as to provide a desired combination of bacteriocins. By way of example, in some embodiments, the genetic guard comprises a combination of 4 different E. coli strains, each of which produces 5 different bacteriocins (for a total of 20 different bacteriocins). In some embodiments, the genetic guard produces a cocktail of bacteriocins that target a particular category of microbial organism, for example lactic acid bacteria.

It can be useful for the genetic guard to be separated from a particular environment or culture medium, for example to maintain an industrial culture environment or feedstock free of genetically modified organisms (GMOs). In some embodiments, the genetic guard is physically separated from the protected microbial organism. Optionally, the protected microbial organism is non-GMO. In some embodiments, the genetic guard is temporally separated from the protected microbial organism. Optionally, the protected microbial organism is non-GMO. For example, temporal separation in accordance with some embodiments can comprise adding the genetic guard to a culture medium to neutralize invading organisms, and subsequently adding the protected microbial organism to the culture medium. Optionally, the genetic guard can be neutralized prior to adding the protected microbial organism, for example via bacteriocins or a poison-antidote system as described herein. Optionally, the genetic guard can be neutralized by their own bacteriocins, for example by repressing expression of the corresponding immunity modulator or immunity modulators in the genetic guard. For example, temporal separation in accordance with some embodiments can comprise culturing the protected microbial organism in a culture medium, and subsequently adding the genetic guard to the culture medium.

In some embodiments, the genetic guard is positioned in a first environment, and the protected microbial organism or organisms are positioned in a second environment. The first environment can be separated from a second environment by a membrane permeable to bacteriocins produced by the genetic guard but not the genetic guard itself. In some embodiments, the membrane is not permeable to the protected microbial organism. In some embodiments, the first environment is in fluid communication with the second environment. Without being limited by any theory it is contemplated that as bacteriocins typically comprise diffusible stable peptide molecules, the bacteriocins can readily move in aqueous solution from the first environment to the second environment. In some embodiments, the first environment comprises a first chamber, tank, or pond and the second environment comprises a second chamber, tank, or pond. In some embodiments, the second environment comprises an open-air environment. Optionally, an industrial process, for example fermentation, is taking place in the second environment. In some embodiments, the first environment comprises a capsule positioned inside of the second environment. A variety of membranes are suitable for arrangements and systems in accordance with embodiments herein, so long as the membranes are permeable to bacteriocins, but not to genetic guards. In some embodiments, the membrane comprises at least one of a mesh, strainer, filter, selective valve, unidirectional valve, or porous membrane. In some embodiments, the membrane comprises one or more pores having a diameter smaller than the diameter of the genetic guard. In some embodiments, the bacteriocins diffuse through the membrane. In some embodiments, fluidic motion from the first environment to the second environment drives the movement of the bacteriocins. In some embodiments, the genetic guard is selected based on known or likely undesired microbial organisms in the culture medium. In some embodiments, the genetic guard is changed after a period of time. For example, in response to changes in the invading undesired microbial organisms, the genetic guard can be adjusted so that additional bacteriocins are added, and/or some bacteriocins are removed.

In some embodiments, an existing microbially-mediated industrial process is performed in a new location, which is characterized by one or more potential undesired microbial organisms. As the microbial organisms of the existing industrial process may not produce bacteriocins against some or all of the undesired microbial organisms of the new location, a genetic guard producing bacteriocins targeting the undesired microbial organisms can be added to the culture medium in the new location. As such, the bacteriocins of the genetic guard can neutralize one or more undesired microbial organisms, if present in the culture medium.

In some embodiments, the genetic guard produces a cocktail of bacteriocins. The cocktail of bacteriocins can be collected while the genetic guard is not, and the cocktail of bacteriocins can be contacted with a culture medium of interest. As such, separation can be maintained between the culture medium and the genetic guard. The skilled artisan will appreciate that a number of methods are suitable for separating the bacteriocins from the genetic guard, so long as the methods do not substantially damage, denature, or destroy the bacteriocins. In some embodiments, the cocktail of bacteriocins is collected by filtering out the genetic guard. In some embodiments, the cocktail of bacteriocins is collected by centrifuging to separate the genetic guard from the bacteriocins. In some embodiments, the cocktail of bacteriocins is collected by neutralizing the genetic guard. In some embodiments, the cocktail is stored prior to contact with the culture medium.

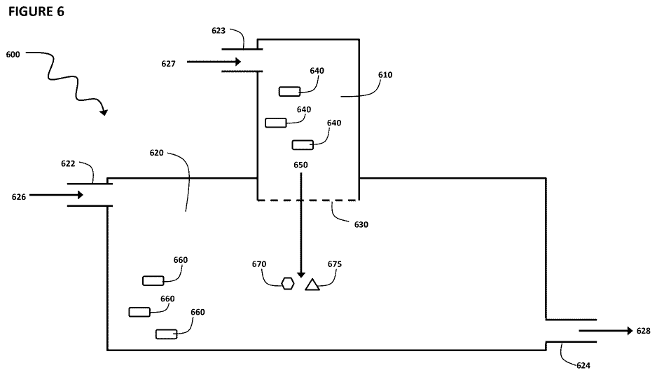

FIG. 6 is a schematic diagram illustrating a system 600 comprising a genetic guard in accordance with some embodiments herein. The system 600 can comprise a first environment 610 and a second environment 620. Optionally, the second environment 620 can comprise an inlet 622 and/or an outlet 624. A fluid or culture medium to be treated, for example polluted water or feedstock can enter 626 via the inlet 622, and exit 628 via the outlet. The first environment 610 can be separated from the second environment 620 by a membrane 630 that is permeable to bacteriocins, but is not permeable to genetic guard microbial organisms 640. The first environment 610 can comprise genetic guard microbial organisms 640, which produce bacteriocins that can move 650 between the first environment 610 and the second environment 620. The second environment 620 can comprise protected microbial organisms 660, which are not susceptible to the neutralizing effects of the bacteriocins produced by the genetic guard 640. Optionally, the protected microbial organisms 660 can be non-GMO. However, if undesired microbial organisms 670, 675 are present, the undesired microbial organisms 670, 675 can be neutralized by the bacteriocins. In some embodiments, the system 600 comprises a treatment system for polluted water. In some embodiments, the system comprises a second inlet 623 so that fluid to be treated enters 627 the first environment 610 before entering the second environment 620. Optionally, the system can comprise the second inlet 623 but not the first inlet 622. Optionally, the system can comprise the second inlet 623 and the first inlet 622. As such, the genetic guard microbial organisms 640 can secrete bacteriocins to neutralize invading undesired organisms 670, 675, while maintaining physical separation between the genetic guard microbial organisms 640 and protected microbial organisms 660.

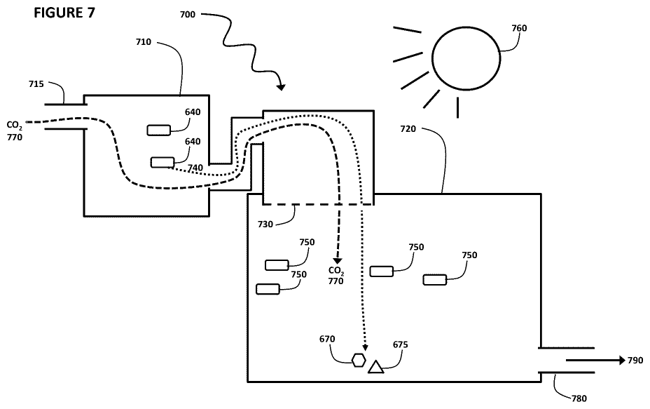

FIG. 7 is a schematic diagram illustrating a genetic guard system 700 that can be useful for photosynthetic production in accordance with some embodiments herein. The system 700 can comprise a first environment 710. Optionally, the first environment 710 can comprise an inlet 715. The first environment 710 and optional inlet 715 can be in fluid and gas communication with a second environment 720. The first environment 710 can be separated from the second environment 720 by a membrane 730 that is permeable to bacteriocins and gas, but is not permeable to genetic guard microbial organisms 640. The first environment 710 can comprise genetic guard microbial organisms 640, which produce bacteriocins 740 that can move between the first environment 710 and the second environment 720. The second environment can comprise photosynthetic microbial organisms 750, for example photosynthetic microalgae. Optionally, the photosynthetic microbial organisms 750 are non-GMO. A source of light 760 can be in optical communication with the second environment 720. It is contemplated that the source of light 760 can comprise sunlight and/or artificial light. CO₂ 770 can enter the second environment 720, and can be used in combination with light from the light source 760 for photosynthetic production by the photosynthetic microbial organisms 750. Optionally the CO₂ 770 can enter the inlet 715 of the first environment 710, and enter the second environment 720 through the membrane 730. Bacteriocins 740 produced by the genetic guard microbial organisms 740 can enter the second environment 720 through the membrane 730, and can neutralize undesired microbial organisms 780, 785 in the second environment. Optionally, the second environment can comprise an outlet 780, and biomass 790 produced by the photosynthetic microbial organism 760 can exit the second environment 720 via the outlet 790. As such, the genetic guard microbial organisms 640 can secrete bacteriocins to neutralize invading undesired organisms 670, 675, while maintaining physical separation between the genetic guard microbial organisms 640 and photosynthetic microbial organisms 750 and biomass 790.

Preservation and/or Storage of Feedstock

It can be useful to store a feedstock without performing an industrial process in the feedstock, for example to build up a reserve in case additional output is needed later on, to decrease output for the time being, and/or to transport the feedstock to a different location. For example, a feedstock for feeding animals can be harvested in the summer, and stored until winter, when it is used to feed animals. For example, a feedstock may undergo an initial round of fermentation to produce a desired component in the feedstock, or to destroy or remove a desired component in the feedstock, and/or to stabilize the feedstock for storage, and the feedstock may then be preserved until it is to be consumed.

It is contemplated herein that undesired microbial organisms can contaminate a feedstock during storage, and/or consume or destroy one or more components of the feedstock. For example, microbial organisms can be selected or engineered to produce glucose from cellulose in a feedstock. However, in a feedstock comprising glucose, undesired microbial organisms can catabolize the glucose. Accordingly, in some embodiments, a genetic guard is added to a feedstock so as to protect the feedstock from one or more undesired microbial organisms during storage. In some embodiments, the feedstock undergoes an initial round of processing (e.g. fermentation) to produce, remove, or destroy at least one component (for example to stabilize the feedstock for storage and/or to provide a desired component in the feedstock such as glucose from cellulose), and the genetic guard then protects the feedstock from subsequent undesired microbial organisms. In some embodiments, the genetic guard is physically separated from the feedstock by a bacteriocin-permeable membrane during fermentation and/or during storage. It is contemplated that bacteriocin-mediated neutralization of undesired microbial organisms in a feedstock in accordance with some embodiments herein can permit a feedstock to be stored stably for long periods of time. In some embodiments, the feedstock is stably stored for at least one month, for example, at least one month, 2, 3, 4, 5, 6, 7, 8, 9, 10, 11, 12, 13, 14, 15, 16, 17, 18, 19, 20, 21, 22, 23, or 24 months.

In some embodiments, the genetic guard is contacted with the feedstock. In some embodiments, the genetic guard is already present in the feedstock, and proliferation of the genetic guard is induced prior to or during storage so that the genetic guard produces bacteriocins to neutralize undesired microbial organisms in the feedstock.

### Methods of Preparing and Using Bacteriocin-Producing Microbial Organisms:

In accordance with some embodiments herein, bacteriocin-producing microbial organisms can be prepared for use in an industrial process which is subject to, or at risk of contamination or interference by undesired microbial organisms. In some embodiments, a circuit for desired production of bacteriocins is designed, nucleic acid sequences are engineered, and the circuit is assembled and introduced to a host microbial organism.

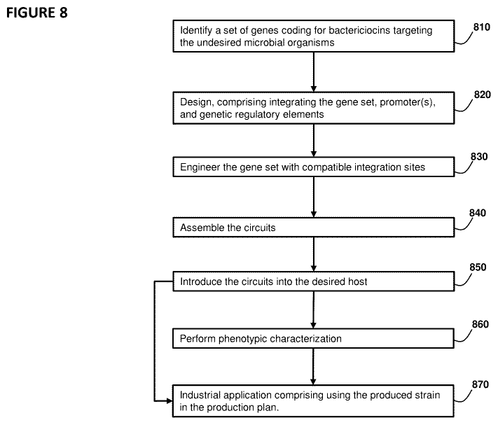

FIG. 8 is a flow diagram illustrating methods of preparing and using bacteriocin. The method can comprise identifying a set of genes coding for bacteriocins targeting the undesired microbial organisms 810. An approach for identifying genes in accordance with some embodiments herein comprises identifying bacteriocin genes using an electronic database, for example bactibase, accessible on the world wide web at bactibase.pfba-lab-tun.org/main.php. The method can comprise designing a construct for expressing a bacteriocin, comprising integrating the gene set, promoter(s), and genetic regulatory elements 820. As such, a construct can be designed. Approaches for designing an appropriate construct in accordance with some embodiments herein can comprise using parts databases, for example electronic databases such as the Biobricks foundation parts database. It is contemplated herein that in accordance with some embodiments, the skilled artisan can selected desired components (including, but not limited to bacteriocin nucleotides, promoters, and genetic regulatory elements) based on their identified functions, and engineer a construct with a desired functionality based upon the identified functionality of these components. By way of example, functionalities of different possible components can be found in one or more databases, such as the Biobricks catalog. A catalog of Biobricks components is accessible on the world wide web at parts.igem.org. The method can comprise engineering the gene set with compatible integration sites 830, which can allow the genes to be assembled in a desired manner and/or appropriately introduced to a desired host. A variety of suitable integration sites can be used, for example restriction sites, substrates for an enzymatic recombination reaction, or sequences for homologous recombination. In some embodiments, the gene set is synthesized. In some embodiments, a nucleic acid comprising the gene set is synthesized. In some embodiments, the gene set is provided in one or more vectors such as plasmids. The method can comprise assembling the circuits 840. The circuits can include one or more bacteriocin nucleic acids, and a suitable promoter(s) and regulatory element(s). A variety of configurations of circuits can be suitable. In some embodiments, a single promoter drives expression of multiple bacteriocins and optional gene products of interest. In some embodiments, different bacteriocin nucleic acids are under the control of different promoters. In some embodiments, a circuit is comprised in a single construct such as a plasmid. In some embodiments, a circuit is comprised in two or more constructs such as plasmids. In some embodiments, a nucleic acid comprising the complete circuit is synthesized. In some embodiments, the circuit is assembled using conventional molecular cloning techniques, or a combination of nucleic acid synthesis and molecular cloning. Molecular cloning techniques are well known to the skilled artisan. Many suitable molecular cloning techniques are described in Green and Sambrook “Molecular Cloning: A Laboratory Manual” (2012) Cold Spring Harbor Laboratory Press; 4th edition, which is hereby incorporated by reference in its entirety. The method can comprise introducing the circuits into the desired host 850. Suitable hosts include, but are not limited to, naturally occurring, genetically engineered, and fully synthetic microbial organisms, including, but not limited to the exemplary microbial organisms described herein. Optionally, the method includes performing phenotypic characterization 860, for example strain behavior. For example, it can be useful to select for desired transformants or recombinants, confirm that a strain is producing the desired bacteriocins, and/or confirm that a regulatory circuit is responsive to an appropriate stimulus such as industrial precursor or product. The method can comprise industrial application comprising using the produced strain in the production plan 870. For example, a bacteriocin-producing strain can be introduced to an existing culture medium, or can be used as a starter culture for a new culture medium.

### Kits

Kits are provided according to some embodiments herein. In some embodiments, the kits contain at least one of bacteriocins, bacteriocin polynucleotides, immunity modulators, immunity modulator polynucleotides, other genetic elements (such as promoters, expression vectors, conjugation plasmids, and the like), genetically engineered microbial cells, and/or culture medium as described herein. In some embodiments, the kits further contain packaging, and/or instructions for use of the contents therein. In some embodiments, the kits comprise a variety of bacteriocins, for example for use in ascertaining the effects of a candidate bacteriocin or combination thereof on a culture environment. In some embodiments, the kits comprise a variety of bacteriocin polynucleotides and immunity modulator polynucleotides, for example for constructing a microbial cell with desired characteristics. In some embodiments, the kits comprise a variety of donor microbial cells that comprise donor plasmids encoding a variety of combinations of at least one bacteriocin and/or at least one immunity modulator.

### Example 1: Protection of Cyanobacteria and Neutralization Upon Escape

A cyanobacterium comprising a biosynthetic pathway for a lipid is provided. The cyanobacterium has been genetically engineered to comprise a bacteriocin polynucleotide under the control of a first promoter that is constitutively active. The cyanobacterium comprises an immunity modulator polynucleotide for an immunity modulator that protects against the bacteriocin, and that is under the control of a second promoter that is only active in the presence of a precursor found in an industrially useful feedstock. The cyanobacterium is placed in the feedstock. While it is producing lipids in the feedstock, the cyanobacterium also secretes active bacteriocin, thus neutralizing invading microorganisms. Upon escape from the feedstock, the cyanobacterium no longer possesses immunity modulator activity, but still produces bacteriocin, and thus is neutralized by the bacteriocin.

### Example 2: Protection of Bacillus, Maintenance of a Plasmid, and Neutralization Upon Escape

A genetically engineered Bacillus cell is provided, comprising a bacteriocin polynucleotide integrated into its chromosomal genome, and a plasmid comprising an immunity modulator polynucleotide for an immunity modulator that protects against the bacteriocin as well as a polynucleotide encoding a polypeptide to be manufactured. The bacteriocin is under the control of a constitutive promoter. The immunity modulator polynucleotide is under the control of a promoter that is only active in the presence of a precursor found in the industrially useful feedstock. As such, when the Bacillus is in the feedstock, it produces the bacteriocin to kill invading microbial cells. Moreover, when Bacillus clones lose the plasmid, they become undesirable (as they no longer can produce the polypeptide to be manufactured), and as a result of also losing the immunity modulator, are killed by the bacteriocin. Upon escape from the feedstock, the Bacillus cell no longer possesses immunity modulator activity, but still produces bacteriocin, and thus is neutralized by the bacteriocin produced by the other genetically engineered Bacillus cells in its environment.

### Example 3: Regulation of Levels of Two Partner Strains of S. cerevisiae

A first S. cerevisiae strain is provided. The first strain comprises a bacteriocin polynucleotide under the control of a first promoter that is induced by the presence of a metabolite. As such, the bacteriocin is expressed more strongly as levels of the metabolite increase. The encoded bacteriocin arrests the S. cerevisiae cell cycle, but is bacteriostatic, not bacteriolytic. The first strain also comprises an immunity modulator polynucleotide for conferring immunity to the first bacteriocin under control of a promoter that is activated by a compound present only in the industrial feedstock. A second, partner strain of S. cerevisiae comprises a polynucleotide encoding an enzyme that produces the metabolite, but does not comprise a corresponding immunity modulator activity. As levels of the metabolite increase through activity of the second strain, the first strain produces more and more bacteriocin, thus arresting the cell cycle of the second strain, and reducing the relative amount of cells of the second strain available. Meanwhile, the first strain continues to proliferate. Accordingly, the relative ratio of the first strain to the second strain is increased, and buildup of the metabolite is reduced.

### Example 4: Regulation of A. ferrooxidans by E. coli

An Acidithiobacillus ferrooxidans strain is engineered to produce stored energy from the oxidation of Fe(II) to Fe(III) in a feedstock comprising an iron source that diffuses Fe(II) into the feedstock. An E. coli strain is engineered to control the growth of the first strain of A. ferrooxidans. The A. ferroxidans strain comprises a nucleic acid encoding Colicin-Ia (SEQ ID NO: 56) under the control of a rus operon promoter (SEQ ID NO: 549), and a nucleic acid encoding a Colicin-Ia immunity modulator (SEQ ID NO: 464) under the control of a constitutive promoter (B. subtilis ctc promoter, SEQ ID NO: 663). However, the ferroxidans strain does not produce any Colicin-E1 immunity modulator. The E. coli strain comprises a nucleic acid encoding Colicin-E1 (SEQ ID NO: 54) and Colicin-E1 immunity modulator (SEQ ID NO: 465) under the control of a constitutive promoter (SEQ ID NO: 651) integrated into its genome. However, the E. coli strain does not produce Colicin-Ia immunity modulator (SEQ ID NO: 464). As the A. ferroxidans oxidizes Fe(II) to Fe(III), levels of Fe(II) decrease. As such, activity of the rus promoter decreases, and the A. ferroxidans produces lower levels of Colicin-Ia (SEQ ID NO: 54). Accordingly, any neutralization of the E. coli strain is minimized. The second strain of E. coli proliferates, producing higher levels of Colicin-E1 (SEQ ID NO: 54). The Colicin-E1 neutralizes the A. ferroxidans, so that less A. ferroxidans is present to oxidize Fe(II) into Fe(III). Accordingly levels of Fe(II) increase again. As Fe(II) accumulates, the A. ferroxidans produce higher levels of Colicin-Ia (SEQ ID NO: 56), neutralizing organisms the second strain of E. coli. Accordingly, there in minimal E. coli producing Colicin-E1, and neutralization of A. ferroxidans is minimal as well. The A. ferroxidans proliferates, oxidizing the Fe(II) into Fe(III) and storing energy.

### Example 5: Genetic Guard for Ethanol Synthesis by Non-GMO Microbial Organism

A genetic guard in accordance with some embodiments herein is used to protect a non-GMO microbial organism that produces ethanol from glucose in a feedstock. The genetic guard comprises an E. coli strain comprising and expressing 20 different bacteriocin nucleic acids under the control of a single constitutive promoter, and as such, produces 20 different bacteriocins in approximately stoichiometric ratios. It is also contemplated that in accordance with some embodiments herein, another suitable option is to provide a genetic guard comprising five different E. coli strains, each of which comprise and express five different bacteriocins. The genetic guard is disposed in the first environment 610 of a system as illustrated in FIG. 6. The bacteriocins diffuse through a porous membrane to enter the second environment. The porous membrane is made of porous polytetrafluoroethylene that is permeable to bacteriocins and liquid, but is not permeable to the genetic guard. Non-GMO fermenting S. cerevisiae are cultured in the second environment. The non-GMO fermenting S. cerevisiae produce ethanol from glucose in the feedstock. The bacteriocins from the genetic guard neutralize invading microbial organisms, preventing contamination of the feedstock and consumption of the ethanol by invading microbial organisms. The porous membrane maintains physical separation between the genetically-engineered genetic guard and non-GMO fermenting yeast. As such, the fermenting yeast is protected from undesired microbial organisms, while a portion of the feedstock is keep free of GMO's.

### Example 6: Protection of Non-GMO Photosynthetic Microalgae by Genetic Guard

A genetic guard in accordance with some embodiments herein is used to protect a non-GMO photosynthetic microalgae that produces biomass. The biomass can be suitable for a variety of downstream applications, for example extracting compounds of interest, energy, or animal feed. The genetic guard comprises a mixture of 50 different B. subtilis strains, each of which produces a different bacteriocin. The genetic guard is disposed in an aqueous first environment 710 of a system as illustrated in FIG. 7. The system further comprises an aqueous second environment 720, which contains non-GMO photosynthetic microalgae, which yield biomass. The first environment is separated from the second environment by a 0.5 μm fiberglass filter, so as to allow gas, liquid, and bacteriocins to pass between the first environment and second environment, while blocking bacteriocins from passing between the first environment and second environment. CO₂ enters the system through an inlet in the first environment, and diffuses through the first environment and second environment. Sunlight enters the second environment, and drives the photosynthetic microalgae to produce biomass. As a result, a high-glucose biomass is produced in the second environment. The 50 different bacteriocins also diffuse from the first environment to the second environment. The bacteriocins neutralize invading undesired microbial organisms, thus preventing contamination the biomass and preventing undesired microbial organisms from interfering with biomass production and/or catabolizing the biomass. Biomass is harvested from the second environment via an outlet. As such, physical separation is maintained between genetically engineered genetic guard and non-GMO photosynthetic microalgae, while neutralizing invading microorganisms in the second environment.

### Example 7: Protection of Saccharomyces cerevisiae Against Lactic Acid Bacteria Family (LAB)

A Saccharomyces cerevisiae is engineered to produce multiple bacteriocins active on Lactic Acid Bacteria (LAB). Leucococin C (SEQ ID NO: 368) and Diversin V41 (SEQ ID NO: 74) are shown to be active on LAB bacteria according to the bactibase database, which is accessible on the world wide web at bactibase.pfba-lab-tun.org/main.php. It is appreciated that as S. cerevisiae are not sensitive to Leucococin or Diversin V41, there is no need to integrate corresponding immunity loci into the S. cerevisiae. As such, Leucococin C (SEQ ID NO: 368) and Diversin V41 (SEQ ID NO: 74) are selected, and polynucleotides are encoding Leucococin C (SEQ ID NO: 369) and Diversin V41 (SEQ ID NO: 75) are provided. The polynucleotides encode Leucococin C (SEQ ID NO: 368) and Diversin V41 (SEQ ID NO: 74), each fused to signal peptide from yeast mating factor alpha to facilitate secretion by the S. cerevisiae. The polynucleotides are integrated into the genome of a single S. cerevisiae strain under the control of a strong constitutive promoter, PPGK1 (3-Phosphoglyceratekinase) (SEQ ID NO: 692). The transformation is performed using standard homologous recombination. It is contemplated herein that other suitable strong constitutive promoters include, but are not limited to PTEF1 (translation elongation factor) and PGAP (glycerinaldehyde-3-phosphate dehydrogenase) (a list of constitutive yeast promoters is accessible on the world wide web at parts.igem.org/Promoters/Catalog/Yeast/Constitutive). The bacteriocin activity expressed by the transformed S. cerevisiae is measured by inhibitory assays on LAB cultures invading the production plan. As the makeup of undesired microbial organisms invading the feedstock changes over time, S. cerevisiae strains producing additional, fewer, and/or different bacteriocins can be produced and introduced into the industrial feedstock.

What is claimed is:

1. A first microbial cell comprising a nucleic acid encoding a secreted bacteriocin which controls the growth of a second microbial cell and a nucleic acid which confers resistance to said secreted bacteriocin, wherein said first microbial cell has been genetically engineered to allow the expression or activity of said nucleic acid which confers resistance to said bacteriocin to be regulated.

2. The first microbial cell of claim 1, wherein the expression or activity of said nucleic acid which confers resistance to said bacteriocin is reduced to a level which causes said first microbial cell to be neutralized by said bacteriocin if said first microbial cell is released from a desired growth environment.

3. The first microbial cell of claim 1, wherein said first microbial cell has been genetically engineered to make a desired product.

4. The first microbial cell of claim 3, wherein said secreted bacteriocin further has been selected to maintain at least one condition within a culture in which said first microbial cell is producing said desired product.

5. The first microbial cell of claim 4, wherein the culture comprises at least one invading microbial organism.

6. The first microbial cell of claim 4, wherein the at least one condition within a culture comprises controlling the growth of the second microbial cell, wherein the second microbial cell is a common contaminate of the culture.

7. The first microbial cell of claim 1, wherein said second microbial cell is a different strain, species or genus than said first microbial cell.

8. The first microbial cell of claim 1, further comprising a nucleic acid encoding a second secreted bacteriocin which controls the growth of a third microbial cell and a nucleic acid which confers resistance to said secreted second bacteriocin, wherein said first microbial cell has been genetically engineered to allow the expression or activity of said nucleic acid which confers resistance to said bacteriocin to be regulated.

9. The first microbial cell of claim 1, wherein said bacteriocin kills said second microbial cell.

10. The first microbial cell of claim 1, wherein said bacteriocin reduces the growth rate of said second microbial cell.

11. The first microbial cell of claim 1, wherein said nucleic acid encoding said bacteriocin is on a chromosome of said microbial cell.

12. The first microbial cell of claim 1, wherein said nucleic acid conferring resistance to said bacteriocin is on a plasmid.

13. The first microbial cell of claim 1, wherein said first microbial cell is selected from the group consisting of bacteria, yeast, and algae.

14. A culture comprising the first microbial cell of claim 1, wherein said culture further comprises the second microbial cell, wherein the presence of the second microbial cell is desired, and wherein the bacteriocin controls the growth rate of the second cell.

15. A method of controlling the growth of a second microbial cell in a culture medium, the method comprising:
culturing a first microbial cell in a culture medium comprising said second microbial cell under conditions in which said first microbial cell produces a bacteriocin at a level sufficient to control the growth of said second microbial cell wherein said first microbial cell comprises a nucleic acid encoding a secreted bacteriocin which controls the growth of said second microbial cell and a nucleic acid which confers resistance to said secreted bacteriocin, and wherein said first microbial cell has been genetically engineered to allow the expression or activity of said nucleic acid which confers resistance to said bacteriocin to be regulated.

16. The method of claim 15, wherein the culture is maintained continually for at least 30 days.

17. The method of claim 15, further comprising:
detecting at least one change in the culture medium, the change comprising a presence or increase in the levels or activity of a third microbial cell; and
reengineering the first microbial cell in response to the change to produce a second bacteriocin at a level sufficient to control the growth of the third microbial cell.

18. A system for neutralizing undesired microbial organisms in a culture medium, the system comprising:
a first environment comprising a culture medium; and
a second environment comprising a second microbial organism that secretes two or more different bacteriocins,
wherein the second microbial organism comprises immunity modulators for each of the two or more different bacteriocins,
wherein the second environment is in fluid communication with the first environment,
wherein the second environment is physically separated from the first environment so that the second microbial organism cannot move from the second environment to the first environment, and
wherein the secreted two or more different bacteriocins enter the culture medium of the first environment.

19. The system of claim 18, further comprising a first microbial organism in the culture medium,
wherein the first microbial organism does not secrete the two or more different bacteriocins, and
wherein the first microbial organism is not neutralized by any of the two or more different bacteriocins.

20. The system of claim 18, wherein the second environment is separated from the first environment by at least one of a membrane, a mesh, a filter, or a valve that is permeable to the two or more different bacteriocins, but is not permeable to the second microbial organisms.
